(19) United States
(12) Patent Application Publication
(10) Pub. No.: US 2024/0365736 A1
(43) Pub. Date: Nov. 7, 2024

(54) COMPOSITIONS AND METHODS FOR TARGETED DEGRADATION OF PROTEINS IN A PLANT CELL

(71) Applicant: Oerth Bio LLC, Durham, NC (US)

(72) Inventors: Jason D. SPEAKE, Mebane, NC (US); Denis FOURCHES, Durham, NC (US); John G. CATALANO, Durham, NC (US); Robert N. HUNTER, III, Garner, NC (US); Christopher KALLUS, Leverkusen (DE); Anne PFEIFFER, Leverkusen (DE); Sophie LAMBERTZ, Leverkusen (DE); Michael BERLIN, New Haven, CT (US)

(73) Assignee: Oerth Bio LLC, Durham, NC (US)

(21) Appl. No.: 18/688,519

(22) PCT Filed: Aug. 31, 2022
(86) PCT No.: PCT/US2022/042198

§ 371 Date: Mar. 1, 2024

Related U.S. Application Data
(60) Provisional application No. 63/244,035, filed on Sep. 14, 2021.
(60) Provisional application No. 63/239,863, filed on Sep. 1, 2021.

(51) Int. Cl. A01H 3/04; A01N 43/40; A01N 43/60; A01N 43/78; A01N 43/84; A01N 43/90

(52) U.S. Cl. CPC A01H 3/04; A01N 43/40; A01N 43/60; A01N 43/78; A01N 43/84; A01N 43/90

(57) ABSTRACT

Compounds, compositions, and methods for controlling the level of a target protein in a cell are described. Compounds of the disclosure include those having according to the formula PTM-L-LTM, wherein PTM is a targeting moiety that binds the target protein, L is a covalent bond or linker moiety, and LTM is a ubiquitin ligase binding moiety that binds a plant ubiquitin ligase. Additionally novel cereblon binding moieties are provided that may be used as molecular glues or in bifunctional compounds to target proteins in plants and mammals.

### CROSS-REFERENCE TO RELATED APPLICATIONS

The present application claims priority to U.S. Provisional Application No. 63/239,863, filed Sep. 1, 2021, and U.S. Provisional Application No. 63/244,035, filed Sep. 14, 2021, the full disclosures of which are incorporated by reference in their entirety for all purposes.

### TECHNOLOGICAL FIELD

The present invention relates to compounds, compositions, and methods for modulating the levels of target proteins in plants and plant cells.

### BACKGROUND

The ubiquitin-proteasome system (UPS) is a major pathway for intracellular protein degradation in eukaryotic cells and provides a mechanism through which the activity of specific proteins can be temporally downregulated. In this system, enzymes known as E1, E2, and E3 promote the post-translational modification of protein substrates with a polyubiquitin chain, which serves as the signal for proteasome-mediated proteolysis. The E3 ubiquitin ligases act at the end of the three-enzyme cascade and play a critical role in both recognizing specific protein targets and facilitating ubiquitin transfer from the E2 enzyme to the substrate.

Cereblon is a substrate adaptor module of E3 ubiquitin ligase. It has no inherent enzymatic activity, but rather controls the substrate specificity of E3-mediated protein ubiquitin modifications. Cereblon forms an E3 complex with damaged DNA-binding protein 1 (DDB1), Cullin 4 (CUL4) and regulator of cullins 1 (ROC1), abbreviated here as CRL4CRBN. Under normal conditions CRL4CRBN recognizes endogenous substrates including glutamine synthetase, MEIS2, and amyloid precursor protein. Exogenous drugs that bind to cereblon are capable of allosterically modifying CRL4CRBN substrate specificity. In particular, the immunomodulatory thalidomide family drugs are known to alter E3 substrate selectivity. Binding of thalidomide to cereblon mediates this drug's teratogenicity, via disruption of fibroblast growth factor 8 expression and limb bud outgrowth. After the discovery that thalidomide causes teratogenicity in the mid-1960s, the compound and related structures were notwithstanding found to be useful as anti-inflammatory, anti-angiogenic, and anti-cancer agents (see Bartlett et al. (2004) The Evolution of Thalidomide and Its Imid Derivatives as Anticancer Agents, Nat. Rev. Cancer 4, 314-322).

Specific protein function has been studied using protein silencing methods. The two main approaches currently used to suppress protein expression include gene knockout by CRISPR/Cas9 and gene knockdown by RNA interference (RNAi). Most silencing systems suffer from limitations. Gene editing by CRISPR/Cas9 acts at the DNA level leading to a complete and irreversible depletion of the protein of interest. RNA interference (RNAi) induces reversible transcript degradation and, in turn, protein reduction. One disadvantage of these approaches is that both CRISPR/Cas9 and RNAi can generate off-target effects. RNAi has been widely used to achieve protein downregulation at the mRNA level, however a disadvantage of RNAi is that, when RNAi is induced, all protein products already translated remain unaffected. Targeting at the protein level allows overcoming the limitations of gene inactivation and loss of function phenotypes with essential genes. While targeted protein degradation has been performed and studied in mammalian systems, it has not been exploited in plants.

### BRIEF SUMMARY

The present invention provides efficient and effective systems in plants for the targeted degradation or inhibition of proteins. Compositions and methods for modulating the level of a protein of interest in a plant cell are provided. Compositions include molecular glues and bifunctional compounds that target a plant protein of interest to a cellular pathway. In some instances, the pathway leads to degradation of the plant protein. The bifunctional compounds are selected from Proteolysis Targeting Chimeras (PROTAC® compounds), phosphatase recruiting chimeras (PhoRCs), deubiquitinase-targeting chimeras (DUBTAC), ribonuclease targeting chimeras (RIBOTACs), autophagy-targeting chimeras (AUTACs), autophagosome-tethering compounds (ATTECs), and lysosome-targeting chimeras (LYTACs).

In some embodiments, the methods utilize bifunctional compounds, such as PROTAC® compounds, to direct degradation of a protein of interest in a plant. PROTAC® compounds as provided herein are a class of bifunctional molecules that utilize the endogenous protein homeostasis machinery of plants to degrade or inhibit a protein of interest (POI). PROTAC® compounds induce proximity between a plant ligase and a POI in the form of a ternary complex that results in the ubiquitination and subsequent degradation of the POI by the proteasome. Thus, the bifunctional PROTAC® compounds comprise a binding ligand for a ubiquitin ligase and a second binding ligand for a POI (i.e. the target protein) to mediate ubiquitin transfer to, and degradation of, the protein of interest through the plant proteasome. The first binding ligand is also referred to as a ligase targeting moiety (LTM) while the second binding ligand is referred to as a protein targeting moiety (PTM). It is recognized that when using the term moiety, the moiety itself, a derivative of the moiety, or an analog of the moiety are intended and can be used.

Provided herein are bifunctional compounds which function to recruit endogenous proteins to a plant ubiquitin ligase for degradation, and methods of using the same. In particular, the present disclosure provides bifunctional or proteolysis targeting chimeric (PROTAC®) compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of activities is possible, consistent with the degradation/inhibition of targeted polypeptides from virtually any protein class or family. The PROTAC® compounds provided herein are designed as heterobifunctional molecules consisting of three basic components: a ligand that recruits a plant E3 ligase, a ligand that binds to the POI, and a suitable linker to connect these two binding ligands. The structure of the PROTAC® compounds enables simultaneous recruitment of the ligase and the POI, forming a ternary complex that provides for sufficient proximity between the ligase and the POI to allow for the transfer of a first ubiquitin molecule from the E2 enzyme to an accessible lysine residue on the POI, followed by rapid transfer of additional ubiquitin units leading to degradation of the POI. Following degradation, the PROTAC® compound is recycled for further ternary complex formation. Thus, smaller amounts of the PROTAC® compounds are needed for administration to plants. In some embodiments, the bifunctional compounds provided herein can include more than one ligase binding moiety, more than one plant targeting moiety, and/or more than one linker.

The PROTAC® compounds provided herein may also function as chemical biology probes providing an alternative to genetic modification strategies such as RNA interference or clustered regularly interspaced short palindromic repeats (CRISPR) for protein knockdown or knockout. In this manner they may be used to study protein function, signaling pathways, cellular activities and the like.

The bifunctional molecules or PROTAC® compounds may be provided in compositions comprising suitable agricultural carriers for delivery to plants. Compositions may further include plants, plant cells, and plant parts comprising the bifunctional compounds provided herein. In some embodiments, compositions comprise plants, plant cells, and plant parts comprising at least one bifunctional compound as described herein.

In one embodiment, the invention encompasses cereblon binding ligands as well as bifunctional compounds comprising cereblon binding ligands. Thus, compositions of the invention further comprise numerous cereblon binding moieties, numerous protein binding moieties, multiple chemical linkers, and combinations thereof. The cereblon binders described in this disclosure may be used in bifunctional compounds as described herein to target proteins of interest. In other embodiments the cereblon binding ligands may be used as molecular glues without the need for a separate protein targeting moiety. Also provided are agricultural compositions comprising at least one compound as described herein and at least one agriculturally acceptable carrier. It is recognized that the cereblon binders of the invention will bind to plant cereblon ligase as well as to cereblon in other organisms, including mammals. Thus, the cereblon binding moieties may be used in bifunctional molecules to target and modulate the activity of proteins in other organisms, including mammals.

Also provided herein are methods of modulating protein ubiquitination and degradation or inhibition of a target protein in a plant, plant cell, and plant tissue. The method comprises administering an effective amount of a compound as described herein or a composition comprising an effective amount of the compound to a plant, wherein the compound or composition is effective in modulating protein ubiquitination and degradation of the protein in the plant.

In some embodiments, compounds according to the present disclosure comprising novel cereblon binders are provided in pharmaceutical compositions for controlling the amount or activity of a target protein in a mammal or other organism.

### DETAILED DESCRIPTION

Provided herein are bifunctional compounds that are useful for modulating the levels of a protein of interest in a plant or plant cell. In some embodiments, the bifunctional compounds are PROTAC® compounds, and methods comprise their use in targeting and modulating the levels of proteins of interest (POI) in a plant cell. The PROTAC® compounds function to recruit endogenous proteins to a plant ubiquitin ligase enzyme for ubiquitination and subsequent degradation or inhibition. The compounds may be designed to target any protein of interest present in a plant cell. In some embodiments, the compounds are provided in agricultural compositions.

Additionally, novel cereblon ligase binders are provided. As cereblon orthologs are highly conserved from plants to humans (only two mutations K⇔E and D⇔H among the nine key binding residues in the cereblon binding site between the Arabidopsis thaliana (plant) and human proteins), bifunctional molecules comprising the cereblon binders may be used in plants or other organisms, such as mammals, to target any protein expressed in a cell of interest for inhibition or degradation. These cereblon binders may be used in bifunctional compounds or as molecular glues to modulate targeted ubiquitination of a variety of polypeptides and other proteins, which are then degraded and/or otherwise inhibited. Polypeptides from any protein class or family may be targeted for degradation/inhibition.

### PROTAC® Compounds and Other Binding Compounds

Compositions of the invention include bifunctional compounds that target a protein of interest for degradation and/or inhibition. Additionally, the novel cereblon binders disclosed herein may be used as components for the bifunctional compounds or as molecular glues. In one embodiment, the compositions and methods of the invention utilize a proteolysis targeting chimera (PROTAC®) compound. A PROTAC® compound is a bifunctional compound having a binding ligand that binds a protein in a plant cell, referred to herein as a protein targeting moiety (PTM), covalently linked via a linker (L) to a binding ligand that binds to a ubiquitin ligase, referred to herein as a ligase targeting moiety (LTM). In some embodiments, the PROTAC® compound is a compound according to Formula I:

PTM-L-LTM (I)

The bound protein is targeted for degradation through the ubiquitin-proteasome system. PROTAC® compounds may be used to degrade essentially any protein of interest in a plant (e.g., target pathogenic proteins), regulate signaling pathways, and modulate phenotypes. In designing the PROTAC® compounds, multiple factors will typically be considered, including the type of ligase being targeted, design of the PROTAC® linker component, the choice of the POI ligand and the binding site, and the nature of the protein-protein interaction interface between the ligase and the POI. These factors may affect the structure and stability of ternary complexes.

As discussed, in one embodiment, cereblon binding ligands are utilized in the bifunctional molecules. Where the LTM binds cereblon, the bifunctional molecules may be used to degrade or inhibit proteins of interest in plant, mammalian, or other organisms.

It will be understood that the general structures provided herein are exemplary and the respective moieties may be arranged spatially in any desired order or configuration. Compounds may comprise a plurality of ligase binding moieties and/or a plurality of the plant POI binding moieties. When using the term moiety or compound, the moiety or compound itself, a derivative of the moiety or compound, or an analog of the moiety or compound are intended and may be used in the practice of the invention.

### Plant Ubiquitin Ligases and Ligase Targeting Moieties (LTMs)

Many ubiquitin ligases are known and may be targeted by the PROTAC® compounds provided herein. E3 ubiquitin ligases are classified into two groups depending on the presence of a HECT (Homology to E6-AP C-Terminus) or a RING (Really Interesting New Gene)/U-box domain. RING proteins may act as single components containing both the active site and the binding pocket for the E2-ubiquitin intermediate, or as components of multisubunit complexes which in plants include SCF (SKP1-CULLIN-F-box), CUL3 (CULLIN 3)-BTB/POZ (Bric a brac, Tramtrack and Broad complex/Pox virus and Zinc finger), CUL4-DDB1 (UV-Damaged DNA-Binding Protein 1) and APC (Anaphase Promoting Complex) complexes. While RING/U-box E3s generally act as molecular adapters between E2 and target proteins, the HECT E3s form a covalent bond with ubiquitin before transferring it to the protein substrate, using a conserved Cys of the HECT domain to form a ubiquitin-E3 thiole-ester intermediate.

Multi-subunit E3 ligases include CULLIN and RING finger-based protein complexes. These enzymes are constituted by two functional modules. The catalytic module comprises a RING-finger subunit (RBX1—RING Box protein 1—or APC11) interacting with the E2 enzyme. The second module (adapter) specifically recognizes target substrates for ubiquitination. The same catalytic module may be associated to many different adapters, therefore many different E3 complexes may be formed, which in turn will catalyze the ubiquitination of different substrates. The two modules are brought together by a CULLIN (or CULLIN-like, APC2) protein which acts as a molecular scaffold and also defines the E3 class. The E3 SCF complexes contain four core components: SKP1 (S-phase Kinase associated Protein 1, named ASK1, for Arabidopsis Skp1-related in plants), CUL1, an F-box protein and RBX1. See, Mazzucotelli et al. (2006) Curr Genomics 7(8): 509-522; and Chen, L. and Hellmann, H. (2013) Molecular Plant 6(5): 1388-1404; each of which are herein incorporated by reference.

Computational approaches may be used to design ligands for plant ligases. Such structures may then be optimized through rational medicinal chemistry optimization. See, for example, Ishida, T. and Ciulli, A. (2021) SLAS Discovery 26(4) 484-502. Binding assays are available for identifying ligase binders including those set forth in Franklin and Pruneda (2019) Front. Chem. 7: 816 available on the World Wide Web at doi.org/10.3389/fchem.2019.00816.

Known ubiquitin ligase binders may also be used in the compounds and methods provided herein. Such binders include, but are not limited to, those disclosed in U.S. Pat. Nos. 9,694,084; 9,938,264; 10,239,888; 10,450,310; 10,464,925; 10,584,101; 10,723,717; 10,730,870; 10,772962; U.S. Application Publication Nos: 2015/0291562; 2015/0119435; 2016/0058872; 2017/0008904; 2018/0147202; 2019/0127359; and PCT Publication Nos: WO2000/047220; WO2018/144649; and WO2021/127278. All of these are herein incorporated by reference in their entirety.

Cereblon is a substrate adaptor module of E3 ubiquitin ligase. It has no inherent enzymatic activity, but rather controls the substrate specificity of E3-mediated protein ubiquitin modifications. Cereblon is the substrate adapter for the Cullin 4a ligase complex whose endogenous substrates have not been fully characterized. The immunomodulatory drug thalidomide and its analogs have been shown to bind to cereblon and induce degradation. Cereblon orthologs are highly conserved from plants to humans, which underscores its physiological importance.

In some embodiments, the plant ubiquitin ligase is cereblon, and the ligase targeting moiety is an N-substituted 1,3-dioxoisoindolinyl moiety, which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

In some embodiments, the ligase targeting moiety is a cereblon targeting moiety containing an N-substituted 1-oxoisoindolinyl moiety a N-substituted 1,3-dioxoisoindolinyl moiety. In some embodiments, provided are compounds according to Formula II

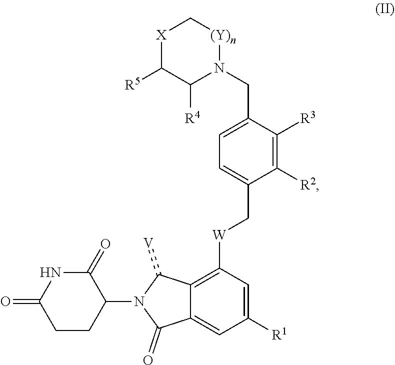

as well as salts and solvates thereof, wherein:
subscript n is an integer ranging from 0 to 3;
V is absent or (O);
R¹ is selected from the group consisting of H and —Z—R^{1a}.
W, X, Y, and Z are independently selected from the group consisting of O, NR⁶, and CHR⁷, provided that Y is CHR⁷ when subscript n is 0 or 1;
R^{1a} is selected from the group consisting of -L-PTM, H, and —(CH₂—CH₂—O)ₘ—CH₃;
R² and R³ are independently selected from the group consisting of -L-PTM, H, —NH—(CH₂—CH₂—O)ₘ—CH₃, and —CONH—(CH₂—CH₂—O)ₘ—CH₃;
R⁴ is selected from the group consisting of -L-PTM, H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ and R⁷ are independently selected from the group consisting of -L-PTM, H, and —CONH—(CH₂—CH₂—O)ₘ—CH₃;
R⁶ is selected from the group consisting of -L-PTM, H, C₁₋₆ acyl, —CH₂—O—(CH₂—CH₂—O)ₘ—CH₃, provided that R⁶ is other than H when X is N;
each subscript m is independently an integer ranging from 1 to 10;
each L is an independently-selected linker moiety; and
each PTM is an independently-selected protein targeting moiety.

In some embodiments, at least one of R¹-R⁷ is other than H. In some embodiments, one and only one of R¹ and R²-R⁷ is -L-PTM. In some embodiments, for example:
R^{1a} is -L-PTM;
R² and R³ are independently selected from the group consisting of H, —NH—(CH₂—CH₂—O)ₘ—CH₃, and —CONH—(CH₂—CH₂—O)ₘ—CH₃;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ and R⁷ are independently selected from the group consisting of H, and —CONH—(CH₂—CH₂—O)ₘ—CH₃; and
R⁶ is selected from the group consisting of H, C₁₋₆ acyl, —CH₂—O—(CH₂—CH₂—O)ₘ—CH₃, provided that R⁶ is other than H when X is N. In some such embodiments, each subscript m is independently 1 or 2.

In some embodiments:
R^{1a} is selected from the group consisting of H and —(CH₂—CH₂—O)ₘ—CH₃;
R² is L-PTM;
R³ is selected from the group consisting of H, —NH—(CH₂—CH₂—O)ₘ—CH₃, and —CONH—(CH₂—CH₂—O)ₘ—CH₃;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ and R⁷ are independently selected from the group consisting of H and —CONH—(CH₂—CH₂—O)ₘ—CH₃; and
R⁶ is selected from the group consisting of H, C₁₋₆ acyl, and —CH₂—O—(CH₂—CH₂—O)ₘ—CH₃, provided that R⁶ is other than H when X is N. In some such embodiments, each subscript m is independently 1 or 2.

In some embodiments:
R^{1a} is selected from the group consisting of H, and —(CH₂—CH₂—O)ₘ—CH₃;
R² is selected from the group consisting of H, —NH—(CH₂—CH₂—O)ₘ—CH₃, and —CONH—(CH₂—CH₂—O)ₘ—CH₃;
R³ is L-PTM;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ and R⁷ are independently selected from the group consisting of H and —CONH—(CH₂—CH₂—O)ₘ—CH₃; and
R⁶ is selected from the group consisting of H, C₁₋₆ acyl, and —CH₂—O—(CH₂—CH₂—O)ₘ—CH₃, provided that R⁶ is other than H when X is N. In some such embodiments, each subscript m is independently 1 or 2.

In some embodiments:
R^{1a} is selected from the group consisting of H, and —(CH₂—CH₂—O)ₘ—CH₃;
R² and R³ are independently selected from the group consisting of H, —NH—(CH₂—CH₂—O)ₘ—CH₃, and —CONH—(CH₂—CH₂—O)ₘ—CH₃;
R⁴ is -L-PTM;
R⁵ and R⁷ are independently selected from the group consisting of H, and —CONH—(CH₂—CH₂—O)ₘ—CH₃; and
R⁶ is selected from the group consisting of H, C₁₋₆ acyl, and —CH₂—O—(CH₂—CH₂—O)ₘ—CH₃, provided that R⁶ is other than H when X is N. In some such embodiments, each subscript m is independently 1 or 2.

In some embodiments:
R^{1a} is selected from the group consisting of H, and —(CH₂—CH₂—O)ₘ—CH₃;
R² and R³ are independently selected from the group consisting of H, —NH—(CH₂—CH₂—O)ₘ—CH₃, and —CONH—(CH₂—CH₂—O)ₘ—CH₃;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ is -L-PTM;
R⁶ is selected from the group consisting of H, C₁₋₆ acyl, and —CH₂—O—(CH₂—CH₂—O)ₘ—CH₃, provided that R⁶ is other than H when X is N; and
R⁷ is selected from the group consisting of H and —CONH—(CH₂—CH₂—O)ₘ—CH₃. In some such embodiments, each subscript m is independently 1 or 2.

In some embodiments:
R^{1a} is selected from the group consisting of H and —(CH₂—CH₂—O)ₘ—CH₃;
R² and R³ are independently selected from the group consisting of H, —NH—(CH₂—CH₂—O)ₘ—CH₃, and —CONH—(CH₂—CH₂—O)ₘ—CH₃;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ and R⁷ are independently selected from the group consisting of H and —CONH—(CH₂—CH₂—O)ₘ—CH₃; and
R⁶ is -L-PTM. In some such embodiments, each subscript m is independently 1 or 2.

In some embodiments:
R^{1a} is selected from the group consisting of H, and —(CH₂—CH₂—O)ₘ—CH₃;
R² and R³ are independently selected from the group consisting of H, —NH—(CH₂—CH₂—O)ₘ—CH₃, and —CONH—(CH₂—CH₂—O)ₘ—CH₃;
R⁴ is selected from the group consisting of H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃;
R⁵ is selected from the group consisting of H and —CONH—(CH₂—CH₂—O)ₘ—CH₃;
R⁶ is selected from the group consisting of H, C₁₋₆ acyl, and —CH₂—O—(CH₂—CH₂—O)ₘ—CH₃, provided that R⁶ is other than H when X is N; and
R⁷ is -L-PTM. In some such embodiments, each subscript m is independently 1 or 2.

In some embodiments, V is absent (i.e., the ligase targeting moiety is an N-substituted 1-oxoisoindolinyl moiety).

In some embodiments, W is O. In some embodiments, W is NR⁶. In some embodiments, W is CHR⁷.

In some embodiments, R¹ is H. In some embodiments, R¹ is —Z—R^{1a}.

In some embodiments, R² and R³ are H. In some embodiments, R² is H and R³ is selected from the group consisting of -L-PTM, —NH—(CH₂—CH₂—O)ₘ—CH₃, and —CONH—(CH₂—CH₂—O)ₘ—CH₃. In some embodiments, R² is selected from the group consisting of -L-PTM, —NH—(CH₂—CH₂—O)ₘ—CH₃, and —CONH—(CH₂—CH₂—O)ₘ—CH₃, and R³ is H. In some such embodiments, each subscript m is independently 1 or 2.

In some embodiments, X is O (e.g., as a member of of an oxazolidine ring, a morpholine ring, a 1,4-oxazepane ring, or a 1,4-oxazocane ring). In some embodiments, X is O, subscript n is 1, Y is —CHR⁷, and R⁷ is H. In some such embodiments, R⁵ is H. In some embodiments, R⁵ is H and R⁴ is H. In some embodiments, R⁵ is H and R⁴ is selected from the group consisting of -L-PTM, —CH₂—O—(CH₂—CH₂—O)ₘ—CH₃, —CH₂NH—(CH₂—CH₂—O)ₘ—CH₃, and —CONH—(CH₂—CH₂—O)ₘ—CH. In some such embodiments, each subscript m is independently 1 or 2.

In some embodiments, X is —CHR⁷ (e.g., as a member of a pyrrolidine ring, a piperidine ring, an azepane ring, or azocane ring). In some embodiments, X is —CHR⁷, subscript n is 0 or 1, Y is —CHR⁷, and R⁷ is H. In some such embodiments, R⁴ is H. In some embodiments, R⁴ is H and R⁵ is H. In some embodiments, R⁴ is H and R⁵ is selected from the group consisting of -L-PTM, and —CONH—(CH₂—CH₂—O)ₘ—CH₃. In some such embodiments, each subscript m is independently 1 or 2.

The oxoisoindolinyl and dioxoisoindolinyl ligase targeting moieties of the present disclosure are characterized by advantageously high binding affinity for E3 ubiquitin ligases such as plant cereblons. Binding affinity may be expressed as a dissociation constant K_{d} and may be assessed using techniques such as microscale thermophoresis, surface plasmon resonance, interferometry, and isothermal titration calorimetry. Compounds according to the present disclosure typically exhibit K_{d} values below 1 μM for a particular cereblon (or the ligand binding domain of the cereblon), e.g., below 750 nM, below 500 nM, below 250 nM, below 100 nM, below 10 nM, or below 1 nM.

### Linker

A linker (L) is used to connect the PTM and ULM. In certain embodiments, L is a bond (i.e., absent). In certain additional embodiments, L is a chemical linker. In certain embodiments, the linker is a connector with a linear non-hydrogen atom number in the range of 1 to 20. The connector may contain functional groups including, but not limited to, ethers, amides, alkanes, alkenes, alkynes, ketones, hydroxyls, carboxylic acids, thioethers, sulfoxides, and sulfones. The linker may contain aromatic, heteroaromatic, cyclic, bicyclic and tricyclic moieties. Substitution with one more halogens, such as CI, F, Br and I may be included in the linker. Examples of suitable linker moieties include, but are not limited to, those disclosed in International Patent Application Nos. WO 2013/106643, WO 2015/160845, WO 2016/149668, WO 2016/197032, WO 2017/011371, WO 2017/011590, WO 2018/144649, and WO 2019/148055, which are incorporated herein by reference in their entirety.

As used herein, the term “alkyl,” by itself or as part of another substituent, refers to a straight or branched, saturated, aliphatic radical having the number of carbon atoms indicated. Alkyl may include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl may also refer to alkyl groups having up to 20 carbons atoms, such as, but not limited to heptyl, octyl, nonyl, decyl, etc. In some aspects, alkyl groups may be substituted as described below. Unless otherwise specified, “substituted alkyl” groups may be substituted with one or more groups selected from halo, hydroxy, amino, alkylamino, amido, acyl, nitro, cyano, and alkoxy.

As used herein, the term “alkenyl,” by itself or as part of another substituent, refers to an alkyl group having at least one carbon-carbon double bond.

As used herein, the term “alkynyl,” by itself or as part of another substituent, refers to an alkyl group having at least one carbon-carbon triple bond.

As used herein, the term “alkoxy,” by itself or as part of another substituent, refers to a group having the formula —OR, wherein R is alkyl.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl may include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups may also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. In some aspects, cycloalkyl groups may be substituted as described below. Unless otherwise specified, “substituted cycloalkyl” groups may be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl.

As used herein, the terms “halo” and “halogen,” by themselves or as part of another substituent, refer to a fluorine, chlorine, bromine, or iodine atom.

As used herein, the term “aryl,” by itself or as part of another substituent, refers to an aromatic ring system having any suitable number of carbon ring atoms and any suitable number of rings. Aryl groups may include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups may be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. In some aspects, alkyl groups may be substituted as described below. Unless otherwise specified, “substituted aryl” groups may be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms may also be useful, including, but not limited to, B, Al, Si and P. The heteroatoms may be oxidized to form moieties such as, but not limited to, —S(O)— and —S(O)₂—. Heteroaryl groups may include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms may be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups may be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group may include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups may also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. In some aspects, heteroaryl groups may be substituted as described below. Unless otherwise specified, “substituted heteroaryl” groups may be substituted with one or more groups selected from halo, hydroxy, amino, alkylamino, amido, acyl, nitro, cyano, and alkoxy.

The heteroaryl groups may be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

As used herein the term “heterocyclyl,” by itself or as part of another substituent, refers to a saturated ring system having from 3 to 12 ring members and from 1 to 4 heteroatoms of N, O and S. Additional heteroatoms may also be useful, including, but not limited to, B, Al, Si and P. The heteroatoms may be oxidized to form moieties such as, but not limited to, —S(O)— and —S(O)₂—. Heterocyclyl groups may include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms may be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group may include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups may also be fused to aromatic or non-aromatic ring systems to form members including, but not limited to, indoline. In some aspects, heterocyclyl groups may be substituted as described below. Unless otherwise specified, “substituted heterocyclyl” groups may be substituted with one or more groups selected from halo, hydroxy, amino, oxo (═O), alkylamino, amido, acyl, nitro, cyano, and alkoxy.

The heterocyclyl groups may be linked via any position on the ring. For example, aziridine may be 1- or 2-aziridine, azetidine may be 1- or 2-azetidine, pyrrolidine may be 1-, 2- or 3-pyrrolidine, piperidine may be 1-, 2-, 3- or 4-piperidine, pyrazolidine may be 1-, 2-, 3-, or 4-pyrazolidine, imidazolidine may be 1-, 2-, 3- or 4-imidazolidine, piperazine may be 1-, 2-, 3- or 4-piperazine, tetrahydrofuran may be 1- or 2-tetrahydrofuran, oxazolidine may be 2-, 3-, 4- or 5-oxazolidine, isoxazolidine may be 2-, 3-, 4- or 5-isoxazolidine, thiazolidine may be 2-, 3-, 4- or 5-thiazolidine, isothiazolidine may be 2-, 3-, 4- or 5-isothiazolidine, and morpholine may be 2-, 3- or 4-morpholine.

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₂, wherein each R group is H or alkyl. An amino moiety may be ionized to form the corresponding ammonium cation.

As used herein, the term “sulfonyl” refers to a moiety —SO₂R, wherein the R group is alkyl, haloalkyl, or aryl. An amino moiety may be ionized to form the corresponding ammonium cation. “Alkylsulfonyl” refers to an amino moiety wherein the R group is alkyl.

As used herein, the term “hydroxy” refers to the moiety —OH.

As used herein, the term “cyano” refers to a carbon atom triple-bonded to a nitrogen atom (i.e., the moiety —C≡N).

As used herein, the term “carboxy” refers to the moiety —C(O)OH. A carboxy moiety may be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

In some embodiments, L comprises one or more ethylene glycol diradical moieties, one or more of which is optionally replaced by a moiety independently selected from:

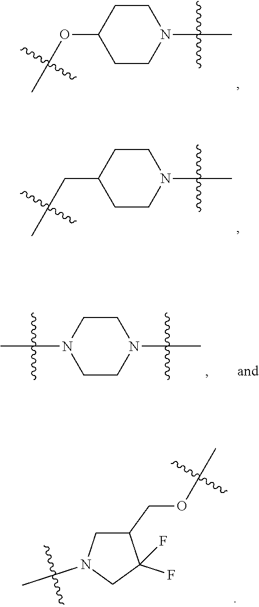 [SMILES: *OC1CCN(*)CC1 ; *CC1CCN(*)CC1 ; *N1CCN(*)CC1 ; *OCC1CN(*)CC1(F)F]

In some embodiments, the linker contains a diradical derived from a compound having two reactive groups (including, but not limited to, aldehydes, carboxylates, activated esters, amines, alcohols, and ionizable C—H bonds), which may be the same or different, that react with complementary moieties in the protein targeting moiety or ligase targeting moiety. Examples of such compounds include, but are not limited to the following.
3-(5-(2-aminoethoxy)-2-fluorophenyl)propanal;
N1-((S)-2-(3-isopropylphenoxy)propyl)-N3-((1s,3R)-3-(m-tolyloxy)cyclobutyl)propane-1,3-diamine;
(1r,3R)—N-(4,4-dimethyl-5-(3-((1s,3S)-3-(methylamino)cyclobutoxy)phenyl)pentyl)-3-((2-ethylbenzofuran-5-yl)oxy)cyclobutan-1-amine;
(1r,3r)-3-((2-(3-((2-((2-ethylbenzofuran-6-yl)oxy)propyl)amino)propyl)benzofuran-6-yl)oxy)-N-methylcyclobutan-1-amine;
1-(2-ethoxyethyl)-4-(4-(1-(methylamino)ethyl)phenyl)pyridin-2(1H)-one;
1-(4-(5-(2-ethoxyethyl)pyrimidin-2-yl)phenyl)-N-methylethan-1-amine;
1-(4-(5-ethoxypenta-1,3-diyn-1-yl)phenyl)-N-methylethan-1-amine;
1-(4-(3-(3-ethoxypropoxy)prop-1-yn-1-yl)phenyl)-N-methylethan-1-amine;
1-(4-(4-(2-ethoxyethyl)piperazin-1-yl)phenyl)-N-methylethan-1-amine;
1-(4-((S)-3-ethoxypyrrolidin-1-yl)phenyl)-N-methylethan-1-amine;
(2-ethoxyphenyl)((3R)-3-((3-(((3R)-1-(4-(1-(methylamino)ethyl)phenyl)pyrrolidin-3-yl)oxy)propyl)amino)pyrrolidin-1-yl)methanone;
1-(4-((R)-3-ethoxypyrrolidin-1-yl)phenyl)-N-methylethan-1-amine;
(Z)—N-methyl-2-(2-(1-(3-methylpent-1-en-1-yl)hydrazineyl)ethoxy)ethan-1-amine;
1-((2R)-1-ethyl-2-methylpiperidin-4-yl)-4-methylpiperazine;
1-((3R)-1-ethyl-3-methylpiperidin-4-yl)-4-methylpiperazine;
(S)—N-ethyl-3-((2-(3-((2-((2-methylbenzofuran-5-yl)oxy)propyl)amino)propyl)benzofuran-5-yl)oxy)propan-1-amine;
(S)-2-(4-(4-ethylphenyl)-1H-imidazol-1-yl)-N-methylpropan-1-amine;
(S)—N-methyl-2-(3-propylphenoxy)propan-1-amine;
(S)-2-((5-ethoxypyridin-2-yl)oxy)-N-methylpropan-1-amine;
(S)—N-methyl-2-((4-(3-((2-(3-propylphenoxy)ethyl)amino)propyl)pyridin-2-yl)oxy)propan-1-amine;
(S)-2-((4-ethylpyridin-2-yl)oxy)-N-methylpropan-1-amine;
1-((2S)-1-ethyl-2-methylpiperidin-4-yl)-4-methylpiperazine;
2-((3S)-1-ethyl-3-methylpiperidin-4-yl)-6-(4-((3R)-3-methyl-4-(4-methylpiperazin-1-yl)piperidin-1-yl)butyl)-2,6-diazaspiro[3.3]heptane;
1-((3S)-1-ethyl-3-methylpiperidin-4-yl)-4-methylpiperazine;
1-((2R,5S)-1-ethyl-2,5-dimethylpiperidin-4-yl)-4-methylpiperazine;
(R)—N1-(2-((4-methylpyridin-2-yl)oxy)propyl)-N3-(2-((1-propylpiperidin-4-yl)oxy)ethyl)propane-1,3-diamine;
(R)—N-ethyl-2-((2-methylbenzofuran-5-yl)oxy)propan-1-amine;
(R)-2-(4-(4-ethylphenyl)-1H-imidazol-1-yl)-N-methylpropan-1-amine;
(R)—N-methyl-2-(3-propylphenoxy)propan-1-amine;
(R)-2-((5-ethoxypyridin-2-yl)oxy)-N-methylpropan-1-amine;
3-(5-(2-phenoxyethoxy)pentyl)isoxazole;
3-(4-(4-phenylpiperazin-1-yl)butyl)isoxazole;
3-(4-(3-phenoxypropoxy)butyl)isoxazole;
3-(3-(4-phenoxybutoxy)propyl)isoxazole;
5-(4-(4-(2-(3-(isoxazol-3-yl)propoxy)ethyl)piperazin-1-yl)phenethyl)-3-(6-(4-phenylpiperazin-1-yl)hexyl)isoxazole;
3-(2-((5-phenoxypentyl)oxy)ethyl)isoxazole;
5-(4-(2-(2-(isoxazol-3-yl)ethoxy)ethoxy)phenethyl)-3-(5-phenoxypentyl)isoxazole;
3-(2-(2-(2-phenoxyethoxy)ethoxy)ethyl)isoxazole;
3-(((6-phenoxyhexyl)oxy)methyl)isoxazole;
5-(4-((5-(isoxazol-3-ylmethoxy)pentyl)oxy)phenethyl)-3-(2-(4-phenoxybutoxy)ethyl)isoxazole;
5-(4-(4-(4-(isoxazol-3-ylmethoxy)butyl)piperazin-1-yl)phenethyl)-3-(2-(3-(4-phenylpiperazin-1-yl)propoxy)ethyl)isoxazole;
3-((2-(4-phenylpiperazin-1-yl)ethoxy)methyl)isoxazole;
3-((2-(3-phenoxypropoxy)ethoxy)methyl)isoxazole;
(1r,3r)-3-phenoxycyclobutan-1-amine;
3-([1,1′-biphenyl]-3-yloxy)propan-1-ol;
6-(4-(2-(isoxazol-3-yl)ethyl)piperazin-1-yl)nicotinaldehyde;
5-(4-aminobutoxy)picolinaldehyde;
4-(4-(8-(2-oxoethyl)-3,8-diazabicyclo[3.2.1]octan-3-yl)butyl)benzaldehyde;
2-(4-((4-(5-(hydroxymethyl)pyridin-2-yl)piperazin-1-yl)methyl)piperidin-1-yl)acetaldehyde;
6-(4-((1-(2-oxoethyl)piperidin-4-yl)methyl)piperazin-1-yl)nicotinaldehyde;
6-(3-(2-(4-(2-oxoethyl)piperazin-1-yl)ethyl)pyrrolidin-1-yl)nicotinaldehyde;
(R)-4-(3-(2-(hydroxymethyl)-4-(2-oxoethyl)piperazin-1-yl)propyl)benzaldehyde;
2-((4-(3-aminophenoxy)benzyl)oxy)acetaldehyde;
2-(3-(3-(4-amino-2-fluorophenoxy)propoxy)propoxy)acetaldehyde;
2-(2-(2-(4-(4-aminophenoxy)butoxy)ethoxy)ethoxy)acetaldehyde;
(1r,3R)-3-ethoxy-N-isopropyl-N-(((1r,4R)-4-methylcyclohexyl)methyl)cyclobutan-1-amine;
N-methyl-2-(6-methylbenzofuran-2-yl)ethan-1-amine;
3-(1-(2-(2-(methylamino)ethoxy)ethyl)-1H-pyrazol-3-yl)-N-(3-((3-methylbenzyl)oxy)propyl)propan-1-amine;
N-methyl-3-((3-methylbenzyl)oxy)propan-1-amine;
(1s,3s)-N-methyl-3-(m-tolyloxy)cyclobutan-1-amine;
4-(((1r,4r)-4-(1H-pyrazol-3-yl)cyclohexyl)oxy)-2-methylbenzonitrile;
(1r,3r)-N-methyl-3-(m-tolyloxy)cyclobutan-1-amine;
N-(3-(2-((1r,3r)-3-(methylamino)cyclobutoxy)pyridin-4-yl)propyl)-1-(p-tolyl)pyrrolidin-3-amine;
(S)—N-methyl-1-(p-tolyl)pyrrolidin-3-amine;
(R)—N-methyl-1-(p-tolyl)pyrrolidin-3-amine;
(R)—N-methyl-1-(6-methylpyridin-3-yl)pyrrolidin-3-amine;
N-methyl-4-(p-tolyl)butan-1-amine;
N-methyl-2-(p-tolyloxy)ethan-1-amine;
N-methyl-2-((6-methylpyridin-3-yl)oxy)ethan-1-amine;
(1s,3s)-N-methyl-3-(p-tolyloxy)cyclobutan-1-amine;
(1r,3r)-N-methyl-3-(p-tolyloxy)cyclobutan-1-amine;
(4-methylpiperazin-1-yl)(m-tolyl)methanone;
1-(2-(4-(5-acetylpyridin-2-yl)piperazin-1-yl)ethoxy)propan-2-one;
2-(3-ethylphenoxy)-N-methylethan-1-amine;
(1s,3s)-3-(3-ethylphenoxy)-N-methylcyclobutan-1-amine;
(1s,3s)-3-((4-ethylpyridin-2-yl)oxy)-N-methylcyclobutan-1-amine;
(1r,3r)-3-(3-ethylphenoxy)-N-methylcyclobutan-1-amine;
(1r,3r)-3-((4-ethylpyridin-2-yl)oxy)-N-methylcyclobutan-1-amine;
2-((5-ethylpyridin-2-yl)oxy)-N-methylethan-1-amine;
(1s,3s)-3-((5-ethylpyridin-2-yl)oxy)-N-methylcyclobutan-1-amine;
(1r,3r)-3-((5-ethylpyridin-2-yl)oxy)-N-methylcyclobutan-1-amine;
4-ethyl-2-(4-methylpiperazin-1-yl)pyrimidine;
1-(4-methylpiperazin-1-yl)pentan-1-one;
N-methyloctan-1-amine;
N-methylhexan-1-amine;
3-butoxy-N-methylpropan-1-amine;
4-(4-(propoxycarbonyl)phenyl)piperazine-1-carboxylic acid;
N-methyl-2-propoxyethan-1-amine;
2-((1-ethylpiperidin-4-yl)oxy)-N-methylethan-1-amine;
3-(4-ethylpiperazin-1-yl)-N-methylpropan-1-amine;
2-(4-ethylpiperazin-1-yl)-N-methylethan-1-amine;
1-(2-ethoxyethyl)-4-methylpiperazine;
2-(2-ethoxyethoxy)-N-methylethan-1-amine;
3-(3-(3-cyclobutoxy-2,2-difluoropropoxy)propoxy)-1-methylazetidine;
3-(3-(3-(3l3-cyclobutoxy)-2,2-difluoropropoxy)propoxy)-1-methylazetidine;
3-((3-(2-((1-methylazetidin-3-yl)oxy)ethoxy)cyclobutyl)methoxy)cyclobutan-1-ol;
1-((1-methylazetidin-3-yl)methyl)-4-(p-tolyl)piperazine;
(4-methylpiperazin-1-yl)(1-(4-(4-(1-methylpiperidin-4-yl)piperazin-1-yl)butyl)piperidin-4-yl)methanone;
1-methyl-4-(1-methylpiperidin-4-yl)piperazine;
1-(1-methylpiperidin-4-yl)piperazine;
(1R,3r)-N-methyl-3-((1-(4-(((1s,3S)-3-((1-methylpiperidin-4-yl)oxy)cyclobutyl)amino)butyl)piperidin-4-yl)oxy)cyclobutan-1-amine;
(1s,3s)-N-methyl-3-((1-methylpiperidin-4-yl)oxy)cyclobutan-1-amine;
(1r,3r)-N-methyl-3-((1-methylpiperidin-4-yl)oxy)cyclobutan-1-amine;
1-((3S)-1,3-dimethylpiperidin-4-yl)piperazine;
1-((3R)-1,3-dimethylpiperidin-4-yl)piperazine;
1-methyl-4-(2-methyl-4-((2-phenylpropan-2-yl)oxy)butan-2-yl)piperazine;
1-((2R,5S)-1,2,5-trimethylpiperidin-4-yl)piperazine;
2-(2-(2-(4-(4-(methylamino)phenoxy)butoxy)ethoxy)ethoxy)acetaldehyde;
2-(2-(2-(2-(4-(methylamino)phenoxy)ethoxy)ethoxy)ethoxy)acetaldehyde;
2-(4-(2-(2-((1S,4S)-5-(2-(2-(methylamino)ethoxy)ethyl)-2,5-diazabicyclo[2.2.1]heptan-2-yl)ethoxy)ethyl)piperazin-1-yl)ethan-1-ol;
2-(3-fluoro-5-((1s,3s)-3-(methylamino)cyclobutoxy)phenoxy)acetaldehyde;
(R)-(2-methoxyphenyl)(3-(methylamino)pyrrolidin-1-yl)methanone;
N-(3-(2-methoxyphenoxy)propyl)-4-(3-(2-(methylamino)ethoxy)phenoxy)butan-1-amine;
3-(2-methoxyphenoxy)-N-methylpropan-1-amine;
1-(4-methoxypyridin-2-yl)-4-methylpiperazine;
2-(3-methoxyphenoxy)-N-methylethan-1-amine;
(1s,3s)-3-(3-methoxyphenoxy)-N-methylcyclobutan-1-amine;
(1s,3s)-3-((4-methoxypyridin-2-yl)oxy)-N-methylcyclobutan-1-amine;
2-((4-methoxyphenyl)sulfonyl)-N-methylethan-1-amine;
1-(5-methoxypyridin-2-yl)-N-methylazetidin-3-amine;
2-((5-methoxypyridin-2-yl)oxy)-N-methylethan-1-amine;
(1s,3s)-3-((5-methoxypyridin-2-yl)oxy)-N-methylcyclobutan-1-amine;
(1r,3r)-3-((5-methoxypyridin-2-yl)oxy)-N-methylcyclobutan-1-amine;
4-methoxy-2-(4-methylpiperazin-1-yl)pyrimidine;
6-(3-hydroxy-3-methylbut-1-yn-1-yl)-4-(4-(3-methoxycyclobutoxy)piperidin-1-yl)nicotinonitrile;
2-((6-(4-(3-methoxycyclobutoxy)piperidin-1-yl)pyridazin-4-yl)oxy)ethan-1-ol;
4-methoxy-1-(2-(p-tolyloxy)ethyl)piperidine;
5-(4-((1-(5-(methoxycarbonyl)pyridin-2-yl)piperidin-4-yl)methyl)piperazin-1-yl)picolinic acid;
methyl 4-(4-carbamoylphenyl)piperazine-1-carboxylate;
methyl 4-(2-(4-(benzyloxy)phenoxy)ethyl)piperazine-1-carboxylate;
methyl 4-(2-(4-hydroxyphenoxy)ethyl)piperazine-1-carboxylate;
(Z)-4-(4-(4-methoxy-4-oxobut-2-en-2-yl)phenyl)piperazine-1-carboxylic acid;
3-(4-(methoxymethyl)phenoxy)-N-methylaniline;
3-((1-(3-(3-methoxyprop-1-yn-1-yl)-5-(trifluoromethyl)phenyl)piperidin-4-yl)oxy)cyclobutan-1-ol;
1-(4-(5-methoxypenta-1,3-diyn-1-yl)phenyl)ethan-1-amine;
N-(4-(2-methoxyethyl)benzyl)-5-(3-(2-(methylamino)ethoxy)phenyl)pentan-1-amine;
1-(4-(2-methoxyethyl)phenyl)-N-methylmethanamine;
3-methoxy-1-(4-(4-((methylamino)methyl)phenyl)piperazin-1-yl)propan-1-one;
(R)-3-methoxy-1-(3-(methylamino)pyrrolidin-1-yl)propan-1-one;
N-(2-((4-(aminomethyl)phenyl)amino)ethyl)-3-methoxypropanamide;
3-methoxy-1-((R)-3-(methylamino)pyrrolidin-1-yl)propan-1-ol;
3-((5-(6-methoxyhex-1-yn-1-yl)pyridin-2-yl)oxy)propan-1-ol;
2-(3-ethoxypropoxy)-5-(6-methoxyhex-1-yn-1-yl)pyridine;
4-methoxy-N-methylbutan-1-amine;
3-methoxy-N-methylpropan-1-amine;
6-(4-(3-methoxypropoxy)cyclohexa-1,3-dien-1-yl)-N-methylpyridin-3-amine;
5-(4-(3-methoxypropoxy)cyclohexa-1,3-dien-1-yl)-N-methylpyrazin-2-amine;
2-(3-(azetidin-3-yloxy)propyl)-5-(3-methoxypropoxy)pyridine;
2-(3-(azetidin-3-yloxy)prop-1-yn-1-yl)-5-(3-methoxypropoxy)pyridine;
3-(3-methoxypropoxy)-1-methylazetidine;
5-(3-methoxypropoxy)-N-methylpentan-1-amine;
3-(3-methoxypropoxy)-N-methylpropan-1-amine;
2-(3-methoxypropoxy)-N-methylethan-1-amine;
2-((3-methoxypropyl)sulfonyl)-N-methylethan-1-amine;
2-((3-methoxypropyl)sulfinyl)-N-methylethan-1-amine;
2-((3-methoxypropyl)thio)-N-methylethan-1-amine;
1-(4-(1-(2-methoxyethyl)piperidin-4-yl)phenyl)-N-methylmethanamine;
1-(2-methoxyethyl)-N-methylpiperidin-4-amine;
1-(4-(4-(2-methoxyethyl)piperazin-1-yl)phenyl)-N-methylmethanamine;
2-(4-(2-(2-((1S,4S)-5-(2-methoxyethyl)-2,5-diazabicyclo[2.2.1]heptan-2-yl)ethoxy)ethyl)piperazin-1-yl)ethan-1-ol;
3-(2-methoxyethoxy)-N-methylpropan-1-amine;
4-(2-(2-methoxyethoxy)ethoxy)-N-methylbutan-1-amine;
N-methyl-2,5,8,12,15-pentaoxaheptadecan-17-amine;
2-(2-(2-methoxyethoxy)ethoxy)-N-methylethan-1-amine;
N-methyl-2,5,8,11-tetraoxatridecan-13-amine;
(1s,3s)-3-(2-methoxyethoxy)-N-methylcyclobutan-1-amine;
(1r,3r)-3-(2-methoxyethoxy)-N-methylcyclobutan-1-amine;
2-((1r,3r)-3-(2-methoxyethoxy)cyclobutoxy)-N-methylethan-1-amine;
(S)-1-methoxy-3-(4-(3-(4-(4-methoxypyrimidin-2-yl)piperazin-1-yl)propyl)piperazin-1-yl)propan-2-ol;
(S)-1-methoxy-3-(4-methylpiperazin-1-yl)propan-2-ol;
(R)-1-methoxy-3-(4-methylpiperazin-1-yl)propan-2-ol;
(Z)-2-((4-methoxybut-2-en-1-yl)oxy)-N-methylethan-1-amine;
1-(4-methylpyridin-2-yl)piperazine;
5-(2-(4-(4-(isoxazol-3-yl)butyl)piperazin-1-yl)ethyl)-3-(3-(piperazin-1-yl)propyl)isoxazole;
1-phenyl-4-(2-(piperazin-1-yl)ethyl)piperazine;
2-(3-(3-(4-(1-aminocyclopropyl)phenoxy)propoxy)propoxy)acetaldehyde;
2-(4-(3-(4-aminophenoxy)propoxy)butoxy)acetaldehyde;
2-(3-(4-(3-(4-aminophenoxy)propoxy)butoxy)propoxy)acetaldehyde;
2-(3-(3-(4-amino-2-fluorophenoxy)propoxy)propoxy)acetaldehyde;
2-(3-(3-(4-amino-3-fluorophenoxy)propoxy)propoxy)acetaldehyde;
14-(4-aminophenoxy)-3,6,9,12-tetraoxatetradecanal;
1-(4-methylpyridin-2-yl)pyrrolidin-3-amine;
1-(2-methoxyethyl)piperidin-4-amine;
1-(4-methylpyridin-2-yl)azetidin-3-amine;
2-(4-(4-phenoxybutyl)piperazin-1-yl)acetamide;
N-(2-(4-(4-(2-amino-2-oxoethyl)piperazin-1-yl)butoxy)ethyl)-2-(4-(3-hydroxypropyl)piperazin-1-yl)acetamide;
2-((5-(2-(piperazin-1-yl)ethoxy)pentyl)oxy)acetamide;
4-(4-(2-carbamoylhydrazine-1-carbonyl)phenyl)piperazine-1-carboxylic acid;
3,3-difluoro-5-methoxypentan-1-amine;
9-(3-((5-(methylamino)pentyl)oxy)propoxy)nonan-1-amine;
5-methoxypentan-1-amine;
4-((6-methylpyridin-3-yl)oxy)butan-1-amine;
3-((2-methylbenzofuran-5-yl)oxy)propan-1-amine;
6-(3-aminopropoxy)-2-methyl-N-(3-(2-(methylamino)ethoxy)benzyl)imidazo[1,2-a]pyridin-8-amine;
3-((2-methylimidazo[1,2-a]pyridin-6-yl)oxy)propan-1-amine;
3-((6-methylpyridin-3-yl)oxy)propan-1-amine;
2-((2-methylbenzofuran-5-yl)oxy)ethan-1-amine;
2-(m-tolyloxy)ethan-1-amine;
3-(6-(2-aminoethoxy)pyridin-2-yl)-N-(2-((2-methylbenzofuran-5-yl)oxy)ethyl)propan-1-amine;
(E)-2-(3-(prop-1-en-1-yl)phenoxy)ethan-1-amine;
3-(6-(2-aminoethoxy)imidazo[1,2-a]pyridin-2-yl)-N-(3-((2-methylimidazo[1,2-a]pyridin-6-yl)oxy)propyl)propan-1-amine;
2-((2-methylimidazo[1,2-a]pyridin-6-yl)oxy)ethan-1-amine;
2-(2-(3-methyl-1H-pyrazol-1-yl)ethoxy)ethan-1-amine;
2-(4-(2-(2-((1S,4S)-5-(2-(2-aminoethoxy)ethyl)-2,5-diazabicyclo[2.2.1]heptan-2-yl)ethoxy)ethyl)piperazin-1-yl)ethan-1-ol;
2,5,8,13,16-pentaoxaoctadecan-18-amine;
2,5,8,12,15-pentaoxaheptadecan-17-amine;
2,5,8,11,14,17-hexaoxanonadecan-19-amine;
2,5,8,11,14-pentaoxahexadecan-16-amine;
2,5,8,11-tetraoxatridecan-13-amine;
2-(2-(2-methoxyethoxy)ethoxy)ethan-1-amine;
2-(2-methoxyethoxy)ethan-1-amine;
(S)-1-(2-methoxyethyl)pyrrolidin-3-amine;
(1r,3r)-3-((2-methylbenzofuran-6-yl)oxy)cyclobutan-1-amine;
(1r,3r)-3-((2-methylbenzofuran-5-yl)oxy)cyclobutan-1-amine;
(1r,3r)-3-(p-tolyloxy)cyclobutan-1-amine;
(1r,3r)-3-((5-methylpyridin-2-yl)oxy)cyclobutan-1-amine;
(1r,3r)-3-(m-tolyloxy)cyclobutan-1-amine;
(1r,3r)-3-((5-methylpyrazin-2-yl)oxy)cyclobutan-1-amine;
(1r,3r)-3-((4-methylpyridin-2-yl)oxy)cyclobutan-1-amine;
(1s,3s)-3-((2-(3-(6-((1s,3s)-3-aminocyclobutoxy)benzofuran-2-yl)propyl)benzofuran-5-yl)oxy)-N-methylcyclobutan-1-amine;
(1s,3s)-3-((2-methylbenzofuran-6-yl)oxy)cyclobutan-1-amine;
(1s,3s)-3-((2-methylbenzofuran-5-yl)oxy)cyclobutan-1-amine;
(1s,3s)-3-(m-tolyloxy)cyclobutan-1-amine;
(1s,3s)-3-((2-methylpyridin-4-yl)oxy)cyclobutan-1-amine;
(1s,3s)-3-((5-methoxypyridin-2-yl)oxy)cyclobutan-1-amine;
(1s,3s)-3-((4-methylpyridin-2-yl)oxy)cyclobutan-1-amine;
(1s,3s)-3-(2-methoxyethoxy)cyclobutan-1-amine;
(R)-2-(3-(3-(4-(1-aminoethyl)phenoxy)propoxy)propoxy)acetaldehyde;
6-(5-hydroxy-2,2,4,4-tetramethylpentyl)pyridin-3-ol;
4-(2-(6-hydroxypyridin-3-yl)-1,3-dioxoisoindolin-5-yl)piperazine-1-carboxylic acid;
3-(2-((2-(2-((1-methylazetidin-3-yl)oxy)ethoxy)ethoxy)methyl)cyclopropyl)cyclobutan-1-01;
3-((6-(3-(2-hydroxyethoxy)-3-methylbut-1-yn-1-yl)pyridin-3-yl)oxy)cyclobutan-1-ol;
3-((6-(3-(2-hydroxyethoxy)but-1-yn-1-yl)pyridin-3-yl)oxy)cyclobutan-1-ol;
3-((6-(3,3-difluoro-3-(2-hydroxyethoxy)prop-1-yn-1-yl)pyridin-3-yl)oxy)cyclobutan-1-ol;
5-(3-hydroxycyclobutoxy)-2-(3-(2-hydroxyethoxy)-3-methylbut-1-yn-1-yl)isonicotinonitrile;
3-((6-(3-(4-methylpiperazin-1-yl)propyl)pyridin-3-yl)oxy)cyclobutan-1-ol;
3-((6-(3-(4-methylpiperazin-1-yl)propyl)pyridin-3-yl)oxy)cyclobutan-1-ol;
3-((1-(5-(3-hydroxyprop-1-yn-1-yl)-2-methylphenyl)piperidin-4-yl)oxy)cyclobutan-1-ol;
4-(4-(3-hydroxycyclobutoxy)piperidin-1-yl)-6-(3-hydroxyprop-1-yn-1-yl)nicotinonitrile;
3-((1-(3-(3-hydroxyprop-1-yn-1-yl)-5-methylphenyl)piperidin-4-yl)oxy)cyclobutan-1-ol;
3-((1-(3-(3-hydroxy-3-methylbut-1-yn-1-yl)phenyl)piperidin-4-yl)oxy)cyclobutan-1-ol;
3-((1-(2-(3-hydroxy-3-methylbut-1-yn-1-yl)pyridin-4-yl)piperidin-4-yl)oxy)cyclobutan-1-01;
3-((1-(2-(3-hydroxy-3-methylbut-1-yn-1-yl)pyrimidin-4-yl)piperidin-4-yl)oxy)cyclobutan-1-ol;
3-((1-(2,2-difluoro-5-methoxypentyl)piperidin-4-yl)oxy)cyclobutan-1-ol;
3-((1-(2,2-difluoro-5-hydroxypentyl)piperidin-4-yl)oxy)cyclobutan-1-ol;
3-((3-((3-(azetidin-3-yloxy)propoxy)methyl)oxetan-3-yl)methoxy)cyclobutan-1-ol;
3-((3-(2-((1-methylazetidin-3-yl)oxy)ethoxy)cyclobutyl)methoxy)cyclobutan-1-ol;
3-((3-((2-((1-ethylazetidin-3-yl)oxy)ethoxy)methyl)bicyclo[1.1.1]pentan-1-yl)methoxy)cyclobutan-1-ol;
3-((3-((2-((1-methylazetidin-3-yl)oxy)ethoxy)methyl)bicyclo[1.1.1]pentan-1-yl)methoxy)cyclobutan-1-ol;
3-(3-(3-((1-ethylazetidin-3-yl)oxy)-2,2-difluoropropoxy)propoxy)cyclobutan-1-ol;
3-(3-(2,2-difluoro-3-((1-methylazetidin-3-yl)oxy)propoxy)propoxy)cyclobutan-1-ol;
3-((3-(2-((1-methylazetidin-3-yl)oxy)ethoxy)cyclobutyl)methoxy)cyclobutan-1-ol;
7-(piperazin-1-yl)decahydronaphthalen-2-ol;
1-(3-(3-(3-(aminomethyl)phenyl)propoxy)propyl)piperidin-3-ol;
4-(4-carboxyphenyl)piperazine-1-carboxylic acid;
4-(6-carboxypyridin-3-yl)piperazine-1-carboxylic acid;
5-(4-((1-(5-carboxypyridin-2-yl)piperidin-4-yl)methyl)piperazin-1-yl)picolinic acid;
(S)-5-amino-4-(4-(3-((1-(4-(methoxycarbonyl)phenyl)piperidin-4-yl)methyl)azetidin-1-yl)-1-oxoisoindolin-2-yl)-5-oxopentanoic acid;
2-((5-((4-(cyclopentylcarbamoyl)phenyl)amino)pentyl)oxy)acetic acid;
4-(4-carboxyphenyl)piperazine-1-carboxylic acid;
4-(4-((2-carboxyethyl)amino)-2-fluorobenzoyl)piperazine-1-carboxylic acid;
3-((1-(2-(3,3-difluoro-3-hydroxyprop-1-yn-1-yl)pyridin-4-yl)piperidin-4-yl)oxy)cyclobutan-1-ol;
3-(4-(3-hydroxycyclobutoxy)piperidin-1-yl)-5-(3-hydroxyprop-1-yn-1-yl)benzonitrile;
3-((1-(2-(3-hydroxyprop-1-yn-1-yl)-6-(trifluoromethyl)pyridin-4-yl)piperidin-4-yl)oxy)cyclobutan-1-ol;
3-((1-(3-(3-hydroxyprop-1-yn-1-yl)phenyl)piperidin-4-yl)oxy)cyclobutan-1-ol;
1-((4-(3-(3-hydroxyprop-1-yn-1-yl)phenyl)piperazin-1-yl)methyl)cyclobutane-1,3-diol;
3-((4-(3-(3-hydroxyprop-1-yn-1-yl)phenyl)piperazin-1-yl)methyl)cyclobutan-1-ol;
3-((1-(2-(3-hydroxyprop-1-yn-1-yl)pyridin-4-yl)piperidin-4-yl)oxy)cyclobutan-1-ol;
3-((1-(4-(3-hydroxyprop-1-yn-1-yl)-6-(trifluoromethyl)pyridin-2-yl)piperidin-4-yl)oxy)cyclobutan-1-ol;
3-(3-(4-(3-ethoxycyclobutoxy)piperidin-1-yl)-4-(trifluoromethyl)phenyl)prop-2-yn-1-ol;
2-(4-(3-hydroxycyclobutoxy)piperidin-1-yl)-4-(3-hydroxyprop-1-yn-1-yl)benzonitrile;
3-((1-(5-(3-hydroxyprop-1-yn-1-yl)-2-(trifluoromethyl)phenyl)piperidin-4-yl)oxy)cyclobutan-1-ol;
3-((1-(2-(3-hydroxyprop-1-yn-1-yl)-5-(trifluoromethyl)pyridin-4-yl)piperidin-4-yl)oxy)cyclobutan-1-ol;
3-((1-(4-(3-hydroxyprop-1-yn-1-yl)pyrimidin-2-yl)piperidin-4-yl)oxy)cyclobutan-1-ol;
3-((1-(3-(3-hydroxypropyl)phenyl)piperidin-4-yl)oxy)cyclobutan-1-ol;
(3-hydroxyazetidin-1-yl)(3-(7-hydroxyheptyl)bicyclo[1.1.1]pentan-1-yl)methanone;
(3-hydroxyazetidin-1-yl)(3-(((5-hydroxypentyl)oxy)methyl)bicyclo[1.1.1]pentan-1-yl)methanone;
3-aminopropan-1-ol;
3-((4′-(2-(3-(3-([1,1′-biphenyl]-4-yloxy)propoxy)propoxy)ethyl)-[1,1′-biphenyl]-3-yl)oxy)propan-1-ol;
3-(4-(5-(2-(4-(3-(isoxazol-3-yl)propoxy)butoxy)ethyl)isoxazol-3-yl)butoxy)propan-1-ol;
3-(2-(2-(phenoxymethyl)phenoxy)ethoxy)propan-1-ol;
2-([1,4′-bipiperidin]-1′-yl)ethan-1-ol;
2-(4-(2-(2-((1S,4S)-5-(2-hydroxyethyl)-2,5-diazabicyclo[2.2.1]heptan-2-yl)ethoxy)ethyl)piperazin-1-yl)ethan-1-ol;
3-((6-((1-(2-hydroxyethoxy)cyclopropyl)ethynyl)pyridin-3-yl)oxy)cyclobutan-1-ol;
3-((1-(2,2-difluoro-3-(2-hydroxyethoxy)propyl)piperidin-4-yl)oxy)cyclobutan-1-ol;
2-(2-(3-(2-phenoxyethoxy)phenoxy)ethoxy)ethan-1-ol;
2-(2-((5-(2-(2-((5-(isoxazol-3-yl)pentyl)oxy)ethoxy)ethyl)isoxazol-3-yl)methoxy)ethoxy)ethan-1-ol; and
2-(2-(2-(5-(2-(4-(isoxazol-3-yl)piperazin-1-yl)ethyl)isoxazol-3-yl)ethoxy)ethoxy)ethan-1-ol.

In some embodiments, the linker -L- in Formula II is selected from the groups listed below, wherein “R₁” represents the point of attachment to R^{1a}, R², R³, R⁴, R⁵, R⁶, and/or R⁷ and “R₂” represents the point of attachment to the PTM. In some embodiments, the linker -L- in Formula II is selected from the groups listed below, wherein “R₁” represents the point of attachment to the PTM and “R₂” represents the point of attachment to R^{1a}, R², R³, R⁴, R⁵, R⁶, and/or R⁷.

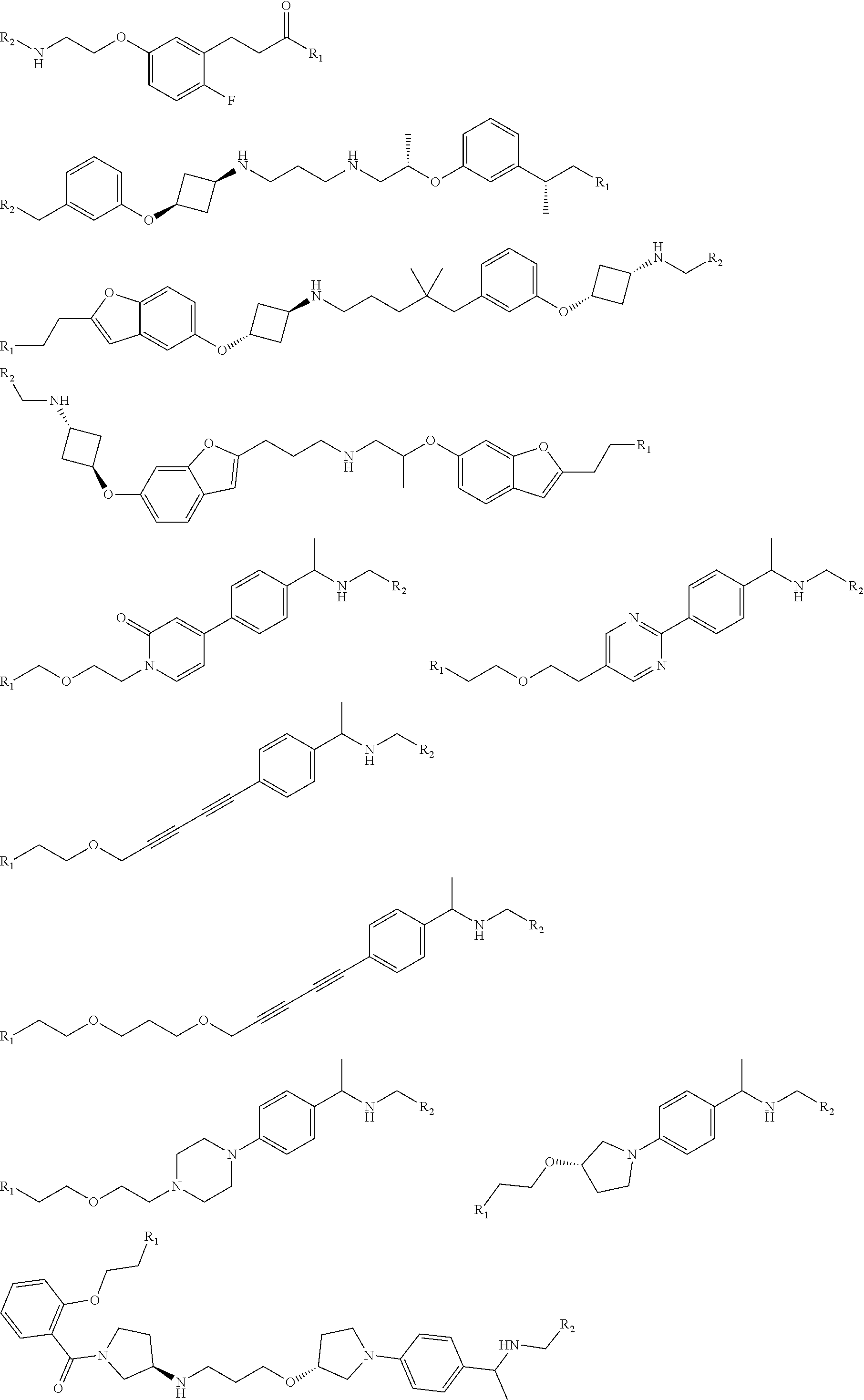

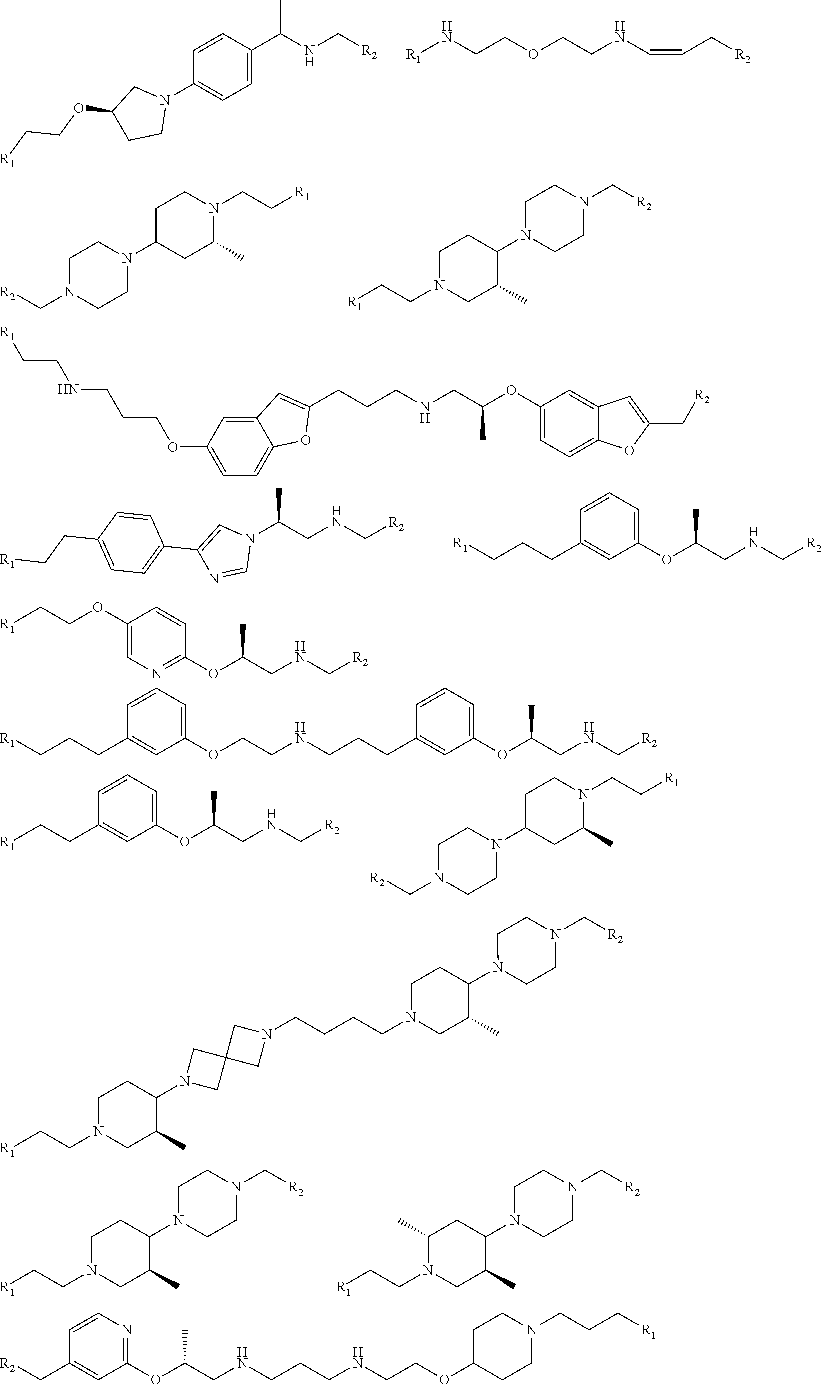

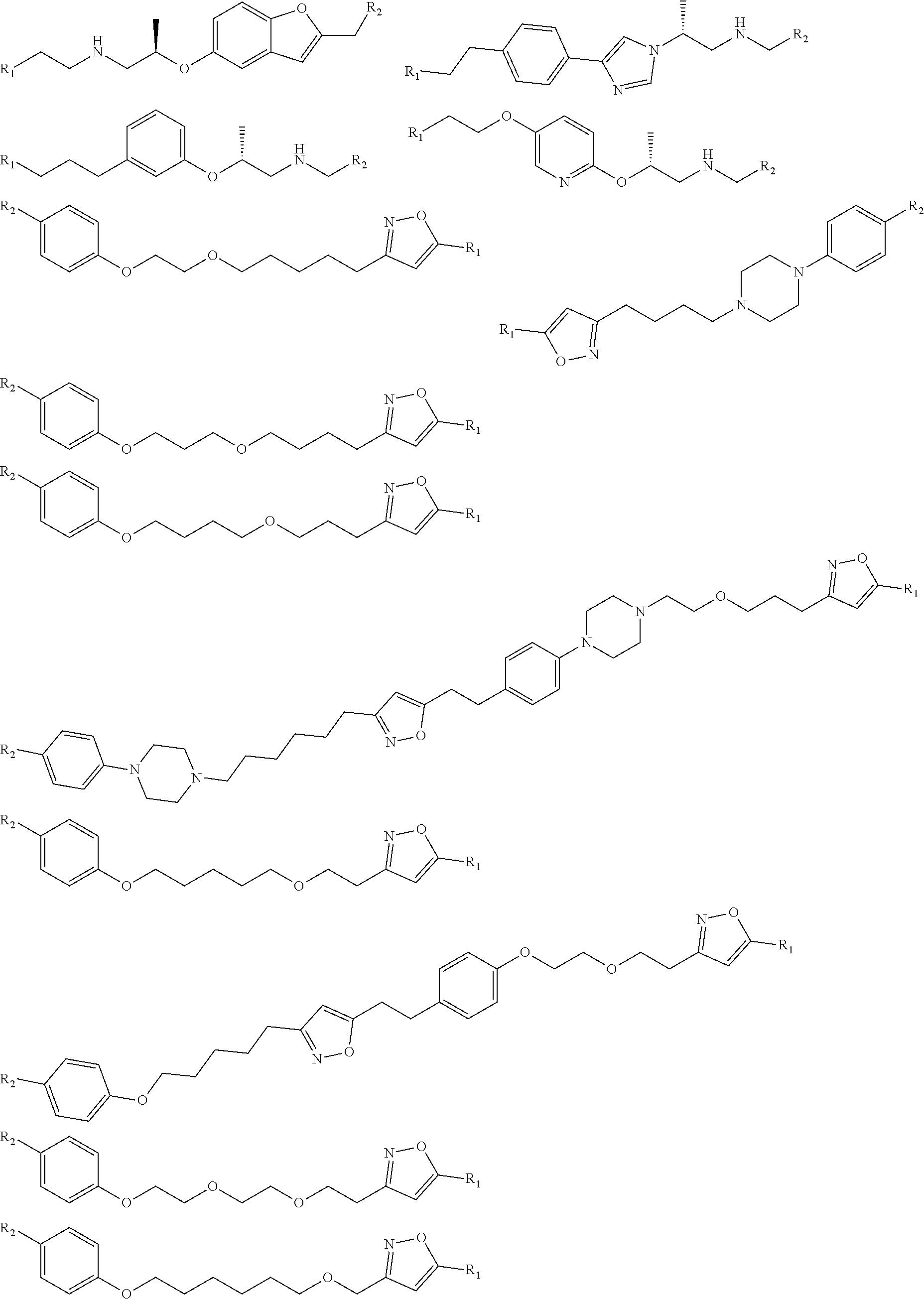

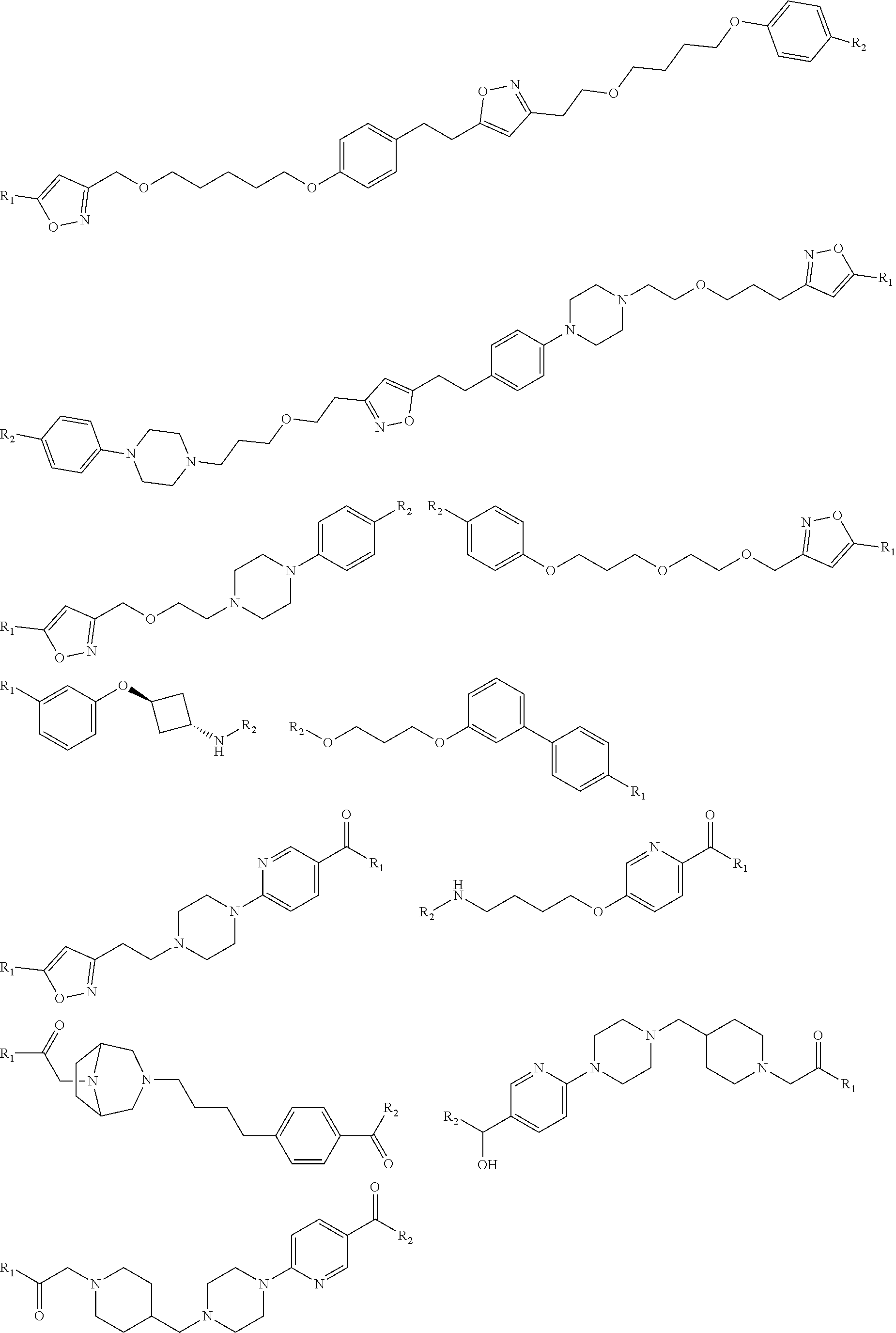

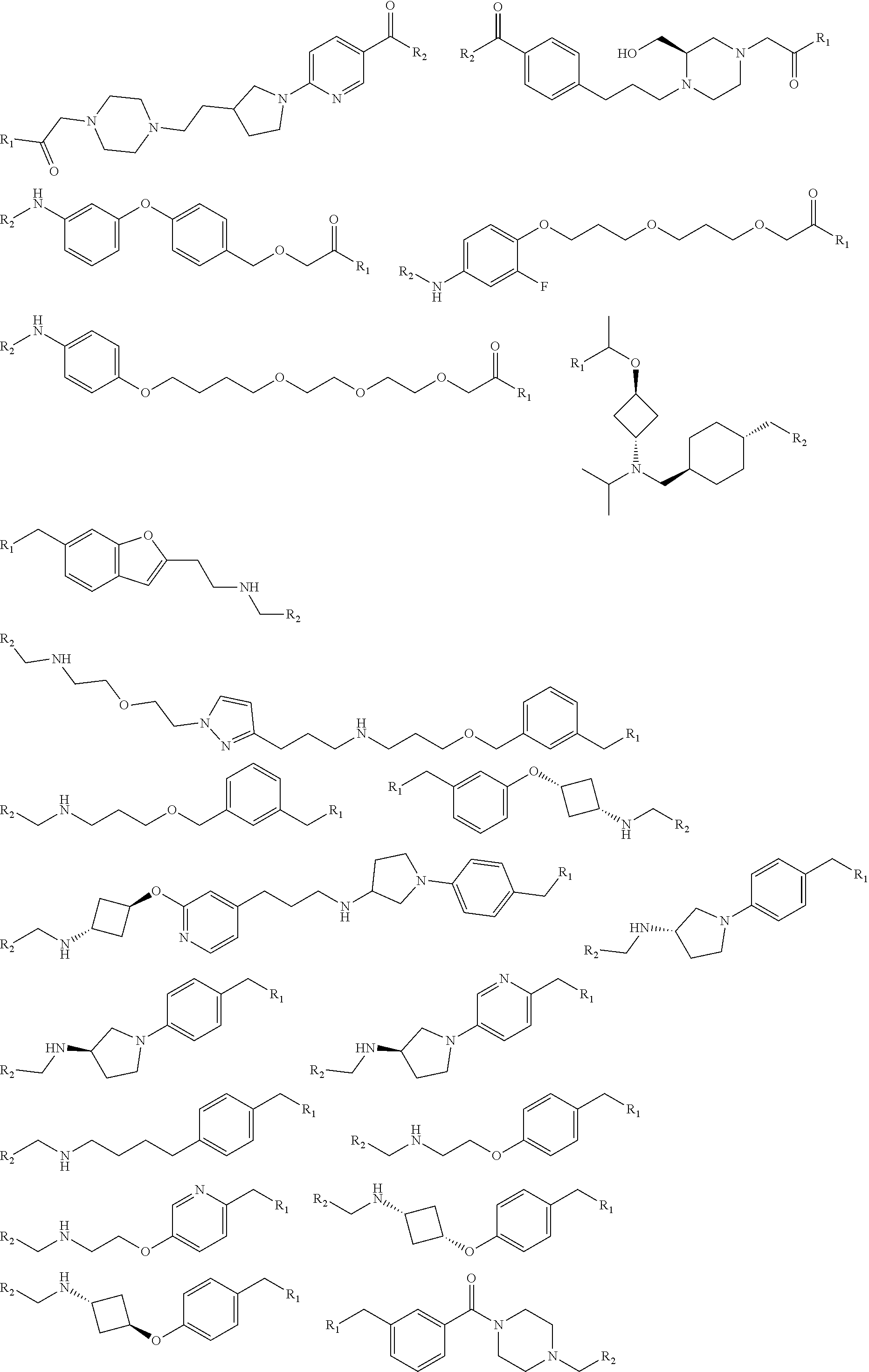

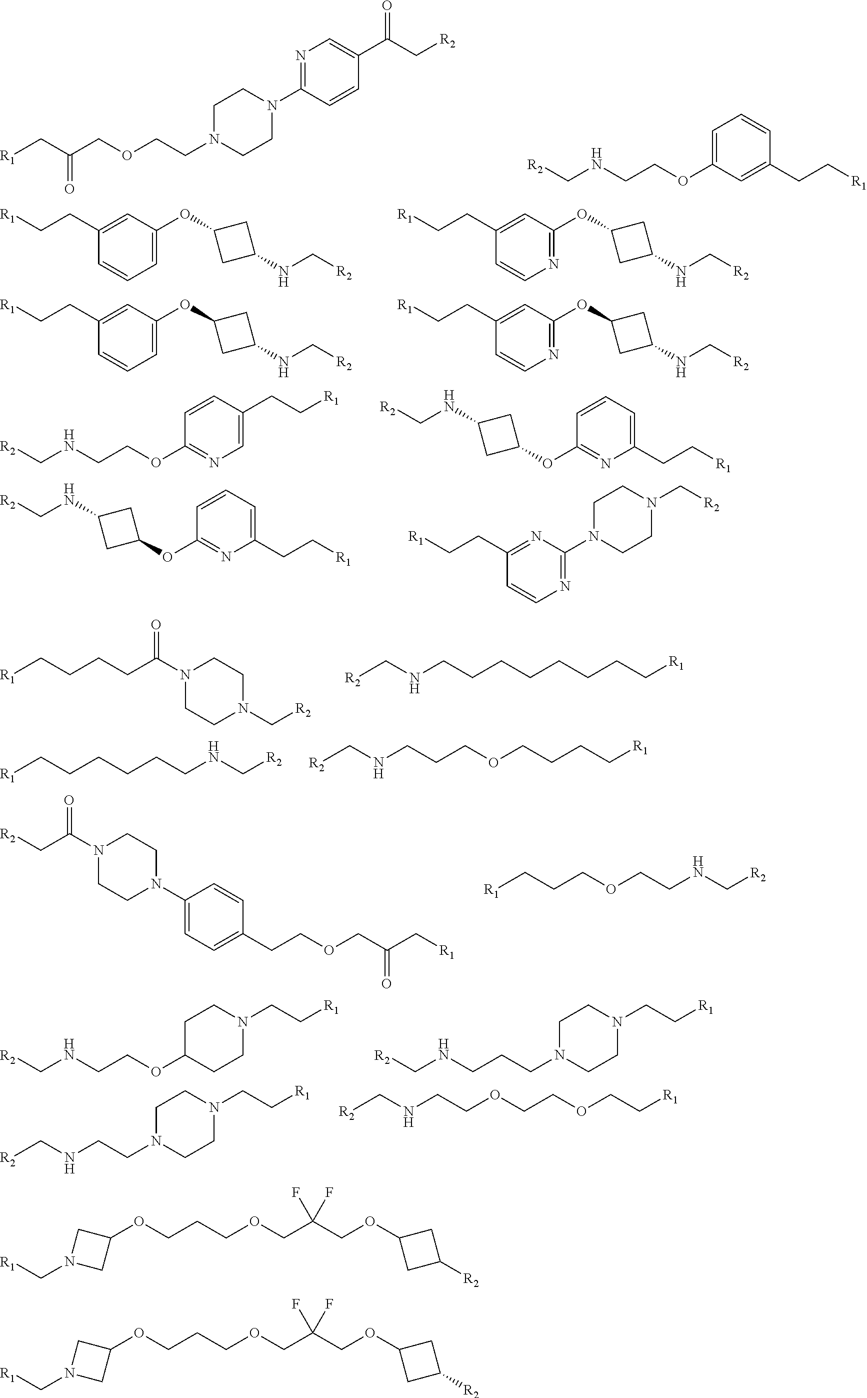

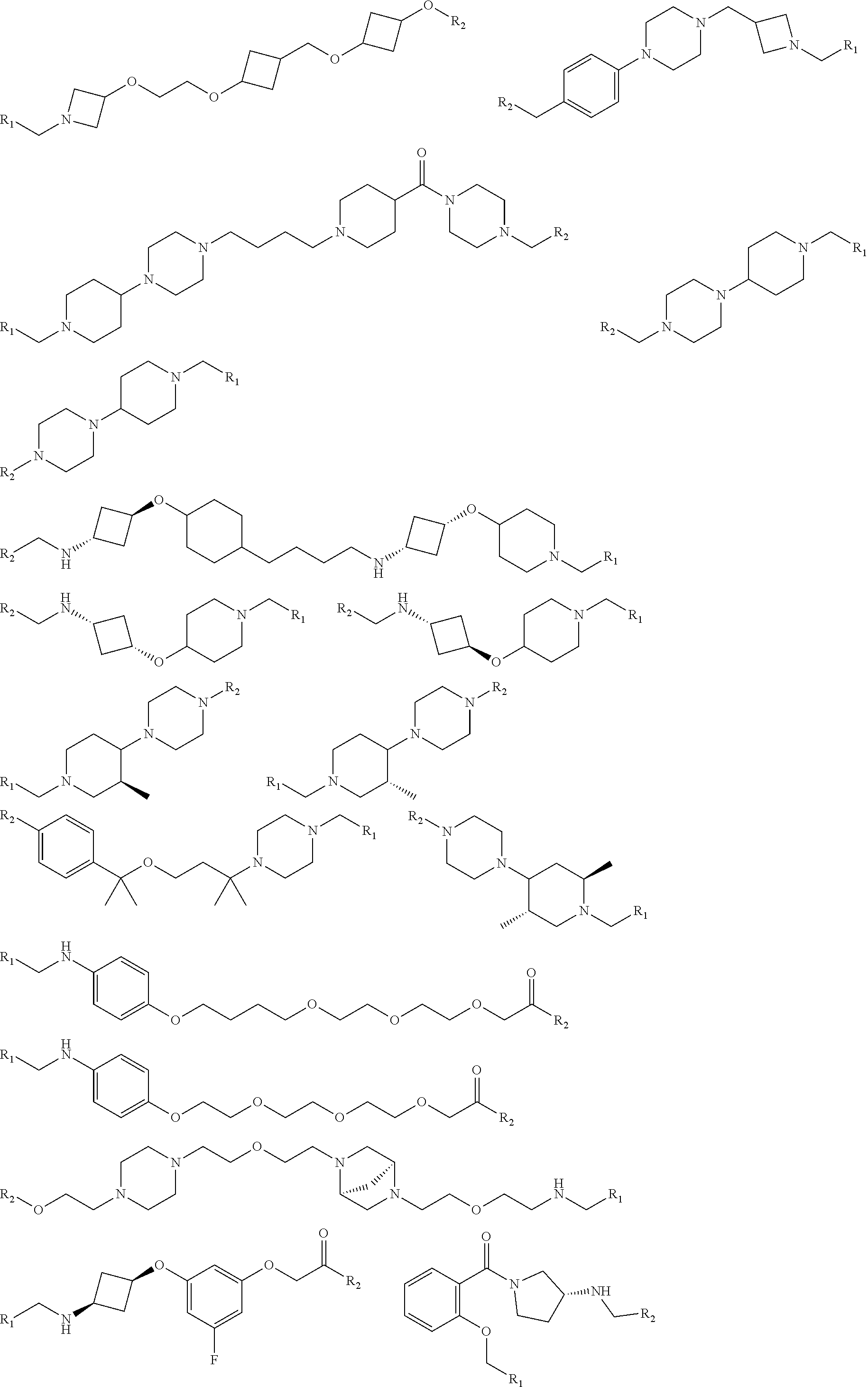

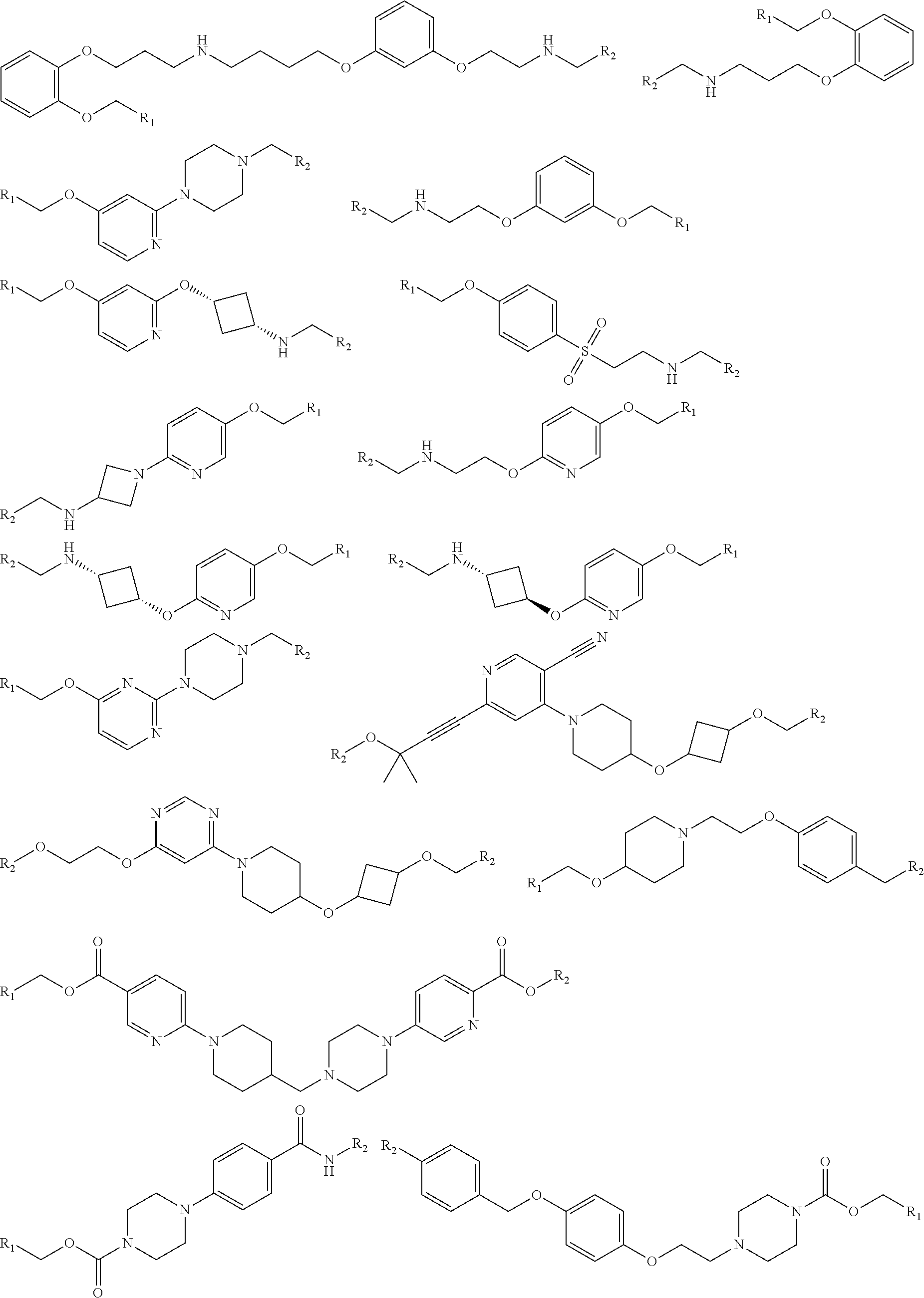

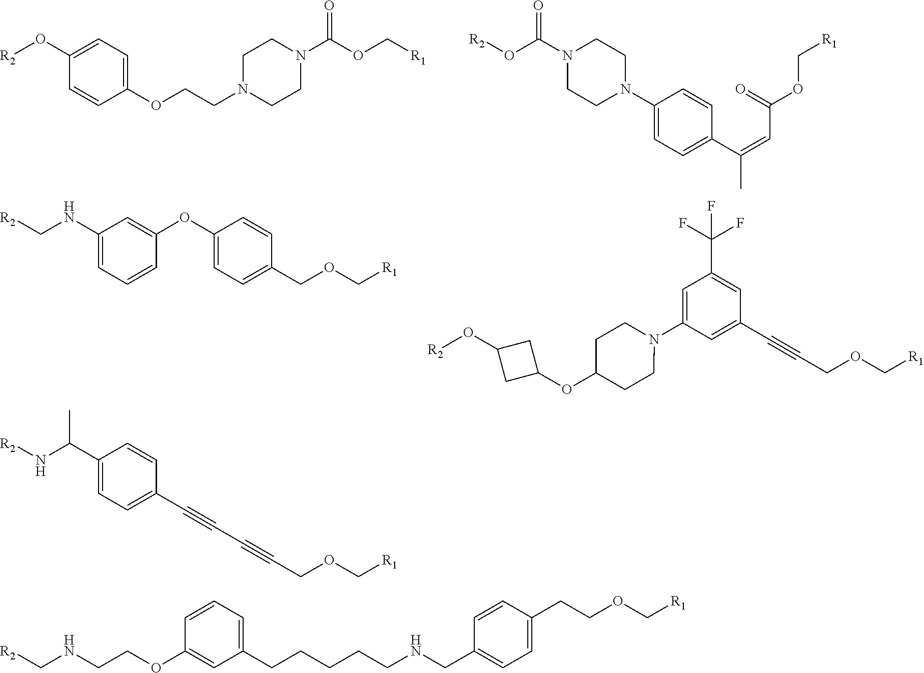

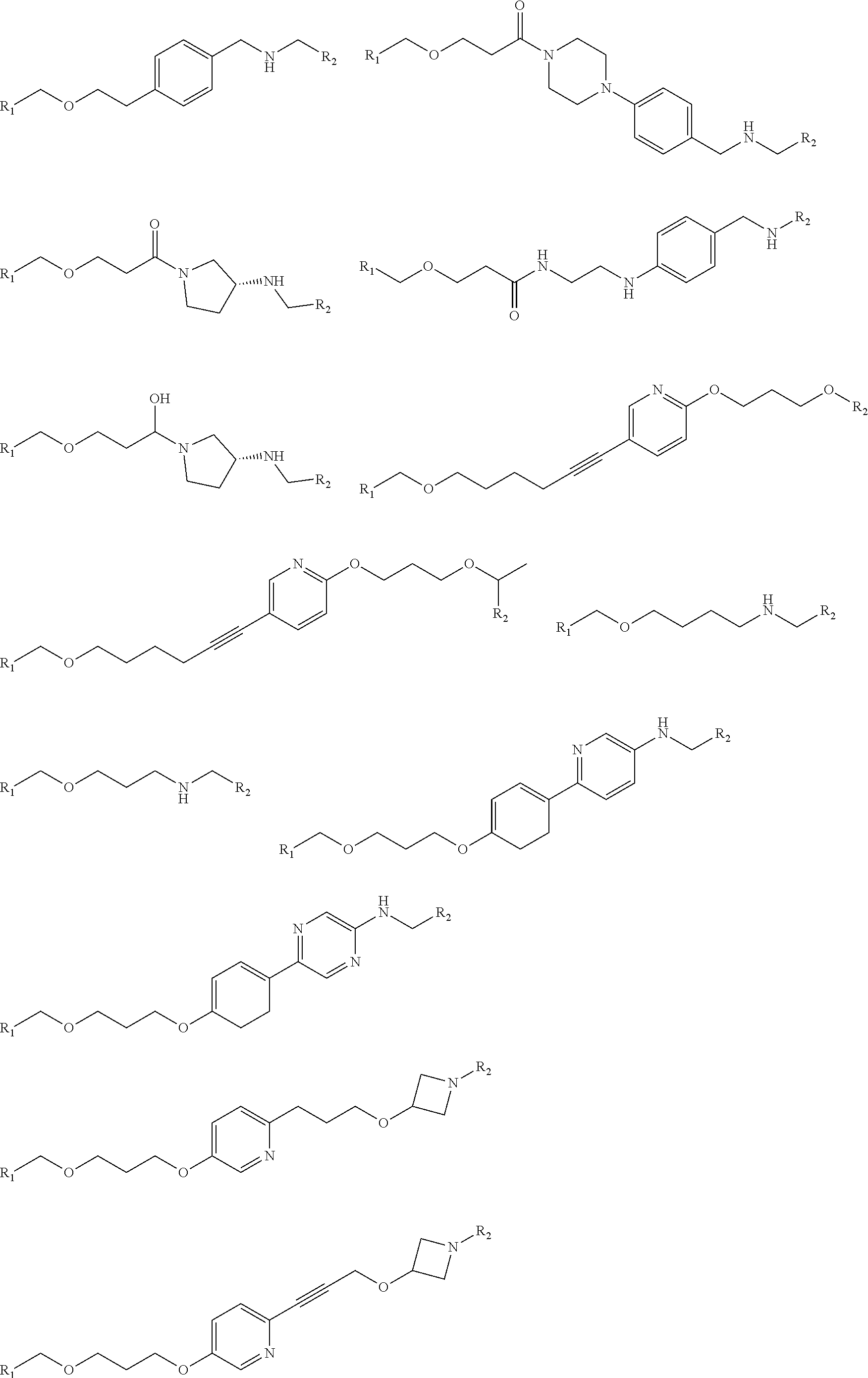

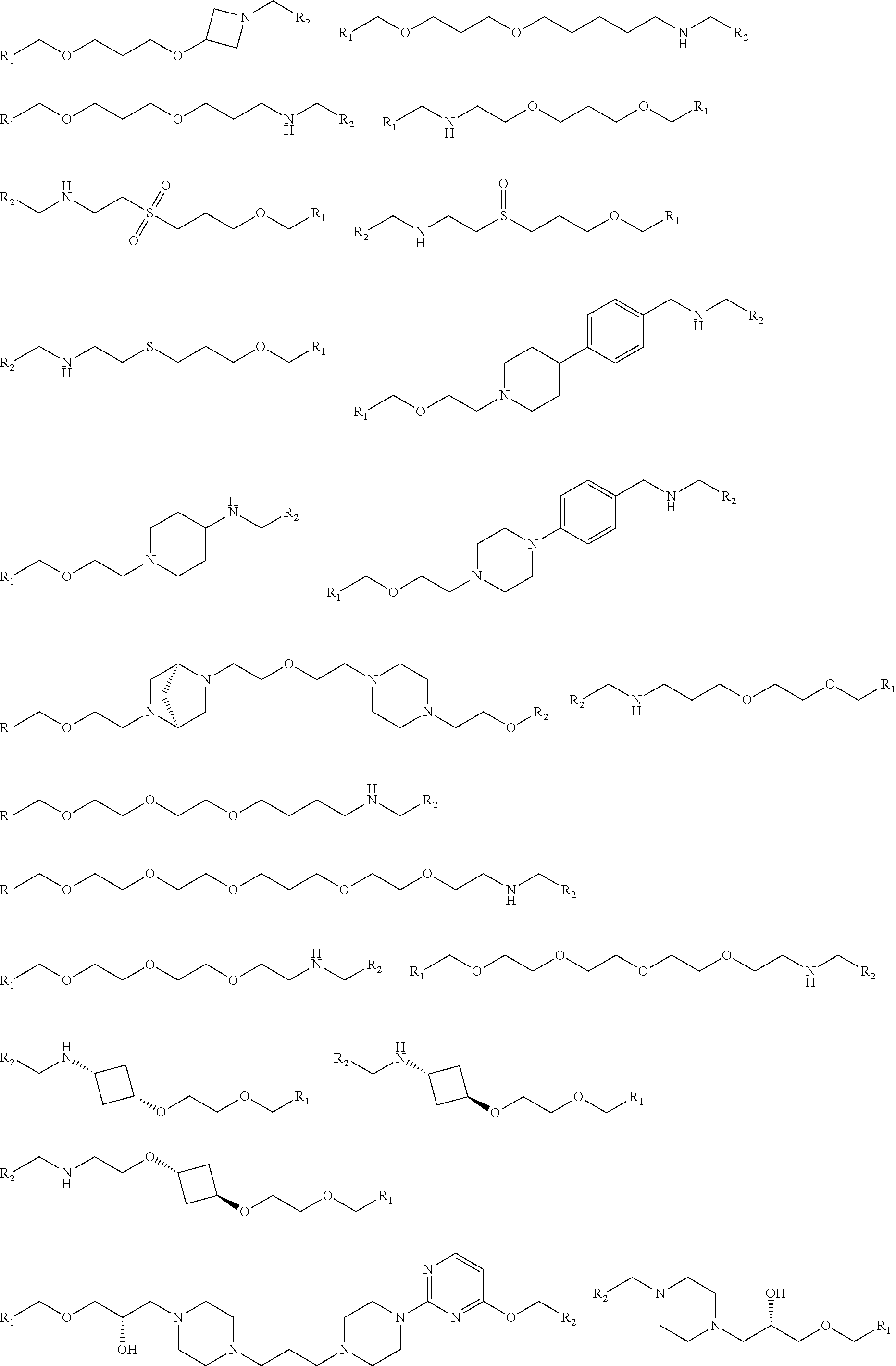

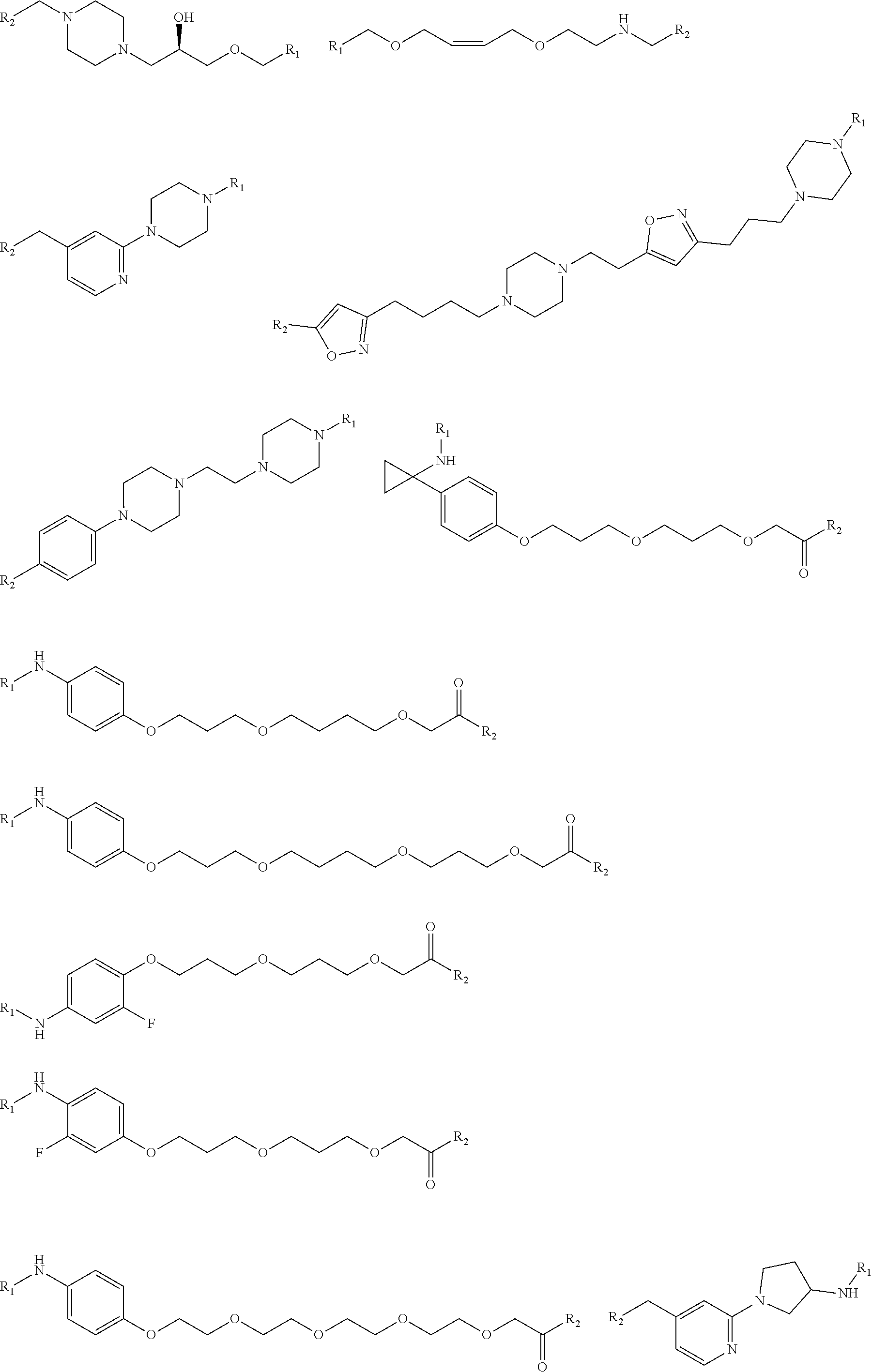

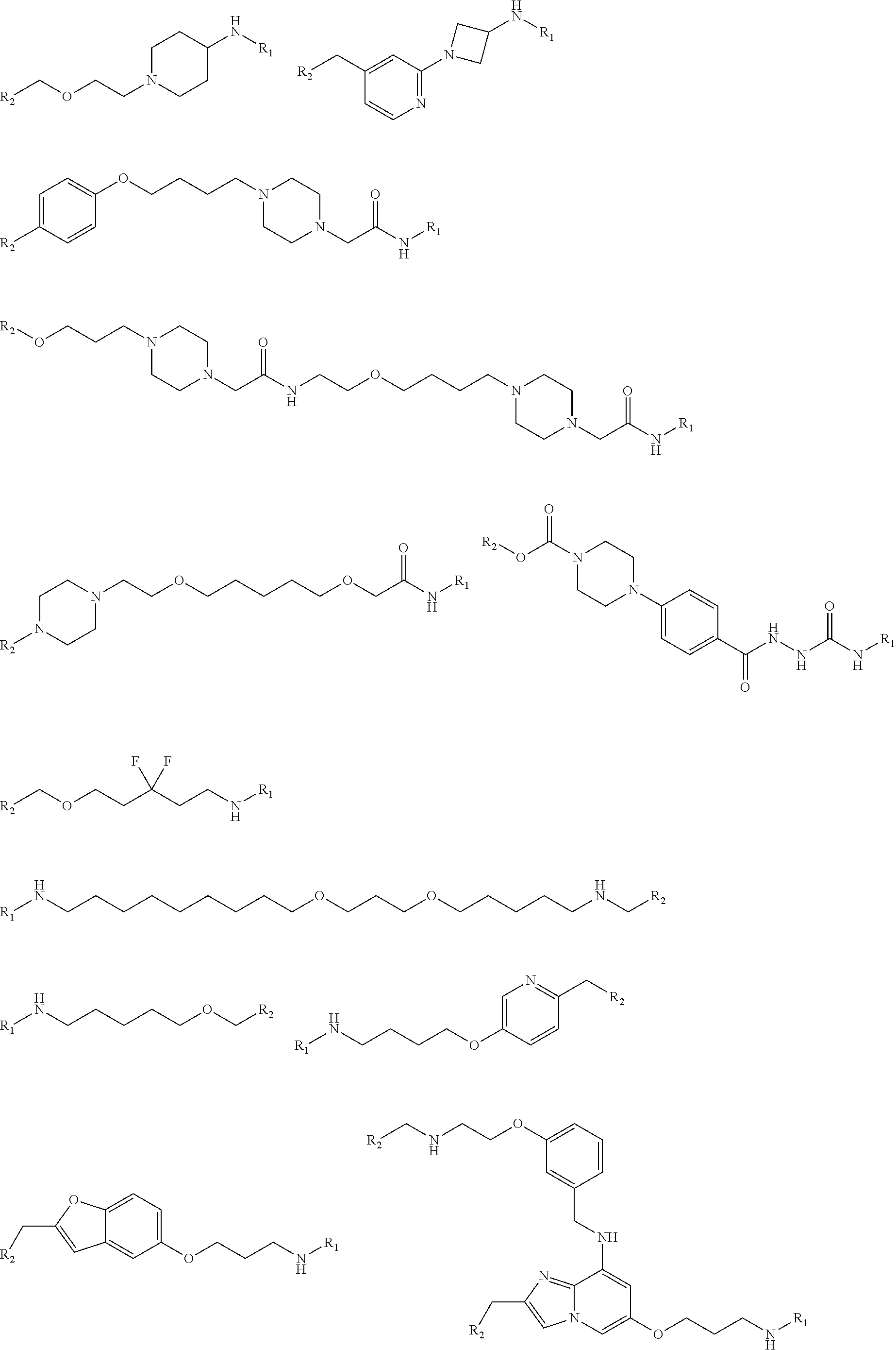

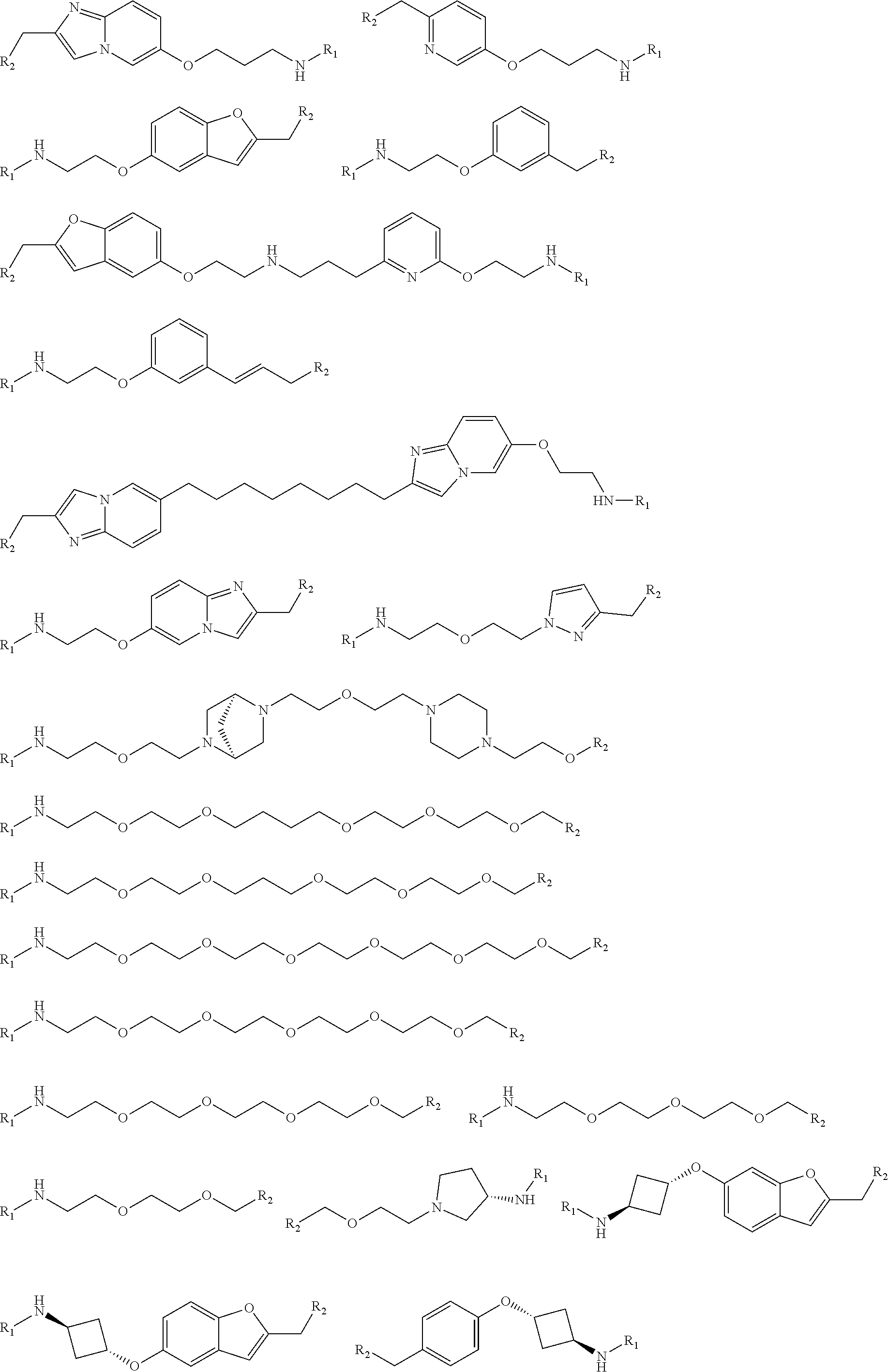

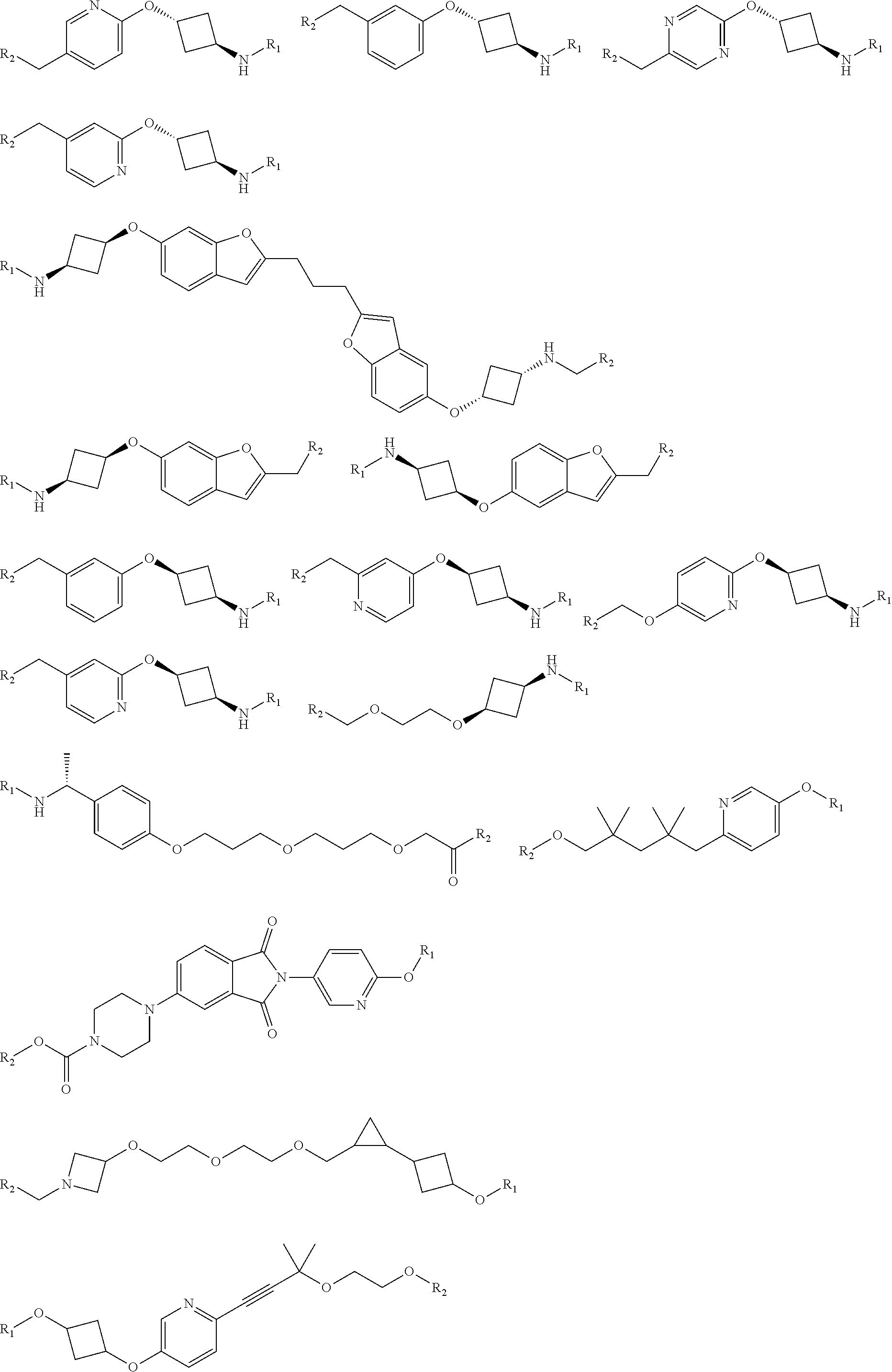

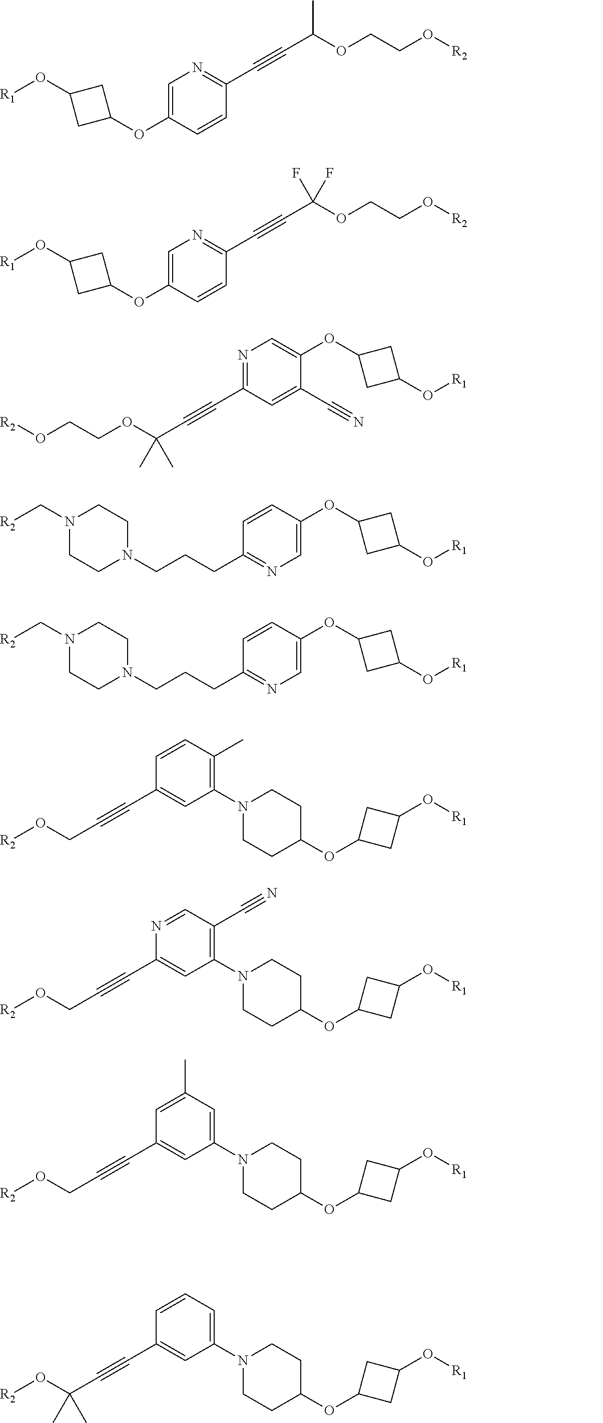

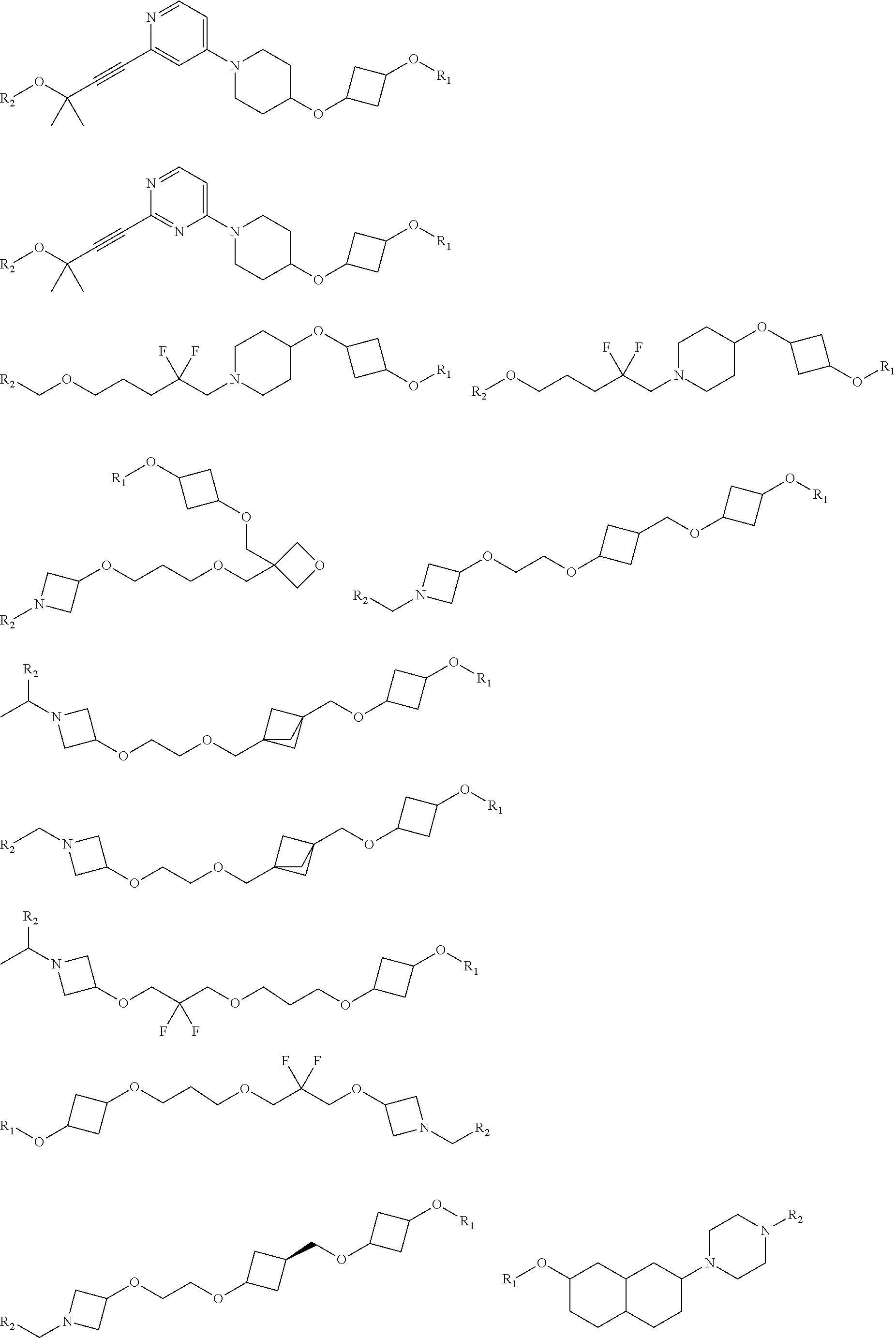

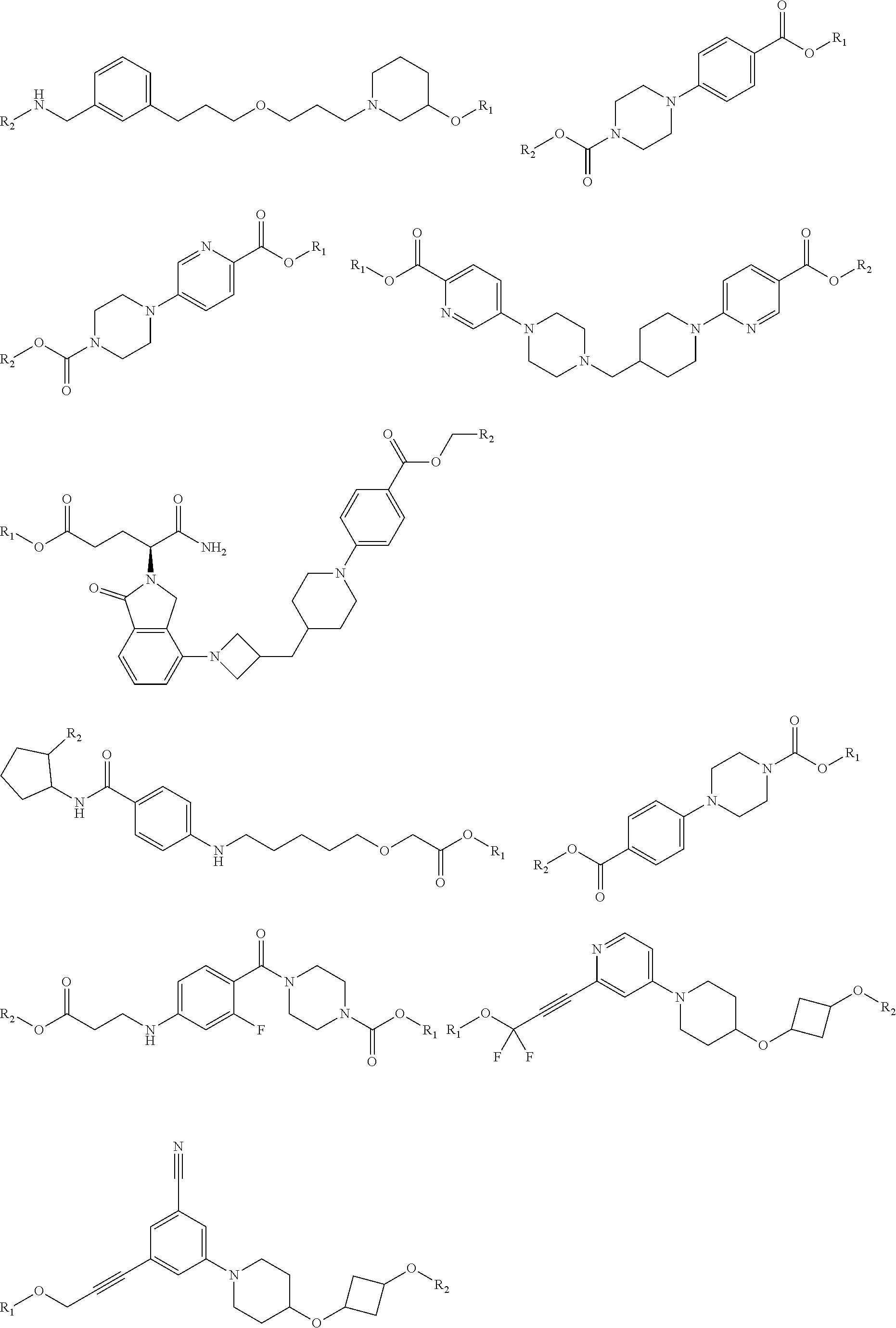

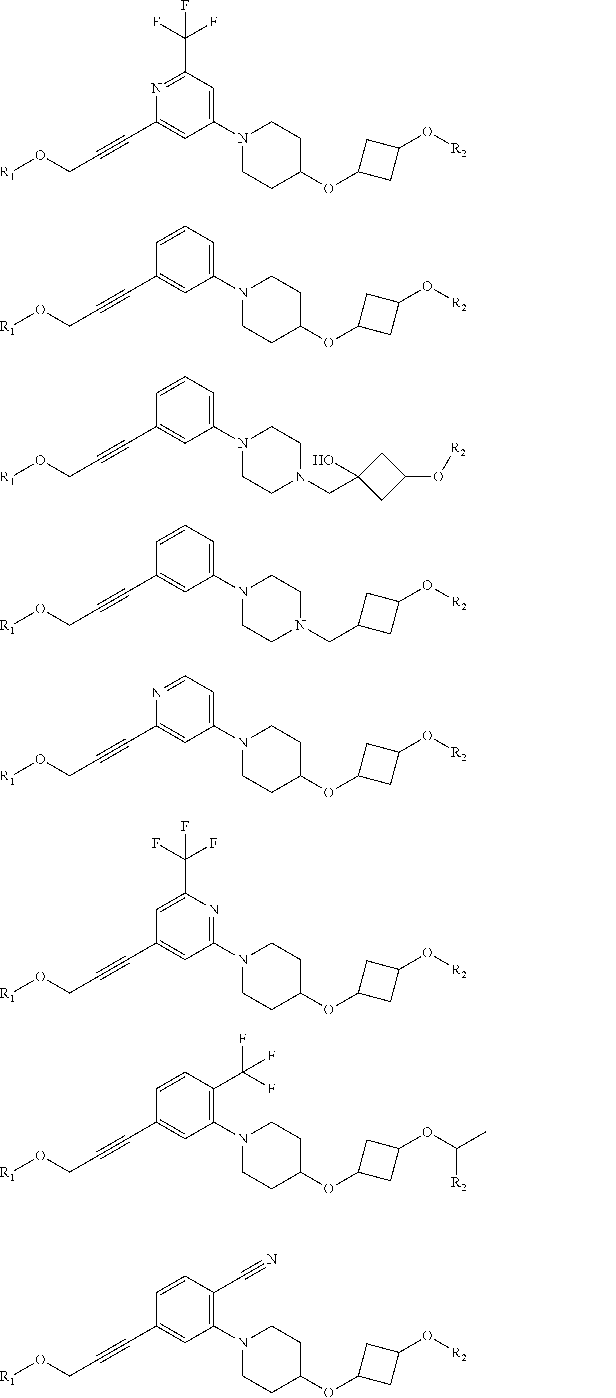

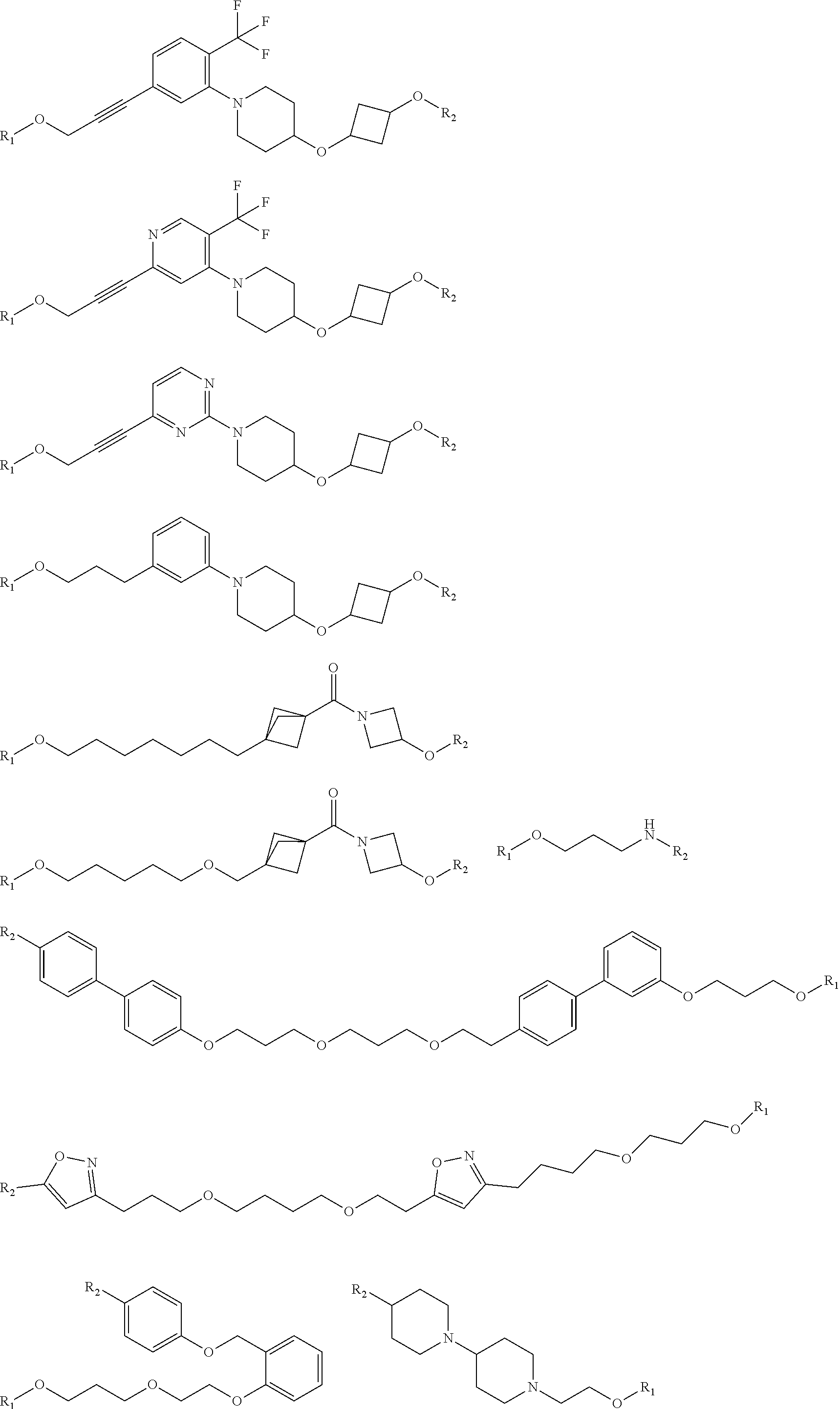

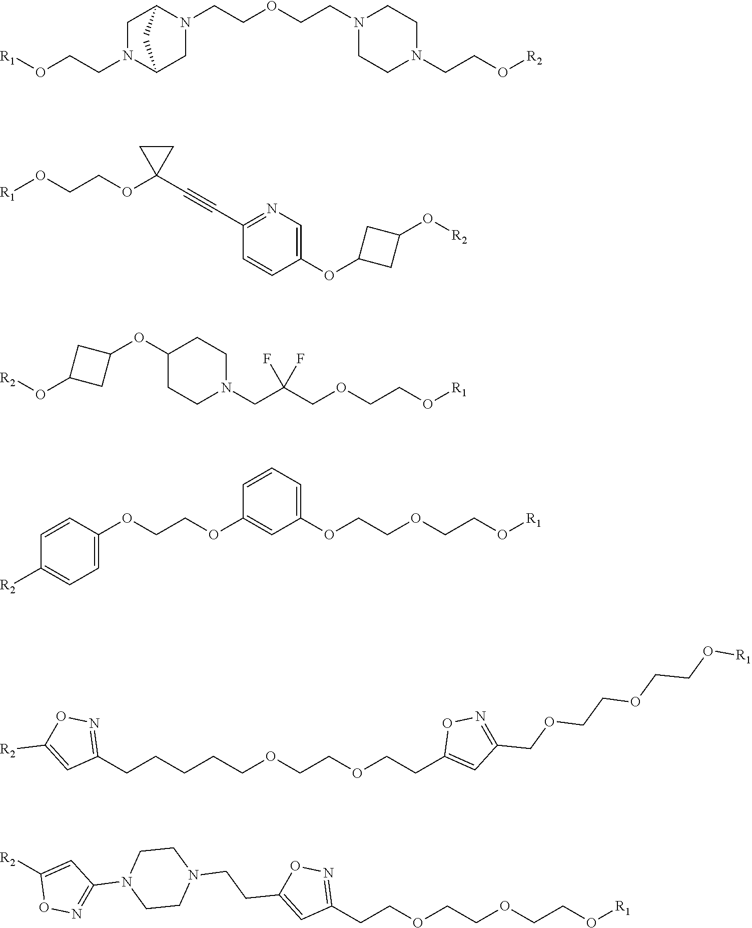

The linker of the bifunctional compound is a critical aspect in designing more effective PROTAC® compounds. The linker influences the pharmacokinetic properties and the pharmacodynamics of a degrader. The linker influences degradation efficiency and has a significant influence on the overall molecular properties of the PROTAC® compound. That is, the linker is paramount in achieving appropriate biological function via the right combination of cellular uptake or permeability, ternary complex geometry, stability, and aqueous solubility. Indeed, appropriate choice of linker and linker attachment sites remains one of the most important aspects of PROTAC® compound design. The linker design, including length and chemical composition, and attachment points of the linker are important design factors. In some embodiments, alkyl, PEG, and extended glycol alkynes, triazoles, saturated heterocycles such as piperazine and piperidine chains may be used as linker motifs. Some advantages to these compositions include their synthetic accessibility, their flexibility, and the ability to easily tune their length and composition via a wide array of robust chemical methods.

While different linkers of different lengths may be used, typically a length between about 1 atom chain length to about 29 atom chain length, including about 5, about 8, about 9, about 10, about 12, about 15, about 16, about 19, about 20, about 21, about 24, about 25, about 27 atom chain length, is used to attach the ligands of the bifunctional molecule. It is recognized that some degradation or inhibition may be achieved with shorter or longer linkers. The linker length may be chosen to maximize degradation efficiency. In general, the linker should be long enough to allow for ternary complex formation, but short enough to discourage “non-ideal” ternary complexes. See, for example, Cyrus et al. (2011) Mol Biosyst 7(2):359-364; Troup et al. (2020) Explor Target Antitumor Ther. 1:273-312, available on the World Wide Web at doi.org/10.37349/etat.2020.00018; Donoghue et al. (2020) European J Med Chem 201(1) 112451; Bemis et al. (2021) J Med Chem 64:8042-8052; all of which are herein incorporated by reference.

A second important function of the linker is establishing binding cooperativity. Cooperativity is defined as the effect that protein association (ex. POI:PROTAC® compound) has on the affinity of the PROTAC® compound to the second protein (the E3 ligase). Mathematically it is defined as (α=KD binary/KD ternary). Positive cooperativity occurs when the KD ternary is smaller than the KD binary. This is often accomplished by the addition of stabilizing interactions between the POI and the E3 ligase. See, for example, Roy et al. (2019) ACS Chem Biol 14(3):361-368, herein incorporated by reference.

Judicious linker design facilitates binding to the target protein and the ubiquitin ligase, influencing degradation efficiency as well as having a significant influence on the overall molecular properties of the PROTAC® compound. The linker structure contributes to achieving appropriate biological function by providing a beneficial combination of cellular uptake, ternary complex geometry, stability, and aqueous solubility. The choice of linker is informed by the E3 ligase ligand, the target-binding ligand, and both the identity and attachment positions of the linker. Linkers as disclosed herein may be tested with ligase targeting moieties and protein targeting moieties in various combinations.

In some embodiments, L comprises one or more ethylene glycol diradical moieties.

### Protein Binding Moieties and Target Proteins

As previously described “protein target moiety” or PTM is used to describe a small molecule that binds to a target protein or other protein or polypeptide of interest and places/presents that protein or polypeptide in proximity to an ubiquitin ligase such that inhibition or degradation of the protein or polypeptide by ubiquitin ligase may occur. Targets of the PTM are numerous in kind and are selected from proteins that are expressed in a cell such that at least a portion of the sequences is found in the cell and may bind to a PTM. As used herein, the term “protein” refers to oligopeptides and polypeptide sequences of sufficient length that they may bind to a PTM. The terms “target protein” and “POI” are used interchangeably in this disclosure. Target proteins which may be bound to the protein target moiety and degraded by the ligase to which the ubiquitin ligase binding moiety is bound include any protein or peptide, including fragments thereof, analogues thereof, and/or homologues thereof. Any protein in a cell may be a target for ubiquitination mediated by the compounds of the invention.

Any protein which may bind to a protein target moiety or PTM group and acted on or degraded by an ubiquitin ligase is a target protein. In general, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catrabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity.

### Mammalian Proteins of Interest and Protein Targeting Moieties (PTMs)

Target proteins which may be bound to the protein target moiety and degraded by the ligase to which the ubiquitin ligase binding moiety is bound include any protein or peptide, including fragments thereof, analogues thereof, and/or homologues thereof. Proteins of interest include those that may be used to restore function in numerous polygenic diseases, including for example B7.1 and B7, TINFRlm, TNFR2, NADPH oxidase, BclIBax and other partners in the apotosis pathway, C₅a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HΓv 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

### Plant Proteins of Interest and Protein Targeting Moieties (PTMs)

The targeted proteolysis approaches of the invention rely on recruiting the POI to an E3 ligase for ubiquitination and subsequent degradation. This target protein recognition is achieved through a POI-specific high-affinity binder or a synthetic small molecule. In one embodiment, the proteins of interest or targeted proteins are selected from proteins that are expressed in a plant cell such that at least a portion of the sequences is found in the cell and may bind to a PTM.

Generally, any protein in a plant may be targeted by the compositions and methods provided herein for ubiquitin. Any protein may be targeted to regulate its level or activity. For example, a target protein may be a protein involved in cell cycle, signal transduction, cell differentiation, cell dedifferentiation, cell growth, production of biological modifiers, regulatory or functional proteins, and the like. Plant protein targets may include herbicide targets including, but not limited to, photosystem II D1 protein, protoporphyrinogen oxidase, glutamine synthetase, hydroxyphenyl pyruvate dioxygenase (HPPD), phytoene desaturase, solanesyl diphosphate synthase, deoxy-D-xyulose phosphate synthase, lycopene cyclase, acetolactate synthase (ALS), Acetyl CoA carboxylase, Very Long Chain Fatty Acid biosynthesis proteins, Fatty Acid Thioesterase, CesA/cellulose biosynthesis proteins, Threonine Protein Phosphatase, Enolpyruvyl Shikimate Phosphate Synthase, Dihydropteroate synthase, tubulin and associated proteins, synthetic auxin targets, Cyclin dependent kinases, lysine tRNA aminoacyl synthetase, Inosine-5′-monophosphate dehydrogenase (IMPDH), AMDPA, Translocase of chloroplast 159 (Toc159), N-myristoyl transferase, and others. In addition, plant protein targets could include transcription factors including, but not limited, toWUS2/wushel, KEG, Topless, JAZ, LEC and the like, for modulating crop performance, drought tolerance, heat tolerance, or other properties.

Protein targeting moieties include but are not limited to: 2,4-D; 2,4-DB; Acetochlor; Acifluorfen; Aclonifen; Alachlor; Ametryn; Amicarbazone; Amidosulfuron; Aminocyclopyrachlor; Aminopyralid; Aminotriazole; Anilofos; Asulam; Atrazine; Azimsulfuron; Beflubutamid; Beflubutamide; Beflubutamid-M; Benazolin; Benfluralin; Benfuresate; Benquitrione; Bensulfuron; Bentazone; Benzobicyclon; Benzofenap; Bicyclopyrone; Bifenox; Bilanafos; Bispyribac; Bixlozone; Bromacil; Bromobutide; Bromoxynil; Butachlor; Butafenacil; Butamifos; Butralin; Butroxydim; Butylate; Cafenstrole; Carbetamide; Carfentrazone; Chloridazon; Chlorimuron; Chlorotoluron; Chlorsulfuron; Chlorthal; Cinidon-ethyl; Cinmethylin; Cinosulfuron; Clethodim; Clodinafop; Clomazone; Clomeprop; Clopyralid; Cloransulam-methyl; Cumyluron; Cyanazine; Cyclopyranil; Cyclopyrimorate; Cyclosulfamuron; Cycloxidim; Cyhalofop-butyl; Daimuron; Desmedipham; Dicamba; Dichlobenil; Dichlorprop; Diclofop; Diclosulam; Difenzoquat; Diflufenican; Diflufenzopyr; Dimesulfazet; Dimethachlor; Dimethametryn; Dimethenamid; Diquat; Dithiopyr; Diuron; Drechslera; Endothal; EPTC; Epyrifenacil; Esprocarb; Ethalfluralin; Ethametsulfuron; Ethofumesate; Ethoxyfen; Ethoxysulfuron; Etobenzanid; Fenoxaprop; Fenoxasulfone; Fenquinotrione; Fentrazamide; Flazasulfuron; Florasulam; Florpyrauxifen; Florpyrauxifen-benzyl; Florpyrauxifen-benzyl; Fluazifop; Flucarbazone; Flucetosulfuron; Flufenacet; Flumetsulam; Flumiclorac-pentyl; Flumioxazin; Fluometuron; Flupoxam; Flupyrsulfuron; Fluridone; Flurochloridone; Fluroxypyr; Flurtamone; Flurtamone; Fluthiacet; Fomesafen; Foramsulfuron; Glufosinate; Glyphosate; Halauxifen; Halosulfuron; Haloxyfop; Hexazinone; Imazamethabenz; Imazamox; Imazapic; Imazapyr; Imazaquin; Imazethapyr; Imazosulfuron; Indanofan; Indaziflam; Iodosulfuron; Ioxynil; Ipfencarbazone; Isoproturon; Isoxaben; Isoxaflutole; Lactofen; Lancotrione-sodium; Lenacil; Linuron; MCPA; MCPB; Mecoprop; Mefenacet; Mesosulfuron; Mesotrione; Metamifop; Metamitron; Metazachlor; Metazosulfuron; Methabenzthiazuron; Metolachlor; Metosulam; Metribuzin; Metsulfuron; Molinate; monoceras; Napropamide; Naptalam; Nicosulfuron; Norflurazon; Orbencarb; Orthosulfamuron; Oryzalin; Oxadiargyl; Oxadiazon; Oxasulfuron; Oxaziclomefone; Oxyfluorfen; Paraquat; Pendimethalin; Penoxsulam; Pentoxazone; Pethoxamid; Phenmedipham; Picloram; Picolinofen; Pinoxaden; Pretilachlor; Primisulfuron; Prodiamine; Profoxydim; Prometryn; Propachlor; Propanil; Propaquizafop; Propoxycarbazone; Propyrisulfuron; Propyzamide; Prosulfocarb; Prosulfuron; Pyraclonil; Pyraflufen-ethyl; Pyrasulfotole; Pyrazolynate; Pyrazosulfuron; Pyrazoxyfen; Pyribenzoxim; Pyributicarb; Pyridate; Pyriftalid; Pyriminobac-methyl; Pyrimisulfan; Pyrithiobac; Pyroxasulfone; Pyroxsulam; Quinclorac; Quinmerac; Quinoclamine; Quizalofop; Quizalofop-P-tefuryl; Rimsulfuron; Saflufenacil; Sethoxydim; Simazine; SL-1201; Sulcotrione; Sulfentrazone; Sulfometuron; Sulfosulfuron; Tebuthiuron; Tefuryltrione; Tembotrione; Tepraloxydim; Terbacil; Terbuthylazine; Terbutryn; Tetflupyrolimet; Thenylchlor; Thiazopyr; Thiencarbazone; Thifensulfuron; Thiobencarb; Tiafenacil; Tolpyralate; Topramezone; Tralkoxydim; Triafamone; Triallate; Triasulfuron; Triaziflam; Tribenuron; Triclopyr; Trifloxysulfuron; Trifludimoxazin; Trifluralin; Triflusulfuron; and Tritosulfuron. Derivatives and analogs of such PTMs may also be employed. Such moieties may be functionalized (e.g., modified to contain a reactive group such as a carboxylate, alcohol, or amine) for bonding to a linker moiety or a ligase targeting moiety as described in more detail below.

In some embodiments, the protein targeting moiety comprises a tembotrione moiety, a penoxsulam moiety, a tralkoxydim moiety, a fluazifop moiety, or a fenoxaprop moiety. That is, in some embodiments, the protein targeting moiety comprises a 2-{2-chloro-4-(methylsulfonyl)-3-[(2,2,2-trifluoroethoxy)methyl]benzoyl}-1,3-cyclohexanedione moiety, a 2-(2,2-difluoroethoxy)-N-(5,8-dimethoxy[1,2,4]triazolo[1,5-c]pyrimidin-2-yl)-6-(trifluoromethyl)benzenesulfonamide moiety, a 2-[(1E)-N-ethoxypropanimidoyl]-3-hydroxy-5-mesityl-2-cyclohexen-1-one moiety, a butyl (2R)-2-(4-{[5-(trifluoromethyl)-2-pyridinyl]oxy}phenoxy)propanoate moiety, or a 2-{4-[(6-chloro-1,3-benzoxazol-2-yl)oxy]phenoxy}propanoic acid moiety.

In some embodiments, the protein targeting moiety is selected from the group consisting of:
a halo-(2,6-dioxocyclohexane-carbonyl)phenyl moiety,
an N-(5,8-dimethoxy-[1,2,4]triazolo[1,5-c]pyrimidin-2-yl)-6-(trifluoromethyl)benz-2-yl-sulfonamide moiety,
a 1-oxo-2-(1-(ethoxyimino)propyl)-3-hydroxy-cyclohex-2-en-5-yl moiety,
a 6-((4-oxy)phenoxy)pyridin-3-yl moiety, and
a 4-(oxy)phenoxy)acetyl moiety,
   each of which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

In some embodiments, the PROTAC® compound is a compound according to Formula I:

PTM-L-LTM (I),
or a salt or hydrate thereof, wherein:
PTM is selected from the group consisting of:
a halo-(2,6-dioxocyclohexane-carbonyl)phenyl moiety,
an N-(5,8-dimethoxy-[1,2,4]triazolo[1,5-c]pyrimidin-2-yl)-6-(trifluoromethyl)-benz-2-yl-sulfonamide moiety,
a 1-oxo-2-(1-(ethoxyimino)propyl)-3-hydroxy-cyclohex-2-en-5-yl moiety,
a 6-((4-oxy)phenoxy)pyridin-3-yl moiety, and
a 4-(oxy)phenoxy)acetyl moiety,
each of which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy;
L comprises one or more ethylene glycol diradical moieties, one or more of which is optionally replaced by a moiety independently selected from:

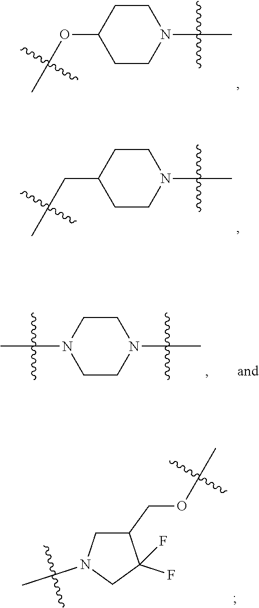 [SMILES: *OC1CCN(*)CC1 ; *CC1CCN(*)CC1 ; *N1CCN(*)CC1 ; *OCC1CN(*)CC1(F)F]

and
LTM is an N-substituted 1,3-dioxoisoindolinyl moiety, which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

In some embodiments, PTM has a structure according to the formula:

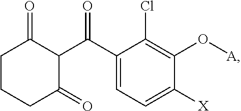

wherein A is the site of attachment to linker moiety L; and
X is H or SO₂Me.

In some embodiments, PTM has a structure according to the formula:

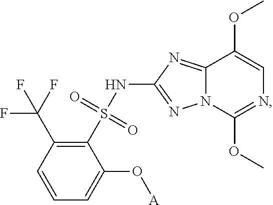

wherein A is the site of attachment to linker moiety L.

In some embodiments, PTM has a structure according to the formula:

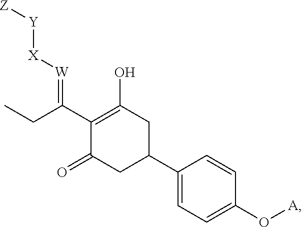

wherein:
A is the site of attachment to linker moiety L; and
W is N, X is O, Y is CH₂ and Z is CH₃; or
W is O, and X, Y and Z are not present.

In some embodiments, PTM has a structure according to the formula:

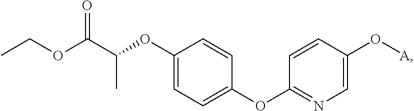

wherein A is the site of attachment to linker moiety L.

In some embodiments, PTM has a structure according to the formula:

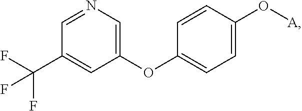

wherein A is the site of attachment to linker moiety L.

In some embodiments, PTM has a structure according to the formula:

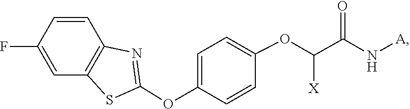

wherein:
A is the site of attachment to linker moiety L; and
X is H or CH₃.

In some embodiments, LTM has a structure according to the formula:

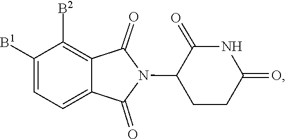

wherein:
B¹ is the site of attachment to linker moiety L and B² is H; or
B¹ is H and B² is the site of attachment to linker moiety L.

As used herein, the term “salt” refers to an acid salt or base salt of an active agent such as a PROTAC® compound. Acid salts of basic active agents include mineral acid salts (e.g., salts formed using hydrochloric acid, hydrobromic acid, phosphoric acid, and the like), and organic acid salts (e.g., salts formed using acetic acid, propionic acid, glutamic acid, citric acid, and the like). Quaternary ammonium salts may be formed using reagents such as methyl iodide, ethyl iodide, and the like.

Acidic active agents may be contacted with bases to provide base salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The neutral forms of the active agents may be regenerated by contacting the salt with a base or acid and isolating the parent compound in the conventional manner if desired. In some embodiments, the parent form of the compound may differ from various salt forms in certain physical properties, such as solubility in polar solvents, but otherwise the salt forms may be equivalent to the parent form of the compound.

The activity of the PROTAC® compounds, and the effects of PTM, L, and LTM structures (individually or in particular combinations) on compound activity, may be assessed using various assays as described, for example, in Paiva and Crews (2019) Curr Opin Chem Biol 50:111-119; Zoppi et al. (2019) J Med Chem 62:699-726; Potjewyd et al. (2020) Cell Chem Biol 27:47-56; Troup et al (2020) Explor Target Antitumor Ther. 1:273-312; Bondeson et al (2018) Cell Chem Biol 25:78-87; and the like. Each of these references is herein incorporated by reference. As a non-limiting example, degradation of a target protein in plant cells (e.g., protoplasts such as Arabidopsis protoplasts) may be assessed via immunoblotting or other immunoassay following treatment with a test compound.

Other protein binding ligands (e.g., orthosteric, allosteric, dualsteric) may be used. DNA-encoded libraries may be used to pan for molecules that bind to a protein. In this manner, a multitude of compounds may be tagged with DNA bar codes and put through a binding assay. Compounds tagged with DNA bar codes may be sequenced to reveal the structure of any hits. Mammalian proteins of interest and protein binding moieties.

Non-limiting examples of small molecule target protein binding moieties include compounds targeting BRD4, estrogen-related receptor alpha (ERRα) binders, RIPK2 binders, AR binders, Hsp90 inhibitors, kinase inhibitors, MDM2 inhibitors, HDAC inhibitors, human lysine methyltransferase inhibitors, angiogenesis inhibitors, immunosuppressive compounds, and compounds targeting the aryl hydrocarbon receptor (AHR), among numerous others. Other drug targets for human therapeutics and disease states of conditions which may be treated are set forth in U.S. Pat. No. 10,772,962, herein incorporated by reference.

### Synthetic Methods

Also provided herein are processes for preparing the compounds of Formula II, as well as salts and solvates thereof. Exemplary processes include those summarized below in Scheme 1. A functionalized starting material such as a phenol or aniline may be reacted, for example, with a suitable alkyl halide, alkyl sulphonate, acyl halide, acid anhydride, silyl halide, or sulphonyl halide to provide compounds of type (A-1) containing a protected nucleophile —W—(PG).

Compounds of type (A-2) are, in individual cases, directly accessible from intermediates of type (A-1), e.g., when Q is a methyl and T is an ester, by reacting (A-1) directly with a suitable halogenating agent such as NBS, wherein, if appropriate, a radical initiating catalyst is included, followed directly by reaction with (S)-tert-butyl 4,5-diamino-5-oxopentanoate in the presence of a suitable base such as DIPEA or TEA in a solvent such as acetonitrile. Such reactions are typically conducted in a solvent commonly used in organic chemistry, for example carbon tetrachloride, toluene, or other solvents, and at elevated temperature, for example between 50° C. and 200° C.

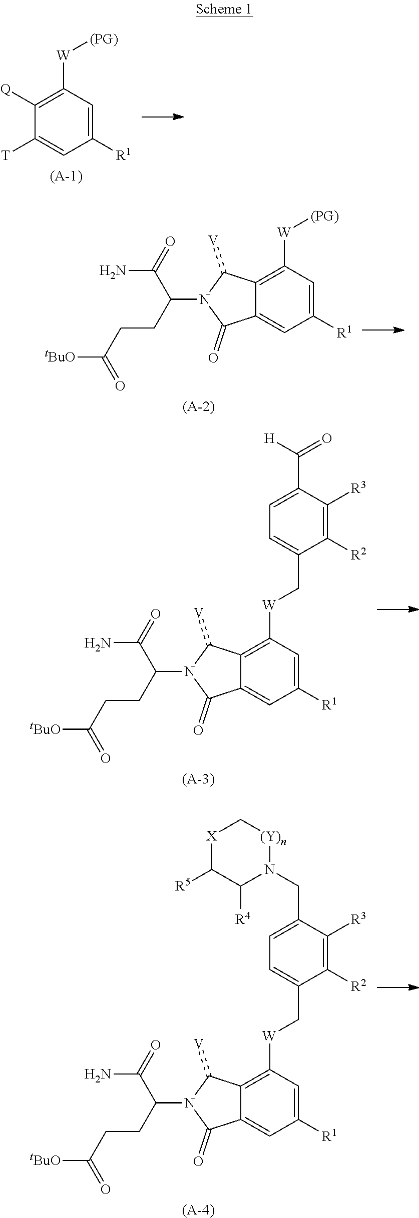

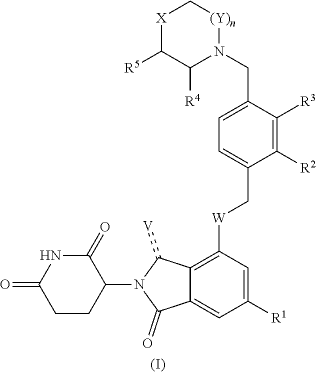

Compounds of type (A-2) are, in individual cases, also directly accessible from compounds of type (A-1), e.g., when Q and T are both acids or esters, by reacting (A-1) with (S)-tert-butyl 4,5-diamino-5-oxopentanoate in the presence of a suitable base such as DIPEA or TEA in a solvent such as acetonitrile.

The conditions for the conversion of structure type (A-2) to structure type (A-3) depend, in part, on the nature of the protecting group (PG) to be removed. Generally used in this connection and preferred in this context, however, are reagents such as TBAF in MeOH followed by formation of the ether bond between the appropriate benzyl halide and the unprotected hydroxyl in the presence of a base, for example K₂CO₃, CS₂CO₃, or other base as required in a solvent such a DMF. Compounds of type (A-3) may be reacted with an amine in the presence of a reducing agent such as borane-2-methylpyridine complex or triacetoxyborohydride, wherein, if appropriate, acetic acid is required resulting in compounds of the type (A-4). Such reactions are usually conducted in a solvent commonly used, for example carbon THF, MeOH or other solvents. The synthesis of the product according to Formula II is effected via the treatment of (A-4) with an acid (e.g., para-toluenesulfonic acid) in a solvent (e.g., methanol) at elevated temperature, for example between 50° C. and 200° C.

The starting materials and reagents used in preparing the compounds of the present disclosure are either available from commercial suppliers or are prepared by methods known to those skilled in the art following procedures set forth in references such as Fieser and Fieser's Reagents for Organic Synthesis, Vol. 1-28 (Wiley, 2016); March's Advanced Organic Chemistry, 7^{th} Ed. (Wiley, 2013); and Larock's Comprehensive Organic Transformations, 2^{nd} Ed. (Wiley, 1999). The starting materials and the intermediates of the reaction may be isolated and purified if desired using conventional techniques including, but not limited to, filtration, distillation, crystallization, chromatography, and the like. Such materials may be characterized using conventional means, including measuring physical constants and obtaining spectral data.

Those skilled in the art will recognize if a stereocenter exists in the compounds disclosed herein. Accordingly, the present disclosure encompasses not only racemic compounds but also individual enantiomers and/or diastereomers. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

### Compositions

The compounds described herein, or salts thereof, may be provided in a formulation or composition. The compositions may include other active ingredients and/or plant or plant product treatment compounds. In general, a composition will include a PROTAC® or other active agent described herein and one or more acceptable adjuvants or suitable agricultural carriers or excipients. In some embodiments, the PROTAC® is present in a concentration of from 0.001 to 98.0 percent by weight, with the remaining content being one or more acceptable carriers. Such compositions, especially those containing less than 50 percent of the PROTAC® by weight, may sometimes be used directly, but these compositions may also be diluted with additional acceptable carriers to form more dilute treating compositions. These diluted compositions may include the PROTAC® or other active agent in lesser concentrations of from 0.001 to 0.1 percent.

Compositions are generally prepared according to procedures and formulas which are conventional in the agricultural art. PROTAC® compounds and other active agents may be dispersed in water, an aqueous solvent mixture, or a non-aqueous solvent. The dispersions are typically aqueous suspensions or emulsions prepared from concentrated compositions of the compounds. The water-soluble or water-suspendable or emulsifiable compositions are either solids, wettable powders, or liquids, known as emulsifiable concentrates or aqueous suspensions. Wettable powders may be agglomerated or compacted to form water dispersible granules. These granules contain mixtures of compounds, inert carriers and surfactants. The concentration of the compound is typically between about 0.1% to about 90% by weight. The inert carrier is typically attapulgite clays, montmorillonite clays, and diatomaceous earths or purified silicates. Other excipients including, for example, adhesives, wetters, dispersants, emulsifiers, preservatives, antifreeze agents, fillers, colorants, carriers, antifoams, evaporation inhibitors, pH modifiers, or viscosity modifiers may also be included in the compositions.

The bifunctional compounds of the invention may be used in combination with other agents such as herbicides, insecticides, fungicides, or other agricultural chemical or biological agents. In some embodiments, the combination of the compounds of the invention with other agents may show synergistic activity, where the combination of the two exceeds that expected from their simple additive effect. The combinations may be used to significantly reduce disease, to promote plant growth and yield, and to reduce the amount of traditional chemical used.

The bifunctional compounds of the invention and other agents may be present in the same mixture or composition or may be applied separately. When applied separately, the agents and bifunctional compounds may be applied concurrently (at the same time), or sequentially.

### Methods of Use

### A. Application in Plants

The compounds described herein may modulate the level of a protein of interest in a plant cell. The compounds and compositions may be provided in agricultural compositions in effective amounts to modulate the level of the target protein. The compounds and compositions described herein generally exhibit one or more advantages including, but not limited to, increased potency; reduced risk to non-target species; lower potential for environmental damage; and minimal cross-resistance to pesticides and herbicides. The compounds and compositions may be targeted to any plant protein of interest.

Methods may include providing a composition that has an effective amount of at least one compound as described herein and at least one of a solvent or an acceptable carrier and administering an effective amount of the composition to plants. Where the composition is a liquid, the method may further include administering an effective amount of the composition such that an effective amount of the composition contacts plants and plant products. Where the composition is a dust or a solid, administering an effective amount of the composition may include placing an effective amount of the composition in a vicinity of plants and plant products to be protected. The compounds and compositions described herein may be applied to the foliage of a plant.

An effective amount of the compound or composition as described herein is an amount to reduce the level of the target protein (e.g., by at least 1%, 5%, 10%, 20%, 30%, 40%, 50%, up to 70%, 80%, 90% including up to 100%. The effective amount may depend on factors including, but not limited to, the particular host crop, the age or condition of the crop, the protein of interest and/or the ubiquitin ligase to be targeted, and climate conditions where the compound or composition is employed.

The compounds described herein, or salts thereof, may be provided in a formulation or composition. The compositions may include other active ingredients and/or plant or plant product treatment compounds. In general, a composition will include a compound of Formula I or II or other active agent described herein and one or more acceptable adjuvants or suitable agricultural carriers. By “acceptable,” it is meant that the adjuvant or carrier is compatible with the other ingredients of the formulation and is not deleterious to the environment or organism (e.g., plant) to which it is applied. In some embodiments, the compound of Formula I or II is present in a concentration of from 0.001 to 98.0 percent by weight, with the remaining content being one or more acceptable carriers. Such compositions, especially those containing less than 50 percent of the compound of Formula I or II by weight, may sometimes be used directly, but these compositions may also be diluted with additional acceptable carriers to form more dilute treating compositions. These diluted compositions may include the compound of Formula I or II or other active agent in lesser concentrations of from 0.001 to 0.1 percent.

Compositions are generally prepared according to procedures and formulas which are conventional in the agricultural art. Compounds of Formula I or II and other active agents may be dispersed in water, an aqueous solvent mixture, or a non-aqueous solvent. The dispersions are typically aqueous suspensions or emulsions prepared from concentrated compositions of the compounds. The water-soluble or water-suspendable or emulsifiable compositions are either solids, wettable powders, or liquids, known as emulsifiable concentrates or aqueous suspensions. Wettable powders may be agglomerated or compacted to form water dispersible granules. These granules contain mixtures of compounds, inert carriers and surfactants. The concentration of the compound is typically between about 0.1% to about 90% by weight. The inert carrier is typically attapulgite clays, montmorillonite clays, and diatomaceous earths or purified silicates. Other excipients including, for example, adhesives, wetters, dispersants, emulsifiers, preservatives, antifreeze agents, fillers, colorants, carriers, antifoams, evaporation inhibitors, pH modifiers, or viscosity modifiers may also be included in the compositions.

### B. Applications in Mammals

In some embodiments, methods for treating or ameliorating a disease, disorder, or symptom thereof in a subject or a patient, e.g., an animal such as a human, are provided. The methods include administering to a subject in need thereof a therapeutically effective amount of a compound, salt, or solvate as described herein. The active agent (e.g., a compound according to Formula I or II) may be administered as a pharmaceutical composition comprising a therapeutically effective amount of a compound as described herein, or a pharmaceutically acceptable salt or solvate thereof, and a pharmaceutically acceptable carrier. Thus, the pharmaceutical composition may be used to treat a disease or condition which is modulated by a target protein.

In some embodiments, methods for treating cell proliferative disorders (e.g., a cancer modulated by a targeted protein), are provided. The methods may be employed for the treatment of neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, other rapidly dividing cells, or the like.

As used herein, the term “cancer” includes, but is not limited to, oral cancers (including those of buccal cavity, lip, tongue, mouth, and pharynx); cardiac cancers (e.g., sarcomas such as angiosarcoma, fibrosarcoma, rhabdomyosarcoma, liposarcoma, as well as myxoma, rhabdomyoma, fibroma, lipoma, and teratoma); lung cancers (e.g., bronchogenic carcinomas (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, and mesothelioma); gastrointestinal cancers (including those of the esophagus (e.g., squamous cell carcinoma, adenocarcinoma, leiomyosarcoma, lymphoma), larynx, stomach (e.g., carcinoma, lymphoma, leiomyosarcoma), pancreas (e.g., ductal adenocarcinoma, insulinoma, glucagonoma, gastrinoma, carcinoid tumors, vipoma), small bowel or small intestines (e.g., adenocarcinoma, lymphoma, carcinoid tumors, Karposi's sarcoma, leiomyoma, hemangioma, lipoma, neurofibroma, fibroma), large bowel or large intestines (e.g., adenocarcinoma, tubular adenoma, villous adenoma, hamartoma, leiomyoma), colon, colon-rectum, and rectum); genitourinary tract cancers (including those of kidney (e.g., adenocarcinoma, Wilm's tumor (nephroblastoma), lymphoma, leukemia), bladder and urethra (e.g., squamous cell carcinoma, transitional cell carcinoma, adenocarcinoma), prostate (e.g., adenocarcinoma, sarcoma), and testis (e.g., seminoma, teratoma, embryonal carcinoma, teratocarcinoma, choriocarcinoma, sarcoma, interstitial cell carcinoma, fibroma, fibroadenoma, adenomatoid tumors, lipoma); liver cancers (e.g., hepatoma (hepatocellular carcinoma), cholangiocarcinoma, hepatoblastoma, angiosarcoma, hepatocellular adenoma, and hemangioma); bone cancers (e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors); nervous system cancers (including those of the skull (e.g., osteoma, hemangioma, granuloma, xanthoma, osteitis deformans), meninges (e.g., meningioma, meningiosarcoma, gliomatosis), brain (e.g., astrocytoma, medulloblastoma, glioma, ependymoma, germinoma (pinealoma), glioblastoma multiform, oligodendroglioma, schwannoma, retinoblastoma, congenital tumors), and spinal cord (e.g., neurofibroma, meningioma, glioma, sarcoma); gynecological cancers (including those of the uterus (e.g., endometrial carcinoma), cervix (e.g., cervical carcinoma, pre-tumor cervical dysplasia), ovaries (e.g., ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (e.g., squamous cell carcinoma, intraepithelial carcinoma, adenocarcinoma, fibrosarcoma, melanoma), vagina (e.g., clear cell carcinoma, squamous cell carcinoma, botryoid sarcoma (embryonal rhabdomyosarcoma)), and fallopian tubes); breast cancers; hematologic cancers (including those of the blood (e.g., myeloid leukemia (acute and chronic), acute lymphoblastic leukemia, chronic lymphocytic leukemia, myeloproliferative diseases, multiple myeloma, myelodysplastic syndrome, Hodgkin lymphoma, non-Hodgkin's lymphoma (including malignant lymphoma), and hairy cell leukemia); lymphoid cancers; skin cancers (e.g., malignant melanoma, basal cell carcinoma, squamous cell carcinoma, Karposi's sarcoma); cancers of the thyroid gland (e.g., papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma, paraganglioma); and cancers of the adrenal glands (e.g., neuroblastoma). The term “cancerous cell,” as used herein, includes a cell afflicted by any one of the above-identified conditions.

For use in mammalian/human therapy, the target protein may be any one of various proteins pertinent to the integrated function of a cell including, but not limited to, a structural protein, a receptor, an enzyme, a cell surface protein (e.g., a cell adhesion protein), a cell cycle regulator protein (including those involved in cell death), or a transport protein. Non-limiting examples of small molecule protein targeting moieties include Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among numerous others.

The term “subject” as used herein typically refers to a mammal, e.g., humans, dogs, cats, horses, cows, pigs, guinea pigs, and the like. The subject may be also be referred to as a patient, particularly when the subject is a human. “Treating” and “treatment” refer to the management and care of a subject for the purpose of combating a disease, condition, or disorder and includes the administration of an active agent (e.g., a compound of Formula I or II as described herein), to alleviate the symptoms or complications of a disease, condition or disorder, or to eliminate the disease, condition or disorder. The treatment or amelioration of symptoms may be based on any objective or subjective parameter; including, e.g., the result of a physical examination or laboratory test.

The term “therapeutically effective amount” of a compound or pharmaceutical composition, as used herein, refers to a sufficient amount of the compound or pharmaceutical composition so as to decrease the symptoms of a disorder in a subject. A therapeutically effective amount of a compound or composition may involve the assessment of a reasonable benefit/risk ratio, as typical of many medical treatments. It will be understood, however, that the total amount of a compound or composition within a given time period (e.g., a typical day of treatment) will be decided within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Compounds as described herein may be administered by any conventional route. Pharmaceutical compositions appropriate for various routes of administration are described, for example, in U.S. Pat. No. 10,239,888, which is incorporated herein by reference in its entirety. The compositions may be formulated, e.g., for oral administration, intravenous administration, intramuscular administration, intraperitoneal administration, subcutaneous administration, intrathecal administration, intraarterial administration, nasal administration, or rectal administration.

The pharmaceutical compositions may be prepared by any of the methods well known in the art of pharmacy and drug delivery. In general, preparation of the compositions includes the step of bringing the active ingredient(s) into association with a carrier containing one or more accessory ingredients. The pharmaceutical compositions are typically prepared by uniformly and intimately bringing the active ingredients into association with a liquid carrier or a finely divided solid carrier or both, and then, if necessary, shaping the product into the desired formulation. The compositions may be conveniently prepared and/or packaged in unit dosage form.

The pharmaceutical compositions may be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Such compositions may contain one or more agents selected from sweetening agents, flavoring agents, coloring agents, antioxidants, and preserving agents in order to provide pharmaceutically elegant and palatable preparations.

Tablets generally contain the active ingredients in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets may be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate may be employed. Tablets may also be coated with a semi-permeable membrane and optional polymeric osmogents according to known techniques to form osmotic pump compositions for controlled release. Compositions for oral administration may be formulated as hard gelatin capsules wherein the active ingredient is mixed with an inert solid diluent (such as calcium carbonate, calcium phosphate, or kaolin), or as soft gelatin capsules wherein the active ingredients are mixed with water or an oil medium (such as peanut oil, liquid paraffin, or olive oil).

The pharmaceutical compositions may also be in the form of an injectable aqueous or oleaginous solution or suspension. Sterile injectable preparations may be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Aqueous suspensions contain the active agents in admixture with excipients suitable for the manufacture of aqueous suspensions. Such excipients include, but are not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example beeswax, hard paraffin, or cetyl alcohol. These compositions may be preserved by the addition of an anti-oxidant such as ascorbic acid. Dispersible powders and granules (suitable for preparation of an aqueous suspension by the addition of water) may contain the active ingredients in admixture with a dispersing agent, wetting agent, suspending agent, or combinations thereof.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Transdermal delivery may be accomplished by means of iontophoretic patches and the like. The active ingredients may also be administered in the form of suppositories for rectal administration of the drug. These compositions may be prepared by mixing the active agents with a suitable non-irritating excipient which is solid at ordinary temperatures but liquid at the rectal temperature and will therefore melt in the rectum to release the drug. Such materials include cocoa butter and polyethylene glycols.

While the invention has generally been discussed in terms of PROTAC® compounds it is recognized that other bifunctional molecules may be useful in the practice of the invention. While not bound by any mechanism of action, it is believed that the bifunctional molecules degrade the POIs by directing them to the proteosome. However, it is recognized that degradation may occur through the lysosome or other degradation pathway.

The PTM are designed to bind to a particular protein expressed in a plant cell. It is recognized that the moieties may in some instances also bind to homologs, derivatives, or similar protein structures. The bifunctional molecules are designed to direct the targeted proteins for degradation. However, in some instances the molecules inhibit the activity of the targeted proteins.

The studies described herein represent the first demonstration in plants of uptake and function of bifunctional compounds that target a ligase and a protein of interest. The transition from mammalian systems to plants has been recognized as often difficult and not straightforward. This difficulty is especially true for protein modulation. Now that it has been demonstrated that PROTAC® compounds are capable of entry and function in a plant cell, other chimeric molecules to control cellular function in plants may be used including, but not limited to, phosphatase recruiting chimeras (PhoRCs) that bring a phosphatase close enough to a target receptor to cause dephosphorylation or to induce phosphorylation by bringing kinases close to targets of interest (Yamazoe et al. (2020) J Med Chem 63(6):2807-2813); deubiquitinase-targeting chimeras (DUBTAC) to stop the degradation of target proteins (Henning et al. (2021) bioRxiv preprint found at doi.org/10.101/2021.04.30.441959; published as1 Henning el al. (2022) Nat. Chem. Biol. 18:412-421); ribonuclease targeting chimeras (RIBOTACs) to trigger nuclease-mediated degradation of specific RNAs (Haniff et al. (2020) ACS Cent Sci 6(10):1713-1721); autophagy-targeting chimeras (AUTACs) to trigger lysine residue 63 ubiquitination inducing degradation by the autophagy pathway (US 2012/0258975, US 2015/0166472, US 2018/0002381, US 20200163970, and U.S. Pat. Nos. 10,618,939, 9,388,626); autophagosome-tethering compounds (ATTECs) that exploit the autophagy pathway by recruiting a key autophagosome protein called LC3 (US 2016/0143994, U.S. Pat. No. 9,545,433); and lysosome-targeting chimaeras (LYTACs) that target extracellular and/or membrane protein to induce degradation by harnessing the endosome/lysosome pathway (Lin et al. (2021) Theranostics 11(17): 8337-8349). All of these references are herein incorporated in their entirety by reference.

### EXEMPLARY EMBODIMENTS

The following embodiments are contemplated. All combinations of features and embodiments are contemplated.

Embodiment 1: A method for controlling the level of a target protein in a plant cell, the method comprising contacting the plant cell with an effective amount of a compound according to Formula I:

PTM-L-LTM (I),

or a salt or hydrate thereof, wherein: PTM is a targeting moiety that binds the target protein; L is a covalent bond or linker moiety; and LTM is a ubiquitin ligase binding moiety that binds a plant ubiquitin ligase.

Embodiment 2: The method of embodiment 1, wherein controlling the level of the target protein comprises targeted degradation of the target protein.

Embodiment 3: The method of embodiment 1 or embodiment 2, wherein the target protein is selected from the group consisting of hydroxyphenylpyruvate dioxygenase, acetolactate synthase, and acetyl CoA carboxylase.

Embodiment 4: The method of any one of embodiments 1-3, wherein the plant ubiquitin ligase is a plant cereblon.

Embodiment 5: The method of any one of embodiments 1-4, wherein PTM is selected from the group consisting of: a halo-(2,6-dioxocyclohexane-carbonyl)phenyl moiety, an N-(5,8-dimethoxy-[1,2,4]triazolo[1,5-c]pyrimidin-2-yl)-6-(trifluoromethyl)benz-2-yl-sulfonamide moiety, a 1-oxo-2-(1-(ethoxyimino)propyl)-3-hydroxy-cyclohex-2-en-5-yl moiety, a 6-((4-oxy)phenoxy)pyridin-3-yl moiety, and a 4-(oxy)phenoxy)acetyl moiety, each of which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

Embodiment 6: The method of any one of embodiments 1-5, wherein L comprises one or more ethylene glycol diradical moieties, one or more of which is optionally replaced by a moiety independently selected from:

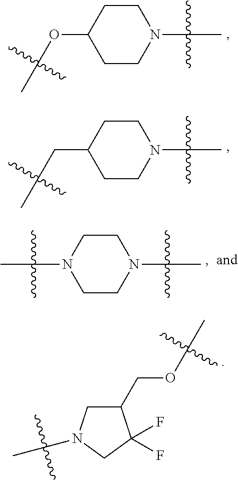 [SMILES: *OC1CCN(*)CC1 ; *CC1CCN(*)CC1 ; *N1CCN(*)CC1 ; *OCC1CN(*)CC1(F)F]

Embodiment 7: The method of any one of embodiments 1-6, wherein LTM is an N-substituted 1,3-dioxoisoindolinyl moiety, which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

Embodiment 8: The method of any one of embodiments 1-7, wherein the compound of Formula I is formulated as a composition comprising or more agriculturally acceptable excipients.

Embodiment 9: A compound according to Formula I:

PTM-L-LTM (I),

or a salt or hydrate thereof, wherein: PTM is a targeting moiety that binds with a protein in a plant cell; L is a covalent bond or linker moiety; and LTM is a ubiquitin ligase binding moiety that binds a plant ubiquitin ligase.

Embodiment 10: The compound of embodiment 9, or a salt or hydrate thereof, wherein PTM is selected from the group consisting of: a halo-(2,6-dioxocyclohexane-carbonyl)phenyl moiety, an N-(5,8-dimethoxy-[1,2,4]triazolo[1,5-c]pyrimidin-2-yl)-6-(trifluoromethyl)benz-2-yl-sulfonamide moiety, a 1-oxo-2-(1-(ethoxyimino)propyl)-3-hydroxy-cyclohex-2-en-5-yl moiety, a 6-((4-oxy)phenoxy)pyridin-3-yl moiety, and a 4-(oxy)phenoxy)acetyl moiety, each of which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

Embodiment 11: The compound of embodiment 9 or embodiment 10, or a salt or hydrate thereof, wherein L comprises one or more ethylene glycol diradical moieties.

Embodiment 12: The compound of any one of embodiments 9-11, wherein LTM is an N-substituted 1,3-dioxoisoindolinyl moiety, which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

Embodiment 13: A composition comprising a compound according to any one of embodiments 9-12, or a salt or hydrate thereof, and an agriculturally acceptable carrier.

Embodiment 14: A plant cell comprising a compound according to any one of embodiments 9-12, or a salt or hydrate thereof.

Embodiment 15: The plant cell of embodiment 14, wherein the compound is present in the plant cell in an amount sufficient to cause degradation of a protein of interest.

Embodiment 16: A compound according to Formula II:

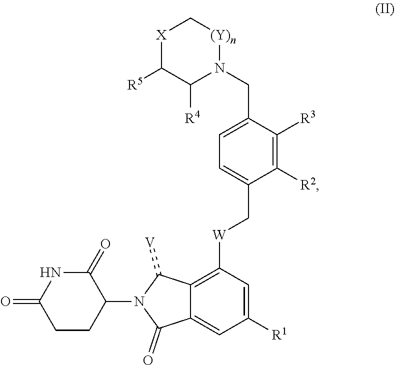

or a salt or solvate thereof, wherein: subscript n is an integer ranging from 0 to 3; V is absent or (0); R¹ is selected from the group consisting of H and —Z—R^{1a}; W, X, Y, and Z are independently selected from the group consisting of O, NR⁶, and CHR⁷, provided that Y is CHR⁷ when subscript n is 0 or 1; R^{1a} is selected from the group consisting of -L-PTM, H, and —(CH₂—CH₂—O)ₘ—CH₃; R² and R³ are independently selected from the group consisting of -L-PTM, H, —NH—(CH₂—CH₂—O)ₘ—CH₃, and —CONH—(CH₂—CH₂—O)ₘ—CH₃; R⁴ is selected from the group consisting of -L-PTM, H, —CH₂—O—(CH₂—CH₂—O)ₘ—CH₃, —CH₂NH—(CH₂—CH₂—O)ₘ—CH₃, and —CONH—(CH₂—CH₂—O)ₘ—CH₃; R⁵ and R⁷ are independently selected from the group consisting of -L-PTM, H, and —CONH—(CH₂—CH₂—O)ₘ—CH₃; R⁶ is selected from the group consisting of -L-PTM, H, C₁₋₆ acyl, and —CH₂—O—(CH₂—CH₂—O)ₘ—CH₃, provided that R⁶ is other than H when X is N; each subscript m is independently an integer ranging from 1 to 10; each L is an independently-selected linker moiety; each PTM is an independently-selected protein targeting moiety; and at least one of R¹-R⁷ is other than H.

Embodiment 17: The compound of embodiment 16, or a salt or hydrate thereof, wherein one and only one of R¹ and R²-R⁷ is -L-PTM.

Embodiment 18: The compound of embodiment 16 or embodiment 17, or a salt or hydrate thereof, wherein V is absent.

Embodiment 19: The compound of any one of embodiments 16-18, or a salt or hydrate thereof, wherein W is O.

Embodiment 20: The compound of any one of embodiments 16-19, or a salt or hydrate thereof, wherein R¹ is H.

Embodiment 21: The compound of any one of embodiments 16-19, or a salt or hydrate thereof, wherein R¹ is —Z—R^{1a}.

Embodiment 22: The compound of any one of embodiments 16-21, or a salt or hydrate thereof, wherein R² and R³ are H.

Embodiment 23: The compound of any one of embodiments 16-21, or a salt or hydrate thereof, wherein R² is H, and R³ is selected from the group consisting of -L-PTM, —NH—(CH₂—CH₂—O)ₘ—CH₃, and —CONH—(CH₂—CH₂—O)ₘ—CH₃.

Embodiment 24: The compound of any one of embodiments 16-21, or a salt or hydrate thereof, wherein R² is selected from the group consisting of -L-PTM, —NH—(CH₂—CH₂—O)ₘ—CH₃, and —CONH—(CH₂—CH₂—O)ₘ—CH₃, and R³ is H.

Embodiment 25: The compound of any one of embodiments 16-24, or a salt or hydrate thereof, wherein X is O, subscript n is 1, Y is —CHR⁷, and R⁷ is H.

Embodiment 26: The compound of embodiment 25, or a salt or hydrate thereof, wherein R⁵ is H.

Embodiment 27: The compound of embodiment 25 or claim 26, or a salt or hydrate thereof, wherein R⁴ is selected from the group consisting of -L-PTM, —CH₂—O—(CH₂—CH₂—O)ₘ—CH₃, —CH₂NH—(CH₂—CH₂—O)ₘ—CH₃, and —CONH—(CH₂—CH₂—O)ₘ—CH.

Embodiment 28: The compound of any one of embodiments 16-24, or a salt or hydrate thereof, wherein X is —CHR⁷, subscript n is 0 or 1, Y is —CHR⁷, and R⁷ is H.

Embodiment 29: The compound of embodiment 28, or a salt or hydrate thereof, wherein R⁴ is H.

Embodiment 30: The compound of embodiment 28 or embodiment 29, or a salt or hydrate thereof, wherein R⁵ is selected from the group consisting of -L-PTM, and —CONH—(CH₂—CH₂—O)ₘ—CH₃.

Embodiment 31: The compound of any one of embodiments 16-30, or a salt or hydrate thereof, wherein each subscript m is independently 1 or 2.

Embodiment 32: The compound of any one of embodiments 16-31, or a salt or hydrate thereof, wherein the protein targeting moiety targets a plant protein.

Embodiment 33: The compound of embodiment 32, or a salt or hydrate thereof, wherein the plant protein is a hydroxyphenylpyruvate dioxygenase, an acetolactate synthase, or an acetyl CoA carboxylase.

Embodiment 34: The compound of any one of embodiments 16-33, or a salt or hydrate thereof, wherein the PTM is selected from the group consisting of: a halo-(2,6-dioxocyclohexane-carbonyl)phenyl moiety, an N-(5,8-dimethoxy-[1,2,4]triazolo[1,5-c]pyrimidin-2-yl)-6-(trifluoromethyl)benz-2-yl-sulfonamide moiety, a 1-oxo-2-(1-(ethoxyimino)propyl)-3-hydroxy-cyclohex-2-en-5-yl moiety, a 6-((4-oxy)phenoxy)pyridin-3-yl moiety, and a 4-(oxy)phenoxy)acetyl moiety, each of which is optionally substituted with one or more substituents independently selected from the group consisting of C₁₋₆ alkyl, halo, hydroxy, amino, C₁₋₆ alkylamino, C₁₋₆ amido, C₁₋₆ acyl, nitro, cyano, and C₁₋₆ alkoxy.

Embodiment 35: The compound of any one of embodiments 16-34, or a salt or hydrate thereof, wherein L comprises one or more ethylene glycol diradical moieties, one or more of which is optionally replaced by a moiety independently selected from:

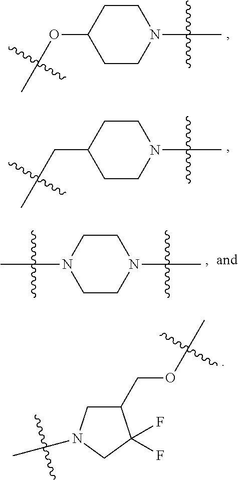 [SMILES: *OC1CCN(*)CC1 ; *CC1CCN(*)CC1 ; *N1CCN(*)CC1 ; *OCC1CN(*)CC1(F)F]

Embodiment 36: A composition comprising a compound according to any one of embodiments 16-35, or a salt or hydrate thereof, and an agriculturally acceptable carrier.

Embodiment 37: A method for controlling the level of a target protein in a cell, the method comprising contacting the cell with an effective amount of a compound according to any one of embodiments 16-35, or a salt or hydrate thereof, or an effective amount of a composition according to embodiment 36.

Embodiment 38: The method of embodiment 37, wherein the cell is a plant cell.

Embodiment 39: The method of embodiment 37 or claim 38, wherein at least one of R^{1a} and R²-R⁷ is -L-PTM.

Embodiment 40: The method of embodiment 39, wherein controlling the level of the target protein comprises targeted degradation of the target protein.

Embodiment 41: A plant cell comprising a compound according to any one of embodiments 16-35, or a salt or hydrate thereof.

### EXAMPLES

### Example 1: Synthesis of 4-[2-[2-[2-[2-chloro-3-(2,6-dioxocyclohexanecarbonyl)phenoxy]ethoxy]ethoxy]-ethoxy]-2-(2,6-dioxo-3-piperidyl)isoindoline-1,3-dione

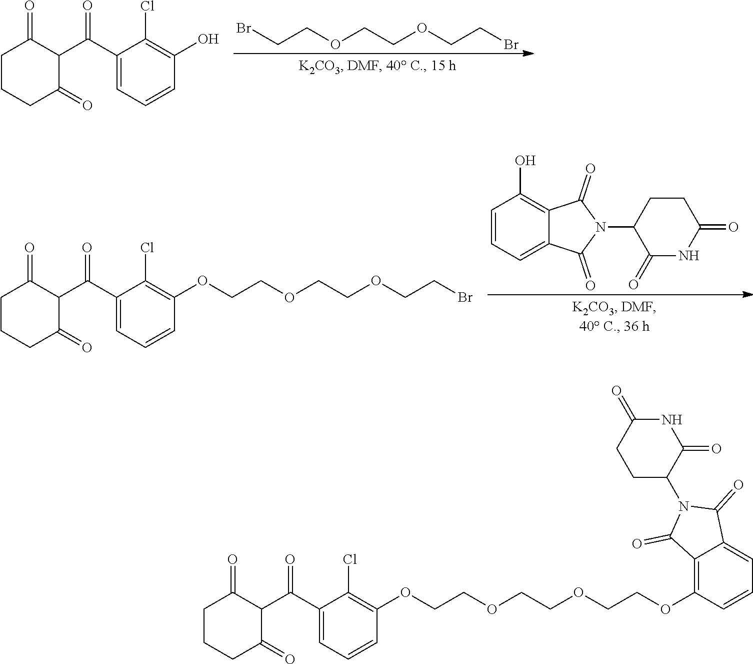 [SMILES: O=C1CCCC(=O)C1C(=O)c1cccc(O)c1Cl ; BrCCOCCOCCBr ; O=C1CCCC(=O)C1C(=O)c1cccc(OCCOCCOCCBr)c1Cl ; O=C1CCC(N2C(=O)c3cccc(O)c3C2=O)C(=O)N1 ; O=C1CCC(N2C(=O)c3cccc(OCCOCCOCCOc4cccc(C(=O)C5C(=O)CCCC5=O)c4Cl)c3C2=O)C(=O)N1]

### Step 1

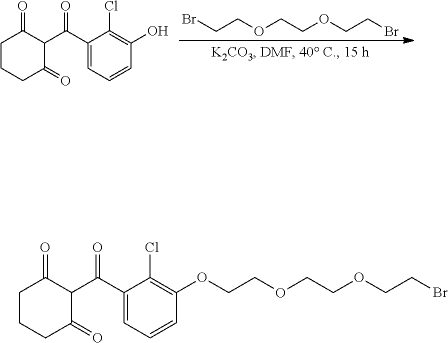 [SMILES: O=C1CCCC(=O)C1C(=O)c1cccc(O)c1Cl ; BrCCOCCOCCBr ; O=C1CCCC(=O)C1C(=O)c1cccc(OCCOCCOCCBr)c1Cl]

To a solution of 2-(2-chloro-3-hydroxy-benzoyl)cyclohexane-1,3-dione (200 mg, 749.97 μmol), 1,2-bis(2-bromoethoxy)ethane (413.93 mg, 1.50 mmol) in DMF (2 mL) was added K₂CO₃ (310.95 mg, 2.25 mmol). The reaction mixture was stirred under N₂ at 40° C. for 15 hours. The reaction mixture was poured into water (10 mL), and the pH value was adjusted to 5 with 2 M HCl (aq). The mixture was extracted with CH₂Cl₂ (20 mL). The organic phase was washed with brine (20 mL×2), and dried over anhydrous Na₂SO₄, filtered, and concentrated under vacuum. The crude material was purified by column chromatography on silica gel (Petroleum ether:Ethyl acetate:EtOH=1:1:0.3) to give 2-[3-[2-[2-(2-bromoethoxy)ethoxy]ethoxy]-2-chloro-benzoyl]cyclohexane-1,3-dione (110 mg, 218.94 μmol, 29.19% yield, 91.9% purity) as a green oil. HNMR: CDCl₃, 400 MHz. δ 7.16-7.06 (m, 1H), 6.83 (br d, J=7.2 Hz, 1H), 6.70 (br d, J=7.2 Hz, 1H), 4.25-4.15 (m, 2H), 4.15-4.08 (m, 1H), 3.95-3.83 (m, 8H), 3.48-3.44 (m, 2H), 2.52 (br d, J=2.4 Hz, 2H), 2.34 (br s, 2H), 1.86 (br d, J=0.8 Hz, 2H). LCMS: RT=0.933 min, m/z 461.1 [M+H]⁺.

### Step 2

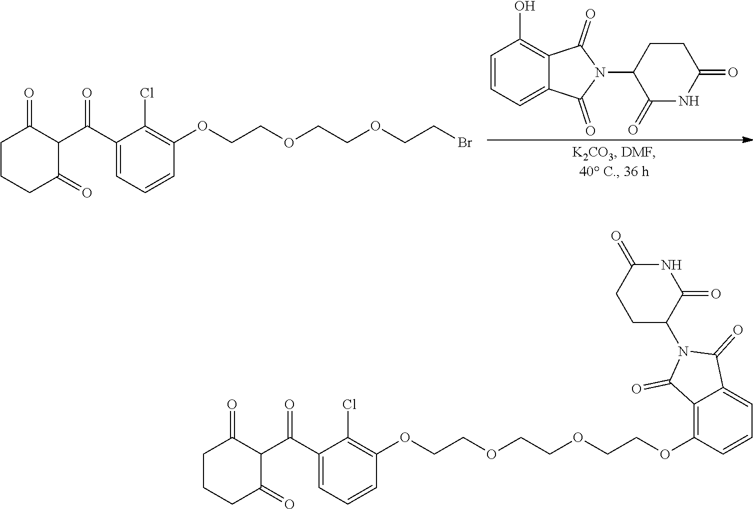 [SMILES: O=C1CCCC(=O)C1C(=O)c1cccc(OCCOCCOCCBr)c1Cl ; O=C1CCC(N2C(=O)c3cccc(O)c3C2=O)C(=O)N1 ; O=C1CCC(N2C(=O)c3cccc(OCCOCCOCCOc4cccc(C(=O)C5C(=O)CCCC5=O)c4Cl)c3C2=O)C(=O)N1]

To a solution of 2-[3-[2-[2-(2-bromoethoxy)ethoxy]ethoxy]-2-chloro-benzoyl]cyclohexane-1,3-dione (90 mg, 194.92 μmol) and 2-(2,6-dioxo-3-piperidyl)-4-hydroxy-isoindoline-1,3-dione (64.14 mg, 233.90 μmol) in DMF (10 mL) was added K₂CO₃ (53.88 mg, 389.84 μmol). The reaction was stirred under N₂ at 40° C. for 36 hours. The reaction mixture was poured into water (60 mL). The pH was adjusted to 4 with 1 M HCl aqueous and extracted with ethyl acetate (60 mL). The organic phase was washed with brine (60 mL×2), dried over anhydrous Na₂SO₄, filtered, and concentrated under vacuum. The crude material was purified by prep-HPLC (column: Phenomenex Synergi C18 150*25 mm*10 μm; mobile phase: [water(0.1% TFA)-ACN]; B %: 41%-71%, 10 min) to give 4-[2-[2-[2-[2-chloro-3-(2,6-dioxocyclohexanecarbonyl)phenoxy]ethoxy]-ethoxy]ethoxy]-2-(2,6-dioxo-3-piperidyl)isoindoline-1,3-dione (18.54 mg, 26.80 μmol, 13.75% yield, 94.7% purity) as a brown gum. HNMR: DMSO-d₆, 400 MHz. δ 11.10 (s, 1H), 7.79 (dd, J=7.2, 8.4 Hz, 1H), 7.52 (d, J=8.4 Hz, 1H), 7.44 (d, J=7.2 Hz, 1H), 7.33-7.27 (m, 1H), 7.17 (dd, J=1.2, 8.4 Hz, 1H), 6.84 (dd, J=1.2, 7.6 Hz, 1H), 5.08 (dd, J=5.2, 13.0 Hz, 1H), 4.38-4.30 (m, 2H), 4.21-4.15 (m, 2H), 3.80 (td, J=4.8, 9.2 Hz, 4H), 3.71-3.53 (m, 8H), 2.93-2.83 (m, 1H), 2.62-2.56 (m, 2H), 2.06-1.98 (m, 1H), 1.92 (quin, J=6.4 Hz, 2H). LCMS: RT=2.939 min, m/z 655.2 [M+H]⁺.

The following compound was prepared in analogous fashion to Example 1.

### Compound 1-A

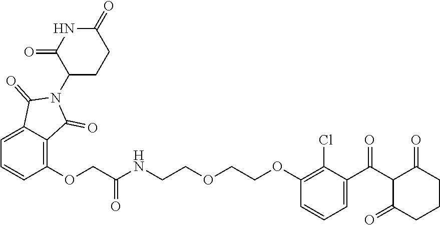 [SMILES: O=C(COc1cccc2c1C(=O)N(C1CCC(=O)NC1=O)C2=O)NCCOCCOc1cccc(C(=O)C2C(=O)CCCC2=O)c1Cl]

N-(2-(2-(2-Chloro-3-(2,6-dioxocyclohexane-1-carbonyl)phenoxy)ethoxy)ethyl)-2-((2-(2,6-dioxopiperidin-3-yl)-1,3-dioxoisoindolin-4-yl)oxy)acetamide. HNMR: DMSO-d6, 400 MHz. δ 11.12 (s, 1H), 8.05 (t, J=5.2 Hz, 1H), 7.83-7.73 (m, 1H), 7.48 (d, J=7.2 Hz, 1H), 7.38 (d, J=8.4 Hz, 1H), 7.31-7.22 (m, 1H), 7.14 (d, J=8.0 Hz, 1H), 6.80 (d, J=7.2 Hz, 1H), 5.15-5.05 (m, 1H), 4.78 (s, 2H), 4.24-4.15 (m, 2H), 3.84-3.77 (m, 2H), 3.58 (t, J=5.6 Hz, 2H), 3.40-3.37 (m, 2H), 2.94-2.84 (m, 1H), 2.64-2.51 (m, 6H), 2.06-1.98 (m, 1H), 1.95-1.85 (m, 2H). LCMS: RT=2.786 min, m/z 668.1. [M+H]⁺.

### Example 2: Synthesis of 5-[4-[2-[2-[2-[2-chloro-3-(2,6-dioxocyclohexane-carbonyl)phenoxy]ethoxy]-ethoxy]ethyl]piperazin-1-yl]-2-(2,6-dioxo-3-piperidyl)isoindoline-1,3-dione

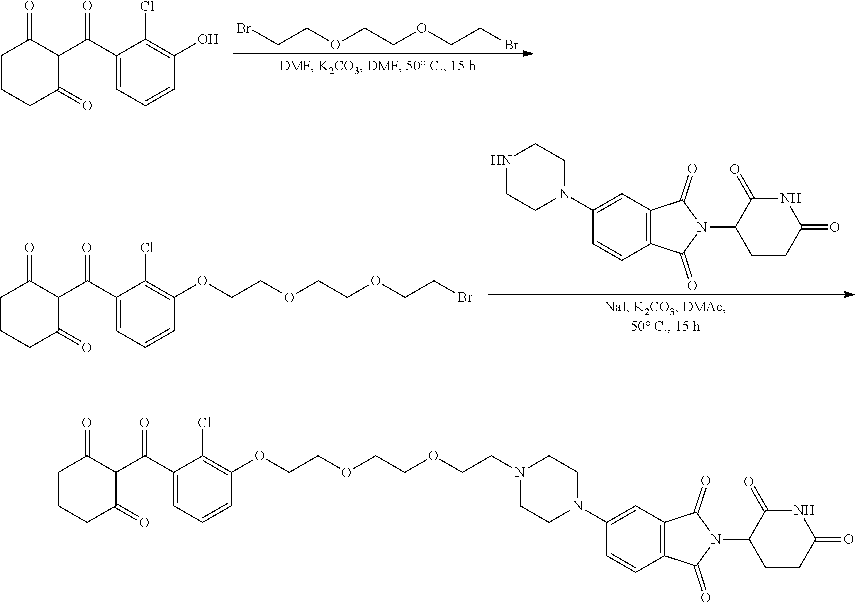 [SMILES: O=C1CCCC(=O)C1C(=O)c1cccc(O)c1Cl ; BrCCOCCOCCBr ; O=C1CCCC(=O)C1C(=O)c1cccc(OCCOCCOCCBr)c1Cl ; O=C1CCC(N2C(=O)c3ccc(N4CCNCC4)cc3C2=O)C(=O)N1 ; O=C1CCC(N2C(=O)c3ccc(N4CCN(CCOCCOCCOc5cccc(C(=O)C6C(=O)CCCC6=O)c5Cl)CC4)cc3C2=O)C(=O)N1]

### Step 1

A mixture of 2-(2-chloro-3-hydroxy-benzoyl)cyclohexane-1,3-dione (500 mg, 1.87 mmol), 1,2-bis(2-bromoethoxy)ethane (1.03 g, 3.75 mmol) and K₂CO₃ (518.25 mg, 3.75 mmol) in DMF (5 mL) was stirred at 50° C. for 15 hours. The mixture was added to water (40 mL) and acidified with HCl aqueous (2 M) to pH=2-3 and extracted with EtOAc (40 mL). The organic phase was washed with brine (40 mL×2), dried over anhydrous Na₂SO₄, filtered and concentrated in vacuo. The residue was purified by flash silica gel chromatography (ISCO®; 25 g SepaFlash® Silica Flash Column, Eluent of 0-1.9% Methanol/Dichloromethane gradient @35 mL/min) to give 2-[3-[2-[2-(2-bromoethoxy)ethoxy]ethoxy]-2-chloro-benzoyl]cyclohexane-1,3-dione (270 mg, 567.21 μmol, 30.25% yield, 97.0% purity) as a yellow oil. HNMR: CDCl₃, 400 MHz. δ 7.30-7.25 (m, 1H), 7.01 (dd, J=1.2, 8.4 Hz, 1H), 6.82 (dd, J=1.2, 7.6 Hz, 1H), 4.24-4.18 (m, 2H), 3.94-3.89 (m, 2H), 3.85-3.81 (m, 2H), 3.80-3.76 (m, 2H), 3.73-3.68 (m, 2H), 3.51-3.45 (m, 2H), 2.78 (t, J=6.4 Hz, 2H), 2.45 (t, J=6.4 Hz, 2H), 2.07-2.04 (m, 2H). LCMS: RT=0.945 min, m/z 463.1 [M+H]⁺.

### Step 2

The mixture of 2-[3-[2-[2-(2-bromoethoxy)ethoxy]ethoxy]-2-chloro-benzoyl]cyclohexane-1,3-dione (100 mg, 216.58 μmol), 2-(2,6-dioxo-3-piperidyl)-5-piperazin-1-yl-isoindoline-1,3-dione (82.04 mg, 216.58 μmol, HCl salt), K₂CO₃ (89.80 mg, 649.73 μmol) and NaI (32.46 mg, 216.58 μmol) in DMAC (1 mL) was stirred at 50° C. for 15 hours. The mixture was filtered. The filtrate was purified by prep-HPLC (column: Phenomenex Synergi™ C18 150*25 mm*10 μm; mobile phase: [water(0.225% FA)-ACN]; B %: 17%-47%, 10 min) to give the desired product 5-[4-[2-[2-[2-[2-chloro-3-(2,6-dioxocyclohexane-carbonyl)phenoxy]ethoxy]ethoxy]ethyl]piperazin-1-yl]-2-(2,6-dioxo-3-piperidyl)isoindoline-1,3-dione (96.0% purity, formic acid salt) (total 35.8 mg) as a yellow solid. HNMR: DMSO-d6, 400 MHz. δ 11.08 (s, 1H), 8.13 (s, 1H), 7.68 (d, J=8.8 Hz, 1H), 7.34 (d, J=2.4 Hz, 1H), 7.28-7.21 (m, 2H), 7.13 (d, J=7.6 Hz, 1H), 6.79 (d, J=7.6 Hz, 1H), 5.07 (dd, J=5.2, 12.8 Hz, 1H), 4.22-4.14 (m, 2H), 3.83-3.77 (m, 2H), 3.67-3.55 (m, 6H), 3.47-3.43 (m, 4H), 2.93-2.84 (m, 1H), 2.75-2.64 (m, 6H), 2.63-2.52 (m, 2H), 2.47-2.41 (m, 4H), 2.06-1.97 (m, 1H), 1.93-1.81 (m, 2H). LCMS: WX-OER-MB-001-B_001_LCMS_EW22043-83, RT=2.244 min, m z 723.3 [M+H]⁺.

The following compounds were prepared in analogous fashion to Example 2

### Compound 2-A

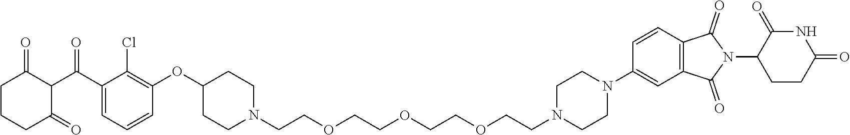 [SMILES: O=C1CCC(N2C(=O)c3ccc(N4CCN(CCOCCOCCOCCN5CCC(Oc6cccc(C(=O)C7C(=O)CCCC7=O)c6Cl)CC5)CC4)cc3C2=O)C(=O)N1]

5-[4-[2-[2-[2-[2-[4-[2-Chloro-3-(2,6-dioxocyclohexanecarbonyl)phenoxy]-1-piperidyl]ethoxy]ethoxy]ethoxy]ethyl]piperazin-1-yl]-2-(2,6-dioxo-3-piperidyl)isoindoline-1,3-dione. HNMR: DMSO-d₆, 400 MHz. δ 11.10 (s, 1H), 8.14 (s, 1H), 7.69-7.65 (m, 1H), 7.34-7.32 (m, 1H), 7.26-7.22 (m, 1H), 7.11-7.09 (m, 1H), 7.01-6.98 (m, 1H), 6.63 (d, J=6.8 Hz, 1H), 5.09-5.04 (m, 1H), 4.55-4.48 (m, 1H), 3.72-3.55 (m, 16H), 3.10-3.00 (m, 2H), 2.95-2.83 (m, 5H), 2.67-2.52 (m, 8H), 2.28-2.16 (m, 4H), 2.06-1.97 (m, 3H), 1.90-1.70 (m, 4H). LCMS: RT=1.986 min, m/z 850.3 [M+H]⁺.

### Compound 2-B

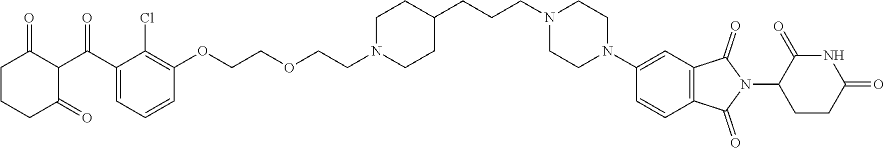 [SMILES: O=C1CCC(N2C(=O)c3ccc(N4CCN(CCCC5CCN(CCOCCOc6cccc(C(=O)C7C(=O)CCCC7=O)c6Cl)CC5)CC4)cc3C2=O)C(=O)N1]

5-(4-(3-(1-(2-(2-(2-Chloro-3-(2,6-dioxocyclohexane-1-carbonyl)phenoxy)ethoxy)ethyl)piperidin-4-yl)propyl)piperazin-1-yl)-2-(2,6-dioxopiperidin-3-yl)isoindoline-1,3-dione. HNMR: DMSO-d₆, 400 MHz. δ 11.08 (s, 1H), 8.15 (s, 1H), 7.68 (d, J=8.8 Hz, 1H), 7.37-7.34 (m, 1H), 7.26 (dd, J=2.0, 8.8 Hz, 1H), 7.14-7.05 (m, 1H), 6.91 (d, J=7.6 Hz, 1H), 6.60 (dd, J=1.2, 7.6 Hz, 1H), 5.07 (dd, J=5.2, 12.8 Hz, 1H), 4.20-4.13 (m, 2H), 3.81-3.74 (m, 4H), 3.54-3.37 (m, 5H), 3.32-3.23 (m, 2H), 3.08-3.01 (m, 2H), 2.92-2.82 (m, 1H), 2.71-2.56 (m, 7H), 2.46-2.39 (m, 2H), 2.18-2.11 (m, 4H), 2.07-1.97 (m, 1H), 1.79-1.66 (m, 4H), 1.56-1.42 (m, 2H), 1.39-1.14 (m, 5H). LCMS: RT=2.038 min, m/z 804.3 [M+H]⁺.

### Example 3: Synthesis of 5-[2-[2-[2-[2-chloro-3-(2,6-dioxocyclohexanecarbonyl)-6-methylsulfonyl-phenoxy]ethoxy]ethoxy]ethoxy]-2-(2,6-dioxo-3-piperidyl)isoindoline-1,3-dione

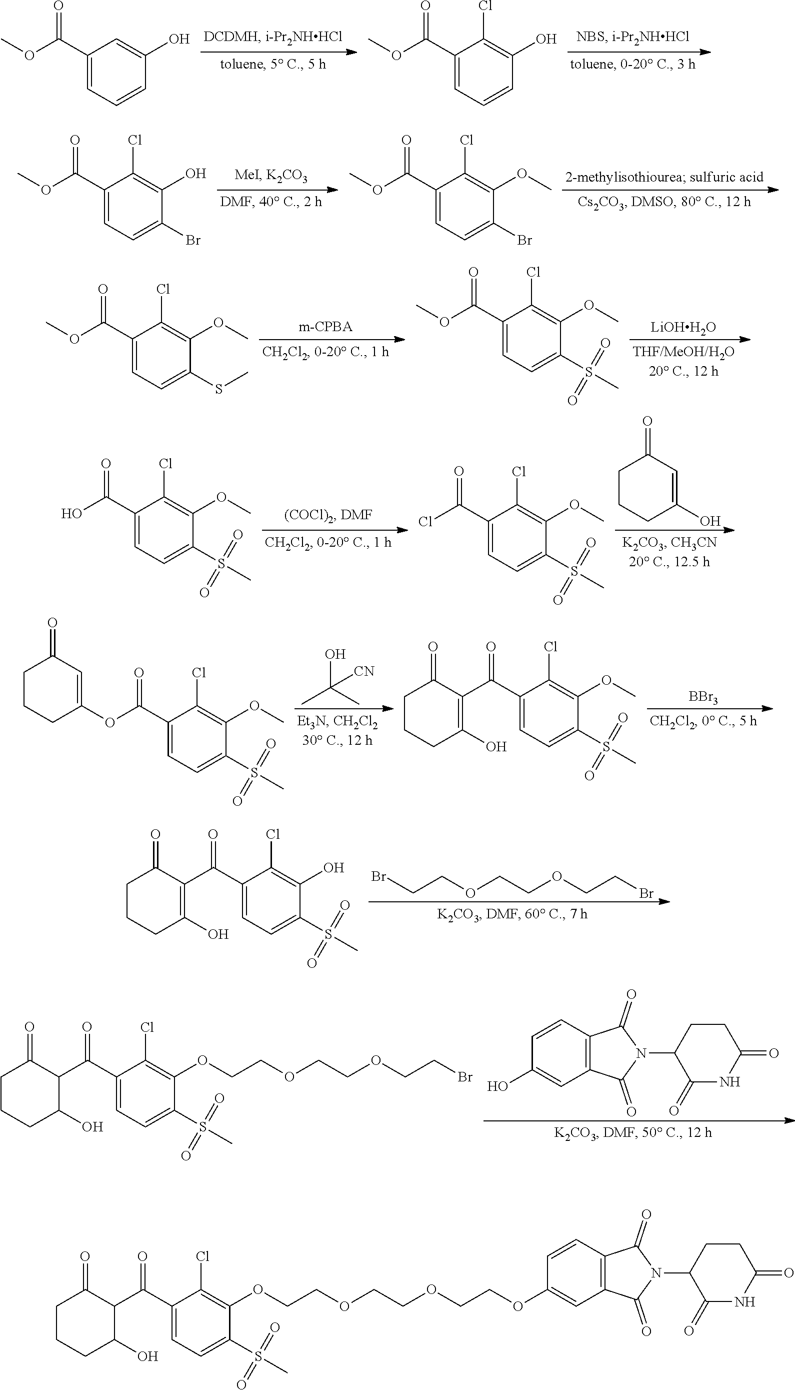

### Step 1

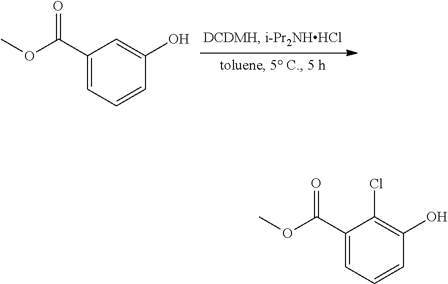

To a mixture of methyl 3-hydroxybenzoate (17.3 g, 113.71 mmol) and N-isopropylpropan-2-amine; hydrochloride (157 mg, 1.14 mmol) in toluene (170 mL) in the absence of light was added 1,3-dichloro-5,5-dimethyl-imidazolidine-2,4-dione (23.5 g, 119.28 mmol) in portions at room temperature (5° C.). The mixture was degassed and purged with nitrogen three times, and then stirred at room temperature (5° C.) for 5 hours in the absence of light. The mixture was concentrated to dryness. The residue was diluted with Ethyl acetate (50 mL), washed with water (100 mL). The organic phase was separated, and the aqueous phase was extracted with Ethyl acetate (20 mL×2). The combined organic phases were washed with brine (100 mL), dried over Na₂SO₄, filtered, and concentrated dryness. The residue was purified by column chromatography (SiO₂, Petroleum ether/Ethyl acetate=1/0 to 50/1) to give methyl 2-chloro-3-hydroxy-benzoate (20 g, 91.11 mmol, 80.13% yield, 85% purity) as a yellow solid. HNMR: CDCl₃, 400 MHz. δ 7.45-7.49 (m, 1H), 7.18-7.29 (m, 2H), 6.04 (s, 1H), 3.95 (s, 3H). LCMS: RT=0.728 min, m/z 187.0 [M+H]⁺.

### Step 2

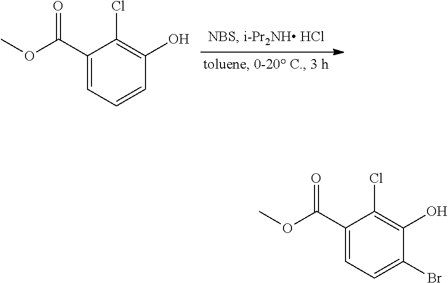

To a mixture of methyl 2-chloro-3-hydroxy-benzoate (20 g, 91.11 mmol, 85% purity) and N-isopropylpropan-2-amine; hydrochloride (627 mg, 4.55 mmol) in toluene (300 mL) was added NBS (16.24 g, 91.27 mmol) in portions at 0° C. in the absence of light. The mixture was stirred at room temperature (20° C.) for 3 hours. The mixture was filtered and the filter cake was washed with Ethyl acetate (100 mL). The filtrate was concentrated to dryness. The residue was diluted with Ethyl acetate (100 mL), washed with water (100 mL). The organic phase was separated, and the aqueous phase was extracted with Ethyl acetate (50 mL×2). The combined organic phases were washed with brine (100 mL), dried over Na₂SO₄, filtered, and concentrated to dryness. The residue was purified by column chromatography (SiO₂, Petroleum ether/Ethyl acetate=1/0 to 10/1) to give 4-bromo-2-chloro-3-hydroxy-benzoate (25 g, crude) as a white solid, which was used without further purification. LCMS: RT=0.838 min, m/z 266.9 [M+H]⁺.

### Step 3

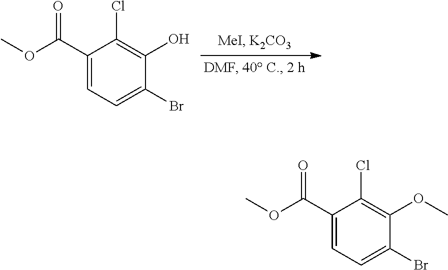

To a mixture of 4-bromo-2-chloro-3-hydroxy-benzoate (25 g, 94.17 mmol) and K₂CO₃ (39.04 g, 282.50 mmol) in DMF (250 mL) was added Mel (20.05 g, 141.25 mmol, 8.79 mL). The mixture was then stirred at 40° C. for 2 hours. The mixture was quenched with water (200 mL), extracted with Ethyl acetate (100 mL×2). The combined organic phases were washed with brine (100 mL×3), dried over Na₂SO₄, filtered, and concentrated to give a residue. The residue was purified by prep-HPLC (column: Phenomenex luna C18 250*80 mm*10 μm; mobile phase: [water (0.225% FA)-ACN]; B %: 60%-90%, 20 min) to give methyl 4-bromo-2-chloro-3-methoxy-benzoate (5.4 g, 19.32 mmol, 20.52% yield) as an off-white solid. HNMR: CDCl₃, 400 MHz. δ 7.54 (d, J=8.4 Hz, 1H), 7.46 (d, J=8.4 Hz, 1H), 3.95 (s, 3H), 3.93 (s, 3H). LCMS: RT=0.934 min, m/z 280.9 [M+H]⁺.

### Step 4

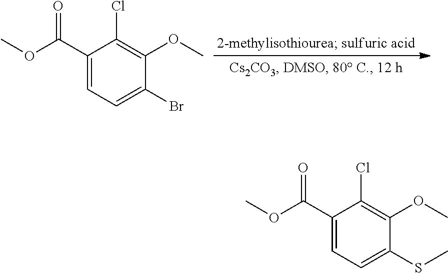

To a mixture of methyl 4-bromo-2-chloro-3-methoxy-benzoate (4.7 g, 16.81 mmol) and 2-methylisothiourea; sulfuric acid (5.62 g, 20.19 mmol) in DMSO (10 mL) was added Cs₂CO₃ (16.45 g, 50.49 mmol). The mixture was then stirred at 80° C. for 12 hours. The reaction mixture was quenched with water (50 mL), and then extracted with Ethyl acetate (20 mL×2). The combined organic layers were washed with brine (50 mL×3), dried over Na₂SO₄, filtered, and concentrated under reduced pressure to give a residue. The residue was purified by column chromatography (SiO₂, Petroleum ether/Ethyl acetate=50/1 to 10/1) to give methyl 2-chloro-3-methoxy-4-methylsulfanyl-benzoate (1.5 g, 3.65 mmol, 21.70% yield, 60% purity) as a white solid. HNMR: CDCl₃, 400 MHz. δ 7.65 (d, J=8.0 Hz, 1H), 7.04 (d, J=8.4 Hz, 1H), 3.94 (s, 3H), 3.93 (s, 3H), 2.48 (s, 3H). LCMS: RT=0.915 min, m/z 247.0 [M+H]⁺.

### Step 5

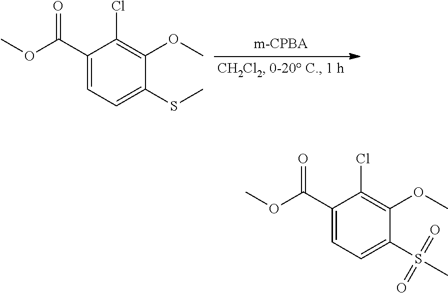

To a mixture of methyl 2-chloro-3-methoxy-4-methylsulfanyl-benzoate (1.5 g, 6.08 mmol) in CH₂Cl₂ (50 mL) was added m-CPBA (3.71 g, 18.25 mmol, 85% purity) at 0° C. The mixture was then stirred at room temperature (20° C.) for 12 hours. The mixture was diluted with CH₂Cl₂ (50 mL) and washed with saturated Na₂SO₃ aqueous (100 mL), saturated NaHCO₃ aqueous (50 mL) and brine (50 mL). Then the organic phase was dried over anhydrous Na₂SO₄, filtered and concentrated to dryness. The residue was purified by column chromatography (SiO₂, Petroleum ether/Ethyl acetate=50/1 to 5/1) to give methyl 2-chloro-3-methoxy-4-methylsulfonyl-benzoate (800 mg, 2.87 mmol, 47.21% yield) as a white solid. HNMR: CDCl₃, 400 MHz. δ 7.94 (d, J=8.4 Hz, 1H), 7.66 (d, J=8.4 Hz, 1H), 4.12 (s, 3H), 4.00 (s, 3H), 3.28 (s, 3H). LCMS: RT=0.762 min, m/z 278.9 [M+H]⁺.

### Step 6

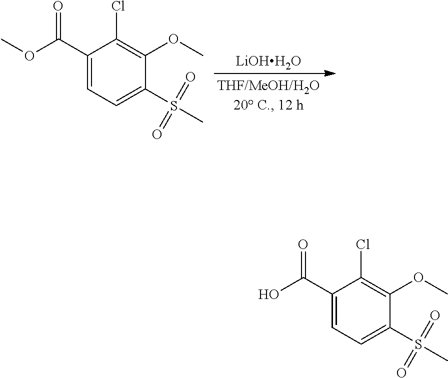

To a mixture of methyl 2-chloro-3-methoxy-4-methylsulfonyl-benzoate (800 mg, 2.87 mmol) in THE (5 mL), MeOH (5 mL) and H₂O (2 mL) was added LiOH H₂O (360 mg, 8.58 mmol). The mixture was then stirred at room temperature (20° C.) for 12 hours. The mixture was quenched with water (20 mL) and 1M HCl aqueous to adjust pH=2-3. The mixture was extracted with CH₂Cl₂ (20 mL×2). The combined organic phase was dried over anhydrous Na₂SO₄, filtered, and concentrated to give 2-chloro-3-methoxy-4-methylsulfonyl-benzoic acid (600 mg, 2.27 mmol, 78.97% yield) as an off-white solid, which was used without further purification.

### Step 7

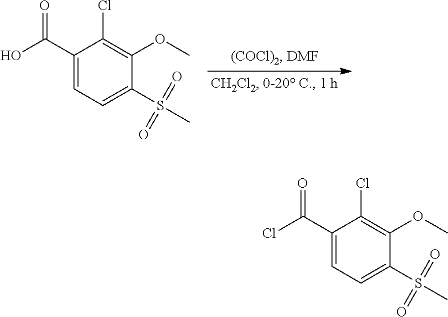 [SMILES: COc1c(S(C)(=O)=O)ccc(C(=O)O)c1Cl ; COc1c(S(C)(=O)=O)ccc(C(=O)Cl)c1Cl]

To a mixture of 2-chloro-3-methoxy-4-methylsulfonyl-benzoic acid (500 mg, 1.89 mmol) and DMF (14 mg, 191.53 μmol) in CH₂Cl₂ (3 mL) was added oxalyl chloride (507.50 mg, 4.00 mmol, 0.35 mL) at 0° C. The mixture was then stirred at room temperature (20° C.) for 1 hour under nitrogen. The mixture was concentrated to give 2-chloro-3-methoxy-4-methylsulfonyl-benzoyl chloride (530 mg, 1.87 mmol, 99.09% yield) as a yellow solid, which was used without further purification.

### Step 8

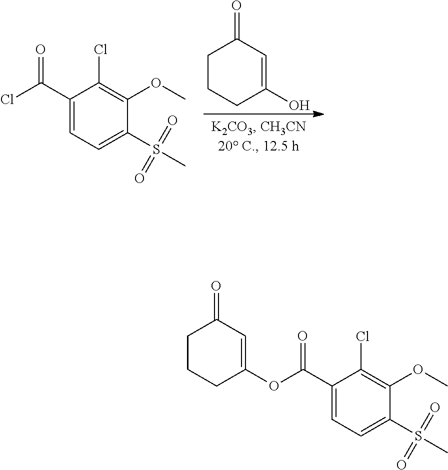

To a mixture of 3-hydroxycyclohex-2-en-1-one (230.00 mg, 2.05 mmol) in CH₃CN (10 mL) was added K₂CO₃ (390.00 mg, 2.82 mmol). The mixture was stirred at room temperature (20° C.) for 10 minutes and then added to 2-chloro-3-methoxy-4-methylsulfonyl-benzoyl chloride (530 mg, 1.87 mmol). The mixture was stirred at room temperature (20° C.) for 12 hours under nitrogen. The reaction mixture was quenched with water (20 mL), and then extracted with Ethyl acetate (10 mL×2). The combined organic layers were washed with brine (20 mL), dried over Na₂SO₄, filtered, and concentrated under reduced pressure to give (3-oxocyclohexen-1-yl) 2-chloro-3-methoxy-4-methylsulfonyl-benzoate (670 mg, crude) as a yellow gum, which was used without further purification. LCMS: RT=0.855 min, m/z 359.0 [M+H]⁺.

### Step 9

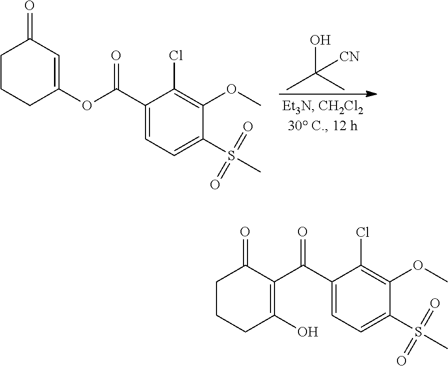

To a mixture of (3-oxocyclohexen-1-yl) 2-chloro-3-methoxy-4-methylsulfonyl-benzoate (670 mg, 1.87 mmol) and Et₃N (378.04 mg, 3.74 mmol, 520.00 μL) in CH₂Cl₂ (10 mL) was added 2-hydroxy-2-methyl-propanenitrile (48 mg, 564.01 μmol, 51.50 μL). The mixture was then stirred at 30° C. for 12 hours under nitrogen. The mixture was diluted with CH₂Cl₂ (20 mL), acidified with 1 N HCl to pH-3. The organic layer was separated, and the aqueous phase was extracted with CH₂Cl₂ (10 mL×2). The combined organic layers were washed with brine (50 mL), dried over Na₂SO₄, filtered, and concentrated under reduced pressure. The residue was purified by column chromatography (SiO₂, Petroleum ether/Ethyl acetate=10/1 to 1/1) to give 2-(2-chloro-3-methoxy-4-methylsulfonyl-benzoyl)-3-hydroxy-cyclohex-2-en-1-one (540 mg, 1.51 mmol, 80.60% yield) as a yellow solid. HNMR: CDCl₃, 400 MHz. δ 7.95 (d, J=8.0 Hz, 1H), 7.10 (d, J=8.0 Hz, 1H), 4.10 (s, 3H), 3.28 (s, 3H), 2.85 (t, J=6.4 Hz, 2H), 2.48 (t, J=6.8 Hz, 2H), 2.10 (t, J=6.4 Hz, 2H). LCMS: RT=0.847 min, m/z 359.0 [M+H]⁺.

### Step 10

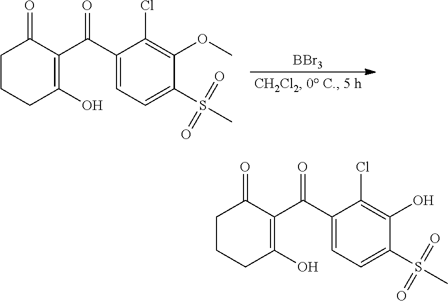

To a mixture of 2-(2-chloro-3-methoxy-4-methylsulfonyl-benzoyl)-3-hydroxy-cyclohex-2-en-1-one (540 mg, 1.51 mmol) in CH₂Cl₂ (5 mL) was added BBr₃ (1.14 g, 4.57 mmol, 0.44 mL) at 0° C. The mixture was then stirred at 0° C. for 3 hours. LC-MS showed the starting material remained. BBr₃ (1.14 g, 4.57 mmol, 0.44 mL) was added drop-wise at 0° C. The mixture was then stirred at 0° C. for another 2 hours. The mixture was diluted with CH₂Cl₂ (20 mL) and poured into water (50 mL). The organic layer was separated, and the aqueous phase was extracted with CH₂Cl₂ (10 mL×2). The combined organic layers were washed with brine (30 mL), dried over Na₂SO₄, filtered, and concentrated under reduced pressure. The residue was purified by prep-HPLC (column: Phenomenex luna C18 150*40 mm*15 μm; mobile phase: [water (0.225% FA)-ACN]; B %: 20%-50%, 10 min) to give 2-(2-chloro-3-hydroxy-4-methylsulfonyl-benzoyl)-3-hydroxy-cyclohex-2-en-1-one (170 mg, 493.09 μmol, 32.76% yield) as a yellow solid. HNMR: CDCl₃, 400 MHz. δ 9.02 (s, 1H), 7.34 (d, J=8.0 Hz, 1H), 6.90 (d, J=8.0 Hz, 1H), 3.21 (s, 3H), 2.84 (t, J=6.4 Hz, 2H), 2.48 (t, J=6.8 Hz, 2H), 2.06-2.15 (m, 2H). LCMS: RT=0.772 min, m/z 345.0 [M+H]⁺.

### Step 11

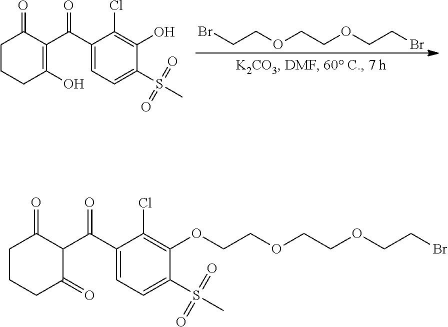 [SMILES: CS(=O)(=O)c1ccc(C(=O)C2=C(O)CCCC2=O)c(Cl)c1O ; BrCCOCCOCCBr ; CS(=O)(=O)c1ccc(C(=O)C2C(=O)CCCC2=O)c(Cl)c1OCCOCCOCCBr]

To a mixture of 2-(2-chloro-3-hydroxy-4-methylsulfonyl-benzoyl)-3-hydroxy-cyclohex-2-en-1-one (160 mg, 464.08 μmol) and K₂CO₃ (192.00 mg, 1.39 mmol) in DMF (5 mL) was added 1,2-bis(2-bromoethoxy)ethane (320.00 mg, 1.16 mmol). The mixture was then stirred at 60° C. for 7 hours. The mixture was diluted with water (50 mL), treated with 2 N HCl aqueous to pH-3. The resulting mixture was extracted with Ethyl acetate (10 mL×3). The combined organic layers were washed with brine (50 mL×3), dried over Na₂SO₄, filtered, and concentrated under reduced pressure. The residue was purified by column chromatography (SiO₂, CH₂Cl₂/MeOH=1/0 to 50/1) to give the desired product 2-[3-[2-[2-(2-bromoethoxy)ethoxy]ethoxy]-2-chloro-4-methylsulfonyl-benzoyl]cyclohexane-1,3-dione (120 mg, 222.30 μmol, 47.90% yield) as a yellow gum. LCMS: RT=0.917 min, m/z 541.0 [M+H]⁺.

### Step 12

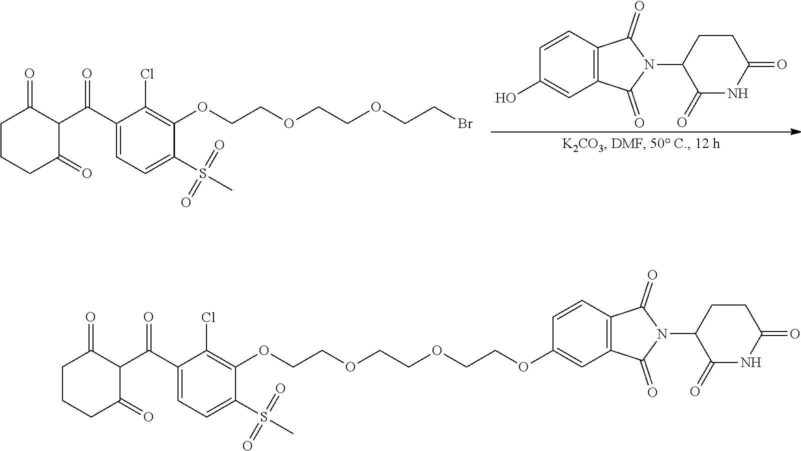 [SMILES: CS(=O)(=O)c1ccc(C(=O)C2C(=O)CCCC2=O)c(Cl)c1OCCOCCOCCBr ; O=C1CCC(N2C(=O)c3ccc(O)cc3C2=O)C(=O)N1 ; CS(=O)(=O)c1ccc(C(=O)C2C(=O)CCCC2=O)c(Cl)c1OCCOCCOCCOc1ccc2c(c1)C(=O)N(C1CCC(=O)NC1=O)C2=O]

To a mixture of 2-[3-[2-[2-(2-bromoethoxy)ethoxy]ethoxy]-2-chloro-4-methylsulfonyl-benzoyl]cyclohexane-1,3-dione (110 mg, 203.77 μmol) and 2-(2,6-dioxo-3-piperidyl)-5-hydroxy-isoindoline-1,3-dione (112 mg, 408.42 μmol) in DMF (3 mL) was added K₂CO₃ (84 mg, 607.79 μmol). The mixture was then stirred at 50° C. for 12 hours. The mixture was filtered. The filtrate concentrated to dryness and purified by prep-HPLC (column: Phenomenex luna C18 150*25 mm*10 μm; mobile phase: [water (0.225% FA)-ACN]; B %: 39%-69%, 10 min) to give 5-[2-[2-[2-[2-chloro-3-(2,6-dioxocyclohexanecarbonyl)-6-methylsulfonyl-phenoxy]ethoxy]ethoxy]ethoxy]-2-(2,6-dioxo-3-piperidyl)-isoindoline-1,3-dione (54.69 mg, 73.55 μmol, 36.10% yield, 98.6% purity) as an off-white solid. HNMR: DMSO-d₆, 400 MHz. δ 11.12 (s, 1H), 7.73-7.87 (m, 2H), 7.44-7.50 (m, 1H), 7.33-7.40 (m, 1H), 7.17-7.23 (m, 1H), 5.07-5.19 (m, 1H), 4.19-4.41 (m, 4H), 3.78-3.95 (m, 4H), 3.54-3.70 (m, 4H), 3.40 (s, 3H), 2.83-2.98 (m, 1H), 2.53-2.64 (m, 6H), 1.99-2.10 (m, 1H), 1.83-1.95 (m, 2H). LCMS: RT=2.911 min, m/z 733.2 [M+H]⁺.

### Example 4: Synthesis of N-(5,8-dimethoxy-[1,2,4]triazolo[1,5-c]pyrimidin-2-yl)-2-[2-[2-(2,6-dioxo-3-piperidyl)-1,3-dioxo-isoindolin-5-yl]oxyethoxy]-6-(trifluoromethyl)benzenesulfonamide

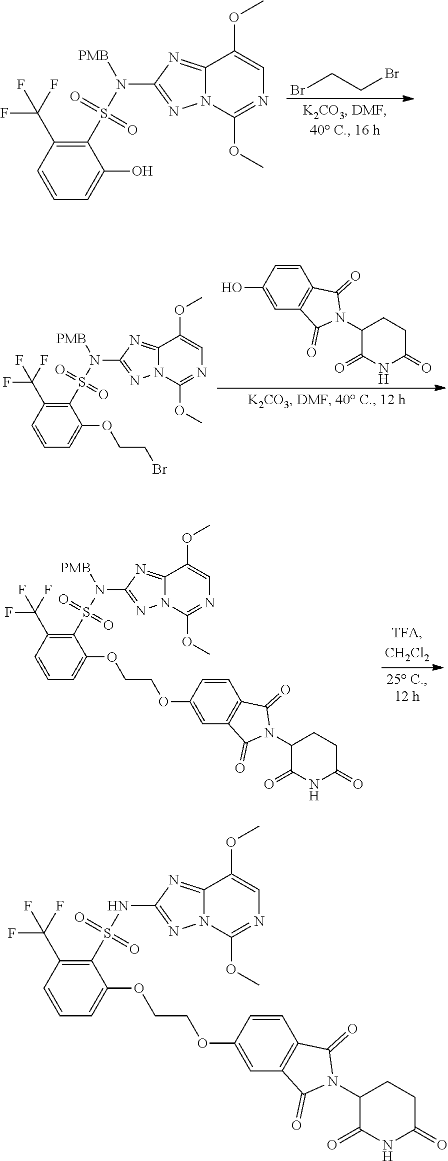

### Step 1

To a mixture of N-(5,8-dimethoxy-[1,2,4]triazolo[1,5-c]pyrimidin-2-yl)-2-hydroxy-N-[(4-methoxyphenyl)methyl]-6-(trifluoromethyl)benzenesulfonamide (0.2 g, 370.72 μmol) in DMF (5 mL) were added 1,2-dibromoethane (208.93 mg, 1.11 mmol, 83.91 μL) and K₂CO₃ (153.71 mg, 1.11 mmol). The mixture was stirred at 40° C. for 12 hours. EtOAc (20 mL) and water (20 mL) were added, and layers were separated. The aqueous phase was extracted with EtOAc (10 mL×3). The combined extracts were washed with brine (20 mL), dried over Na₂SO₄, filtered, and concentrated under vacuum to give the crude product. The residue was purified by prep-TLC (SiO₂, PE:EtOAc=1:1) to give 2-(2-bromoethoxy)-N-(5,8-dimethoxy-[1,2,4]triazolo[1,5-c]pyrimidin-2-yl)-N-[(4-methoxyphenyl)methyl]-6-(trifluoromethyl)benzene-sulfonamide (0.11 g, 170.16 μmol, 45.90% yield) as a white solid. LCMS: RT=1.000 min, m z 648.3 [M+H]⁺.

### Step 2

To a mixture of 2-(2-bromoethoxy)-N-(5,8-dimethoxy-[1,2,4]triazolo[1,5-c]pyrimidin-2-yl)-N-[(4-methoxyphenyl)methyl]-6-(trifluoromethyl)benzenesulfonamide (0.11 g, 170.16 μmol) in DMF (3 mL) were added 2-(2,6-dioxo-3-piperidyl)-5-hydroxy-isoindoline-1,3-dione (93.33 mg, 340.33 μmol) and K₂CO₃ (70.55 mg, 510.49 μmol). The mixture was stirred at 40° C. for 12 hours. EtOAc (20 mL) and water (20 mL) were added, and the layers were separated. The aqueous phase was extracted with EtOAc (10 mL×3). The combined extracts were washed with brine (20 mL×3), dried over Na₂SO₄, filtered, and concentrated under vacuum to give the crude product. The residue was purified by prep-TLC (SiO₂, DCM:MeOH=10:1) to give N-(5,8-dimethoxy-[1,2,4]triazolo[1,5-c]pyrimidin-2-yl)-2-[2-[2-(2,6-dioxo-3-piperidyl)-1,3-dioxo-isoindolin-5-yl]oxyethoxy]-N-[(4-methoxyphenyl)methyl]-6-(trifluoromethyl)benzenesulfonamide (80 mg, 95.27 μmol, 55.98% yield) as a white solid. LCMS: RT=0.722 min, m/z 840.3 [M+H]⁺.

### Step 3

To a mixture of N-(5,8-dimethoxy-[1,2,4]triazolo[1,5-c]pyrimidin-2-yl)-2-[2-[2-(2,6-dioxo-3-piperidyl)-1,3-dioxo-isoindolin-5-yl]oxyethoxy]-N-[(4-methoxyphenyl)methyl]-6-(trifluoromethyl)benzenesulfonamide (80 mg, 95.27 μmol) in DCM (3 mL) was added TFA (4.62 g, 40.52 mmol, 3 mL). The mixture was stirred at 25° C. for 12 hours. The solvent was evaporated under reduced pressure. The crude product was purified by prep-HPLC (column: 3_Phenomenex Luna C18 75*30 mm*3 μm; mobile phase: [water (0.1% TFA)-ACN]; B %: 40%-60%, 7 min) to give N-(5,8-dimethoxy-[1,2,4]triazolo[1,5-c]pyrimidin-2-yl)-2-[2-[2-(2,6-dioxo-3-piperidyl)-1,3-dioxo-isoindolin-5-yl]oxyethoxy]-6-(trifluoromethyl)benzenesulfonamide (28.84 mg, 39.87 μmol, 41.85% yield, 99.49% purity) as a white solid. HNMR: DMSO-d6, 400 MHz. δ 11.92 (s, 1H), 11.13 (s, 1H), 7.86-7.78 (m, 2H), 7.63-7.57 (m, 2H), 7.33 (s, 1H), 6.85-6.76 (m, 2H), 5.17-5.08 (m, 1H), 4.75 (s, 2H), 4.42-4.35 (m, 2H), 3.94 (s, 3H), 3.74 (s, 3H), 2.99-2.83 (m, 1H), 2.69-2.54 (m, 2H), 2.15-2.06 (m, 1H). LCMS: RT=2.834 min, m z 720.0 [M+H]⁺.

The following compounds were prepared in analogous fashion to Example 4.

### Compound 4-A

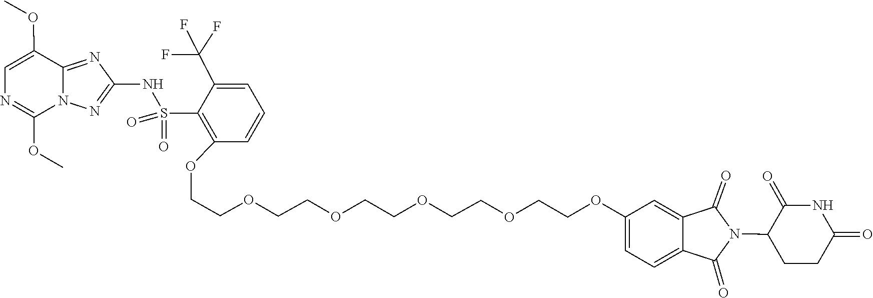 [SMILES: COc1cnc(OC)n2nc(NS(=O)(=O)c3c(OCCOCCOCCOCCOCCOc4ccc5c(c4)C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-((14-((2-(2,6-dioxopiperidin-3-yl)-1,3-dioxoisoindolin-5-yl)oxy)-3,6,9,12-tetraoxatetradecyl)oxy)-6-(trifluoromethyl)benzenesulfonamide. HNMR: CDCl₃, 400 MHz. δ 9.78 (brs, 1H), 8.04 (s, 1H), 7.76 (d, J=8.4 Hz, 1H), 7.60-7.53 (m, 1H), 7.52-7.48 (m, 1H), 7.37-7.33 (m, 2H), 7.25-7.18 (m, 2H), 5.00-4.92 (m, 1H), 4.32-4.24 (m, 4H), 4.11 (s, 3H), 4.05-4.00 (m, 2H), 3.93-3.85 (m, 9H), 3.75-3.68 (m, 8H), 2.95-2.73 (m, 3H), 2.20-2.11 (m, 1H). LCMS: RT=2.987 min, m/z 896.2 [M+H]⁺.

### Compound 4-B

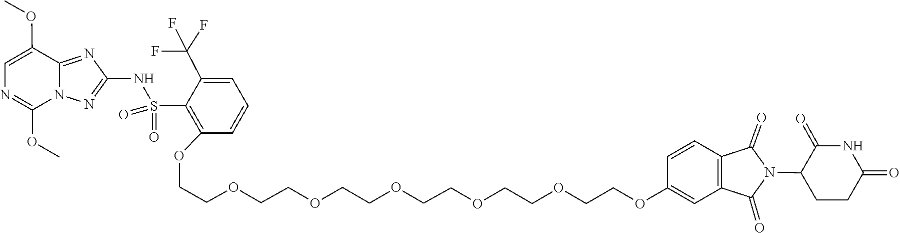 [SMILES: COc1cnc(OC)n2nc(NS(=O)(=O)c3c(OCCOCCOCCOCCOCCOCCOc4ccc5c(c4)C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-((17-((2-(2,6-dioxopiperidin-3-yl)-1,3-dioxoisoindolin-5-yl)oxy)-3,6,9,12,15-pentaoxaheptadecyl)oxy)-6-(trifluoromethyl)benzenesulfonamide. HNMR: DMSO-d₆, 400 MHz. δ 11.18-11.06 (m, 2H), 7.84-7.79 (m, 1H), 7.76-7.68 (m, 1H), 7.65-7.48 (m, 3H), 7.44 (d, J=2.0 Hz, 1H), 7.38-7.33 (m, 1H), 5.16-5.07 (m, 1H), 4.36-4.23 (m, 4H), 4.08-4.01 (m, 3H), 3.87-3.82 (m, 3H), 3.81-3.72 (m, 4H), 3.59-3.43 (m, 16H), 2.94-2.83 (m, 1H), 2.64-2.53 (m, 2H), 2.11-1.99 (m, 1H). LCMS: RT=2.971 min, m/z=940.3 [M+H]⁺.

### Compound 4-C

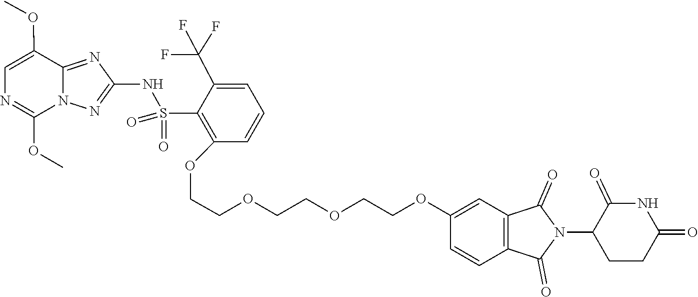 [SMILES: COc1cnc(OC)n2nc(NS(=O)(=O)c3c(OCCOCCOCCOc4ccc5c(c4)C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-(2-(2-(2-((2-(2,6-dioxopiperidin-3-yl)-1,3-dioxoisoindolin-5-yl)oxy)ethoxy)ethoxy)ethoxy)-6-(trifluoromethyl)benzenesulfonamide. HNMR: DMSO-d₆, 400 MHz. δ 9.81-9.67 (m, 1H), 8.08-7.99 (m, 1H), 7.71 (d, J=8.4 Hz, 1H), 7.60-7.49 (m, 2H), 7.35-7.31 (m, 2H), 7.24-7.17 (m, 2H), 4.94 (dd, J=5.2, 12.0 Hz, 1H), 4.35-4.28 (m, 4H), 4.10 (s, 3H), 4.06-4.02 (m, 2H), 4.01-3.90 (m, 9H), 2.95-2.86 (m, 1H), 2.85-2.69 (m, 2H), 2.20-2.12 (m, 1H). LCMS: RT=2.922 min, m/z 808.1 [M+H]⁺.

### Compound 4-D

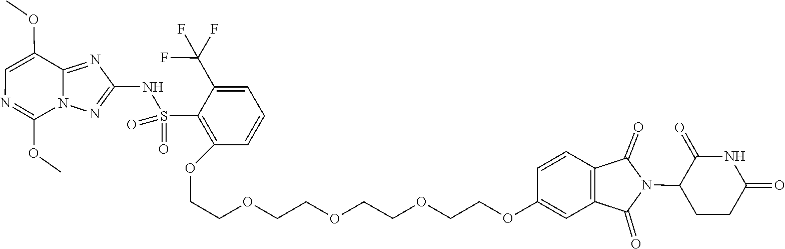 [SMILES: COc1cnc(OC)n2nc(NS(=O)(=O)c3c(OCCOCCOCCOCCOc4ccc5c(c4)C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-(2-(2-(2-(2-((2-(2,6-dioxopiperidin-3-yl)-1,3-dioxoisoindolin-5-yl)oxy)ethoxy)ethoxy)ethoxy)ethoxy)-6-(trifluoromethyl)benzenesulfonamide. HNMR: CDCl₃, 400 MHz. δ 9.77 (s, 1H), 8.02 (brs, 1H), 7.74 (d, J=8.4 Hz, 1H), 7.57-7.50 (m, 2H), 7.35-7.34 (m, 2H), 7.23-7.21 (m, 2H), 4.96 (dd, J=12.0, 5.6 Hz, 1H), 4.28-4.26 (m, 4H), 4.11 (s, 3H), 4.02-3.99 (m, 2H), 3.94-3.93 (m, 2H), 3.92 (s, 3H), 3.90-3.88 (m, 4H), 3.81-3.79 (m, 2H), 3.76-3.75 (m, 2H), 2.94-2.72 (m, 3H), 2.18-2.15 (m, 1H). LCMS: RT=2.956 min, m/z 852.2 [M+H]⁺.

### Compound 4-E

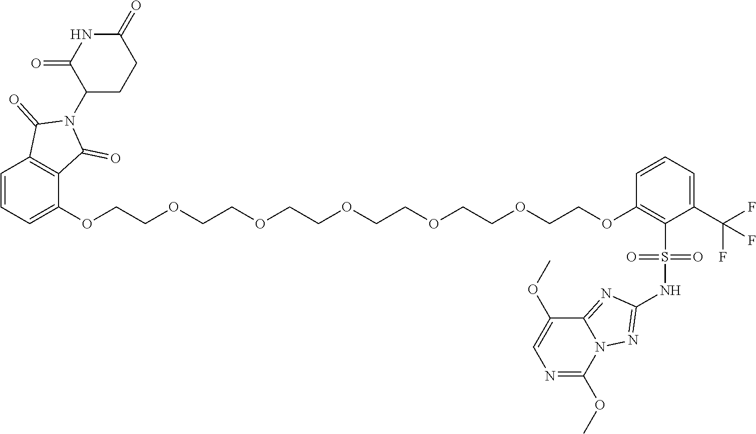 [SMILES: COc1cnc(OC)n2nc(NS(=O)(=O)c3c(OCCOCCOCCOCCOCCOCCOc4cccc5c4C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-((17-((2-(2,6-dioxopiperidin-3-yl)-1,3-dioxoisoindolin-4-yl)oxy)-3,6,9,12,15-pentaoxaheptadecyl)oxy)-6-(trifluoromethyl)benzenesulfonamide. HNMR: DMSO-d₆, 400 MHz. δ 11.06 (s, 1H), 7.86-7.42 (m, 7H), 5.07 (dd, J=5.2, 12.8 Hz, 1H), 4.43-4.17 (m, 4H), 4.05 (s, 3H), 3.84 (s, 3H), 3.82-3.71 (m, 4H), 3.65-3.60 (m, 2H), 3.54-3.43 (m, 14H), 2.94-2.82 (m, 1H), 2.63-2.55 (m, 2H), 2.08-1.98 (m, 1H). LCMS: =2.975 min, m/z 940.2 [M+H]⁺.

### Compound 4-F

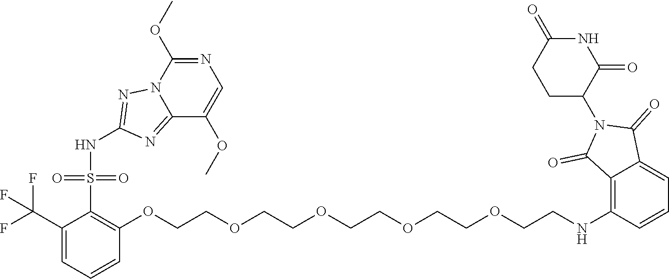 [SMILES: COc1cnc(OC)n2nc(NS(=O)(=O)c3c(OCCOCCOCCOCCOCCNc4cccc5c4C(=O)N(C4CCC(=O)NC4=O)C5=O)cccc3C(F)(F)F)nc12]

N-(5,8-Dimethoxy-[1,2,4]triazolo[1,5-c]pyrimidin-2-yl)-2-((14-((2-(2,6-dioxopiperidin-3-yl)-1,3-dioxoisoindolin-4-yl)amino)-3,6,9,12-tetraoxatetradecyl)oxy)-6-(trifluoromethyl)benzenesulfonamide. HNMR: CDCl₃, 400 MHz. δ 10.08-9.46 (m, 1H), 8.23 (s, 1H), 7.56-7.41 (m, 3H), 7.37-7.27 (m, 1H), 7.23-7.13 (m, 1H), 7.07 (d, J=6.8 Hz, 1H), 6.89 (d, J=6.4 Hz, 1H), 6.50-6.40 (m, 1H), 4.99-4.85 (m, 1H), 4.42-4.12 (m, 2H), 4.09 (s, 3H), 3.91 (s, 3H), 3.86-3.59 (m, 16H), 3.49-3.43 (m, 2H), 2.91-2.73 (m, 3H), 2.19-2.11 (m, 1H), 1.37-1.20 (m, 2H). LCMS: RT=3.062 min, m/z 895.2 [M+H]⁺.

### Compound 4-G

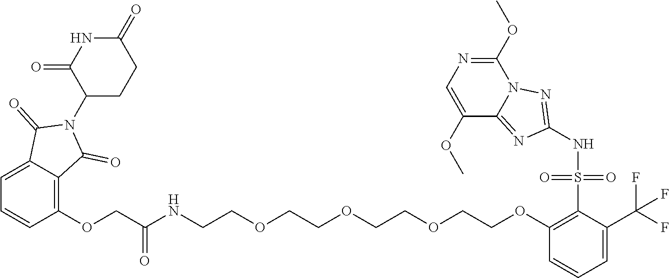 [SMILES: COc1cnc(OC)n2nc(NS(=O)(=O)c3c(OCCOCCOCCOCCNC(=O)COc4cccc5c4C(=O)N(C4CCC(=O)NC4=O)C5=O)cccc3C(F)(F)F)nc12]

N-(2-(2-(2-(2-(2-(N-(5,8-Dimethoxy-[1,2,4]triazolo[1,5-c]pyrimidin-2-yl)sulfamoyl)-3-(trifluoromethyl)phenoxy)ethoxy)ethoxy)ethoxy)ethyl)-2-((2-(2,6-dioxopiperidin-3-yl)-1,3-dioxoisoindolin-4-yl)oxy)acetamide. HNMR: DMSO-d₆, 400 MHz. δ 11.11 (s, 1H), 8.03-7.94 (m, 1H), 7.83-7.69 (m, 2H), 7.65-7.51 (m, 3H), 7.48 (d, J=7.6 Hz, 1H), 7.38 (d, J=8.8 Hz, 1H), 5.16-5.06 (m, 1H), 4.84-4.72 (m, 2H), 4.35-4.26 (m, 2H), 4.10-3.99 (m, 3H), 3.88-3.77 (m, 5H), 3.56-3.51 (m, 2H), 3.49-3.40 (m, 8H), 3.30-3.26 (m, 2H), 2.95-2.84 (m, 1H), 2.61-2.56 (m, 2H), 2.10-1.98 (m, 1H). LCMS: RT=2.827 min, m/z 909.3 [M+H]⁺.

### Compound 4-H

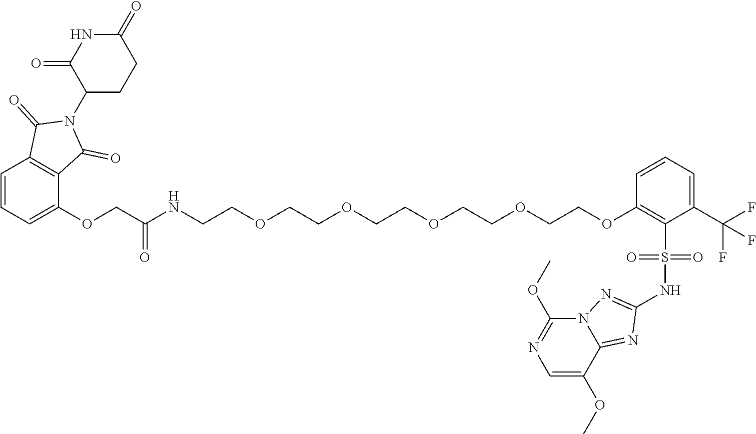 [SMILES: COc1cnc(OC)n2nc(NS(=O)(=O)c3c(OCCOCCOCCOCCOCCNC(=O)COc4cccc5c4C(=O)N(C4CCC(=O)NC4=O)C5=O)cccc3C(F)(F)F)nc12]

N-(14-(2-(N-(5,8-Dimethoxy-[1,2,4]triazolo[1,5-c]pyrimidin-2-yl)sulfamoyl)-3-(trifluoromethyl)phenoxy)-3,6,9,12-tetraoxatetradecyl)-2-((2-(2,6-dioxopiperidin-3-yl)-1, 3-dioxoisoindolin-4-yl)oxy)acetamide. HNMR: DMSO-d₆, 400 MHz. δ 11.11 (d, J=3.6 Hz, 2H), 7.99 (t, J=5.2 Hz, 1H), 7.83-7.77 (m, 1H), 7.76-7.70 (m, 1H), 7.62 (d, J=8.4 Hz, 1H), 7.58 (s, 1H), 7.54 (d, J=8.0 Hz, 1H), 7.48 (d, J=7.2 Hz, 1H), 7.38 (d, J=8.4 Hz, 1H), 5.11 (dd, J=5.2, 12.8 Hz, 1H), 4.78 (s, 2H), 4.31 (t, J=4.4 Hz, 2H), 4.05 (s, 3H), 3.85 (s, 3H), 3.79 (t, J=4.8 Hz, 2H), 3.55-3.51 (m, 2H), 3.49-3.43 (m, 12H), 3.33-3.27 (m, 2H), 2.94-2.84 (m, 1H), 2.63-2.52 (m, 2H), 2.07-2.00 (m, 1H). LCMS: RT=3.426 min, m/z 953.3 [M+H]⁺.

### Compound 4-I

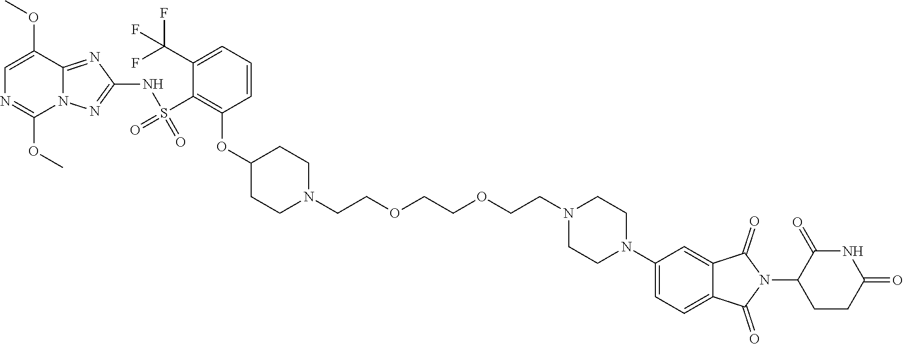 [SMILES: COc1cnc(OC)n2nc(NS(=O)(=O)c3c(OC4CCN(CCOCCOCCN5CCN(c6ccc7c(c6)C(=O)N(C6CCC(=O)NC6=O)C7=O)CC5)CC4)cccc3C(F)(F)F)nc12]

N-(5,8-Dimethoxy-[1,2,4]triazolo[1,5-c]pyrimidin-2-yl)-2-((1-(2-(2-(2-(4-(2-(2,6-dioxopiperidin-3-yl)-1,3-dioxoisoindolin-5-yl)piperazin-1-yl)ethoxy)ethoxy)ethyl)piperidin-4-yl)oxy)-6-(trifluoromethyl)benzenesulfonamide. HNMR: DMSO-d₆, 400 MHz. δ 11.08 (s, 1H), 8.14 (s, 1H), 7.73-7.61 (m, 2H), 7.52-7.43 (m, 3H), 7.33 (d, J=2.0 Hz, 1H), 7.23 (dd, J=2.0, 8.8 Hz, 1H), 5.16-5.09 (m, 1H), 5.06 (dd, J=5.2, 12.8 Hz, 1H), 3.85-3.80 (m, 6H), 3.59-3.49 (m, 10H), 3.45-3.40 (m, 6H), 2.92-2.83 (m, 1H), 2.75-2.67 (m, 2H), 2.60-2.53 (m, 8H), 2.05-1.94 (m, 3H), 1.81-1.69 (m, 2H). LCMS: RT=2.286 min, m/z 959.3[M+H]⁺.

### Compound 4-J

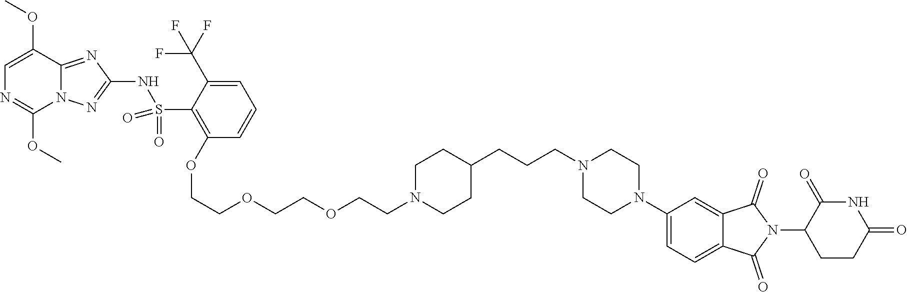 [SMILES: COc1cnc(OC)n2nc(NS(=O)(=O)c3c(OCCOCCOCCN4CCC(CCCN5CCN(c6ccc7c(c6)C(=O)N(C6CCC(=O)NC6=O)C7=O)CC5)CC4)cccc3C(F)(F)F)nc12]

N-(5,8-Dimethoxy-[1,2,4]triazolo[1,5-c]pyrimidin-2-yl)-2-(2-(2-(2-(4-(3-(4-(2-(2,6-dioxopiperidin-3-yl)-1,3-dioxoisoindolin-5-yl)piperazin-1-yl)propyl)piperidin-1-yl)ethoxy)ethoxy)ethoxy)-6-(trifluoromethyl)benzenesulfonamide. HNMR: DMSO-d₆, 400 MHz. δ 11.10 (s, 1H), 11.02-10.87 (m, 1H), 10.07-9.75 (m, 1H), 9.46-9.11 (m, 1H), 7.81-7.72 (m, 2H), 7.66-7.60 (m, 2H), 7.57 (d, J=7.6 Hz, 1H), 7.48 (s, 1H), 7.40-7.33 (m, 1H), 5.09 (dd, J=5.2, 12.4 Hz, 1H), 4.35-4.29 (m, 2H), 4.27-4.16 (m, 2H), 4.06 (s, 3H), 3.87-3.82 (m, 5H), 3.75-3.70 (m, 2H), 3.67-3.46 (m, 14H), 3.27-3.21 (m, 2H), 3.14-3.06 (m, 2H), 2.96-2.81 (m, 2H), 2.64-2.53 (m, 1H), 2.08-1.97 (m, 1H), 1.89-1.73 (m, 2H), 1.72-1.61 (m, 2H), 1.52-1.40 (m, 1H), 1.39-1.26 (m, 2H), 1.25-1.11 (m, 2H). LCMS: RT=2.139 min, m/z 1001.3 [M+H]⁺.

### Compound 4-K

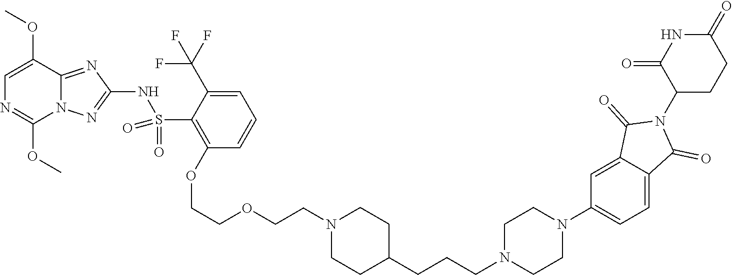 [SMILES: COc1cnc(OC)n2nc(NS(=O)(=O)c3c(OCCOCCN4CCC(CCCN5CCN(c6ccc7c(c6)C(=O)N(C6CCC(=O)NC6=O)C7=O)CC5)CC4)cccc3C(F)(F)F)nc12]

N-(5,8-Dimethoxy-[1,2,4]triazolo[1,5-c]pyrimidin-2-yl)-2-(2-(2-(4-(3-(4-(2-(2,6-dioxopiperidin-3-yl)-1,3-dioxoisoindolin-5-yl)piperazin-1-yl)propyl)piperidin-1-yl)ethoxy)ethoxy)-6-(trifluoromethyl)benzenesulfonamide. HNMR: DMSO-d₆, 400 MHz. δ 11.11-11.01 (m, 1H), 8.17-8.11 (m, 1H), 7.71-7.65 (m, 1H), 7.57-7.50 (m, 1H), 7.46-7.38 (m, 2H), 7.37-7.29 (m, 2H), 7.28-7.23 (m, 1H), 5.12-5.02 (m, 1H), 4.06-4.01 (m, 2H), 4.00 (s, 3H), 3.96-3.91 (m, 2H), 3.80 (s, 3H), 3.76-3.61 (m, 8H), 3.01-2.84 (m, 4H), 2.64-2.52 (m, 6H), 2.43-2.36 (m, 2H), 2.08-1.99 (m, 1H), 1.95-1.71 (m, 5H), 1.57-1.47 (m, 3H), 1.32-1.24 (m, 2H). LCMS: RT=2.435 min, m/z=957.3 [M+H]⁺.

### Compound 4-L

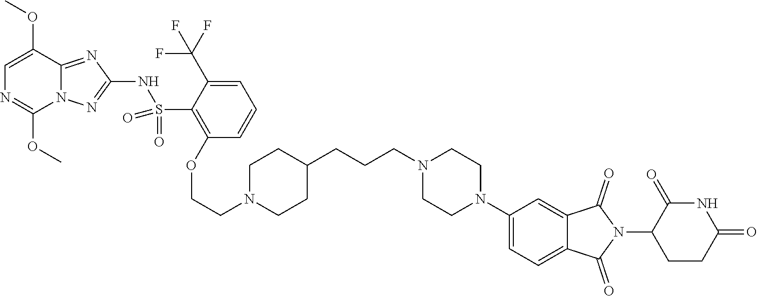 [SMILES: COc1cnc(OC)n2nc(NS(=O)(=O)c3c(OCCN4CCC(CCCN5CCN(c6ccc7c(c6)C(=O)N(C6CCC(=O)NC6=O)C7=O)CC5)CC4)cccc3C(F)(F)F)nc12]

N-(5,8-Dimethoxy-[1,2,4]triazolo[1,5-c]pyrimidin-2-yl)-2-(2-(4-(3-(4-(2-(2,6-dioxopiperidin-3-yl)-1,3-dioxoisoindolin-5-yl)piperazin-1-yl)propyl)piperidin-1-yl)ethoxy)-6-(trifluoromethyl)benzenesulfonamide. HNMR: CDCl₃, 400 MHz. δ 8.02 (s, 1H), 7.74-7.70 (m, 1H), 7.58-7.54 (m, 1H), 7.51-7.46 (m, 1H), 7.33-7.32 (m, 1H), 7.31-7.30 (m, 1H), 7.21-7.17 (m, 1H), 7.09 (dd, J=2.0, 8.4 Hz, 1H), 5.00-4.86 (m, 1H), 4.38-4.31 (m, 2H), 4.10 (s, 3H), 3.95 (s, 3H), 3.79-3.70 (m, 2H), 3.54-3.46 (m, 4H), 3.13-3.06 (m, 2H), 2.96-2.89 (m, 1H), 2.87-2.83 (m, 1H), 2.81-2.77 (m, 1H), 2.75-2.69 (m, 4H), 2.54-2.45 (m, 4H), 2.17-2.12 (m, 2H), 1.90-1.83 (m, 2H), 1.64-1.59 (m, 2H), 1.54-1.41 (m, 2H), 1.36-1.27 (m, 2H). LCMS: RT=2.078 min, m/z 913.3 [M+H]⁺.

### Compound 4-M

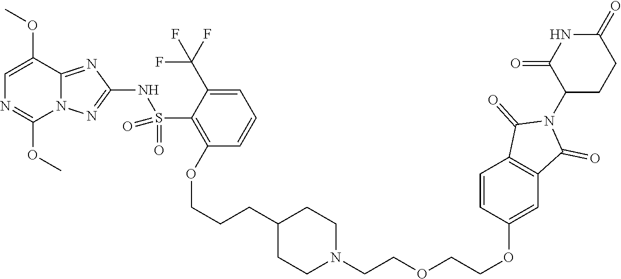 [SMILES: COc1cnc(OC)n2nc(NS(=O)(=O)c3c(OCCCC4CCN(CCOCCOc5ccc6c(c5)C(=O)N(C5CCC(=O)NC5=O)C6=O)CC4)cccc3C(F)(F)F)nc12]

N-(5,8-Dimethoxy-[1,2,4]triazolo[1,5-c]pyrimidin-2-yl)-2-(3-(1-(2-(2-((2-(2,6-dioxopiperidin-3-yl)-1,3-dioxoisoindolin-5-yl)oxy)ethoxy)ethyl)piperidin-4-yl)propoxy)-6-(trifluoromethyl)benzenesulfonamide. HNMR: DMSO-d₆, 400 MHz. δ 11.96-11.72 (m, 1H), 11.25-11.01 (m, 1H), 9.34-8.97 (m, 1H), 7.88-7.64 (m, 2H), 7.59-7.30 (m, 5H), 5.18-4.98 (m, 1H), 4.47-4.24 (m, 4H), 3.89-3.78 (m, 10H), 3.25-3.13 (m, 4H), 2.99-2.81 (m, 5H), 2.04-1.99 (m, 1H), 1.84-1.70 (m, 4H), 1.51-1.43 (m, 1H), 1.36-1.20 (m, 4H). LCMS: RT=2.354 min, m/z=889.2 [M+H]⁺.

### Compound 4-N

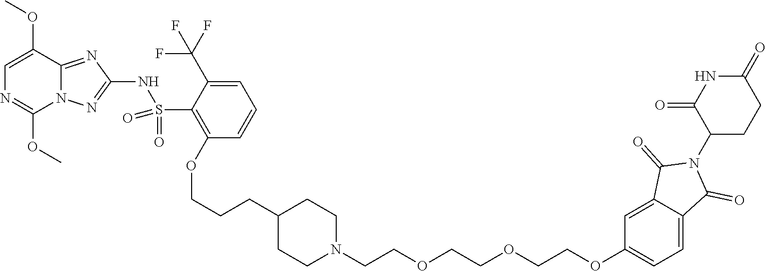 [SMILES: COc1cnc(OC)n2nc(NS(=O)(=O)c3c(OCCCC4CCN(CCOCCOCCOc5ccc6c(c5)C(=O)N(C5CCC(=O)NC5=O)C6=O)CC4)cccc3C(F)(F)F)nc12]

N-(5,8-Dimethoxy-[1,2,4]triazolo[1,5-c]pyrimidin-2-yl)-2-(3-(1-(2-(2-(2-((2-(2,6-dioxopiperidin-3-yl)-1,3-dioxoisoindolin-5-yl)oxy)ethoxy)ethoxy)ethyl)piperidin-4-yl)propoxy)-6-(trifluoromethyl)benzenesulfonamide. HNMR: DMSO-d₆, 400 MHz. δ 11.88 (s, 1H,) 11.12 (s, 1H), 9.14 (br s, 1H), 7.87-7.85 (m, 1H), 7.75 (d, J=8.0 Hz, 1H), 7.56-7.51 (m, 3H), 7.47 (d, J=2.0 Hz, 1H), 7.37 (dd, J=8.4, 2.4 Hz, 1H), 5.12 (dd, J=12.8, 5.2 Hz, 1H), 4.43-4.41 (m, 2H), 4.34-4.32 (m, 2H), 3.87 (d, J=7.2 Hz, 6H), 3.81-3.76 (m, 4H), 3.66-3.65 (m, 4H), 3.52-3.49 (m, 2H), 3.26-3.25 (m, 2H), 2.97-2.86 (m, 3H), 2.62-2.57 (m, 2H), 2.06-2.03 (m, 1H), 1.83-1.73 (m, 4H), 1.50-1.46 (brs, 1H), 1.33-1.29 (m, 4H). LCMS: RT=2.373 min, m/z 933.3 [M+H]⁺.

### Compound 4-0

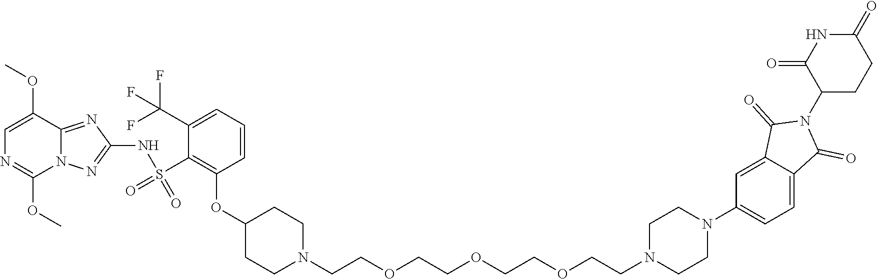 [SMILES: COc1cnc(OC)n2nc(NS(=O)(=O)c3c(OC4CCN(CCOCCOCCOCCN5CCN(c6ccc7c(c6)C(=O)N(C6CCC(=O)NC6=O)C7=O)CC5)CC4)cccc3C(F)(F)F)nc12]

N-(5,8-Dimethoxy-[1,2,4]triazolo[1,5-c]pyrimidin-2-yl)-2-((1-(2-(2-(2-(2-(4-(2-(2,6-dioxopiperidin-3-yl)-1,3-dioxoisoindolin-5-yl)piperazin-1-yl)ethoxy)ethoxy)ethoxy)ethyl)piperidin-4-yl)oxy)-6-(trifluoromethyl)benzenesulfonamide. HNMR: CDCl₃, 400 MHz. δ 8.28 (brs, 1H), 7.68 (d, J=8.8 Hz, 1H), 7.61-7.55 (m, 1H), 7.52-7.50 (m, 1H), 7.32 (s, 1H), 7.28-7.27 (m, 1H), 7.25-7.23 (m, 1H), 7.05 (dd, J=8.8, 2.4 Hz, 1H), 5.20-5.18 (m, 1H), 4.94 (dd, J=12.0, 5.2 Hz, 1H), 4.00 (s, 3H), 3.94 (s, 3H), 3.69-3.65 (m, 12H), 3.46-3.45 (m, 4H), 2.80-2.67 (m, 13H), 2.56-2.53 (m, 2H), 2.14-2.10 (m, 3H), 2.00-1.98 (m, 2H). LCMS: RT=2.325 min, m/z 1003.3 [M+H]⁺.

### Compound 4-P

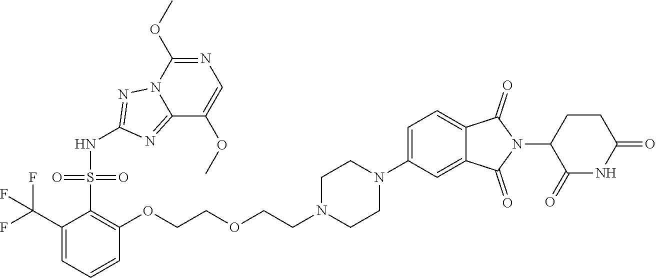 [SMILES: COc1cnc(OC)n2nc(NS(=O)(=O)c3c(OCCOCCN4CCN(c5ccc6c(c5)C(=O)N(C5CCC(=O)NC5=O)C6=O)CC4)cccc3C(F)(F)F)nc12]

N-(5,8-dimethoxy-[1,2,4]triazolo[1,5-c]pyrimidin-2-yl)-2-(2-(2-(4-(2-(2,6-dioxopiperidin-3-yl)-1,3-dioxoisoindolin-5-yl)piperazin-1-yl)ethoxy)ethoxy)-6-(trifluoromethyl)benzenesulfonamide. HNMR: DMSO-d₆, 400 MHz. δ 11.39-11.26 (m, 1H), 11.11 (s, 1H), 10.00-9.73 (m, 1H), 7.80-7.73 (m, 2H), 7.63-7.55 (m, 3H), 7.47-7.43 (m, 1H), 7.31 (dd, J=2.4, 8.8 Hz, 1H), 5.10 (dd, J=5.2, 12.8 Hz, 1H), 4.38-4.31 (m, 2H), 4.22-4.08 (m, 2H), 4.04 (s, 3H), 3.93-3.83 (m, 7H), 3.66-3.60 (m, 2H), 3.43-3.38 (m, 2H), 3.32-3.11 (m, 4H), 2.94-2.83 (m, 1H), 2.64-2.52 (m, 2H), 2.07-1.99 (m, 1H). LCMS: RT=2.668 min, m/z 832.2 [M+H]⁺.

### Compound 4-Q

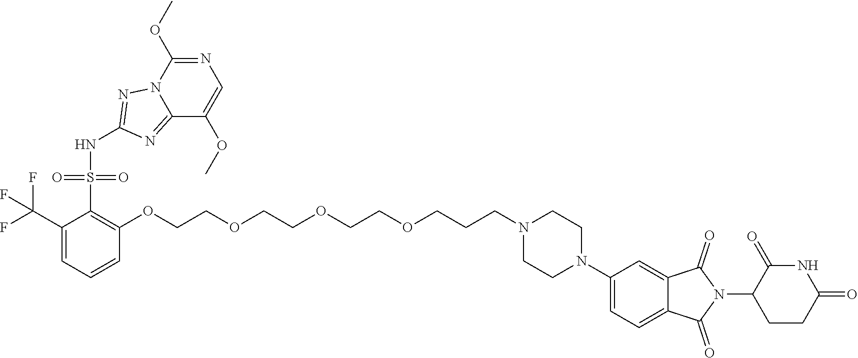 [SMILES: COc1cnc(OC)n2nc(NS(=O)(=O)c3c(OCCOCCOCCOCCCN4CCN(c5ccc6c(c5)C(=O)N(C5CCC(=O)NC5=O)C6=O)CC4)cccc3C(F)(F)F)nc12]

N-(5,8-Dimethoxy-[1,2,4]triazolo[1,5-c]pyrimidin-2-yl)-2-(2-(2-(2-(3-(4-(2-(2,6-dioxopiperidin-3-yl)-1,3-dioxoisoindolin-5-yl)piperazin-1-yl)propoxy)ethoxy)ethoxy)ethoxy)-6-(trifluoromethyl)benzenesulfonamide. HNMR: DMSO-d₆, 400 MHz. δ 11.08 (s, 1H), 8.18 (s, 1H), 7.66 (d, J=8.8 Hz, 1H), 7.57-7.51 (m, 1H), 7.47-7.39 (m, 3H), 7.32 (d, J=2.0 Hz, 1H), 7.23 (dd, J=2.0, 8.8 Hz, 1H), 5.07 (dd, J=5.2, 12.8 Hz, 1H), 4.12-4.06 (m, 2H), 4.02 (s, 3H), 3.81 (s, 3H), 3.65-3.45 (m, 16H), 2.92-2.84 (m, 1H), 2.62-2.54 (m, 2H), 2.49-2.46 (m, 4H), 2.38-2.33 (m, 2H), 2.07-1.97 (m, 1H), 1.71-1.62 (m, 2H). LCMS: RT=2.687 min, m/z 934.3 [M+H]⁺.

### Compound 4-R

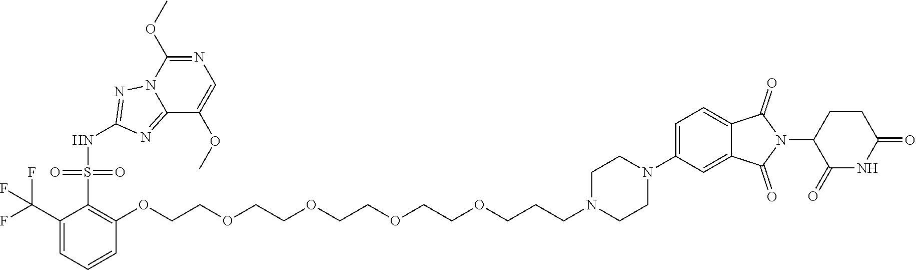 [SMILES: COc1cnc(OC)n2nc(NS(=O)(=O)c3c(OCCOCCOCCOCCOCCCN4CCN(c5ccc6c(c5)C(=O)N(C5CCC(=O)NC5=O)C6=O)CC4)cccc3C(F)(F)F)nc12]

N-(5,8-Dimethoxy-[1,2,4]triazolo[1,5-c]pyrimidin-2-yl)-2-((15-(4-(2-(2,6-dioxopiperidin-3-yl)-1,3-dioxoisoindolin-5-yl)piperazin-1-yl)-3,6,9,12-tetraoxapentadecyl)oxy)-6-(trifluoromethyl)benzenesulfonamide. HNMR: CDCl₃, 400 MHz. δ 11.10 (s, 1H), 10.87 (brs, 1H), 9.93-9.64 (m, 1H), 7.78-7.71 (m, 2H), 7.64 (d, J=8.0 Hz, 1H), 7.56-7.45 (m, 3H), 7.34 (dd, J=2.0, 8.8 Hz, 1H), 5.09 (dd, J=5.6, 12.8 Hz, 1H), 4.34-4.32 (m, 1H), 4.37-4.30 (m, 2H), 4.22 (br d, J=10.0 Hz, 2H), 3.86-3.80 (m, 4H), 3.72 (s, 3H), 3.59-3.57 (m, 2H), 3.53-3.47 (m, 11H), 3.44 (s, 3H), 3.30-3.09 (m, 6H), 2.96-2.83 (m, 1H), 2.64-2.52 (m, 2H), 2.08-1.89 (m, 3H). LCMS: RT=2.593 min, m/z 978.3 [M+H]⁺.

### Compound 4-S

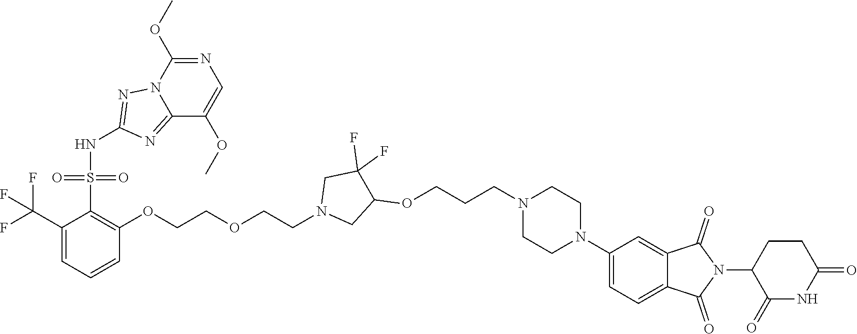 [SMILES: COc1cnc(OC)n2nc(NS(=O)(=O)c3c(OCCOCCN4CC(OCCCN5CCN(c6ccc7c(c6)C(=O)N(C6CCC(=O)NC6=O)C7=O)CC5)C(F)(F)C4)cccc3C(F)(F)F)nc12]

N-(5,8-Dimethoxy-[1,2,4]triazolo[1,5-c]pyrimidin-2-yl)-2-(2-(2-(4-(3-(4-(2-(2,6-dioxopiperidin-3-yl)-1,3-dioxoisoindolin-5-yl)piperazin-1-yl)propoxy)-3,3-difluoropyrrolidin-1-yl)ethoxy)ethoxy)-6-(trifluoromethyl)benzenesulfonamide. HNMR: CDCl₃, 400 MHz. δ 8.02 (s, 1H), 7.73 (d, J=8.4 Hz, 1H), 7.55-7.61 (m, 1H), 7.48-7.53 (m, 1H), 7.37 (s, 1H), 7.32 (d, J=2.0 Hz, 1H), 7.18-7.23 (m, 1H), 7.09-7.15 (m, 1H), 4.93-5.01 (m, 1H), 4.25-4.30 (m, 2H), 4.13 (s, 3H), 3.94-4.05 (m, 4H), 3.92 (s, 3H), 3.73-3.89 (m, 7H), 3.63-3.69 (s, 1H), 3.34-3.55 (m, 5H), 3.06-3.26 (m, 4H), 2.77-3.02 (m, 5H), 2.13-2.28 (m, 3H). LCMS: RT=2.170 min, m/z 995.3 [M+H]⁺.

### Example 5: Synthesis of 2-(2,6-dioxo-3-piperidyl)-5-[2-[2-[2-[4-[4-[(E)-N-ethoxy-C-ethyl-carbonimidoyl]-3-hydroxy-5-oxo-cyclohex-3-en-1-yl]phenoxy]ethoxy]ethoxy]ethoxy]isoindoline-1,3-dione

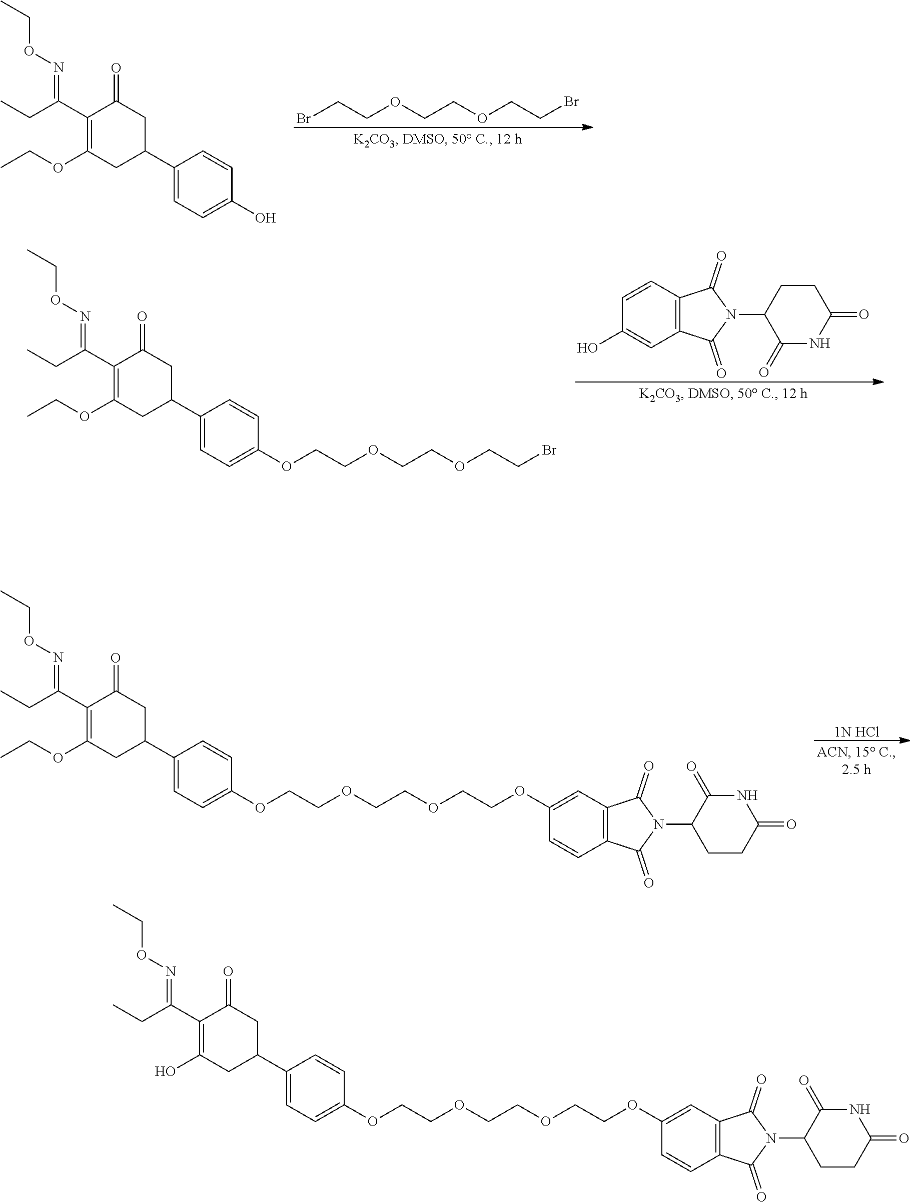

### Step 1

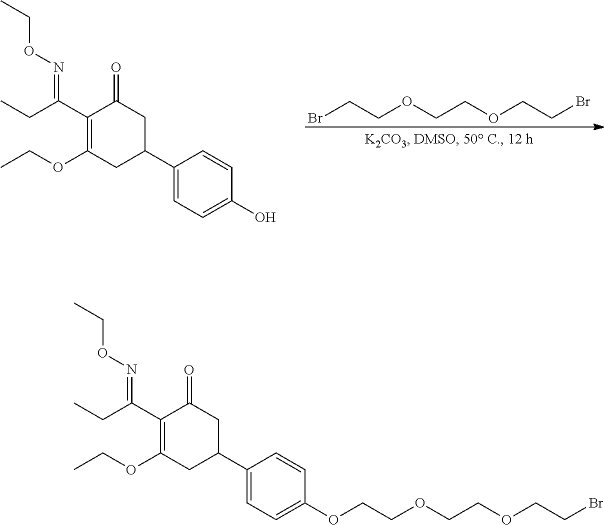 [SMILES: CCO/N=C(\CC)C1=C(OCC)CC(c2ccc(O)cc2)CC1=O ; BrCCOCCOCCBr ; CCO/N=C(\CC)C1=C(OCC)CC(c2ccc(OCCOCCOCCBr)cc2)CC1=O]

To a mixture of 3-ethoxy-2-[(E)-N-ethoxy-C-ethyl-carbonimidoyl]-5-(4-hydroxyphenyl)cyclohex-2-en-1-one (500 mg, 1.51 mmol) and 1,2-bis(2-bromoethoxy)ethane (833 mg, 3.02 mmol) in DMSO (10 mL) was added K₂CO₃ (417 mg, 3.02 mmol). The mixture was then stirred at 50° C. for 12 hours. The reaction mixture was diluted with water (50 mL), and then extracted with Ethyl acetate (20 mL×2). The combined organic phases were washed with brine (50 mL×3), dried over Na₂SO₄, filtered, and concentrated under reduced pressure. The residue was purified by column chromatography (SiO₂, Petroleum ether/Ethyl acetate=10/1 to 2/1) to give 5-[4-[2-[2-(2-bromoethoxy)ethoxy]ethoxy]phenyl]-3-ethoxy-2-[(E)-N-ethoxy-C-ethyl-carbonimidoyl]cyclohex-2-en-1-one (410 mg, 739.85 μmol, 49.04% yield, 95% purity) as a yellow solid. LCMS: RT=0.921 min, m/z 526.1 [M+H]⁺.

### Step 2

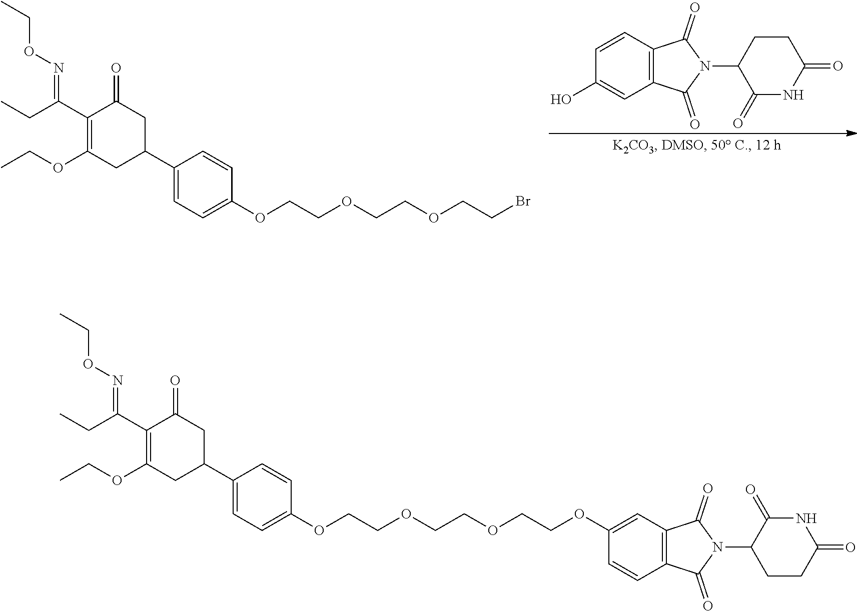 [SMILES: CCO/N=C(\CC)C1=C(OCC)CC(c2ccc(OCCOCCOCCBr)cc2)CC1=O ; O=C1CCC(N2C(=O)c3ccc(O)cc3C2=O)C(=O)N1 ; CCO/N=C(\CC)C1=C(OCC)CC(c2ccc(OCCOCCOCCOc3ccc4c(c3)C(=O)N(C3CCC(=O)NC3=O)C4=O)cc2)CC1=O]

The mixture of 2-(2,6-dioxo-3-piperidyl)-5-hydroxy-isoindoline-1,3-dione (210 mg, 765.78 μmol), 5-[4-[2-[2-(2-bromoethoxy)ethoxy]ethoxy]phenyl]-3-ethoxy-2-[(E)-N-ethoxy-C-ethyl-carbonimidoyl]cyclohex-2-en-1-one (410 mg, 778.79 μmol) and K₂CO₃ (316 mg, 2.29 mmol) in DMSO (5 mL) was stirred at 50° C. for 12 hours. The reaction mixture was diluted with water (50 mL), extracted with Ethyl acetate (20 mL×2). The combined organic phases were washed with brine (50 mL×3), dried over Na₂SO₄, filtered, and concentrated under reduced pressure. The residue was purified by prep-HPLC (column: Phenomenex luna C18 150*25 mm*10 μm; mobile phase: [water (0.05% HCl)-ACN]; B %: 41%-71%, 10 min), prep-HPLC (column: Waters Xbridge 150*25 mm*5 μm; mobile phase: [water (10 mM NH₄HCO₃)-ACN]; B %: 17%-47%, 10 min) and prep-HPLC (column: Waters Xbridge 150*25 mm*5 μm; mobile phase: [water (10 mM NH₄HCO₃)-ACN]; B %: 41%-71%, 10 min) to give 2-(2,6-dioxo-3-piperidyl)-5-[2-[2-[2-[4-[3-ethoxy-4-[(E)-N-ethoxy-C-ethyl-carbonimidoyl]-5-oxo-cyclohex-3-en-1-yl]phenoxy]ethoxy]ethoxy]ethoxy]isoindoline-1,3-dione (40 mg, 55.57 μmol, 7.29% yield) as a yellow solid. HNMR: CDCl₃, 400 MHz. δ 7.78 (d, J=8.0 Hz, 1H), 7.38 (d, J=2.4 Hz, 1H), 7.16-7.25 (m, 3H), 6.91 (d, J=8.4 Hz, 2H), 4.96-5.00 (m, 1H), 4.23-4.27 (m, 2H), 4.05-4.20 (m, 6H), 3.90-3.96 (m, 2H), 3.82-3.88 (m, 2H), 3.73-3.77 (m, 4H), 3.23-3.45 (m, 1H), 2.30-2.95 (m, 10H), 2.15-2.20 (m, 1H), 1.25-1.37 (m, 10H), 0.96-1.12 (m, 4H). LCMS: RT=0.848 min, m/z 720.4 [M+H]⁺.

### Step 3

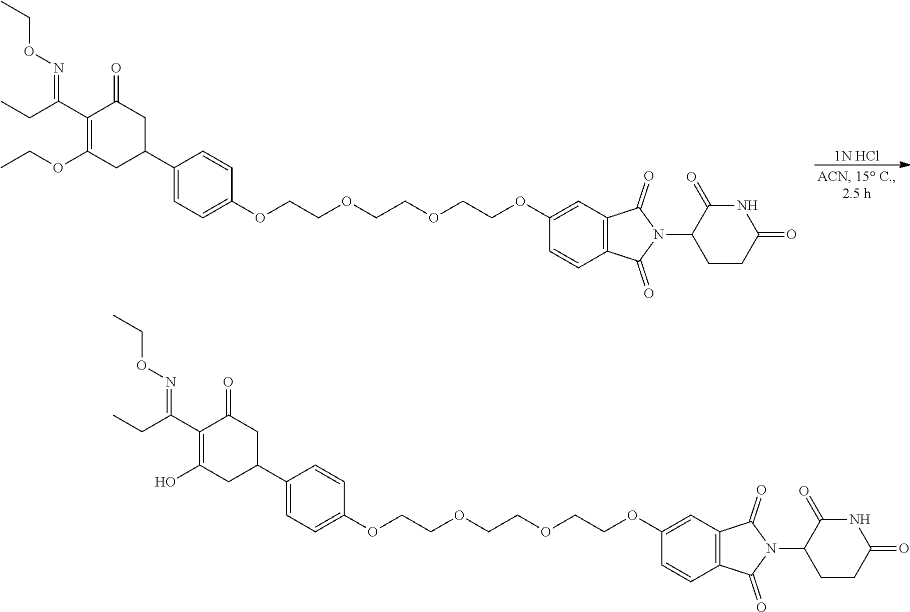 [SMILES: CCO/N=C(\CC)C1=C(OCC)CC(c2ccc(OCCOCCOCCOc3ccc4c(c3)C(=O)N(C3CCC(=O)NC3=O)C4=O)cc2)CC1=O ; "1N HCl" ; CCO/N=C(\CC)C1=C(O)CC(c2ccc(OCCOCCOCCOc3ccc4c(c3)C(=O)N(C3CCC(=O)NC3=O)C4=O)cc2)CC1=O]

To a mixture of 2-(2,6-dioxo-3-piperidyl)-5-[2-[2-[2-[4-[3-ethoxy-4-[(E)-N-ethoxy-C-ethyl-carbonimidoyl]-5-oxo-cyclohex-3-en-1-yl]phenoxy]ethoxy]ethoxy]ethoxy]isoindoline-1,3-dione (40 mg, 55.57 μmol) in ACN (3 mL) was added HCl aqueous (1 M, 666.67 μL). The mixture was then stirred at 15° C. for 2.5 hours. The mixture was poured into water (20 mL), adjusted with saturated NaHCO₃aqueous to pH-7. The mixture was extracted with Ethyl acetate (10 mL×2). The combined organic phases were washed with brine (40 mL), dried over Na₂SO₄, filtered, and concentrated under reduced pressure. The residue was purified by prep-TLC (Ethyl acetate:Petroleum ether=2:1) to give 2-(2,6-dioxo-3-piperidyl)-5-[2-[2-[2-[4-[4-[(E)-N-ethoxy-C-ethyl-carbonimidoyl]-3-hydroxy-5-oxo-cyclohex-3-en-1-yl]phenoxy]ethoxy]ethoxy]-ethoxy]isoindoline-1,3-dione (26.53 mg, 38.12 μmol, 68.60% yield, 99.4% purity) as a yellow gum. HNMR: CDCl₃, 400 MHz. δ 15.05 (s, 1H), 8.28 (s, 1H), 7.77 (d, J=8.4 Hz, 1H), 7.37 (s, 1H), 7.22-7.25 (m, 1H), 7.16 (d, J=8.8 Hz, 2H), 6.90 (d, J=8.4 Hz, 2H), 4.96-5.00 (m, 1H), 4.23-4.27 (m, 2H), 4.11-4.14 (m, 4H), 3.90-3.96 (m, 2H), 3.82-3.88 (m, 2H), 3.77 (s, 4H), 3.23-3.45 (m, 1H), 2.52-2.95 (m, 9H), 1.34 (t, J=6.8 Hz, 3H), 1.18 (t, J=7.2 Hz, 3H). LCMS: RT=1.768 min, m/z 692.3 [M+H]⁺.

The following compounds were prepared in analogous fashion to Example 5.

### Compound 5-A

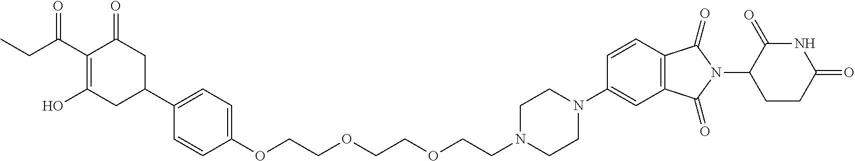 [SMILES: CCC(=O)C1=C(O)CC(c2ccc(OCCOCCOCCN3CCN(c4ccc5c(c4)C(=O)N(C4CCC(=O)NC4=O)C5=O)CC3)cc2)CC1=O]

2-(2,6-Dioxopiperidin-3-yl)-5-(4-(2-(2-(2-((5′-hydroxy-3′-oxo-4′-propionyl-1′,2′,3′,6′-tetrahydro-[1,1′-biphenyl]-4-yl)oxy)ethoxy)ethoxy)ethyl)piperazin-1-yl)isoindoline-1,3-dione. HNMR: CDCl₃, 400 MHz. δ 8.11 (s, 1H), 7.70 (d, J=8.4 Hz, 1H), 7.27 (d, J=2.4 Hz, 1H), 7.12-7.19 (m, 2H), 7.02-7.08 (m, 1H), 6.88-6.96 (m, 2H), 4.89-5.01 (m, 1H), 4.11-4.17 (m, 2H), 3.85-3.92 (m, 2H), 3.65-3.80 (m, 6H), 3.40-3.51 (m, 4H), 3.26-3.38 (m, 1H), 3.06-3.17 (m, 2H), 2.71-2.94 (m, 12H), 2.11-2.20 (m, 1H), 1.17 (t, J=7.2 Hz, 3H). LCMS: RT=2.675 min, m/z 717.3 [M+H]⁺.

### Compound 5-B

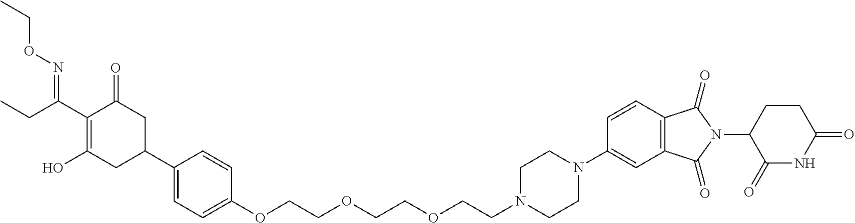 [SMILES: CCO/N=C(\CC)C1=C(O)CC(c2ccc(OCCOCCOCCN3CCN(c4ccc5c(c4)C(=O)N(C4CCC(=O)NC4=O)C5=O)CC3)cc2)CC1=O]

(E)-2-(2,6-Dioxopiperidin-3-yl)-5-(4-(2-(2-(2-((4′-(1-(ethoxyimino)propyl)-5′-hydroxy-3′-oxo-1′,2′,3′,6′-tetrahydro-[1,1′-biphenyl]-4-yl)oxy)ethoxy)ethoxy)ethyl)piperazin-1-yl)isoindoline-1,3-dione. HNMR: CDCl₃, 400 MHz. δ 15.05 (s, 1H), 8.04 (s, 1H), 7.70 (d, J=8.8 Hz, 1H), 7.36 (d, J=8.0 Hz, 1H), 7.15-7.20 (m, 2H), 7.02-7.08 (m, 1H), 6.88-6.94 (m, 2H), 4.92-5.01 (m, 1H), 4.11-4.18 (m, 4H), 3.86-3.91 (m, 2H), 3.67-3.78 (m, 6H), 3.40-3.47 (m, 4H), 3.26-3.37 (m, 1H), 2.66-3.02 (m, 15H), 2.11-2.20 (m, 1H), 1.35 (t, J=7.2 Hz, 3H), 1.19 (t, J=7.2 Hz, 3H). LCMS: RT=0.992 min, m/z 760.5 [M+H]⁺.

### Compound 5-C

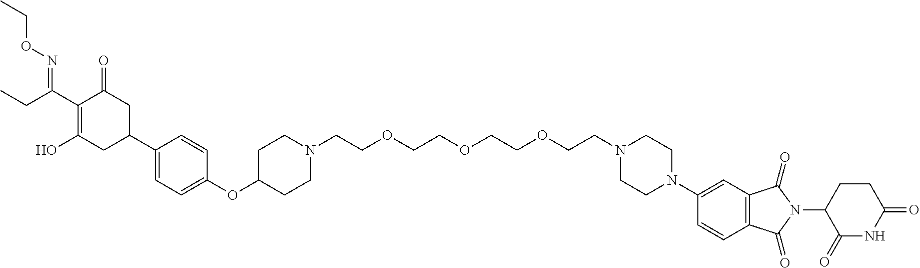 [SMILES: CCO/N=C(\CC)C1=C(O)CC(c2ccc(OC3CCN(CCOCCOCCOCCN4CCN(c5ccc6c(c5)C(=O)N(C5CCC(=O)NC5=O)C6=O)CC4)CC3)cc2)CC1=O]

(E)-2-(2,6-Dioxopiperidin-3-yl)-5-(4-(2-(2-(2-(2-(4-((4′-(1-(ethoxyimino)propyl)-5′-hydroxy-3′-oxo-1′,2′,3′,6′-tetrahydro-[1,1′-biphenyl]-4-yl)oxy)piperidin-1-yl)ethoxy)ethoxy)ethoxy)ethyl)piperazin-1-yl)isoindoline-1,3-dione. HNMR: CDCl₃, 400 MHz. δ 7.61 (d, J=8.8 Hz, 1H), 7.21 (d, J=2.0 Hz, 1H), 7.07-7.05 (m, 2H), 6.98 (dd, J=8.8, 2.4 Hz, 1H), 6.80-6.78 (m, 2H), 4.86 (dd, J=12.4, 5.6 Hz, 1H), 4.23-4.21 (m, 1H), 4.05 (q, J=7.2 Hz, 2H), 3.60-3.55 (m, 12H), 3.37-3.36 (m, 4H), 3.25-3.17 (m, 1H), 2.88-2.54 (m, 20H), 2.32-2.28 (m, 2H), 2.10-2.03 (m, 1H), 1.95-1.91 (m, 2H), 1.78-1.76 (m, 2H), 1.26 (t, J=7.2 Hz, 3H), 1.09 (t, J=7.6 Hz, 3H). LCMS: RT=1.902 min, m/z 887.4 [M+H]⁺.

### Compound 5-D

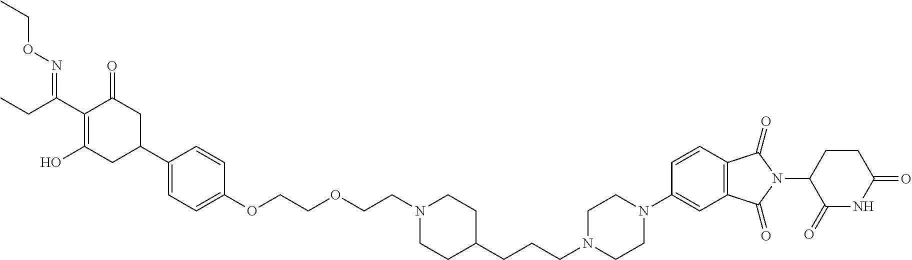 [SMILES: CCO/N=C(\CC)C1=C(O)CC(c2ccc(OCCOCCN3CCC(CCCN4CCN(c5ccc6c(c5)C(=O)N(C5CCC(=O)NC5=O)C6=O)CC4)CC3)cc2)CC1=O]

(E)-2-(2,6-Dioxopiperidin-3-yl)-5-(4-(3-(1-(2-(2-((4′-(1-(ethoxyimino)propyl)-5′-hydroxy-3′-oxo-1′,2′,3′,6′-tetrahydro-[1,1′-biphenyl]-4-yl)oxy)ethoxy)ethyl)piperidin-4-yl)propyl)piperazin-1-yl)isoindoline-1,3-dione. HNMR: CDCl₃, 400 MHz. δ 7.69 (d, J=8.4 Hz, 1H), 7.28 (d, J=2.4 Hz, 1H), 7.14 (d, J=8.4 Hz, 2H), 7.06 (dd, J=2.4, 8.4 Hz, 1H), 6.90 (d, J=8.4 Hz, 2H), 4.96-4.91 (m, 1H), 4.14-4.09 (m, 4H), 3.80 (t, J=8.8 Hz, 2H), 3.68 (t, J=6.0 Hz, 2H), 3.44-3.42 (m, 4H), 3.33-3.25 (m, 1H), 2.98-2.57 (m, 18H), 2.39-2.35 (m, 2H), 2.14-2.10 (m, 1H), 1.99-1.96 (m, 2H), 1.68-1.65 (m, 2H), 1.57-1.49 (m, 2H), 1.34-1.24 (m, 7H), 1.16 (t, J=7.6 Hz, 3H). LCMS: RT=2.296 min, m/z 841.4 [M+H]⁺.

### Example 6: Synthesis of Ethyl (2R)-2-(4-((5-(2-(2-(2-((2-(2,6-dioxopiperidin-3-yl)-1,3-dioxoisoindolin-5-yl)oxy)ethoxy)ethoxy)ethoxy)pyridin-2-yl)oxy)phenoxy)propanoate

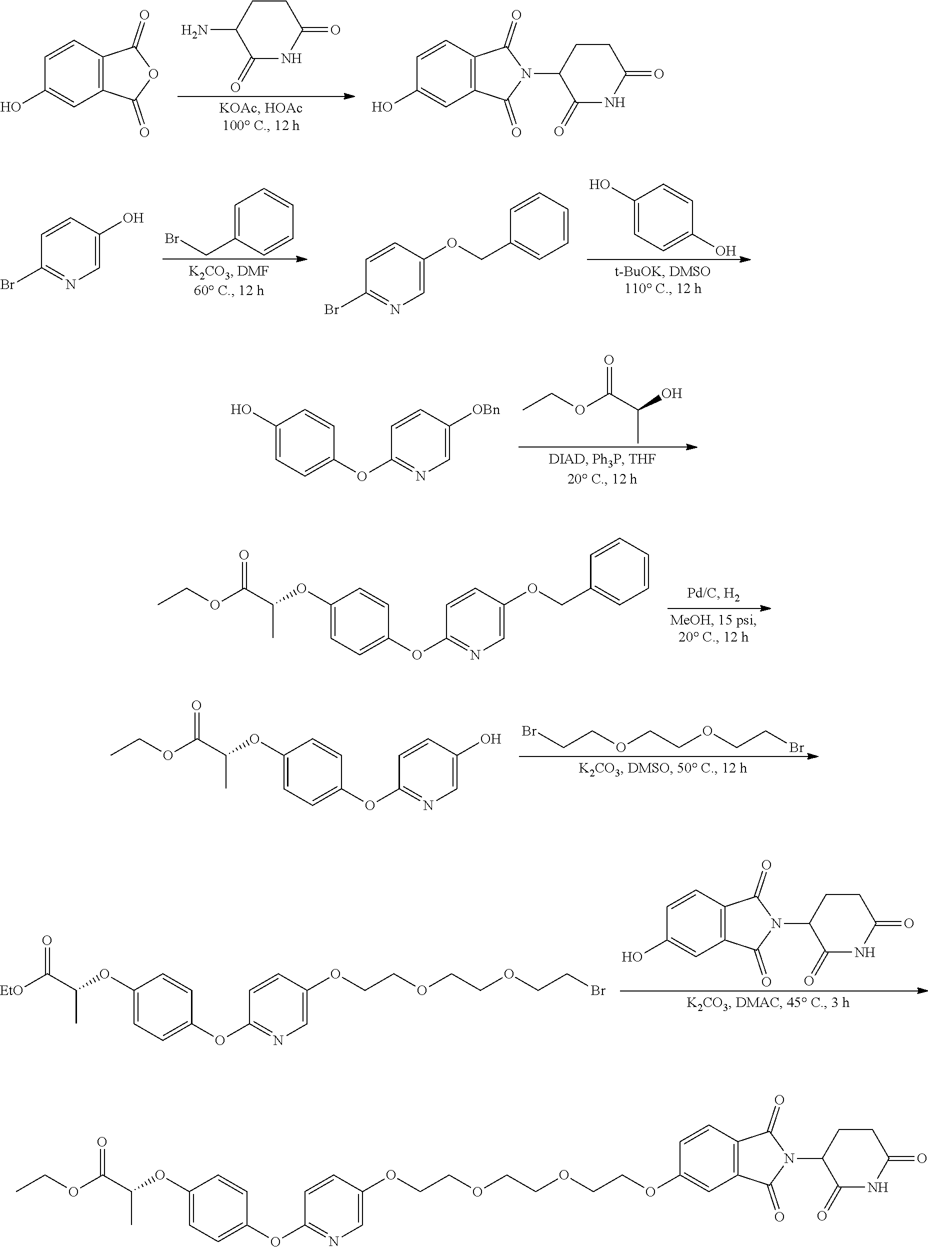

### Step 1: Synthesis of 2-(2,6-dioxopiperidin-3-yl)-5-hydroxyisoindoline-1,3-dione

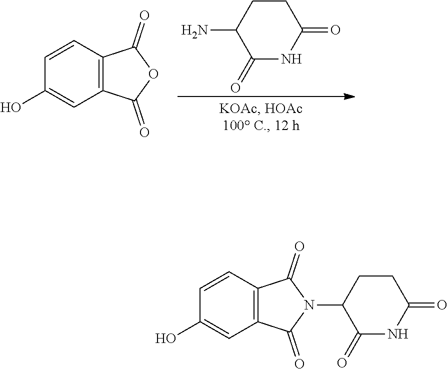 [SMILES: O=C1OC(=O)c2cc(O)ccc21 ; NC1CCC(=O)NC1=O ; O=C1CCC(N2C(=O)c3ccc(O)cc3C2=O)C(=O)N1]

To a solution of 5-hydroxyisobenzofuran-1,3-dione (1 g, 6.09 mmol, 1 eq) and 3-amino-piperidine-2,6-dione (1.05 g, 6.40 mmol, 1.05 eq, HCl) in AcOH (20 mL) was added KOAc (1.79 g, 18.28 mmol, 3 eq). The mixture was stirred at 100° C. for 12 h. The reaction was concentrated under reduced pressure. The mixture was poured into ice water (100 mL), then the solid was precipitated and filtered. The residue was washed with H₂O (3×50 mL) to afford 2-(2,6-dioxopiperidin-3-yl)-5-hydroxyisoindoline-1,3-dione (1.3 g, 4.74 mmol, 77.80% yield) as a black brown solid. HNMR: DMSO-d₆, 400 MHz. δ 11.37-10.56 (m, 2H), 7.75 (d, J=8.0 Hz, 1H), 7.31-7.04 (m, 2H), 5.19-4.92 (m, 1H), 3.00-2.77 (m, 1H), 2.66-2.52 (m, 2H), 2.12-1.95 (m, 1H).

### Step 2

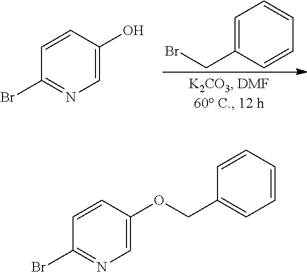 [SMILES: Oc1ccc(Br)nc1 ; BrCc1ccccc1 ; Brc1ccc(OCc2ccccc2)cn1]

To a solution of benzyl bromide (45 g, 263.11 mmol, 31.25 mL, 1 eq) and 6-bromopyridin-3-ol (50.36 g, 289.42 mmol, 1.1 eq) in DMF (400 mL) was added K₂CO₃ (72.73 g, 526.21 mmol, 2 eq). The mixture was stirred at 60° C. for 12 h. The reaction mixture was quenched with H₂O (400 mL) and then extracted with ethyl acetate (500 mL×3). The combined organic layer was dried over anhydrous Na₂SO₄, filtered, and concentrated under reduced pressure. The residue was purified by column chromatography (SiO₂, petroleum ether/ethyl acetate=20:1 to 10:1) to afford 5-(benzyloxy)-2-bromopyridine (44 g, 166.59 mmol, 63.32% yield) as a white solid. HNMR: CDCl₃, 400 MHz. δ 8.16 (d, J=2.8 Hz, 1H), 7.49-7.33 (m, 6H), 7.24-7.11 (m, 1H), 5.11 (s, 2H).

### Step 3

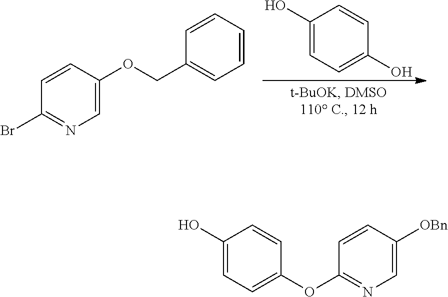

To a solution of 5-(benzyloxy)-2-bromopyridine (20 g, 75.72 mmol, 1 eq) and hydroquinone (12.51 g, 113.59 mmol, 16.90 mL, 1.5 eq) in DMSO (200 mL) was added t-BuOK (12.75 g, 113.59 mmol, 1.5 eq). The mixture was stirred at 110° C. for 12 h under N₂. The reaction mixture was quenched with H₂O (500 mL) and then extracted with ethyl acetate (500 mL×3). The combined organic layer was dried over anhydrous Na₂SO₄, filtered, and concentrated under reduced pressure. The residue was purified by column chromatography (SiO₂, DCM:MeOH=50:1 to 30:1) to afford 4-((5-(benzyloxy)pyridin-2-yl)oxy)phenol (7.4 g, 25.23 mmol, 33.32% yield) as a yellow oil. HNMR: CDCl₃, 400 MHz. δ 8.16 (d, J=2.8 Hz, 1H), 7.49-7.33 (m, 6H), 7.24-7.11 (m, 1H), 5.11 (s, 2H). LCMS: RT=0.920 min, m/z 294.0 [M+H]⁺.

### Step 4

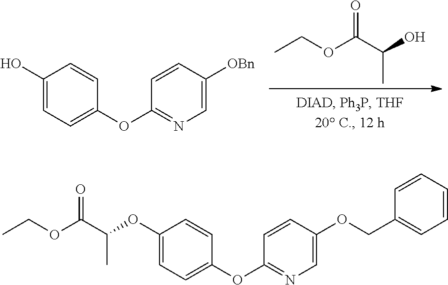

To a solution of 4-((5-(benzyloxy)pyridin-2-yl)oxy)phenol (3 g, 10.23 mmol, 1 eq), ethyl (2S)-2-hydroxypropanoate (1.33 g, 11.25 mmol, 1.29 mL, 1.1 eq) and PPh₃ (4.02 g, 15.34 mmol, 1.5 eq) in THE (30 mL) was added DIAD (4.14 g, 20.46 mmol, 3.98 mL, 2.0 eq). Then the mixture was stirred at 20° C. for 12 h. The reaction mixture was quenched with H₂O (100 mL) and then extracted with ethyl acetate (100 mL×3). The combined organic layer was dried over anhydrous Na₂SO₄, filtered, and concentrated under reduced pressure. The residue was purified by column chromatography (SiO₂, petroleum ether/ethyl acetate=20:1 to 10:1) to afford ethyl (R)-2-(4-((5-(benzyloxy)pyridin-2-yl)oxy)phenoxy)propanoate (2 g, 5.08 mmol, 49.70% yield) as a yellow oil. HNMR: CDCl₃, 400 MHz. δ 7.93 (d, J=2.8 Hz, 1H), 7.48-7.31 (m, 6H), 7.08-6.99 (m, 2H), 6.95-6.87 (m, 2H), 6.83 (d, J=9.2 Hz, 1H), 5.08 (s, 2H), 4.77-4.67 (m, 1H), 1.64-1.62 (m, 3H), 1.30-1.20 (m, 3H). LCMS: RT=1.109 min, m/z 394.2 [M+H]⁺. SFC: 98% ee.

### Step 5

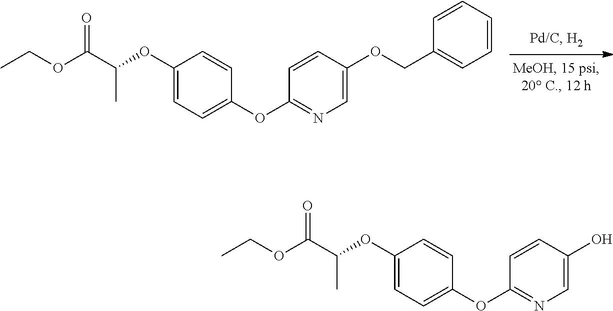 [SMILES: CCOC(=O)[C@@H](C)Oc1ccc(Oc2ccc(OCc3ccccc3)cn2)cc1 ; CCOC(=O)[C@@H](C)Oc1ccc(Oc2ccc(O)cn2)cc1]

To a solution of ethyl (R)-2-(4-((5-(benzyloxy)pyridin-2-yl)oxy)phenoxy)propanoate (2 g, 5.08 mmol, 1 eq) in MeOH (20 mL) was added Pd/C (200 mg, 279.59 μmol, 10% purity). The mixture was stirred at 20° C. for 12 h under H₂ (15 Psi). The reaction mixture was filtered and concentrated under reduced pressure. The residue was purified by column chromatography (SiO₂, petroleum ether/ethyl acetate=10:1 to 5:1 to 2:1) to afford ethyl (R)-2-(4-((5-hydroxypyridin-2-yl)oxy)phenoxy)propanoate (1.3 g, 4.29 mmol, 84.31% yield) as a colorless oil. HNMR: CDCl₃, 400 MHz. δ 7.82 (d, J=3.2 Hz, 1H), 7.26-7.21 (m, 1H), 7.04-7.00 (m, 2H), 6.93-6.87 (m, 2H), 6.79 (d, J=9.2 Hz, 1H), 5.36 (s, 1H), 4.78-4.62 (m, 1H), 4.32-4.19 (m, 2H), 1.63 (d, J=6.8 Hz, 3H), 1.31-1.27 (m, 3H).

### Step 6

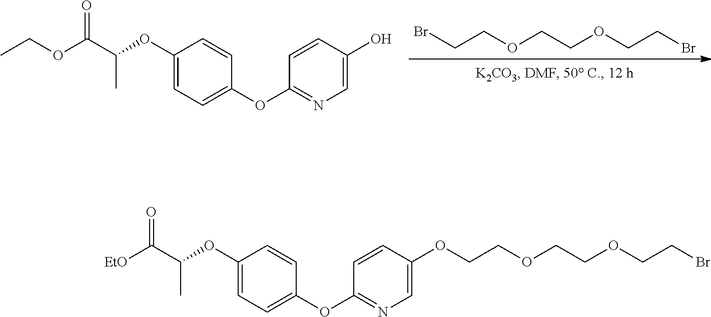 [SMILES: CCOC(=O)[C@@H](C)Oc1ccc(Oc2ccc(O)cn2)cc1 ; BrCCOCCOCCBr ; CCOC(=O)[C@@H](C)Oc1ccc(Oc2ccc(OCCOCCOCCBr)cn2)cc1]

To a solution of ethyl (R)-2-(4-((5-hydroxypyridin-2-yl)oxy)phenoxy)propanoate (500 mg, 1.65 mmol, 1 eq) and 1,2-bis(2-bromoethoxy)ethane (909.85 mg, 3.30 mmol, 2 eq) in DMF (5 mL) was added K₂CO₃ (455.67 mg, 3.30 mmol, 2 eq). The mixture was stirred at 50° C. for 12 h. The reaction mixture was quenched with H₂O (20 mL) and then extracted with ethyl acetate (20 mL×3). The combined organic layer was dried with anhydrous Na₂SO₄, filtered, and concentrated under reduced pressure. The residue was purified by column chromatography (SiO₂, petroleum ether/ethyl acetate=3:1 to 2:1) to afford ethyl (R)-2-(4-((5-(2-(2-(2-bromoethoxy)ethoxy)-ethoxy)pyridin-2-yl)oxy)phenoxy)propanoate (600 mg, 1.20 mmol, 73.03% yield) was obtained as a colorless oil. HNMR: CDCl₃, 400 MHz. δ 7.88 (d, J=2.8 Hz, 1H), 7.35-7.29 (m, 1H), 7.07-7.00 (m, 2H), 6.95-6.87 (m, 2H), 6.82 (d, J=9.2 Hz, 1H), 4.79-4.64 (m, 1H), 4.31-4.20 (m, 2H), 4.18-4.12 (m, 2H), 3.91-3.81 (m, 4H), 3.79-3.68 (m, 4H), 3.49 (t, J=6.4 Hz, 2H), 1.63 (d, J=6.8 Hz, 3H), 1.29 (t, J=7.2 Hz, 3H). LCMS: RT=0.902 min, m/z 500.1 [M+H]⁺.

### Step 7

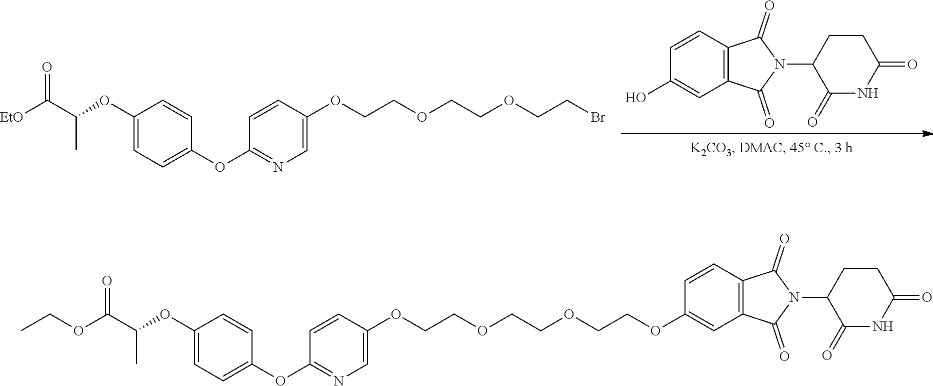 [SMILES: CCOC(=O)[C@@H](C)Oc1ccc(Oc2ccc(OCCOCCOCCBr)cn2)cc1 ; O=C1CCC(N2C(=O)c3ccc(O)cc3C2=O)C(=O)N1 ; CCOC(=O)[C@@H](C)Oc1ccc(Oc2ccc(OCCOCCOCCOc3ccc4c(c3)C(=O)N(C3CCC(=O)NC3=O)C4=O)cn2)cc1]

To a solution of ethyl (R)-2-(4-((5-(2-(2-(2-bromoethoxy)ethoxy)-ethoxy)pyridin-2-yl)oxy)phenoxy)propanoate (200 mg, 401.31 μmol, 1 eq) and 2-(2,6-dioxopiperidin-3-yl)-5-hydroxyisoindoline-1,3-dione (110.05 mg, 401.31 μmol, 1 eq) in DMAC (5 mL) was added K₂CO₃ (110.93 mg, 802.62 μmol, 2 eq). The mixture was stirred at 45° C. for 3 h. The reaction mixture was quenched with H₂O (20 mL) and then extracted with ethyl acetate (30 mL×3). The combined organic layer was dried over anhydrous Na₂SO₄, filtered, and concentrated under reduced pressure. The residue was purified by prep-HPLC (column: Unisil 3-100 C18 Ultra 150*50 mm*3 μm; mobile phase: [water(0.225% FA)-ACN]; B %: 48%-68%, 10 min). LCMS showed two peaks. The material was re-purified by prep-HPLC (column: Phenomenex Gemini NX—C18 (75*30 mm*3 μm); mobile phase: [water(10 mM NH₄HCO₃)-ACN]; B %: 34%-64%, 8 min) to afford Ethyl (2R)-2-(4-((5-(2-(2-(2-((2-(2,6-dioxopiperidin-3-yl)-1,3-dioxoisoindolin-5-yl)oxy)ethoxy)ethoxy)ethoxy)-pyridin-2-yl)oxy)phenoxy)propanoate (40 mg, 57.83 μmol, 14.41% yield) as a white solid. HNMR: DMSO-d₆, 400 MHz. δ 7.90-7.73 (m, 2H), 7.54-7.43 (m, 2H), 7.40-7.34 (m, 1H), 7.03-6.96 (m, 2H), 6.94-6.85 (m, 3H), 5.17-5.05 (m, 1H), 4.95-4.85 (m, 1H), 4.36-4.28 (m, 2H), 4.18-4.09 (m, 4H), 3.83-3.77 (m, 2H), 3.76-3.71 (m, 2H), 3.67-3.58 (m, 4H), 2.97-2.82 (m, 1H), 2.71-2.52 (m, 2H), 2.13-1.97 (m, 1H), 1.51 (d, J=6.8 Hz, 3H), 1.19 (t, J=7.2 Hz, 3H). LCMS: RT=2.781 min, m/z 692.2 [M+H]⁺.

The following compounds were prepared in analogous fashion to Example 6.

### Compound 6-A

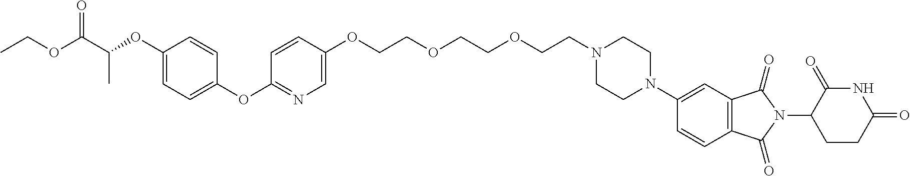 [SMILES: CCOC(=O)[C@@H](C)Oc1ccc(Oc2ccc(OCCOCCOCCN3CCN(c4ccc5c(c4)C(=O)N(C4CCC(=O)NC4=O)C5=O)CC3)cn2)cc1]

Ethyl (2R)-2-(4-((5-(2-(2-(2-(4-(2-(2,6-dioxopiperidin-3-yl)-1,3-dioxoisoindolin-5-yl)piperazin-1-yl)ethoxy)ethoxy)ethoxy)pyridin-2-yl)oxy)phenoxy)propanoate. HNMR: DMSO-d6, 400 MHz. δ 11.08 (s, 1H), 8.39 (s, 1H), 7.86 (d, J=3.2 Hz, 1H), 7.67 (d, J=8.4 Hz, 1H), 7.54-7.47 (m, 1H), 7.33 (d, J=2.0 Hz, 1H), 7.27-7.19 (m, 1H), 7.00-6.86 (m, 5H), 5.13-5.03 (m, 1H), 4.96-4.85 (m, 1H), 4.20-4.09 (m, 4H), 3.76-3.72 (m, 2H), 3.61-3.54 (m, 6H), 3.42-3.39 (m, 4H), 2.92-2.83 (m, 1H), 2.64-2.53 (m, 8H), 2.09-1.95 (m, 1H), 1.51 (d, J=6.8 Hz, 3H), 1.19 (t, J=7.2 Hz, 3H). LCMS: RT=2.384 min, m/z 760.3 [M+H]⁺.

### Compound 6-B

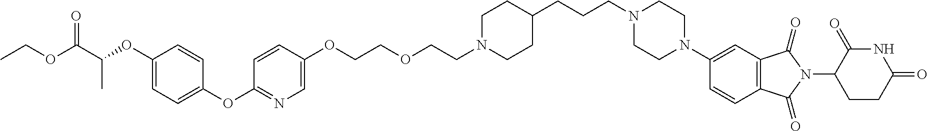 [SMILES: CCOC(=O)[C@@H](C)Oc1ccc(Oc2ccc(OCCOCCN3CCC(CCCN4CCN(c5ccc6c(c5)C(=O)N(C5CCC(=O)NC5=O)C6=O)CC4)CC3)cn2)cc1]

Ethyl (2R)-2-[4-[[5-[2-[2-[4-[3-[4-[2-(2,6-dioxo-3-piperidyl)-1,3-dioxo-isoindolin-5-yl]piperazin-1-yl]propyl]-1-piperidyl]ethoxy]ethoxy]-2-pyridyl]oxy]phenoxy]propanoate. HNMR: DMSO-d₆, 400 MHz. δ 1.12˜1.26 (m, 8H), 1.44-1.51 (m, 5H), 1.64-1.67 (m, 2H), 1.99-2.05 (m, 1H), 2.17-2.23 (m, 2H), 2.27-2.33 (m, 2H), 2.46-2.49 (m, 4H), 2.55-2.60 (m, 1H), 2.67˜2.69 (m, 2H), 2.84˜2.93 (m, 1H), 2.99˜3.02 (m, 2H), 3.40˜3.44 (m, 5H), 3.61 (t, J=6.0 Hz, 2H), 3.71˜3.73 (m, 2H), 4.11˜4.17 (m, 4H), 4.90 (q, J=6.8 Hz, 1H), 5.07 (dd, J=5.6, 12.8 Hz, 1H), 6.86-6.90 (m, 2H), 6.93 (d, J=9.2 Hz, 1H), 6.97-7.01 (m, 2H), 7.25 (dd, J=2.0, 8.4 Hz, 1H), 7.33 (d, J=2.0 Hz, 1H), 7.49 (dd, J=3.2, 8.8 Hz, 1H), 7.67 (d, J=8.4 Hz, 1H), 7.84 (d, J=2.8 Hz, 1H), 8.16 (s, 1H). LCMS: RT=1.599 min, m/z 841.3 [M+H]⁺.

### Compound 6-C

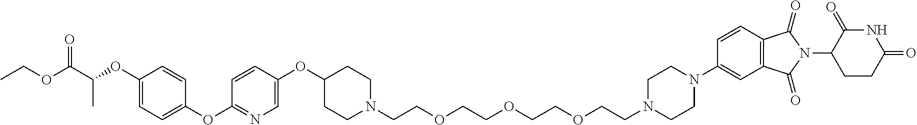 [SMILES: CCOC(=O)[C@@H](C)Oc1ccc(Oc2ccc(OC3CCN(CCOCCOCCOCCN4CCN(c5ccc6c(c5)C(=O)N(C5CCC(=O)NC5=O)C6=O)CC4)CC3)cn2)cc1]

Ethyl (2R)-2-(4-((5-((1-(2-(2-(2-(2-(4-(2-(2,6-dioxopiperidin-3-yl)-1,3-dioxoisoindolin-5-yl)piperazin-1-yl)ethoxy)ethoxy)ethoxy)ethyl)piperidin-4-yl)oxy)pyridin-2-yl)oxy)phenoxy)-propanoate. HNMR: DMSO-d₆, 400 MHz. δ 7 11.06 (s, 1H), 7.82 (d, J=2.8 Hz, 1H), 7.67 (d, J=8.4 Hz, 1H), 7.54-7.46 (m, 1H), 7.33 (d, J=2.0 Hz, 1H), 7.28-7.21 (m, 1H), 7.02-6.96 (m, 2H), 6.94-6.86 (m, 3H), 5.11-5.02 (m, 1H), 4.95-4.86 (m, 1H), 4.35-4.23 (m, 1H), 4.20-4.08 (m, 2H), 3.57-3.48 (m, 12H), 3.46-3.38 (m, 4H), 2.95-2.82 (m, 1H), 2.78-2.67 (m, 2H), 2.63-2.52 (m, 8H), 2.48-2.45 (m, 2H), 2.28-2.17 (m, 2H), 2.07-1.96 (m, 1H), 1.95-1.83 (m, 2H), 1.64-1.55 (m, 2H), 1.51 (d, J=6.8 Hz, 3H), 1.19 (t, J=7.2 Hz, 3H). LCMS: RT=2.146 min, m/z 887.3 [M+H]⁺.

### Example 7: Synthesis of N-[3-[2-[2-[2-[2-[2-(2,6-dioxo-3-piperidyl)-1,3-dioxo-isoindolin-5-yl]oxyethoxy]-ethoxy]ethoxy]ethoxy]propyl]-2-[4-[[5-(trifluoromethyl)-3-pyridyl]oxy]phenoxy]acetamide

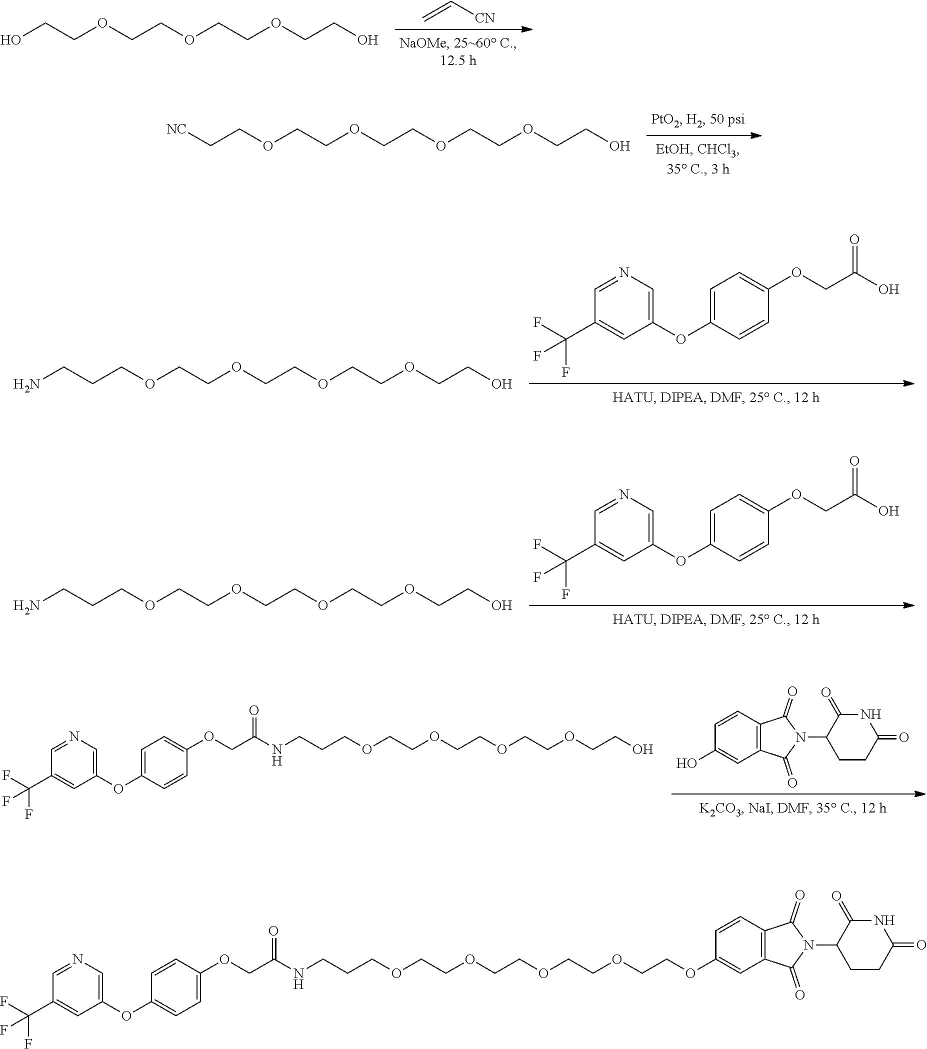

### Step 1

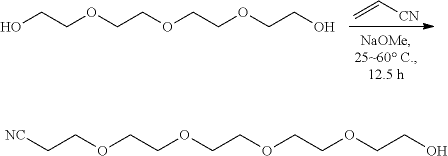

To a mixture of prop-2-enenitrile (5 g, 94.23 mmol, 6.25 mL) in 2-[2-[2-(2-hydroxyethoxy)-ethoxy]ethoxy]ethanol (18.30 g, 94.23 mmol, 16.20 mL) was added NaOMe (20 mg, 370.21 μmol). The mixture was stirred at 60° C. for 0.5 hour. The mixture was cooled to 25° C. and stirred for 12 hours. Acetic acid (0.5 mL) was added and the mixture was concentrated in vacuo. The residue was diluted with water (100 mL) and extracted with DCM (100 mL×3). The combined organic layer was washed with brine (100 mL×2), dried over Na₂SO₄, filtered, and concentrated under reduced pressure to give the crude product. The crude product was purified by column chromatography (SiO₂, PE:EtOAc=10:1 to 1:1) to give 3-[2-[2-[2-(2-hydroxyethoxy)ethoxy]ethoxy]ethoxy]propanenitrile (2.2 g, 8.90 mmol, 9.44% yield) as a pink oil. HNMR: CDCl₃, 400 MHz. δ 3.72-3.55 (m, 18H), 2.63-2.57 (m, 2H).

### Step 2

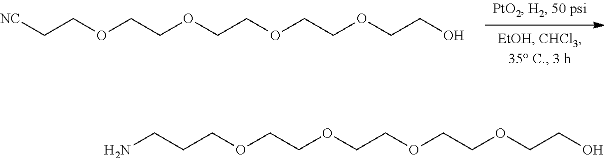 [SMILES: N#CCCOCCOCCOCCOCCO ; NCCCOCCOCCOCCOCCO]

To a mixture of 3-[2-[2-[2-(2-hydroxyethoxy)ethoxy]ethoxy]ethoxy]propanenitrile (2.2 g, 8.90 mmol) in EtOH (50 mL) and CHCl₃ (1 mL) was added PtO₂ (220 mg) under the protection of N₂. The mixture was degassed and purged with H₂ three times, and then stirred at 35° C. for 3 hours under H₂ atmosphere (50 psi). The mixture was filtered and the filtrate was concentrated in vacuo to give 2-[2-[2-[2-(3-amino-propoxy)ethoxy]ethoxy]ethoxy]ethanol (2.1 g, 8.36 mmol, 93.92% yield) as a colorless oil. HNMR: CDCl₃, 400 MHz. δ 3.81-3.76 (m, 2H), 3.76-3.70 (m, 2H), 3.67-3.59 (m, 14H), 3.29-3.18 (m, 2H), 2.09-2.01 (m, 2H).

### Step 3

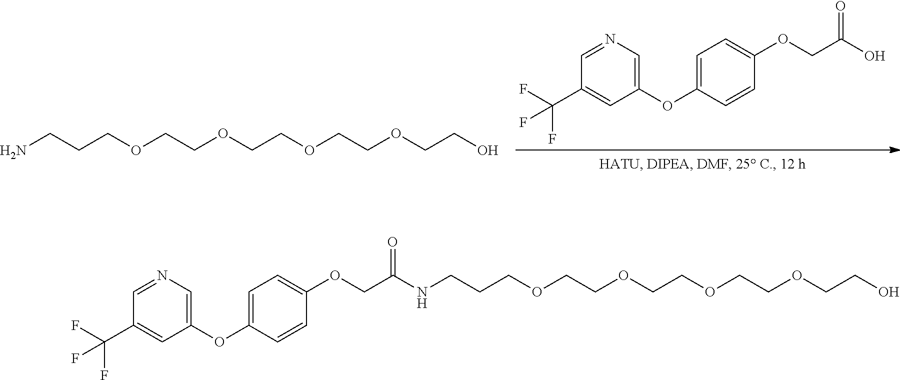 [SMILES: NCCCOCCOCCOCCOCCO ; O=C(O)COc1ccc(Oc2cncc(C(F)(F)F)c2)cc1 ; O=C(COc1ccc(Oc2cncc(C(F)(F)F)c2)cc1)NCCCOCCOCCOCCOCCO]

To a mixture of 2-[2-[2-[2-(3-aminopropoxy)ethoxy]ethoxy]ethoxy]ethanol (361.06 mg, 1.44 mmol) in DMF (5 mL) were added 2-[4-[[5-(trifluoromethyl)-3-pyridyl]oxy]phenoxy]acetic acid (0.3 g, 957.77 μmol), HATU (437.01 mg, 1.15 mmol) and DIPEA (371.35 mg, 2.87 mmol, 500.48 μL). The mixture was stirred at 25° C. for 12 hours. EtOAc (20 mL) and water (20 mL) were added and layers were separated. The aqueous phase was extracted with EtOAc (10 mL×3). The combined extracts were washed with brine (20 mL), dried over Na₂SO₄, filtered, and concentrated under vacuum to give the crude product. The crude product was purified by prep-TLC (SiO₂, DCM:MeOH=10:1) to give N-[3-[2-[2-[2-(2-hydroxyethoxy)ethoxy]ethoxy]ethoxy]propyl]-2-[4-[[5-(trifluoromethyl)-3-pyridyl]oxy]phenoxy]acetamide (0.33 g, 603.81 μmol, 63.04% yield) as a pink oil. LCMS: RT=0.890 min, m/z 547.5 [M+H]⁺.

### Step 4

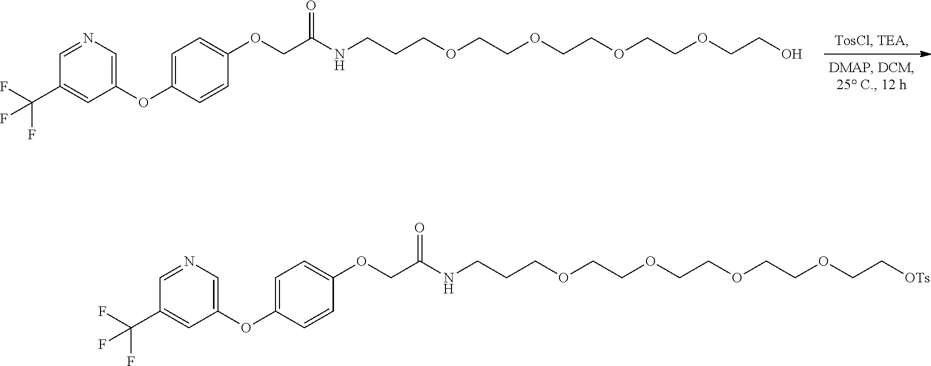 [SMILES: O=C(COc1ccc(Oc2cncc(C(F)(F)F)c2)cc1)NCCCOCCOCCOCCOCCO ; Cc1ccc(S(=O)(=O)OCCOCCOCCOCCOCCCNC(=O)COc2ccc(Oc3cncc(C(F)(F)F)c3)cc2)cc1]

To a mixture of N-[3-[2-[2-[2-(2-hydroxyethoxy)ethoxy]ethoxy]ethoxy]propyl]-2-[4-[[5-(trifluoromethyl)-3-pyridyl]oxy]phenoxy]acetamide (0.33 g, 603.81 μmol) in DCM (5 mL) were added TosCl (230.23 mg, 1.21 mmol), Et₃N (183.30 mg, 1.81 mmol, 252.13 μL) and DMAP (7.38 mg, 60.38 μmol). The mixture was stirred at 25° C. for 12 hours. The solvent was evaporated under reduced pressure. The residue was purified by prep-TLC (SiO₂, DCM:MeOH=20:1) to give 2-[2-[2-[2-[3-[[2-[4-[[5-(trifluoromethyl)-3-pyridyl]oxy]phenoxy]acetyl]amino]propoxy]ethoxy]ethoxy]ethoxy]ethyl 4-methylbenzenesulfonate (0.23 g, 328.23 μmol, 54.36% yield) as a colorless oil. LCMS: RT=0.883 min, m/z 701.2 [M+H]⁺.

### Step 5

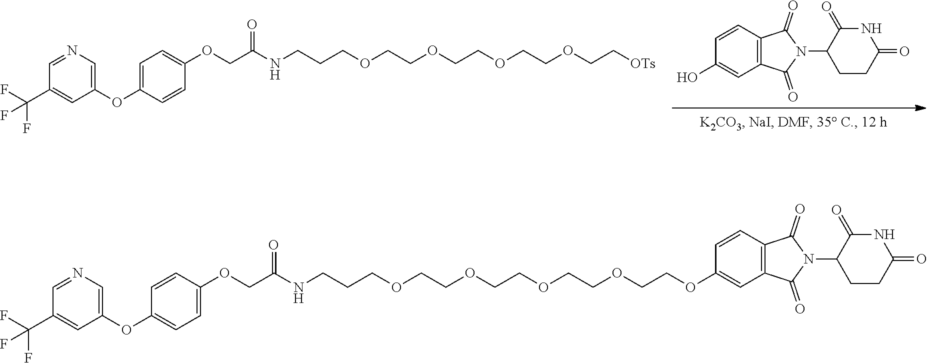 [SMILES: Cc1ccc(S(=O)(=O)OCCOCCOCCOCCOCCCNC(=O)COc2ccc(Oc3cncc(C(F)(F)F)c3)cc2)cc1 ; O=C1CCC(N2C(=O)c3ccc(O)cc3C2=O)C(=O)N1 ; O=C(COc1ccc(Oc2cncc(C(F)(F)F)c2)cc1)NCCCOCCOCCOCCOCCOc1ccc2c(c1)C(=O)N(C1CCC(=O)NC1=O)C2=O]

To a mixture of 2-[2-[2-[2-[3-[[2-[4-[[5-(trifluoromethyl)-3-pyridyl]oxy]phenoxy]acetyl]amino]propoxy]-ethoxy]ethoxy]ethoxy]ethyl 4-methylbenzenesulfonate (100 mg, 142.71 μmol) in DMF (3 mL) were added 2-(2,6-dioxo-3-piperidyl)-5-hydroxy-isoindoline-1,3-dione (58.70 mg, 214.07 μmol), K₂CO₃ (59.17 mg, 428.13 μmol), NaI (21.39 mg, 142.71 μmol). The mixture was stirred at 35° C. for 12 hours. EtOAc (20 mL) and water (20 mL) were added and layers were separated. The aqueous phase was extracted with EtOAc (10 mL×3). The combined extracts were washed with brine (20 mL), dried over Na₂SO₄, filtered, and concentrated under vacuum. The crude product was purified by prep-HPLC (column: Phenomenex luna C18 150*25 mm*10 μm; mobile phase: [water (0.225% FA)-ACN]; B %: 39%-69%, 10 min) to afford N-[3-[2-[2-[2-[2-[2-(2,6-dioxo-3-piperidyl)-1,3-dioxo-isoindolin-5-yl]oxyethoxy]ethoxy]ethoxy]ethoxy]propyl]-2-[4-[[5-(trifluoromethyl)-3-pyridyl]oxy]phenoxy]acetamide (45.32 mg, 56.06 230 μmol, 39.28% yield, 99.3% purity) as a brown gum. HNMR: DMSO-d₆, 400 MHz. δ 11.11 (s, 1H), 8.70 (s, 1H), 8.60-8.58 (m, 1H), 8.08 (t, J=5.6 Hz, 1H), 7.84-7.80 (m, 1H), 7.62 (s, 1H), 7.45-7.43 (m, 1H), 7.38-7.34 (m, 1H), 7.19-7.16 (m, 2H), 7.06-7.03 (m, 2H), 5.14-5.09 (m, 1H), 4.47 (s, 2H), 4.32-4.29 (m, 2H), 3.79-3.77 (m, 2H), 3.60-3.57 (m, 2H), 3.54-3.52 (m, 2H), 3.50-3.48 (m, 6H), 3.46-3.44 (m, 2H), 3.39-3.38 (m, 2H), 3.19-3.15 (m, 2H), 2.92-2.84 (m, 1H), 2.67-2.56 (m, 2H), 2.07-2.01 (m, 1H), 1.68-1.68 (t, J=6.4 Hz, 2H). LCMS: RT=3.080 min, m/z 803.3 [M+H]⁺.

The following compounds were prepared in analogous fashion to Example 7.

### Compound 7-A

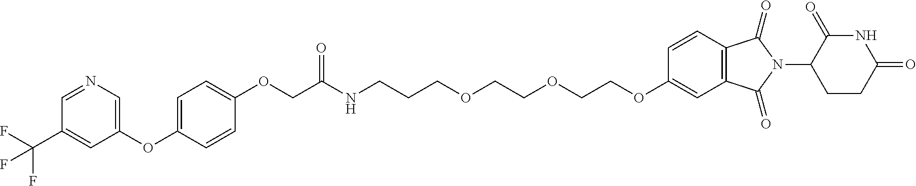 [SMILES: O=C(COc1ccc(Oc2cncc(C(F)(F)F)c2)cc1)NCCCOCCOCCOc1ccc2c(c1)C(=O)N(C1CCC(=O)NC1=O)C2=O]

N-(3-(2-(2-((2-(2,6-Cioxopiperidin-3-yl)-1,3-dioxoisoindolin-5-yl)oxy)ethoxy)ethoxy)propyl)-2-(4-((5-(trifluoromethyl)pyridin-3-yl)oxy)phenoxy)acetamide. HNMR: DMSO-d₆, 400 MHz. δ 11.12 (s, 1H), 8.70 (s, 1H), 8.59 (d, J=2.4 Hz, 1H), 8.09 (t, J=6.0 Hz, 1H), 7.82 (d, J=8.4 Hz, 1H), 7.62 (s, 1H), 7.44 (d, J=2.0 Hz, 1H), 7.35 (dd, J=8.0, 2.4 Hz, 1H), 7.18-7.16 (m, 2H), 7.06-7.03 (m, 2H), 5.12 (dd, J=12.8, 5.2 Hz, 1H), 4.48 (s, 2H), 4.30-4.29 (m, 2H), 3.78-0.374 (m, 2H), 3.59-3.58 (m, 2H), 3.50-3.48 (m, 2H), 3.39 (t, J=6.4 Hz, 2H), 3.19 (q, J=6.4 Hz, 2H), 2.88-2.84 (m, 1H), 2.61-2.57 (m, 2H), 2.06-2.04 (m, 1H), 1.66 (quin, J=6.4 Hz, 2H). LCMS: RT=2.736 min, m/z 715.2 [M+H]⁺.

### Compound 7-B

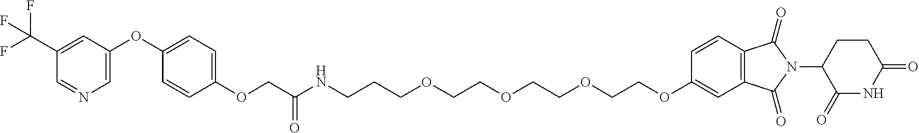 [SMILES: O=C(COc1ccc(Oc2cncc(C(F)(F)F)c2)cc1)NCCCOCCOCCOCCOc1ccc2c(c1)C(=O)N(C1CCC(=O)NC1=O)C2=O]

N-(3-(2-(2-(2-((2-(2,6-dioxopiperidin-3-yl)-1,3-dioxoisoindolin-5-yl)oxy)ethoxy)ethoxy)ethoxy)propyl)-2-(4-((5-(trifluoromethyl)pyridin-3-yl)oxy)phenoxy)acetamide. HNMR: DMSO-d₆, 400 MHz. δ 11.20-11.06 (m, 1H), 8.70 (s, 1H), 8.60 (d, J=2.8 Hz, 1H), 8.15-8.05 (m, 1H), 7.83 (d, J=8.4 Hz, 1H), 7.62 (s, 1H), 7.45 (d, J=2.0 Hz, 1H), 7.36 (dd, J=2.0, 8.4 Hz, 1H), 7.20-7.15 (m, 2H), 7.07-7.03 (m, 2H), 5.12 (dd, J=5.2, 12.8 Hz, 1H), 4.48 (s, 2H), 4.36-4.27 (m, 2H), 3.81-3.75 (m, 2H), 3.61-3.57 (m, 2H), 3.56-3.43 (m, 8H), 3.21-3.16 (m, 2H), 2.95-2.83 (m, 1H), 2.63-2.53 (m, 2H), 2.09-2.00 (m, 1H), 1.70-1.62 (m, 2H). LCMS: RT=3.098 min, m/z 759.2 [M+H]⁺.

### Compound 7-C

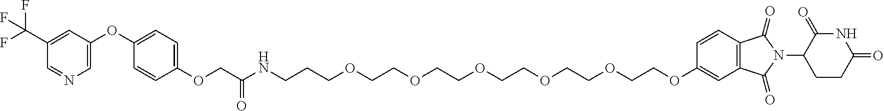 [SMILES: O=C(COc1ccc(Oc2cncc(C(F)(F)F)c2)cc1)NCCCOCCOCCOCCOCCOCCOc1ccc2c(c1)C(=O)N(C1CCC(=O)NC1=O)C2=O]

N-(1-((2-(2,6-dioxopiperidin-3-yl)-1,3-dioxoisoindolin-5-yl)oxy)-3,6,9,12,15-pentaoxaoctadecan-18-yl)-2-(4-((5-(trifluoromethyl)pyridin-3-yl)oxy)phenoxy)acetamide. HNMR: DMSO-d₆, 400 MHz. δ 11.21-11.00 (m, 1H), 8.67 (s, 1H), 8.65-8.55 (m, 1H), 8.12-8.02 (m, 1H), 7.83 (d, J=8.4 Hz, 1H), 7.63 (s, 1H), 7.45 (d, J=2.0 Hz, 1H), 7.41-7.33 (m, 1H), 7.22-7.14 (m, 2H), 7.09-7.01 (m, 2H), 5.20-5.04 (m, 1H), 4.49 (s, 2H), 4.36-4.27 (m, 2H), 3.83-3.75 (m, 2H), 3.62-3.57 (m, 2H), 3.56-3.52 (m, 2H), 3.51-3.49 (m, 10H), 3.47-3.44 (m, 2H), 3.38 (t, J=6.4 Hz, 2H), 3.24-3.15 (m, 2H), 2.96-2.83 (m, 1H), 2.63-2.54 (m, 2H), 2.10-2.00 (m, 1H), 1.72-1.60 (m, 2H). LCMS: RT=3.085 min, m/z=847.3 [M+H]⁺.

### Example 8: Synthesis of N-[3-[2-[2-[2-[2-[2-(2,6-dioxo-3-piperidyl)-1,3-dioxo-isoindolin-5-yl]oxyethoxy]-ethoxy]ethoxy]ethoxy]propyl]-2-[4-[(6-fluoro-1,3-benzothiazol-2-yl)oxy]phenoxy]acetamide

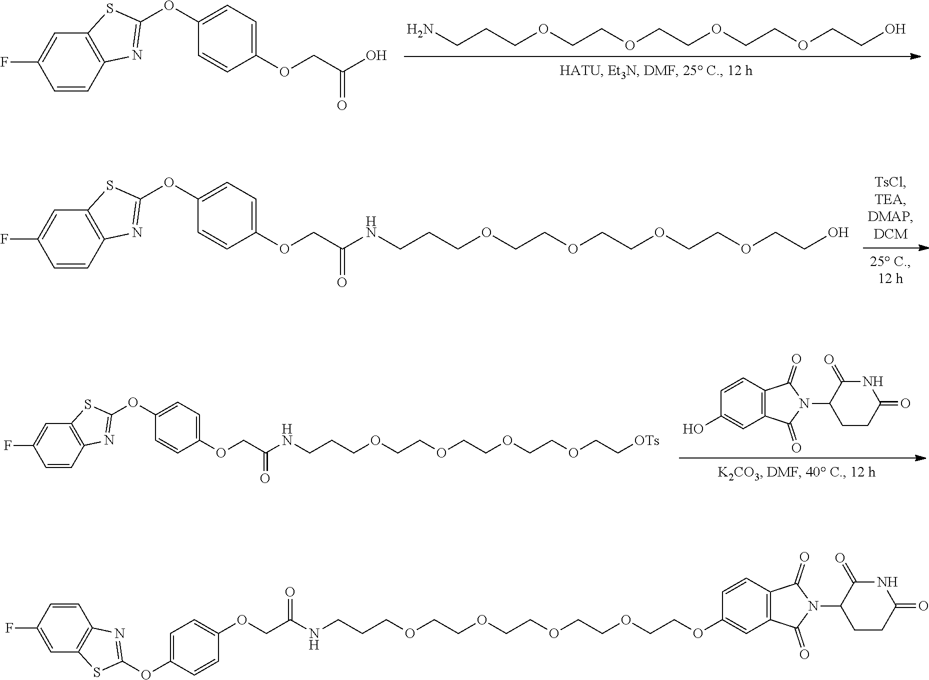 [SMILES: O=C(O)COc1ccc(Oc2nc3ccc(F)cc3s2)cc1 ; NCCCOCCOCCOCCOCCO ; O=C(COc1ccc(Oc2nc3ccc(F)cc3s2)cc1)NCCCOCCOCCOCCOCCO ; Cc1ccc(S(=O)(=O)OCCOCCOCCOCCOCCCNC(=O)COc2ccc(Oc3nc4ccc(F)cc4s3)cc2)cc1 ; O=C1CCC(N2C(=O)c3ccc(O)cc3C2=O)C(=O)N1 ; O=C(COc1ccc(Oc2nc3ccc(F)cc3s2)cc1)NCCCOCCOCCOCCOCCOc1ccc2c(c1)C(=O)N(C1CCC(=O)NC1=O)C2=O]

### Step 1

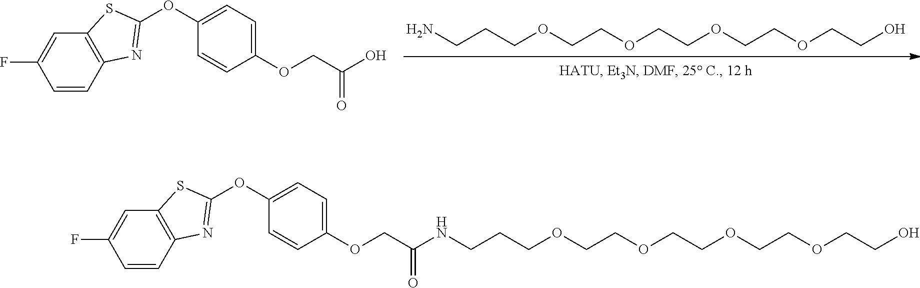 [SMILES: O=C(O)COc1ccc(Oc2nc3ccc(F)cc3s2)cc1 ; NCCCOCCOCCOCCOCCO ; O=C(COc1ccc(Oc2nc3ccc(F)cc3s2)cc1)NCCCOCCOCCOCCOCCO]

To a mixture of 2-[4-[(6-fluoro-1,3-benzothiazol-2-yl)oxy]phenoxy]acetic acid (0.3 g, 939.53 μmol) in DMF (5 mL) were added 2-[2-[2-[2-(3-aminopropoxy)ethoxy]ethoxy]ethoxy]ethanol (354.18 mg, 1.41 mmol), HATU (428.69 mg, 1.13 mmol) and DIPEA (364.28 mg, 2.82 mmol, 490.95 μL). The mixture was stirred at 25° C. for 12 hours. EtOAc (20 mL) and water (20 mL) were added and layers were separated. The aqueous phase was extracted with EtOAc (10 mL×3). The combined extracts were washed with brine (20 mL), dried over Na₂SO₄, filtered, and concentrated under vacuum. The residue was purified by prep-TLC (SiO₂, DCM:MeOH=10:1) to give 2-[4-[(6-fluoro-1,3-benzothiazol-2-yl)oxy]phenoxy]-N-[3-[2-[2-[2-(2-hydroxyethoxy)ethoxy]ethoxy]ethoxy]propyl]acetamide (0.13 g, 235.25 μmol, 25.04% yield) as a colorless oil. LCMS: RT=0.912 min, m/z 553.5 [M+H]⁺.

### Step 2

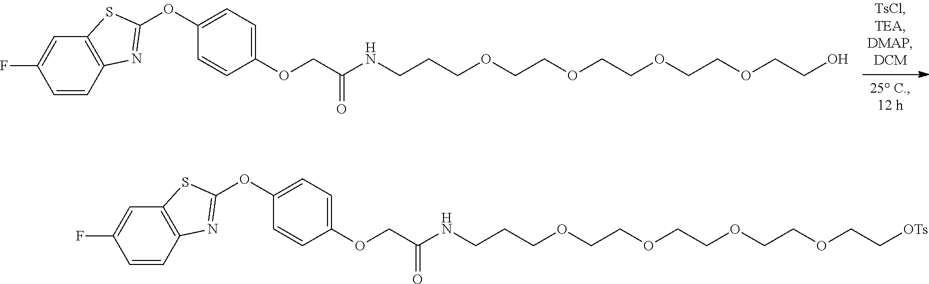 [SMILES: O=C(COc1ccc(Oc2nc3ccc(F)cc3s2)cc1)NCCCOCCOCCOCCOCCO ; Cc1ccc(S(=O)(=O)OCCOCCOCCOCCOCCCNC(=O)COc2ccc(Oc3nc4ccc(F)cc4s3)cc2)cc1]

To a mixture of 2-[4-[(6-fluoro-1,3-benzothiazol-2-yl)oxy]phenoxy]-N-[3-[2-[2-[2-(2-hydroxyethoxy)ethoxy]ethoxy]ethoxy]propyl]acetamide (0.13 g, 235.25 μmol) in DCM (5 mL) were added TosCl (89.70 mg, 470.49 μmol), Et₃N (71.41 mg, 705.74 μmol, 98.23 μL) and DMAP (2.87 mg, 23.52 μmol). The mixture was stirred at 25° C. for 12 hours. The solvent was evaporated under reduced pressure. The residue was purified by prep-TLC (SiO₂, DCM:MeOH=20:1) to give 2-[2-[2-[2-[3-[[2-[4-[(6-fluoro-1,3-benzothiazol-2-yl)oxy]phenoxy]acetyl]amino]propoxy]ethoxy]ethoxy]ethoxy]ethyl 4-methylbenzenesulfonate (0.123 g, 174.02 μmol, 73.98% yield) as a colorless oil. LCMS: RT=0.931 min, m/z 707.3 [M+H]⁺.

### Step 3

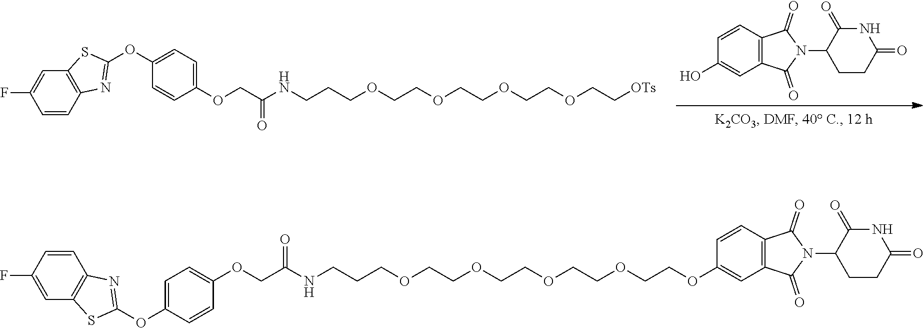 [SMILES: Cc1ccc(S(=O)(=O)OCCOCCOCCOCCOCCCNC(=O)COc2ccc(Oc3nc4ccc(F)cc4s3)cc2)cc1 ; O=C1CCC(N2C(=O)c3ccc(O)cc3C2=O)C(=O)N1 ; O=C(COc1ccc(Oc2nc3ccc(F)cc3s2)cc1)NCCCOCCOCCOCCOCCOc1ccc2c(c1)C(=O)N(C1CCC(=O)NC1=O)C2=O]

To a mixture of 2-[2-[2-[2-[3-[[2-[4-[(6-fluoro-1,3-benzothiazol-2-yl)oxy]phenoxy]acetyl]amino]propoxy]ethoxy]ethoxy]ethoxy]ethyl 4-methylbenzenesulfonate (0.12 g, 169.78 μmol) in DMF (3 mL) were added 2-(2,6-dioxo-3-piperidyl)-5-hydroxy-isoindoline-1,3-dione (69.84 mg, 254.67 μmol), K₂CO₃ (70.40 mg, 509.34 μmol) and NaI (25.45 mg, 169.78 μmol). The mixture was stirred at 40° C. for 12 hours. The solvent was evaporated under reduced pressure. The crude product was purified by prep-HPLC (column: Phenomenex Luna® C18 150*25 mm*10 μm; mobile phase: [water (0.225% FA)-ACN]; B %: 1%-30%, 10 min) to afford N-[3-[2-[2-[2-[2-[2-(2,6-dioxo-3-piperidyl)-1,3-dioxo-isoindolin-5-yl]oxyethoxy]ethoxy]ethoxy]ethoxy]propyl]-2-[4-[(6-fluoro-1,3-benzothiazol-2-yl)oxy]phenoxy]acetamide (33.77 mg, 41.71 μmol, 24.57% yield, 99.9% purity) as an off-white solid. HNMR: DMSO-d₆, 400 MHz. δ 11.12 (s, 1H), 8.11 (t, J=5.6 Hz, 1H), 7.88-7.80 (m, 2H), 7.69 (dd, J=4.8, 8.8 Hz, 1H), 7.45-7.34 (m, 4H), 7.31-7.24 (m, 1H), 7.06 (d, J=8.8 Hz, 2H), 5.15-5.08 (m, 1H), 4.51 (s, 2H), 4.32-4.27 (m, 2H), 3.79-3.75 (m, 2H), 3.60-3.57 (m, 2H), 3.55-3.44 (m, 12H), 3.22-3.16 (m, 2H), 2.92-2.83 (m, 1H), 2.70-2.57 (m, 2H), 2.07-2.00 (m, 1H), 1.71-1.64 (m, 2H). LCMS: RT=2.781 min, m/z 809.2 [M+H]⁺.

The following compounds were prepared in analogous fashion to Example 8.

### Compound 8-A

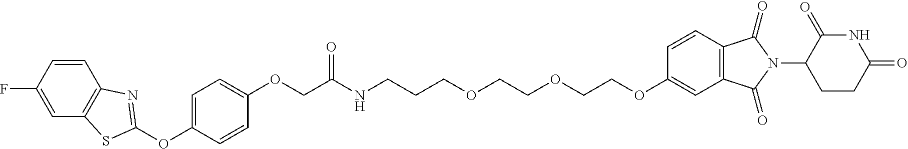 [SMILES: O=C(COc1ccc(Oc2nc3ccc(F)cc3s2)cc1)NCCCOCCOCCOc1ccc2c(c1)C(=O)N(C1CCC(=O)NC1=O)C2=O]

N-(3-(2-(2-((2-(2,6-Dioxopiperidin-3-yl)-1,3-dioxoisoindolin-5-yl)oxy)ethoxy)ethoxy)propyl)-2-(4-((6-fluorobenzo[d]thiazol-2-yl)oxy)phenoxy)acetamide. HNMR: DMSO-d₆, 400 MHz. δ 11.12 (brs, 1H), 8.12 (t, J=5.2 Hz, 1H), 7.84-7.80 (m, 2H), 7.69 (dd, J=8.8, 4.8 Hz, 1H), 7.44-7.36 (m, 4H), 7.30-7.27 (m, 1H), 7.07-7.05 (m, 2H), 5.12 (dd, J=12.8, 5.2 Hz, 1H), 4.51 (s, 2H), 4.31-4.30 (m, 2H), 3.80-3.74 (m, 2H), 3.61-3.58 (m, 2H), 3.51-3.50 (m, 2H), 3.40 (t, J=6.0 Hz, 2H), 3.19 (q, J=6.4 Hz, 2H), 2.95-2.82 (m, 1H), 2.61-2.54 (m, 2H), 2.07-2.00 (m, 1H), 1.67 (quin, J=6.4 Hz, 2H). LCMS: RT=2.777 min, m z 721.2 [M+H]⁺.

### Compound 8-B

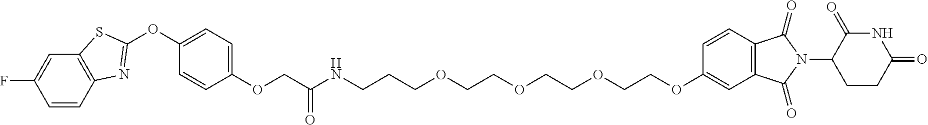 [SMILES: O=C(COc1ccc(Oc2nc3ccc(F)cc3s2)cc1)NCCCOCCOCCOCCOc1ccc2c(c1)C(=O)N(C1CCC(=O)NC1=O)C2=O]

N-(3-(2-(2-(2-((2-(2,6-Dioxopiperidin-3-yl)-1,3-dioxoisoindolin-5-yl)oxy)ethoxy)ethoxy)ethoxy)propyl)-2-(4-((6-fluorobenzo[d]thiazol-2-yl)oxy)phenoxy)acetamide. HNMR: DMSO-d₆, 400 MHz. δ 11.11 (s, 1H), 8.16-8.05 (m, 1H), 7.90-7.78 (m, 2H), 7.69 (dd, J=4.8, 8.8 Hz, 1H), 7.44 (d, J=2.0 Hz, 1H), 7.42-7.34 (m, 3H), 7.31-7.24 (m, 1H), 7.09-7.04 (m, 2H), 5.12 (dd, J=5.2, 12.8 Hz, 1H), 4.51 (s, 2H), 4.34-4.27 (m, 2H), 3.82-3.76 (m, 2H), 3.62-3.57 (m, 2H), 3.55-3.50 (m, 4H), 3.49-3.45 (m, 2H), 3.41-3.39 (m, 2H), 3.23-3.16 (m, 2H), 2.95-2.83 (m, 1H), 2.62-2.54 (m, 2H), 2.11-2.00 (m, 1H), 1.73-1.62 (m, 2H). LCMS: RT=2.779 min, m/z 765.2 [M+H]⁺.

### Compound 8-C

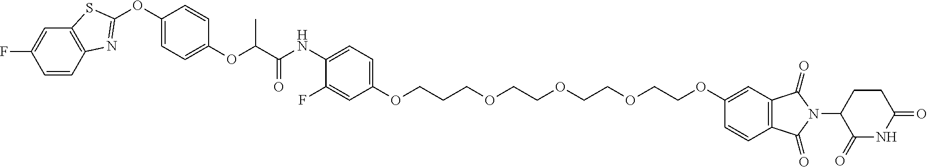 [SMILES: CC(Oc1ccc(Oc2nc3ccc(F)cc3s2)cc1)C(=O)Nc1ccc(OCCCOCCOCCOCCOc2ccc3c(c2)C(=O)N(C2CCC(=O)NC2=O)C3=O)cc1F]

N-(4-(3-(2-(2-(2-((2-(2,6-Dioxopiperidin-3-yl)-1,3-dioxoisoindolin-5-yl)oxy)ethoxy)ethoxy)-ethoxy)propoxy)-2-fluorophenyl)-2-(4-((6-fluorobenzo[d]thiazol-2-yl)oxy)phenoxy)propanamide. HNMR: DMSO-d₆, 400 MHz. δ 11.19-11.02 (y, 1H), 9.79 (s, 1H), 8.46 (s, 1H), 7.91-7.78 (m, 2H), 7.70 (dd, J=4.8, 8.8 Hz, 1H), 7.50-7.32 (m, 5H), 7.27 (dt, J=2.8, 9.2 Hz, 1H), 7.08 (d, J=9.2 Hz, 2H), 6.88 (dd, J=2.4, 12.4 Hz, 1H), 6.75 (br d, J=8.8 Hz, 1H), 5.11 (dd, J=5.6, 12.8 Hz, 1H), 4.99 (q, J=6.8 Hz, 1H), 4.36-4.24 (m, 2H), 4.00 (t, J=6.4 Hz, 2H), 3.77 (br d, J=4.0 Hz, 2H), 3.59-3.55 (m, 2H), 3.54-3.48 (m, 8H), 2.94-2.83 (m, 1H), 2.68-2.54 (m, 2H), 2.09-2.00 (m, 1H), 1.91 (quin, J=6.0 Hz, 2H), 1.56 (d, J=6.8 Hz, 3H). LCMS: RT=0.776 min, m/z 889.1 [M+H]⁺.

TABLE 1
List of compounds from Examples 1-8

| Ex- | | |
|---|---|---|
| ample | Structure | Name |
| 1 | 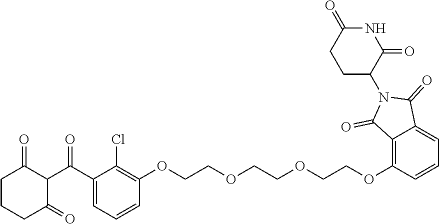 [SMILES: O=C1CCC(N2C(=O)c3cccc(OCCOCCOCCOc4cccc(C(=O)C5C(=O)CCCC5=O)c4Cl)c3C2=O)C(=O)N1] | 4-[2-[2-[2-[2-chloro-3-(2,6- dioxocyclohexanecarbonyl)phenoxy] ethoxy]ethoxy]ethoxy]-2-(2,6-dioxo-3- piperidyl)isoindoline-1,3-dione |
| 1-A | 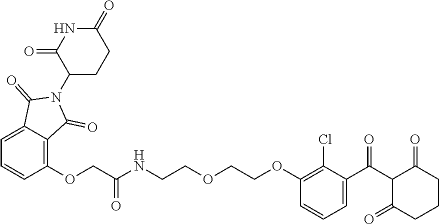 [SMILES: O=C(COc1cccc2c1C(=O)N(C1CCC(=O)NC1=O)C2=O)NCCOCCOc1cccc(C(=O)C2C(=O)CCCC2=O)c1Cl] | N-(2-(2-(2-chloro-3-(2,6- dioxocyclohexane-1- carbonyl)phenoxy)ethoxy)ethyl)-2- ((2-(2,6-dioxopiperidin-3-yl)-1,3- dioxoisoindolin-4-yl)oxy)acetamide |
| 2 | 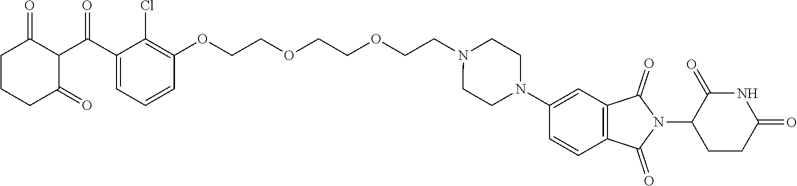 [SMILES: O=C1CCC(N2C(=O)c3ccc(N4CCN(CCOCCOCCOc5cccc(C(=O)C6C(=O)CCCC6=O)c5Cl)CC4)cc3C2=O)C(=O)N1] | 5-[4-[2-[2-[2-[2-chloro-3-(2,6- dioxocyclohexane- carbonyl)phenoxy]ethoxy]ethoxy] ethyl]piperazin-1-yl]-2-(2,6-dioxo-3- piperidyl)isoindoline-1,3-dione |
| 2-A | 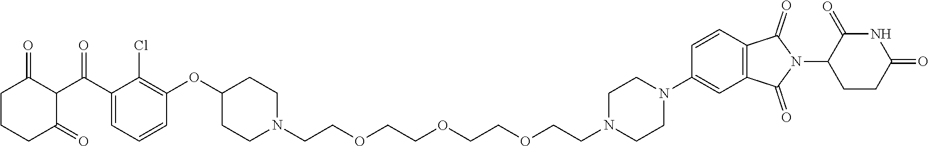 [SMILES: O=C1CCC(N2C(=O)c3ccc(N4CCN(CCOCCOCCOCCN5CCC(Oc6cccc(C(=O)C7C(=O)CCCC7=O)c6Cl)CC5)CC4)cc3C2=O)C(=O)N1] | 5-[4-[2-[2-[2-[2-[4-[2-chloro-3-(2,6- dioxocyclohexanecarbonyl)phenoxy]- 1- piperidyl]ethoxy]ethoxy]ethoxy]ethyl] piperazin-1-yl]-2-(2,6-dioxo-3- piperidyl)isoindoline-1,3-dione |
| 2-B | 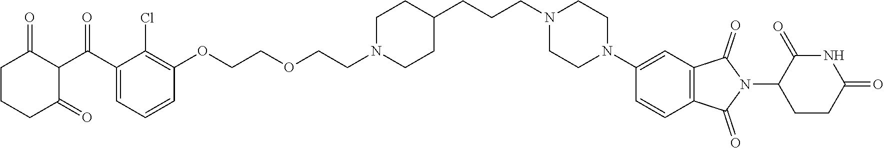 [SMILES: O=C1CCC(N2C(=O)c3ccc(N4CCN(CCCC5CCN(CCOCCOc6cccc(C(=O)C7C(=O)CCCC7=O)c6Cl)CC5)CC4)cc3C2=O)C(=O)N1] | 5-(4-(3-(1-(2-(2-(2-chloro-3-(2,6- dioxocyclohexane-1- carbonyl)phenoxy)ethoxy)ethyl) piperidin-4-yl)propyl)piperazin-1-yl)-2- (2,6-dioxopiperidin-3-yl)isoindoline- 1,3-dione |
| 3 | 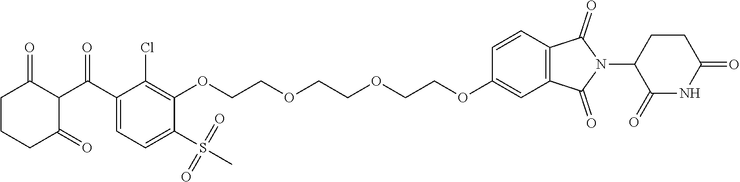 [SMILES: CS(=O)(=O)c1ccc(C(=O)C2C(=O)CCCC2=O)c(Cl)c1OCCOCCOCCOc1ccc2c(c1)C(=O)N(C1CCC(=O)NC1=O)C2=O] | 5-[2-[2-[2-[2-chloro-3-(2,6- dioxocyclohexanecarbonyl)-6- methylsulfonyl- phenoxy]ethoxy]ethoxy]ethoxy]-2- (2,6-dioxo-3-piperidyl)isoindoline- 1,3-dione |
| 4 | 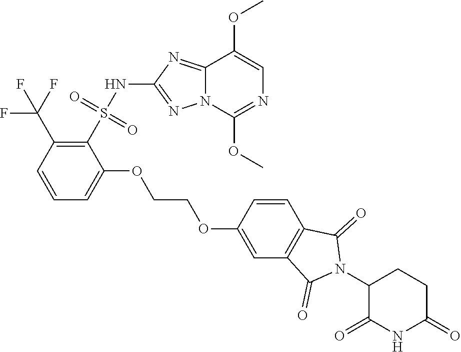 [SMILES: COc1cnc(OC)n2nc(NS(=O)(=O)c3c(OCCOc4ccc5c(c4)C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-[2-[2-(2,6-dioxo- 3-piperidyl)-1,3-dioxo-isoindolin-5- yl]oxyethoxy]-6- (trifluoromethyl)benzenesulfonamide |
| 4-A | 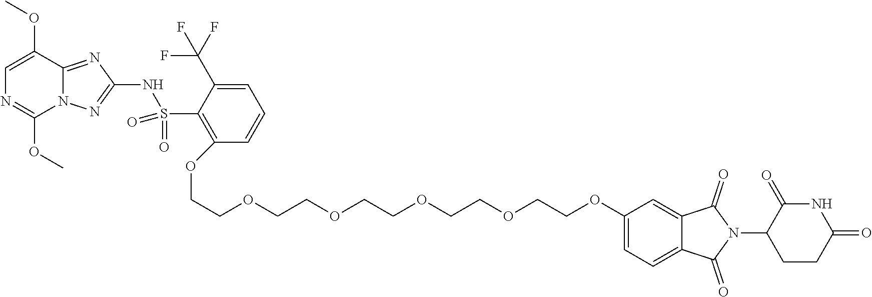 [SMILES: COc1cnc(OC)n2nc(NS(=O)(=O)c3c(OCCOCCOCCOCCOCCOc4ccc5c(c4)C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-((14-((2-(2,6- dioxopiperidin-3-yl)-1,3- dioxoisoindolin-5-yl)oxy)-3,6,9,12- tetraoxatetradecyl)oxy)-6- (trifluoromethyl)benzenesulfonamide |
| 4-B | 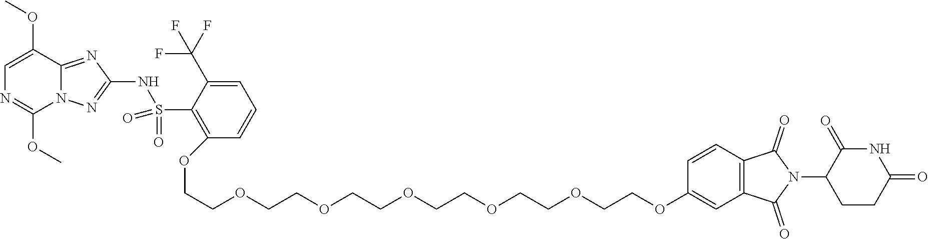 [SMILES: COc1cnc(OC)n2nc(NS(=O)(=O)c3c(OCCOCCOCCOCCOCCOCCOc4ccc5c(c4)C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-((17-((2-(2,6- dioxopiperidin-3-yl)-1,3- dioxoisoindolin-5-yl)oxy)- 3,6,9,12,15-pentaoxaheptadecyl)oxy)- 6- (trifluoromethyl)benzenesulfonamide |
| 4-C | 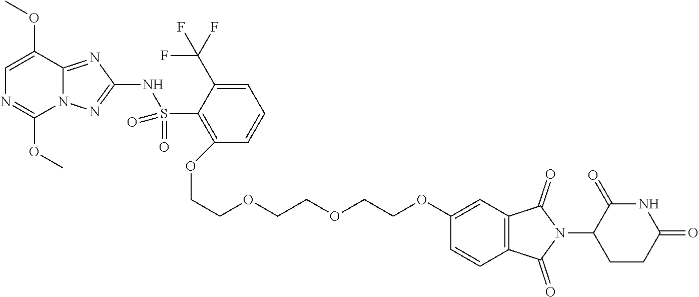 [SMILES: COc1cnc(OC)n2nc(NS(=O)(=O)c3c(OCCOCCOCCOc4ccc5c(c4)C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-(2-(2-(2-((2-(2,6- dioxopiperidin-3-yl)-1,3- dioxoisoindolin-5- yl)oxy)ethoxy)ethoxy)ethoxy)-6- (trifluoromethyl)benzenesulfonamide |
| 4-D | 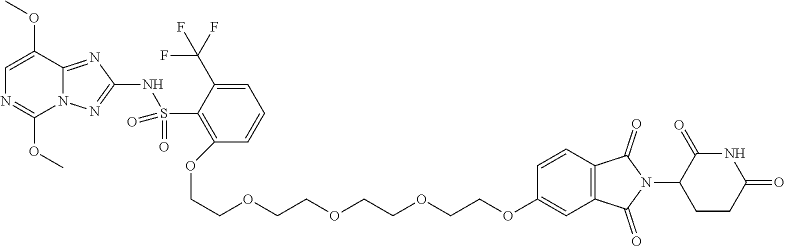 [SMILES: COc1cnc(OC)n2nc(NS(=O)(=O)c3c(OCCOCCOCCOCCOc4ccc5c(c4)C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-(2-(2-(2-(2-((2- (2,6-dioxopiperidin-3-yl)-1,3- dioxoisoindolin-5- yl)oxy)ethoxy)ethoxy)ethoxy) ethoxy)-6- (trifluoromethyl)benzenesulfonamide |
| 4-E | 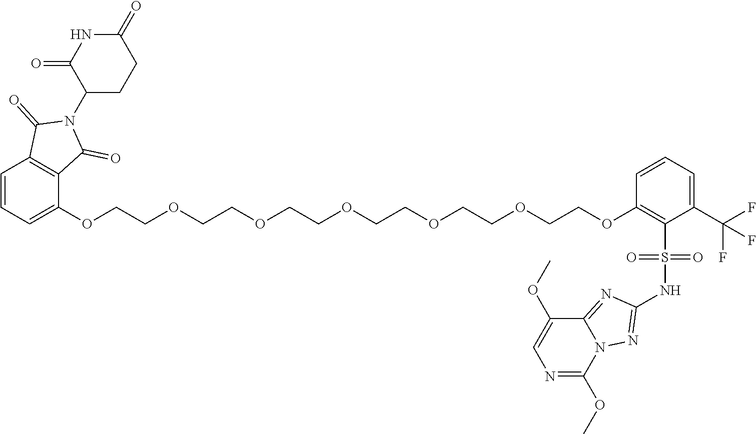 [SMILES: COc1cnc(OC)n2nc(NS(=O)(=O)c3c(OCCOCCOCCOCCOCCOCCOc4cccc5c4C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-((17-((2-(2,6- dioxopiperidin-3-yl)-1,3- dioxoisoindolin-4-yl)oxy)- 3,6,9,12,15-pentaoxaheptadecyl)oxy)- 6- (trifluoromethyl)benzenesulfonamide |
| 4-F | 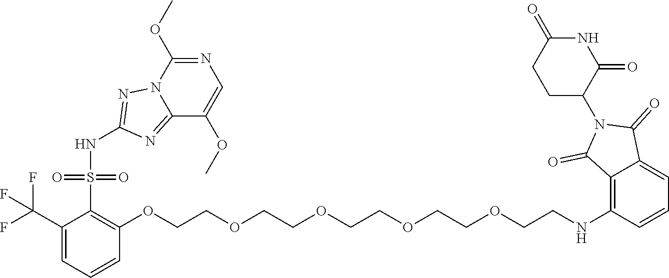 [SMILES: COc1cnc(OC)n2nc(NS(=O)(=O)c3c(OCCOCCOCCOCCOCCNc4cccc5c4C(=O)N(C4CCC(=O)NC4=O)C5=O)cccc3C(F)(F)F)nc12] | N-(5,8-dimethoxy-[1,2,4]triazolo[1,5- c]pyrimidin-2-yl)-2-((14-((2-(2,6- dioxopiperidin-3-yl)-1,3- dioxoisoindolin-4-yl)amino)-3,6,9,12- tetraoxatetradecyl)oxy)-6- (trifluoromethyl)benzenesulfonamide |
| 4-G | 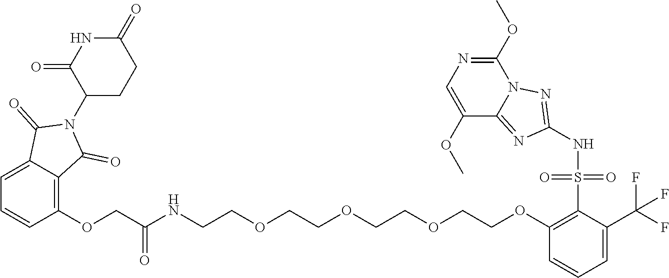 [SMILES: COc1cnc(OC)n2nc(NS(=O)(=O)c3c(OCCOCCOCCOCCNC(=O)COc4cccc5c4C(=O)N(C4CCC(=O)NC4=O)C5=O)cccc3C(F)(F)F)nc12] | N-(2-(2-(2-(2-(2-(N-(5,8-dimethoxy- [1,2,4]triazolo[1,5-c]pyrimidin-2- yl)sulfamoyl)-3- (trifluoromethyl)phenoxy)ethoxy) ethoxy)ethoxy)ethyl)-2-((2-(2,6- dioxopiperidin-3-yl)-1,3- dioxoisoindolin-4-yl)oxy)acetamide |
| 4-H | 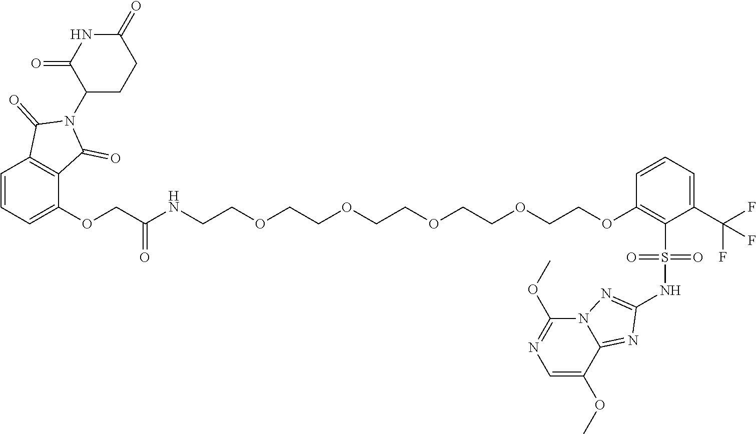 [SMILES: COc1cnc(OC)n2nc(NS(=O)(=O)c3c(OCCOCCOCCOCCOCCNC(=O)COc4cccc5c4C(=O)N(C4CCC(=O)NC4=O)C5=O)cccc3C(F)(F)F)nc12] | N-(14-(2-(N-(5,8-dimethoxy- [1,2,4]triazolo[1,5-c]pyrimidin-2- yl)sulfamoyl)-3- (trifluoromethyl)phenoxy)-3,6,9,12- tetraoxatetradecyl)-2-((2-(2,6- dioxopiperidin-3-yl)-1,3- dioxoisoindolin-4-yl)oxy)acetamide |
| 4-I | 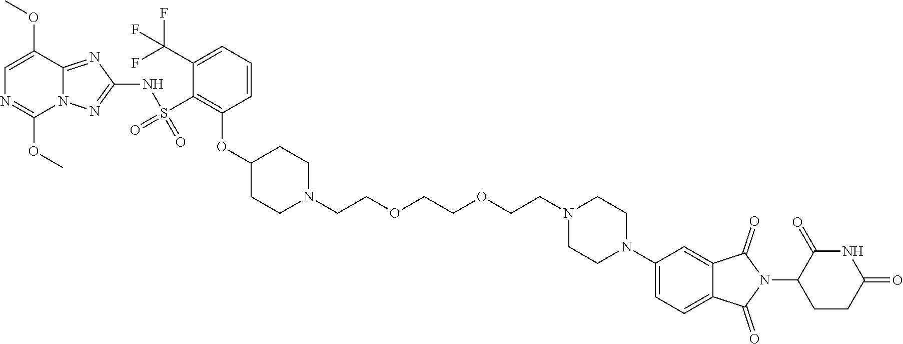 [SMILES: COc1cnc(OC)n2nc(NS(=O)(=O)c3c(OC4CCN(CCOCCOCCN5CCN(c6ccc7c(c6)C(=O)N(C6CCC(=O)NC6=O)C7=O)CC5)CC4)cccc3C(F)(F)F)nc12] | N-(5,8-dimethoxy-[1,2,4]triazolo[1,5- c]pyrimidin-2-yl)-2-((1-(2-(2-(2-(4- (2-(2,6-dioxopiperidin-3-yl)-1,3- dioxoisoindolin-5-yl)piperazin-1- yl)ethoxy)ethoxy)ethyl)piperidin-4- yl)oxy)-6- (trifluoromethyl)benzenesulfonamide |
| 4-J | 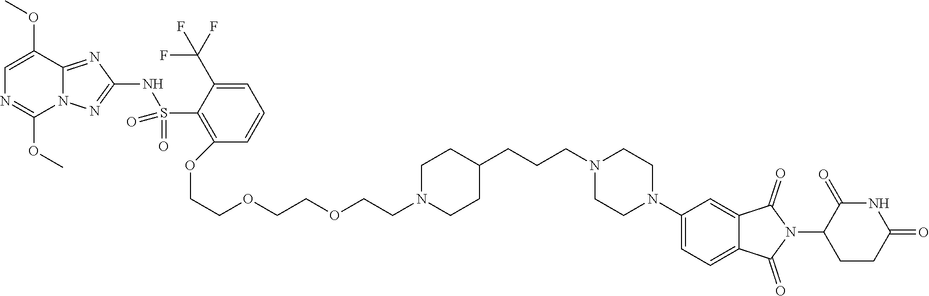 [SMILES: COc1cnc(OC)n2nc(NS(=O)(=O)c3c(OCCOCCOCCN4CCC(CCCN5CCN(c6ccc7c(c6)C(=O)N(C6CCC(=O)NC6=O)C7=O)CC5)CC4)cccc3C(F)(F)F)nc12] | N-(5,8-dimethoxy-[1,2,4]triazolo[1,5- c]pyrimidin-2-yl)-2-(2-(2-(2-(4-(3-(4- (2-(2,6-dioxopiperidin-3-yl)-1,3- dioxoisoindolin-5-yl)piperazin-1- yl)propyl)piperidin-1- yl)ethoxy)ethoxy)ethoxy)-6- (trifluoromethyl)benzenesulfonamide |
| 4-K | 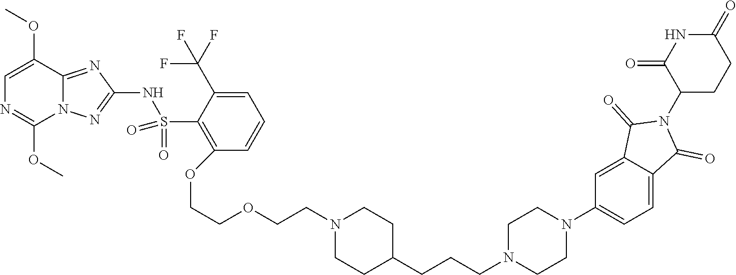 [SMILES: COc1cnc(OC)n2nc(NS(=O)(=O)c3c(OCCOCCN4CCC(CCCN5CCN(c6ccc7c(c6)C(=O)N(C6CCC(=O)NC6=O)C7=O)CC5)CC4)cccc3C(F)(F)F)nc12] | N-(5,8-dimethoxy-[1,2,4]triazolo[1,5- c]pyrimidin-2-yl)-2-(2-(2-(4-(3-(4-(2- (2,6-dioxopiperidin-3-yl)-1,3- dioxoisoindolin-5-yl)piperazin-1- yl)propyl)piperidin-1- yl)ethoxy)ethoxy)-6- (trifluoromethyl)benzenesulfonamide |
| 4-L | 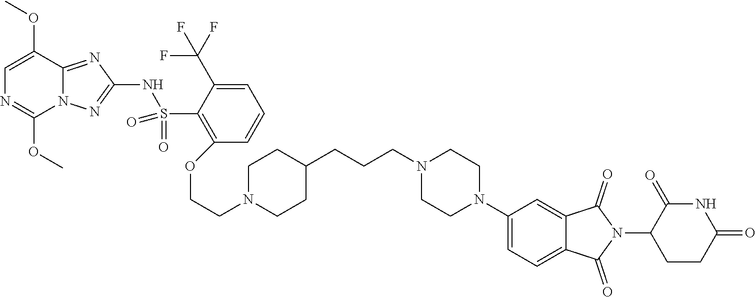 [SMILES: COc1cnc(OC)n2nc(NS(=O)(=O)c3c(OCCN4CCC(CCCN5CCN(c6ccc7c(c6)C(=O)N(C6CCC(=O)NC6=O)C7=O)CC5)CC4)cccc3C(F)(F)F)nc12] | N-(5,8-dimethoxy-[1,2,4]triazolo[1,5- c]pyrimidin-2-yl)-2-(2-(4-(3-(4-(2- (2,6-dioxopiperidin-3-yl)-1,3- dioxoisoindolin-5-yl)piperazin-1- yl)propyl)piperidin-1-yl)ethoxy)-6- (trifluoromethyl)benzenesulfonamide |
| 4-M | 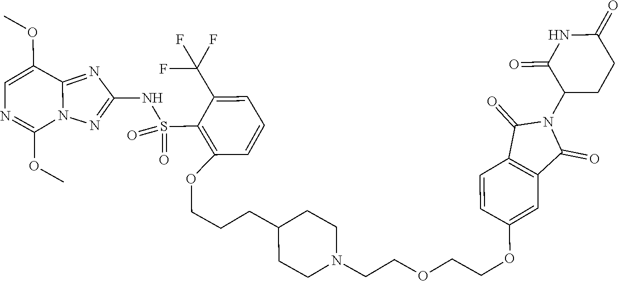 [SMILES: COc1cnc(OC)n2nc(NS(=O)(=O)c3c(OCCCC4CCN(CCOCCOc5ccc6c(c5)C(=O)N(C5CCC(=O)NC5=O)C6=O)CC4)cccc3C(F)(F)F)nc12] | N-(5,8-dimethoxy-[1,2,4]triazolo[1,5- c]pyrimidin-2-yl)-2-(3-(1-(2-(2-((2- (2,6-dioxopiperidin-3-yl)-1,3- dioxoisoindolin-5- yl)oxy)ethoxy)ethyl)piperidin-4- yl)propoxy)-6- (trifluoromethyl)benzenesulfonamide |
| 4-N | 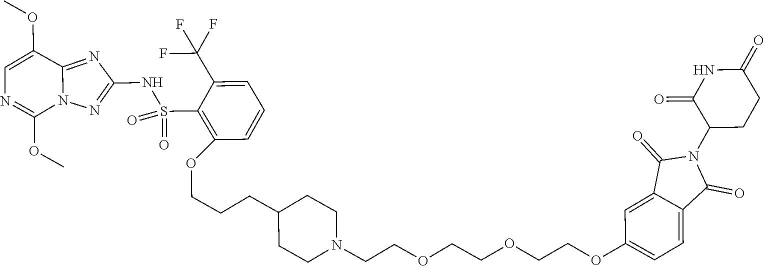 [SMILES: COc1cnc(OC)n2nc(NS(=O)(=O)c3c(OCCCC4CCN(CCOCCOCCOc5ccc6c(c5)C(=O)N(C5CCC(=O)NC5=O)C6=O)CC4)cccc3C(F)(F)F)nc12] | N-(5,8-dimethoxy-[1,2,4]triazolo[1,5- c]pyrimidin-2-yl)-2-(3-(1-(2-(2-(2- ((2-(2,6-dioxopiperidin-3-yl)-1,3- dioxoisoindolin-5- yl)oxy)ethoxy)ethoxy)ethyl)piperidin- 4-yl)propoxy)-6- (trifluoromethyl)benzenesulfonamide |
| 4-O | 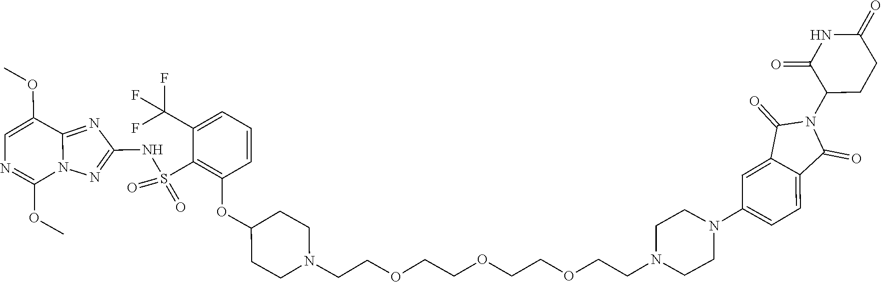 [SMILES: COc1cnc(OC)n2nc(NS(=O)(=O)c3c(OC4CCN(CCOCCOCCOCCN5CCN(c6ccc7c(c6)C(=O)N(C6CCC(=O)NC6=O)C7=O)CC5)CC4)cccc3C(F)(F)F)nc12] | N-(5,8-dimethoxy-[1,2,4]triazolo[1,5- c]pyrimidin-2-yl)-2-((1-(2-(2-(2-(2- (4-(2-(2,6-dioxopiperidin-3-yl)-1,3- dioxoisoindolin-5-yl)piperazin-1- yl)ethoxy)ethoxy)ethoxy)ethyl) piperidin-4-yl)oxy)-6- (trifluoromethyl)benzenesulfonamide |
| 4-P | 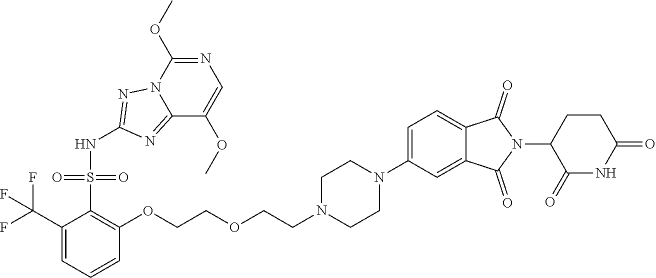 [SMILES: COc1cnc(OC)n2nc(NS(=O)(=O)c3c(OCCOCCN4CCN(c5ccc6c(c5)C(=O)N(C5CCC(=O)NC5=O)C6=O)CC4)cccc3C(F)(F)F)nc12] | N-(5,8-dimethoxy-[1,2,4]triazolo[1,5- c]pyrimidin-2-yl)-2-(2-(2-(4-(2-(2,6- dioxopiperidin-3-yl)-1,3- dioxoisoindolin-5-yl)piperazin-1- yl)ethoxy)ethoxy)-6- (trifluoromethyl)benzenesulfonamide |
| 4-Q | 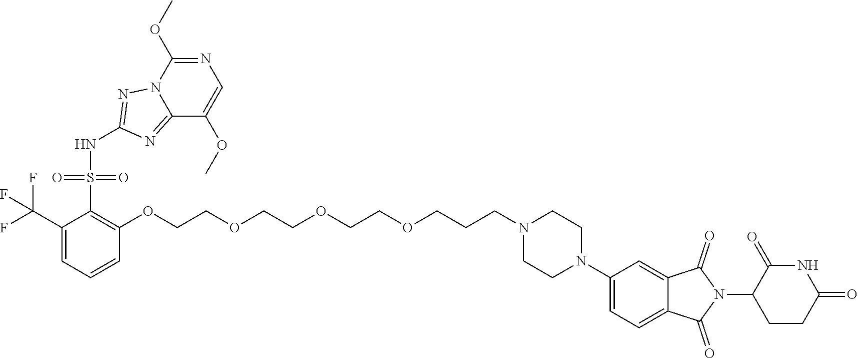 [SMILES: COc1cnc(OC)n2nc(NS(=O)(=O)c3c(OCCOCCOCCOCCCN4CCN(c5ccc6c(c5)C(=O)N(C5CCC(=O)NC5=O)C6=O)CC4)cccc3C(F)(F)F)nc12] | N-(5,8-dimethoxy-[1,2,4]triazolo[1,5- c]pyrimidin-2-yl)-2-(2-(2-(2-(3-(4-(2- (2,6-dioxopiperidin-3-yl)-1,3- dioxoisoindolin-5-yl)piperazin-1- yl)propoxy)ethoxy)ethoxy)ethoxy)-6- (trifluoromethyl)benzenesulfonamide |
| 4-R | 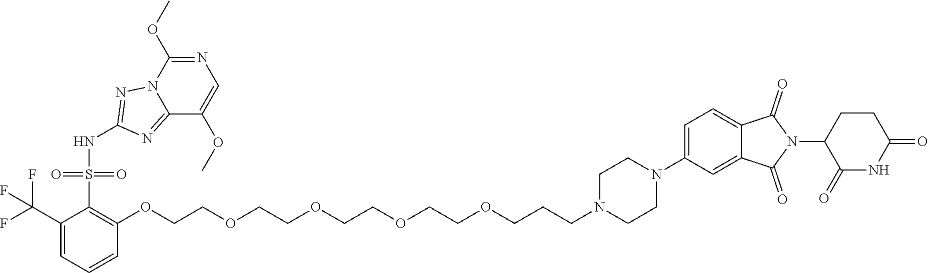 [SMILES: COc1cnc(OC)n2nc(NS(=O)(=O)c3c(OCCOCCOCCOCCOCCCN4CCN(c5ccc6c(c5)C(=O)N(C5CCC(=O)NC5=O)C6=O)CC4)cccc3C(F)(F)F)nc12] | N-(5,8-dimethoxy-[1,2,4]triazolo[1,5- c]pyrimidin-2-yl)-2-((15-(4-(2-(2,6- dioxopiperidin-3-yl)-1,3- dioxoisoindolin-5-yl)piperazin-1-yl)- 3,6,9,12-tetraoxapentadecyl)oxy)-6- (trifluoromethyl)benzenesulfonamide |
| 4-S | 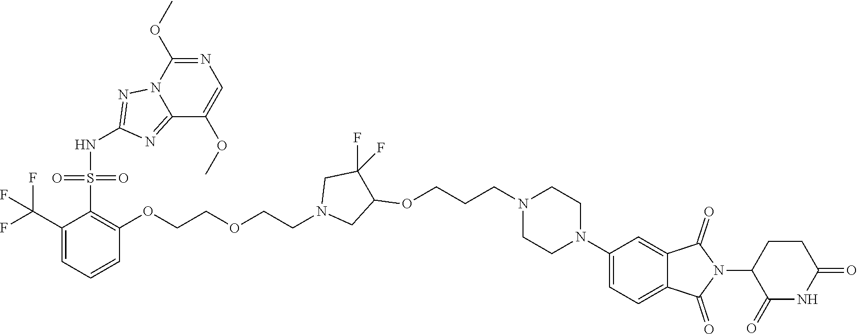 [SMILES: COc1cnc(OC)n2nc(NS(=O)(=O)c3c(OCCOCCN4CC(OCCCN5CCN(c6ccc7c(c6)C(=O)N(C6CCC(=O)NC6=O)C7=O)CC5)C(F)(F)C4)cccc3C(F)(F)F)nc12] | N-(5,8-dimethoxy-[1,2,4]triazolo[1,5- c]pyrimidin-2-yl)-2-(2-(2-(4-(3-(4-(2- (2,6-dioxopiperidin-3-yl)-1,3- dioxoisoindolin-5-yl)piperazin-1- yl)propoxy)-3,3-difluoropyrrolidin-1- yl)ethoxy)ethoxy)-6- (trifluoromethyl)benzenesulfonamide |
| 5 | 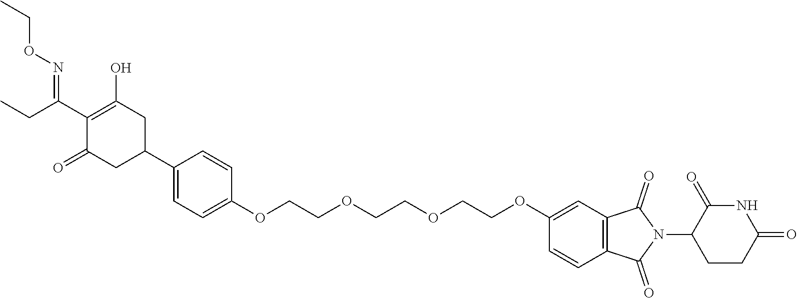 [SMILES: CCO/N=C(\CC)C1=C(O)CC(c2ccc(OCCOCCOCCOc3ccc4c(c3)C(=O)N(C3CCC(=O)NC3=O)C4=O)cc2)CC1=O] | 2-(2,6-dioxo-3-piperidyl)-5-[2-[2-[2- [4-[4-[(E)-N-ethoxy-C-ethyl- carbonimidoyl]-3-hydroxy-5-oxo- cyclohex-3-en-1- yl]phenoxy]ethoxy]ethoxy]ethoxy] isoindoline-1,3-dione |
| 5-A | 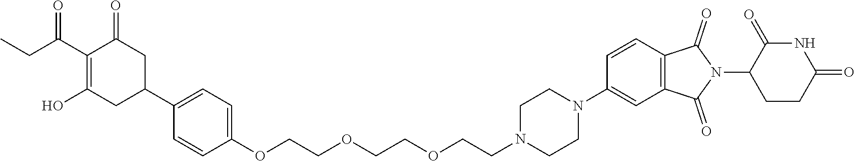 [SMILES: CCC(=O)C1=C(O)CC(c2ccc(OCCOCCOCCN3CCN(c4ccc5c(c4)C(=O)N(C4CCC(=O)NC4=O)C5=O)CC3)cc2)CC1=O] | 2-(2,6-dioxopiperidin-3-yl)-5-(4-(2- (2-(2-((5′-hydroxy-3′-oxo-4′- propionyl-1′,2′,3′,6′-tetrahydro-[1,1′- biphenyl]-4- yl)oxy)ethoxy)ethoxy)ethyl)piperazin- 1-yl)isoindoline-1,3-dione |
| 5-B | 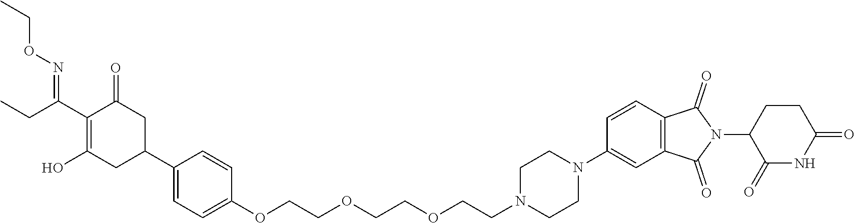 [SMILES: CCO/N=C(\CC)C1=C(O)CC(c2ccc(OCCOCCOCCN3CCN(c4ccc5c(c4)C(=O)N(C4CCC(=O)NC4=O)C5=O)CC3)cc2)CC1=O] | (E)-2-(2,6-dioxopiperidin-3-yl)-5-(4- (2-(2-(2-((4′-(1-(ethoxyimino)propyl)- 5′-hydroxy-3′-oxo-1′,2′,3′,6′- tetrahydro-[1,1′-biphenyl]-4- yl)oxy)ethoxy)ethoxy)ethyl)piperazin- 1-yl)isoindoline-1,3-dione |
| 5-C | 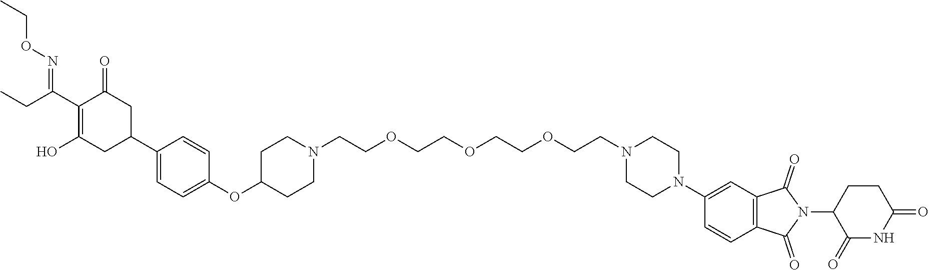 [SMILES: CCO/N=C(\CC)C1=C(O)CC(c2ccc(OC3CCN(CCOCCOCCOCCN4CCN(c5ccc6c(c5)C(=O)N(C5CCC(=O)NC5=O)C6=O)CC4)CC3)cc2)CC1=O] | (E)-2-(2,6-dioxopiperidin-3-yl)-5-(4- (2-(2-(2-(2-(4-((4′-(1- (ethoxyimino)propyl)-5′-hydroxy-3′- oxo-1′,2′,3′,6′-tetrahydro-[1,1′- biphenyl]-4-yl)oxy)piperidin-1- yl)ethoxy)ethoxy)ethoxy)ethyl) piperazin-1-yl)isoindoline-1,3-dione |
| 5-D | 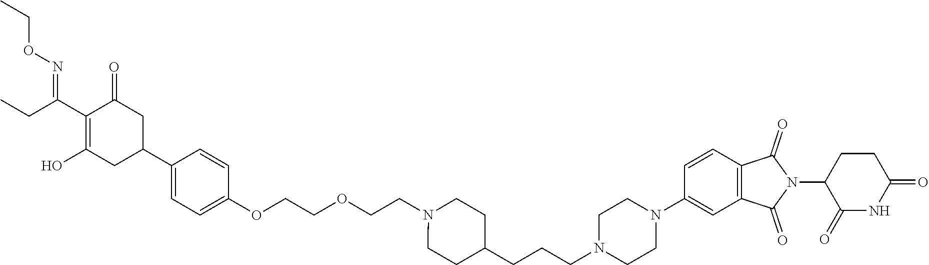 [SMILES: CCO/N=C(\CC)C1=C(O)CC(c2ccc(OCCOCCN3CCC(CCCN4CCN(c5ccc6c(c5)C(=O)N(C5CCC(=O)NC5=O)C6=O)CC4)CC3)cc2)CC1=O] | (E)-2-(2,6-dioxopiperidin-3-yl)-5-(4- (3-(1-(2-(2-((4′-(1- (ethoxyimino)propyl)-5′-hydroxy-3′- oxo-1′,2′,3′,6′-tetrahydro-[1,1′- biphenyl]-4- yl)oxy)ethoxy)ethyl)piperidin-4- yl)propyl)piperazin-1-yl)isoindoline- 1,3-dione |
| 6 | 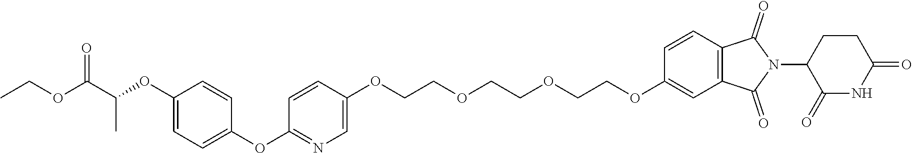 [SMILES: CCOC(=O)[C@@H](C)Oc1ccc(Oc2ccc(OCCOCCOCCOc3ccc4c(c3)C(=O)N(C3CCC(=O)NC3=O)C4=O)cn2)cc1] | Ethyl (2R)-2-(4-((5-(2-(2-(2-((2-(2,6- dioxopiperidin-3-yl)-1,3- dioxoisoindolin-5- yl)oxy)ethoxy)ethoxy)ethoxy)pyridin- 2-yl)oxy)phenoxy)propanoate |
| 6-A | 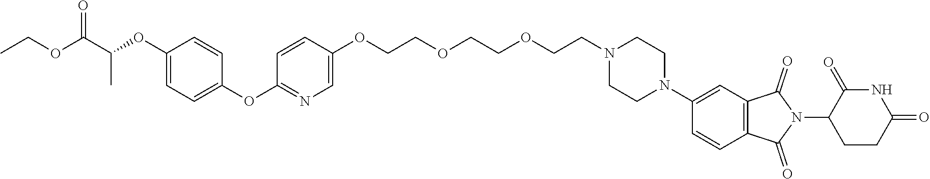 [SMILES: CCOC(=O)[C@@H](C)Oc1ccc(Oc2ccc(OCCOCCOCCN3CCN(c4ccc5c(c4)C(=O)N(C4CCC(=O)NC4=O)C5=O)CC3)cn2)cc1] | ethyl (2R)-2-(4-((5-(2-(2-(2-(4-(2- (2,6-dioxopiperidin-3-yl)-1,3- dioxoisoindolin-5-yl)piperazin-1- yl)ethoxy)ethoxy)ethoxy)pyridin-2- yl)oxy)phenoxy)propanoate |
| 6-B | 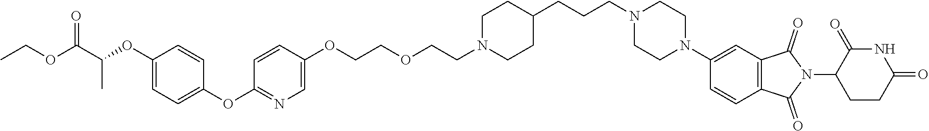 [SMILES: CCOC(=O)[C@@H](C)Oc1ccc(Oc2ccc(OCCOCCN3CCC(CCCN4CCN(c5ccc6c(c5)C(=O)N(C5CCC(=O)NC5=O)C6=O)CC4)CC3)cn2)cc1] | ethyl (2R)-2-[4-[[5-[2-[2-[4-[3-[4-[2- (2,6-dioxo-3-piperidyl)-1,3-dioxo- isoindolin-5-yl]piperazin-1- yl]propyl]-1- piperidyl]ethoxy]ethoxy]-2- pyridyl]oxy]phenoxy\|propanoate |
| 6-C | 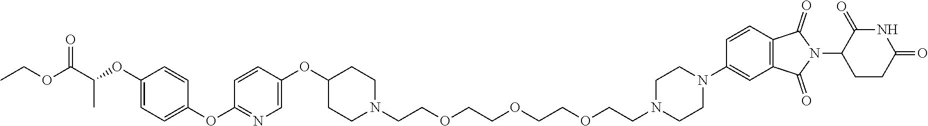 [SMILES: CCOC(=O)[C@@H](C)Oc1ccc(Oc2ccc(OC3CCN(CCOCCOCCOCCN4CCN(c5ccc6c(c5)C(=O)N(C5CCC(=O)NC5=O)C6=O)CC4)CC3)cn2)cc1] | ethyl (2R)-2-(4-((5-((1-(2-(2-(2-(2-(4- (2-(2,6-dioxopiperidin-3-yl)-1,3- dioxoisoindolin-5-yl)piperazin-1- yl)ethoxy)ethoxy)ethoxy)ethyl) piperidin-4-yl)oxy)pyridin-2- yl)oxy)phenoxy)-propanoate |
| 7 | 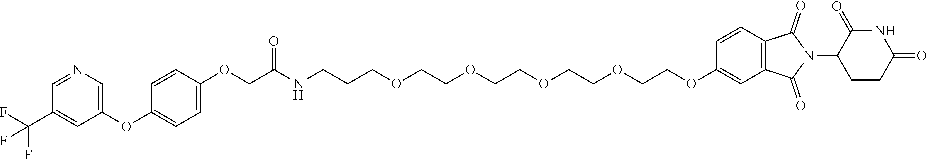 [SMILES: O=C(COc1ccc(Oc2cncc(C(F)(F)F)c2)cc1)NCCCOCCOCCOCCOCCOc1ccc2c(c1)C(=O)N(C1CCC(=O)NC1=O)C2=O] | N-[3-[2-[2-[2-[2-[2-(2,6-dioxo-3- piperidyl)-1,3-dioxo-isoindolin-5- yl]oxyethoxy]- ethoxy]ethoxy]ethoxy]propyl]-2-[4- [[5-(trifluoromethyl)-3- pyridyl]oxy]phenoxy]acetamide |
| 7-A | 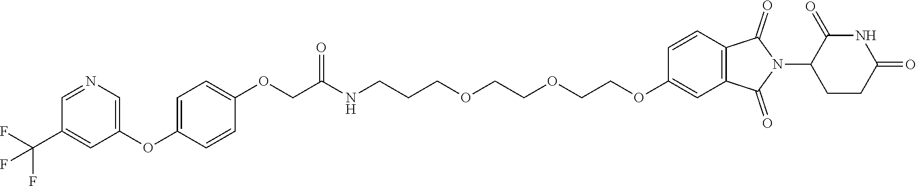 [SMILES: O=C(COc1ccc(Oc2cncc(C(F)(F)F)c2)cc1)NCCCOCCOCCOc1ccc2c(c1)C(=O)N(C1CCC(=O)NC1=O)C2=O] | N-(3-(2-(2-((2-(2,6-dioxopiperidin-3- yl)-1,3-dioxoisoindolin-5- yl)oxy)ethoxy)ethoxy)propyl)-2-(4- ((5-(trifluoromethyl)pyridin-3- yl)oxy)phenoxy)acetamide |
| 7-B | 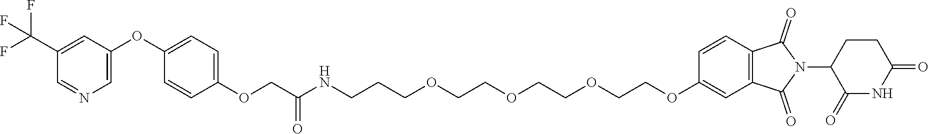 [SMILES: O=C(COc1ccc(Oc2cncc(C(F)(F)F)c2)cc1)NCCCOCCOCCOCCOc1ccc2c(c1)C(=O)N(C1CCC(=O)NC1=O)C2=O] | N-(3-(2-(2-(2-((2-(2,6-dioxopiperidin- 3-yl)-1,3-dioxoisoindolin-5- yl)oxy)ethoxy)ethoxy)ethoxy)propyl)- 2-(4-((5-(trifluoromethyl)pyridin-3- yl)oxy)phenoxy)acetamide |
| 7-C | 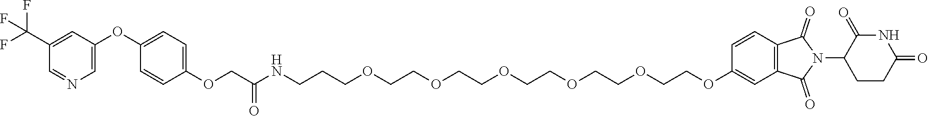 [SMILES: O=C(COc1ccc(Oc2cncc(C(F)(F)F)c2)cc1)NCCCOCCOCCOCCOCCOCCOc1ccc2c(c1)C(=O)N(C1CCC(=O)NC1=O)C2=O] | N-(1-((2-(2,6-dioxopiperidin-3-yl)- 1,3-dioxoisoindolin-5-yl)oxy)- 3,6,9,12,15-pentaoxaoctadecan-18- yl)-2-(4-((5-(trifluoromethyl)pyridin- 3-yl)oxy)phenoxy)acetamide |
| 8 | 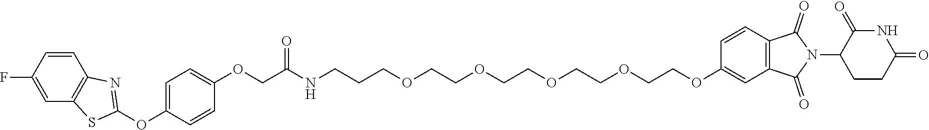 [SMILES: O=C(COc1ccc(Oc2nc3ccc(F)cc3s2)cc1)NCCCOCCOCCOCCOCCOc1ccc2c(c1)C(=O)N(C1CCC(=O)NC1=O)C2=O] | N-[3-[2-[2-[2-[2-[2-(2,6-dioxo-3- piperidyl)-1,3-dioxo-isoindolin-5- yl]oxyethoxy]- ethoxy]ethoxy]ethoxy]propyl]-2-[4- [(6-fluoro-1,3-benzothiazol-2- yl)oxy]phenoxy]acetamide |
| 8-A | 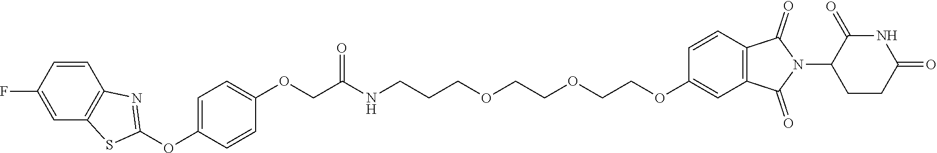 [SMILES: O=C(COc1ccc(Oc2nc3ccc(F)cc3s2)cc1)NCCCOCCOCCOc1ccc2c(c1)C(=O)N(C1CCC(=O)NC1=O)C2=O] | N-(3-(2-(2-((2-(2,6-dioxopiperidin-3- yl)-1,3-dioxoisoindolin-5- yl)oxy)ethoxy)ethoxy)propyl)-2-(4- ((6-fluorobenzo[d]thiazol-2- yl)oxy)phenoxy)acetamide |
| 8-B | 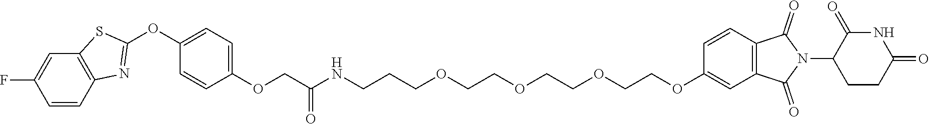 [SMILES: O=C(COc1ccc(Oc2nc3ccc(F)cc3s2)cc1)NCCCOCCOCCOCCOc1ccc2c(c1)C(=O)N(C1CCC(=O)NC1=O)C2=O] | N-(3-(2-(2-(2-((2-(2,6-dioxopiperidin- 3-yl)-1,3-dioxoisoindolin-5- yl)oxy)ethoxy)ethoxy)ethoxy)propyl)- 2-(4-((6-fluorobenzo[d]thiazol-2- yl)oxy)phenoxy)acetamide |
| 8-C | 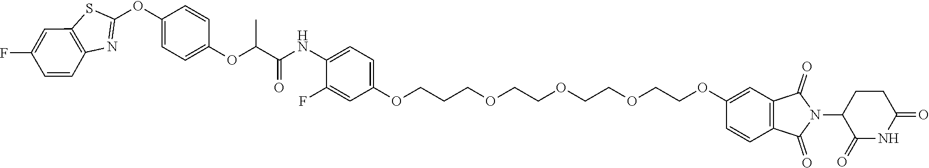 [SMILES: CC(Oc1ccc(Oc2nc3ccc(F)cc3s2)cc1)C(=O)Nc1ccc(OCCCOCCOCCOCCOc2ccc3c(c2)C(=O)N(C2CCC(=O)NC2=O)C3=O)cc1F] | N-(4-(3-(2-(2-(2-((2-(2,6- dioxopiperidin-3-yl)-1,3- dioxoisoindolin-5- yl)oxy)ethoxy)ethoxy)- ethoxy)propoxy)-2-fluorophenyl)-2- (4-((6-fluorobenzo[d]thiazol-2- yl)oxy)phenoxy)propanamide |

### Example 9: Synthesis of INT-1: (S)-tert-Butyl 5-amino-4-(4-((4-formylbenzyl)oxy)-1-oxoisoindolin-2-yl)-5-oxopentanoate

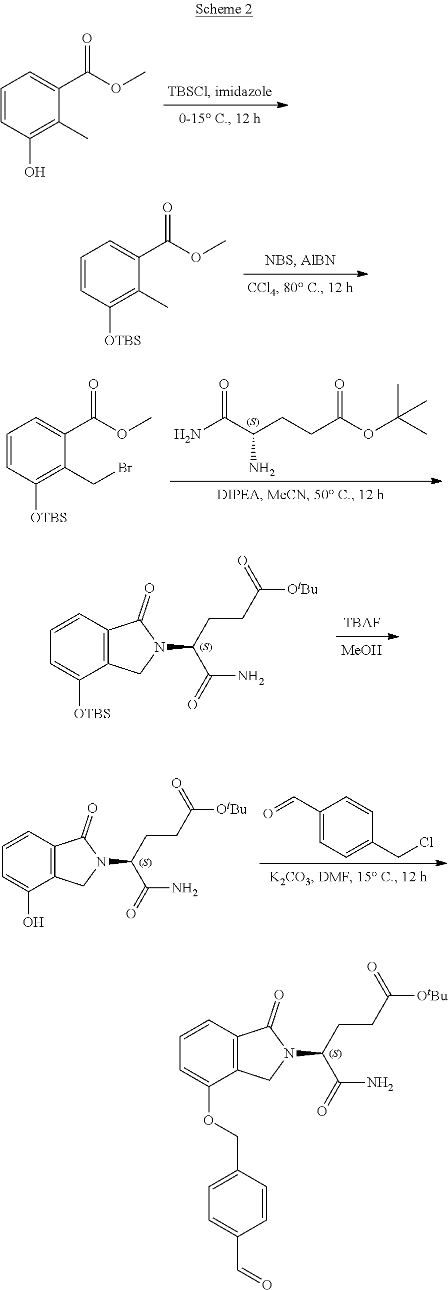

### Step 1: Methyl 3-((tert-butyldimethylsilyl)oxy)-2-methylbenzoate

To a solution of methyl 3-hydroxy-2-methylbenzoate (98.00 g, 590.00 mmol) and imidazole (100.37 g, 1.470 mol) in DMF (800 mL) was added TBSCl (97.78 g, 648.72 mmol) in portions at 0° C. The reaction mixture was stirred at 15° C. for 12 hours. LCMS showed the desired mass, and the reaction was complete. The reaction mixture was poured into water (2 L) and extracted with ethyl acetate (600 mL×3). The combined organic phase was washed with brine (200 mL×3) and saturated NH₄Cl aqueous (200 mL×3), dried over anhydrous sodium sulfate and concentrated under reduced pressure. The residue was purified by flash silica gel column chromatography (SiO₂, 0-1% ethyl acetate in Petroleum ether, Petroleum ether:Ethyl Acetate=10:1, Rf=0.68) to afford methyl 3-((tert-butyldimethylsilyl)oxy)-2-methylbenzoate (165.00 g, 588.37 mmol, 99.77% yield) as a brown oil. HNMR: CDCl₃, 400 MHz. δ 7.45-7.42 (dd, J=8.0, 0.8 Hz, 1H), 7.12-7.08 (t, J=8.0 Hz, 1H), 6.95-6.93 (dd, J=8.0, 0.8 Hz, 1H), 3.89 (s, 3H), 2.43 (s, 3H), 1.04 (s, 9H), 0.23 (s, 6H). LCMS: RT=1.127 min, m/z 281.1 [M+H]⁺.

### Step 2: Methyl 2-(bromomethyl)-3-((tert-butyldimethylsilyl)oxy)benzoate

To a solution of methyl 3-((tert-butyldimethylsilyl)oxy)-2-methylbenzoate (5.00 g, 17.83 mmol) in CCl₄ (50 mL) were added NBS (3.17 g, 17.83 mmol) and AIBN (146.39 mg, 891.48 μmol). The reaction mixture was stirred at 80° C. for 12 hours under nitrogen atmosphere. TLC (Petroleum ether:Ethyl Acetate=10:1, Rf=0.73) showed the reaction was complete. The solution was cooled to room temperature and filtered through a pad of Celite. The filter cake was washed with Petroleum ether (50 mL). The filtrate was concentrated and diluted with Ethyl acetate (80 mL). The solution was washed with saturated NH₄Cl aqueous (30 mL×2), brine (30 mL×2), dried over anhydrous sodium sulfate, filtered, and concentrated under reduced pressure. The residue was purified by flash silica gel chromatography (SiO₂, 0-3% Ethyl acetate in Petroleum ether, Petroleum ether:Ethyl Acetate=10:1, Rf=0.73) to afford methyl 2-(bromomethyl)-3-((tert-butyldimethylsilyl)oxy)benzoate (5.30 g, 14.75 mmol, 82.73% yield) as a brown oil. HNMR: CDCl₃, 400 MHz. δ 7.54-7.52 (dd, J=8.0, 0.8 Hz, 1H), 7.26-7.22 (t, J=8.0 Hz, 1H), 7.02-7.00 (dd, J=8.0, 0.8 Hz, 1H), 5.03 (s, 2H), 3.94 (s, 3H), 1.08 (s, 9H), 0.31 (s, 6H).

### Step 3: (S)-tert-Butyl 5-amino-4-(4-((tert-butyldimethylsilyl)oxy)-1-oxoisoindolin-2-yl)-5-oxopentanoate

To a mixture of methyl 2-(bromomethyl)-3-((tert-butyldimethylsilyl)oxy)benzoate (5.300 g, 14.75 mmol) and (S)-tert-butyl 4,5-diamino-5-oxopentanoate; hydrochloride (3.700 g, 15.49 mmol) in MeCN (50 mL) was added DIPEA (5.72 g, 44.25 mmol) and the reaction mixture was stirred for 12 hours at 50° C. LCMS showed the desired mass. The reaction mixture was concentrated under reduced pressure. The residue was purified by flash silica gel column chromatography (SiO₂, 0-60% Ethyl Acetate in Petroleum ether, Petroleum ether:Ethyl Acetate=1:1, Rf=0.31) to afford (S)-tert-butyl 5-amino-4-(4-((tert-butyldimethylsilyl)oxy)-1-oxoisoindolin-2-yl)-5-oxopentanoate (3.700 g, 8.25 mmol, 55.9% yield) as a yellow oil. HNMR: CDCl₃, 400 MHz. δ 7.46-7.44 (d, J=7.2 Hz, 1H), 7.37-7.33 (m, 1H), 6.98-6.96 (dd, J=8.0, 0.8 Hz, 1H), 6.37 (s, 1H), 5.39 (s, 1H), 4.91-4.87 (m, 1H), 4.43-4.32 (m, 2H), 2.44-2.18 (m, 4H), 1.43 (s, 9H), 1.01 (s, 9H), 0.27-0.26 (d, d, J=7.2 Hz, 6H). LCMS: RT=0.973 min, m z 449.2 [M+H]⁺.

### Step 4: (S)-tert-Butyl 5-amino-4-(4-hydroxy-1-oxoisoindolin-2-yl)-5-oxopentanoate

To a solution of (S)-tert-butyl 5-amino-4-(4-((tert-butyldimethylsilyl)oxy)-1-oxoisoindolin-2-yl)-5-oxopentanoate (3.700 g, 8.25 mmol) in MeOH (40 mL) was added TBAF solution in THE (1 M, 1.65 mL) and the reaction mixture was stirred for 12 hours at 15° C. LCMS showed the desired mass. The reaction mixture was concentrated under reduced pressure. The residue was purified by flash silica gel column chromatography (SiO₂, 0-85% Ethyl Acetate in Petroleum ether, Petroleum ether:Ethyl Acetate=1:3, Rf=0.31) to afford the desired product (S)-tert-butyl 5-amino-4-(4-hydroxy-1-oxoisoindolin-2-yl)-5-oxopentanoate (2.500 g, 7.48 mmol, 90.7% yield) as a brown solid. HNMR: CDCl₃, 400 MHz. δ 8.69 (s, 1H), 7.37-7.35 (d, J=7.2 Hz, 1H), 7.32-7.29 (m, 1H), 7.03-7.01 (d, J=8.0 Hz, 1H), 6.88 (s, 1H), 6.01 (s, 1H), 5.02-4.96 (m, 1H), 4.62-4.57 (m, 1H), 4.51-4.46 (m, 1H), 2.43-2.27 (m, 3H), 2.25-2.17 (m, 1H), 1.43 (s, 9H). LCMS: RT=0.707 min, m/z 357.1 [M+23]⁺.

### Step 5: INT-1: (S)-tert-Butyl 5-amino-4-(4-((4-formylbenzyl)oxy)-1-oxoisoindolin-2-yl)-5-oxopentanoate

To a solution of (S)-tert-butyl 5-amino-4-(4-hydroxy-1-oxoisoindolin-2-yl)-5-oxopentanoate (1.8 g, 5.38 mmol) and 4-(chloromethyl)benzaldehyde (0.999 g, 6.46 mmol) in DMF (20 mL) was added K₂CO₃ (1.860 g, 13.46 mmol) and the reaction mixture was stirred for 12 hours at 15° C. LCMS showed the reaction was completed. The reaction mixture was poured into Ethyl Acetate (100 mL) and filtered. The filtrate was washed with brine (30 mL×3), dried over anhydrous sodium sulfate, and concentrated to dryness. The residue was purified by flash silica gel chromatography (Petroleum ether:Ethyl Acetate=1:1 to 1:5, Rf=0.37) to afford (S)-tert-butyl 5-amino-4-(4-((4-formylbenzyl)oxy)-1-oxoisoindolin-2-yl)-5-oxopentanoate (2.000 g, 4.42 mmol, 82.1% yield) as a white solid. HNMR: CDCl₃, 400 MHz. δ 10.05 (s, 1H), 7.95-7.93 (d, J=8.0 Hz, 2H), 7.61-7.59 (d, J=7.6 Hz, 2H), 7.49-7.35 (m, 2H), 7.05-7.03 (d, J=8.0 Hz, 1H), 6.39 (s, 1H), 5.52 (s, 1H), 5.25 (s, 2H), 4.94-4.90 (m, 1H), 4.58-4.44 (m, 2H), 2.44-2.15 (m, 4H), 1.42 (s, 9H). LCMS: RT=0.839 min, m/z 449.3 [M−3]⁺.

### Example 10: Synthesis of INT-2: 3-((2-Methoxyethoxy)methyl)morpholine

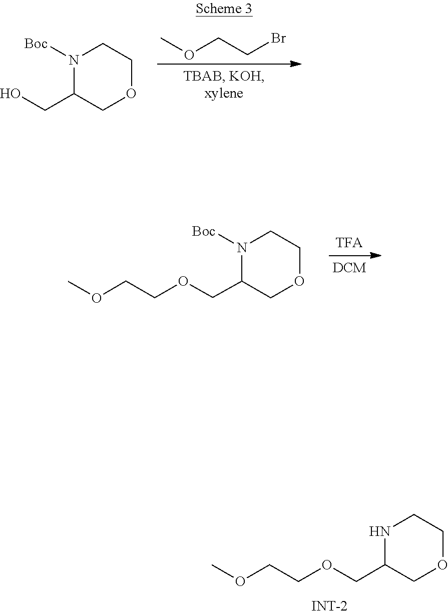

### Step 1: tert-Butyl 3-((2-methoxyethoxy)methyl)morpholine-4-carboxylate

To a solution of tert-butyl 3-(hydroxymethyl)morpholine-4-carboxylate (1.000 g, 4.60 mmol) and 1-bromo-2-methoxyethane (959.61 mg, 6.90 mmol, 648.39 μL) in xylene (10 mL) was added KOH (1.29 g, 23.01 mmol) and TBAB (148.38 mg, 460.28 μmol). The reaction mixture was stirred for 12 hours at 30° C. TLC (Petroleum ether:Ethyl Acetate=2: 1, Rf=0.34) showed the reaction was completed. The reaction mixture was poured into Ethyl Acetate (30 mL) and filtered. The filtrate was concentrated to dryness under reduced pressure. The residue was purified by column chromatography (SiO₂, Petroleum ether:Ethyl Acetate=2:1 to 1:1, Rf=0.34) to afford tert-butyl 3-((2-methoxyethoxy)methyl)morpholine-4-carboxylate (0.700 g, 2.54 mmol, 55.23% yield) as a colorless oil. HNMR: CDCl₃, 400 MHz. δ 4.15-4.07 (m, 1H), 3.99-3.97 (m, 1H), 3.85-3.83 (m, 1H), 3.75-3.71 (m, 2H), 3.65 (m, 2H), 3.57-3.38 (m, 8H), 3.10-3.07 (m, 1H), 1.47 (s, 9H).

### Step 2: INT-2: 3-((2-Methoxyethoxy)methyl)morpholine

To a solution of tert-butyl 3-((2-methoxyethoxy)methyl)morpholine-4-carboxylate (0.700 g, 2.54 mmol) in DCM (3 mL) was added TFA (1 mL) and the reaction mixture was stirred for 12 hours at 15° C. TLC (Petroleum ether:Ethyl Acetate=1:1, Rf=0.01) showed the reaction was complete. The reaction mixture was concentrated under reduced pressure to afford a residue. Water (20 mL) was added and the pH value was adjust to 7-8 with saturated NaHCO_{3 (aq)}, extracted with Ethyl Acetate (20 mL×3), dichloromethane (20 mL×5), dried over anhydrous sodium sulfate, and concentrated under reduced pressure to afford 3-((2-methoxyethoxy)methyl)morpholine (0.400 g, 2.28 mmol, 89.79% yield) as a brown oil, which was used in next step directly without further purification. HNMR: CDCl₃, 400 MHz. δ 3.85-3.79 (m, 2H), 3.66-3.52 (m, 5H), 3.49-3.46 (m, 1H), 3.42-3.32 (m, 5H), 3.18-3.13 (m, 1H), 3.02-3.94 (m, 2H).

### Example 11: Synthesis of 3-((2-Methoxyethoxy)methyl)morpholine

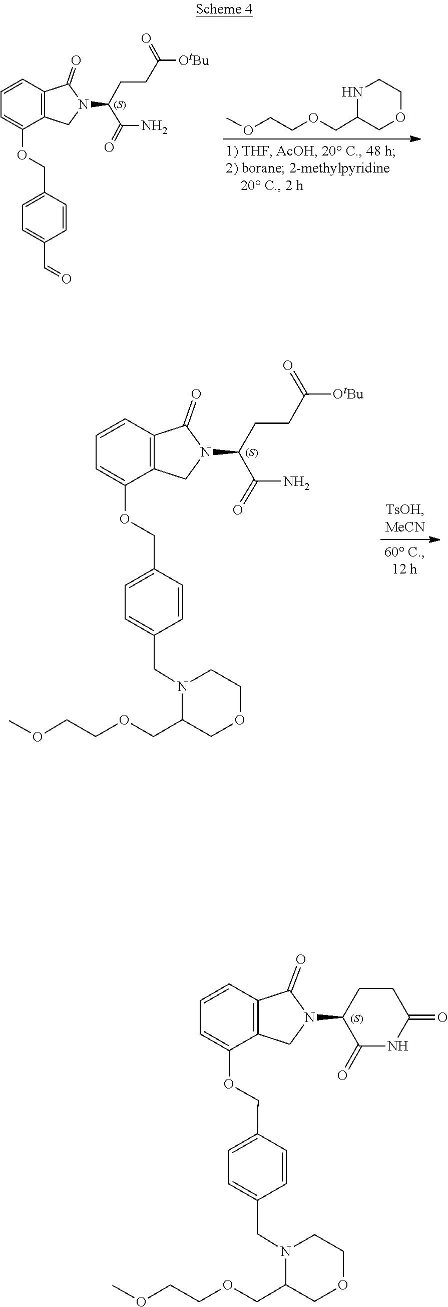

### Step 1: (4S)-tert-Butyl 5-amino-4-(4-((4-((3-((2-methoxyethoxy)methyl) morpholino)-methyl)benzyl)oxy)-1-oxoisoindolin-2-yl)-5-oxopentanoate

To a solution of (S)-tert-butyl 5-amino-4-(4-((4-formylbenzyl)oxy)-1-oxoisoindolin-2-yl)-5-oxopentanoate (INT-1) (0.350 g, 773.48 μmol) and 3-((2-methoxyethoxy)methyl)morpholine (INT-2) (0.271 g, 1.55 mmol) in THF (5 mL) and AcOH (0.5 mL). The reaction mixture was stirred for 48 hours at 20° C. borane; 2-methylpyridine (0.248 g, 2.32 mmol) was added and stirred for 2 hours at 20° C. LCMS showed the desired mass and a trace of the starting material. The reaction mixture was concentrated under reduced pressure. The residue was purified by semi-preparative reverse phase HPLC (13%-43% acetonitrile in water+0.225% formic acid, 10 min) followed by lyophilization to afford (4S)-tert-butyl 5-amino-4-(4-((4-((3-((2-methoxyethoxy)methyl)-morpholino)methyl)benzyl)oxy)-1-oxoisoindolin-2-yl)-5-oxopentanoate (0.250 g, 380.08 μmol, 49.14% yield, formic acid salt) as a brown solid. HNMR: DMSO-d₆, 400 MHz. δ 8.14 (s, 1H), 7.59 (s, 1H), 7.48-7.44 (m, 3H), 7.38-7.36 (m, 2H), 7.31-7.29 (m, 2H), 7.20 (s, 1H), 5.23 (s, 2H), 4.74-4.70 (m, 1H), 4.56-4.51 (m, 1H), 4.43-4.39 (m, 1H), 4.02 (m, 1H), 3.74-3.67 (m, 2H), 3.59 (m, 1H), 3.51-3.43 (m, 6H), 3.35 (m, 3H), 3.24 (s, 3H), 2.58 (m, 1H), 2.17-2.08 (m, 4H), 2.05-1.97 (m, 1H), 1.33 (s, 9H). LCMS: RT=0.723 min, m/z 613.3 [M+H]⁺.

### Step 2: (3S)-3-(4-((4-((3-((2-Methoxyethoxy)methyl)morpholino)methyl)benzyl) oxy)-1-oxoisoindolin-2-yl)piperidine-2,6-dione

To a solution of (4S)-tert-butyl 5-amino-4-(4-((4-((3-((2-methoxyethoxy)methyl)morpholino)-methyl)benzyl)oxy)-1-oxoisoindolin-2-yl)-5-oxopentanoate (0.150 g, 228.05 μmol, formic acid salt) in MeCN (2 mL) was added TsOH (98.18 mg, 570.13 μmol) and the reaction mixture was stirred for 12 hours at 60° C. LCMS showed the desired mass. This reaction was poured into water (10 mL) and pH value was adjusted to 7-8 with saturated NaHCO₃(aq), extracted with Ethyl Acetate (20 mL×3), dried over anhydrous sodium sulfate, and concentrated under reduced pressure to afford the crude product, which was purified by semi-preparative reverse phase HPLC (20%-50% MeCN in water, water (10 mM NH₄HCO₃)-MeCN, 10 min) followed by lyophilization to afford (3S)-3-(4-((4-((3-((2-methoxyethoxy)methyl)morpholino)methyl)benzyl)-oxy)-1-oxoisoindolin-2-yl)piperidine-2,6-dione (54.18 mg, 100.68 μmol, 44.15% yield, 99.9% purity) as a white solid. HNMR: DMSO-d6, 400 MHz. δ 10.98 (s, 1H), 7.50-7.42 (m, 3H), 7.35-7.31 (m, 4H), 5.22 (s, 2H), 5.13-5.09 (m, 1H), 4.43-4.39 (m, 1H), 4.27-4.22 (m, 1H), 4.02-3.99 (m, 1H), 3.72-3.65 (m, 2H), 3.60-3.56 (m, 1H), 3.50-3.47 (m, 2H), 3.45-3.41 (m, 4H), 3.36 (m, 1H), 3.32-3.30 (m, 2H), 3.22 (s, 3H), 2.95-2.86 (m, 1H), 2.59-2.54 (m, 2H), 2.47-2.39 (m, 1H), 2.17-2.11 (m, 1H), 1.99-1.96 (m, 1H). LCMS: RT=0.664 min, m/z 538.2 [M+H]⁺. QC_LCMS: RT=2.261 min, m z 538.2 [M+H]⁺. SFC: 52.7 ee %.

### Example 12: Synthesis of (3S)-3-[4-[[4-[[3-[(2-Methoxyethylamino)methyl]morpholin-4-yl]methyl]-phenyl]methoxy]-1-oxo-isoindolin-2-yl]piperidine-2,6-dione

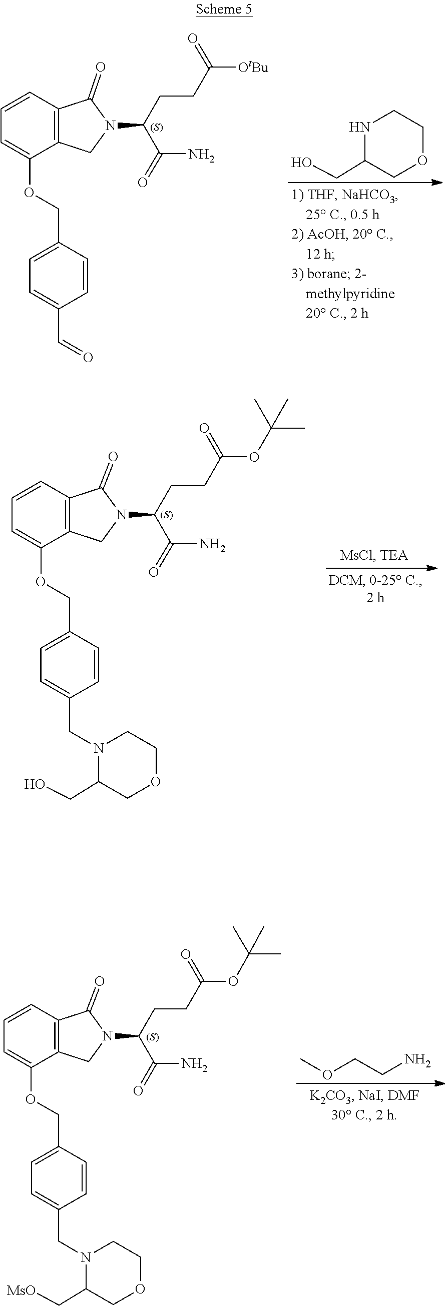

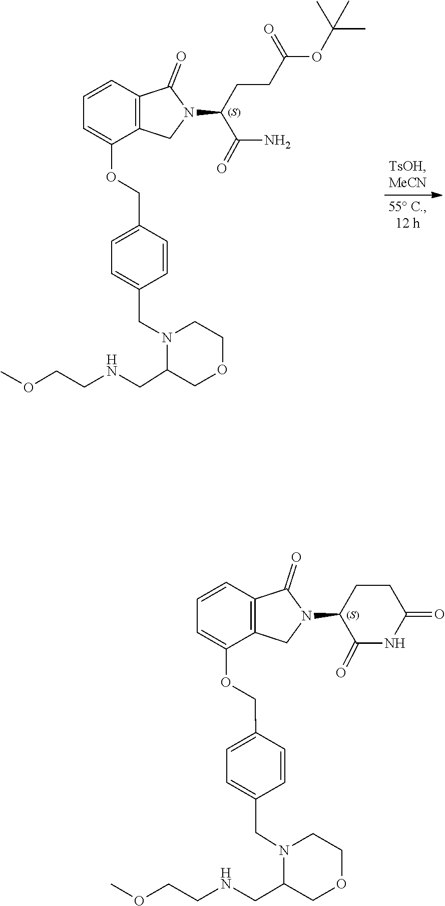

### Step 1: tert-Butyl (4S)-5-amino-4-[4-[[4-[[3-(hydroxymethyl)morpholin-4-yl]methyl]-phenyl]methoxy]-1-oxo-isoindolin-2-yl]-5-oxo-pentanoate

To a solution of morpholin-3-ylmethanol (700 mg, 4.56 mmol, HCl salt) in THF (10 mL) was added NaHCO₃ (668.34 mg, 7.96 mmol, 309.42 μL) and stirred for 0.5 hour at 20° C. Tert-butyl (4S)-5-amino-4-[4-[(4-formylphenyl)methoxy]-1-oxo-isoindolin-2-yl]-5-oxo-pentanoate (INT-1) (1.2 g, 2.65 mmol) and AcOH (1 mL) was added and the reaction mixture was stirred for 12 hours at 20° C. borane; 2-methylpyridine (709.14 mg, 6.63 mmol) was added and the reaction mixture was stirred for 2 hours at 20° C. LCMS showed the desired mass. The reaction mixture was poured into Ethyl Acetate (40 mL) and filtered. The filtrate was concentrated under reduced pressure. The residue was purified by semi-preparative reverse phase HPLC (20%-50% acetonitrile in water+0.225% formic acid, 21 min) and the pH value was adjusted to 7-8 with saturated NaHCO₃(aq), extracted with Ethyl Acetate (50 mL*3), dried over anhydrous sodium sulfate, and concentrated under reduced pressure to afford tert-butyl (4S)-5-amino-4-[4-[[4-[[3-(hydroxymethyl)morpholin-4-yl]methyl]phenyl]methoxy]-1-oxo-isoindolin-2-yl]-5-oxo-pentanoate (850 mg, 1.54 mmol, 57.89% yield) as a white solid. LCMS: RT=0.704 min, m/z 554.2 [M+H]⁺.

### Step 2: Synthesis of tert-Butyl(4S)-5-amino-4-[4-[[4-[[3-(methylsulfonyloxy methyl)morpholin-4-yl]methyl]phenyl]methoxy]-1-oxoisoindolin-2-yl]-5-oxo-pentanoate

To a solution of tert-butyl (4S)-5-amino-4-[4-[[4-[[3-(hydroxymethyl)morpholin-4-yl]methyl]-phenyl]methoxy]-1-oxoisoindolin-2-yl]-5-oxo-pentanoate (440 mg, 794.73 μmol) and TEA (120.63 mg, 1.19 mmol, 165.92 μL) in DCM (4 mL) was added MsCl (104 mg, 907.89 μmol, 70.27 μL) at 0° C. The mixture was stirred at 25° C. for 2 h. LCMS showed the desired mass. The reaction mixture was diluted with H₂O (30 mL) and extracted with DCM (40 mL×3). The combined organic layers were dried over anhydrous Na₂SO₄, filtered and concentrated under reduced pressure to afford tert-butyl(4S)-5-amino-4-[4-[[4-[[3-(methylsulfonyloxymethyl)-morpholin-4-yl]methyl]-phenyl]methoxy]-1-oxoisoindolin-2-yl]-5-oxo-pentanoate (500 mg, crude) as a yellow oil, which was used for the next step directly without further purification. LCMS: RT=0.734 min, m/z 632.1 [M+H]⁺.

### Step 3: Synthesis of tert-Butyl (4S)-5-amino-4-[4-[[4-[[3-[(2-methoxyethylamino) methyl]morpholin-4-yl]methyl]phenyl]methoxy]-1-oxo-isoindolin-2-yl]-5-oxo-pentanoate

To a solution of tert-butyl (4S)-5-amino-4-[4-[[4-[[3-(methylsulfonyloxymethyl)morpholin-4-yl]methyl]phenyl]methoxy]-1-oxoisoindolin-2-yl]-5-oxo-pentanoate (500 mg, 791.47 μmol) and 2-methoxyethanamine (118.89 mg, 1.58 mmol, 137.61 μL) in DMF (6 mL) were added K₂CO₃ (328.16 mg, 2.37 mmol) and NaI (118.64 mg, 791.47 μmol). The mixture was stirred at 30° C. for 2 h. LCMS showed the desired mass. The reaction mixture was diluted with H₂O (40 mL) and extracted with Ethyl Acetate (50 mL×3). The combined organic layers were washed with brine (60 mL×3), dried over anhydrous Na₂SO₄, filtered and concentrated under reduced pressure to give a residue. The residue was purified by prep-HPLC (formic acid condition; column: Phenomenex luna C_{18 150*25} mm*10 μm; mobile phase: [water(0.225% formic acid)-MeCN]; B %: 14%-44%, 10 min) to afford tert-butyl (4S)-5-amino-4-[4-[[4-[[3-[(2-methoxyethylamino)methyl]morpholin-4-yl]methyl]-phenyl]methoxy]-1-oxo-isoindolin-2-yl]-5-oxo-pentanoate (130 mg, 212.86 μmol, 26.89% yield) as a colorless oil. LCMS: RT=0.797 min, m/z 611.2 [M+H]⁺.

### Step 4: Synthesis of (3S)-3-[4-[[4-[[3-[(2-methoxyethylamino)methyl]morpholin-4-yl]methyl]-phenyl]methoxy]-1-oxo-isoindolin-2-yl]piperidine-2,6-dione

To a solution of tert-butyl (4S)-5-amino-4-[4-[[4-[[3-[(2-methoxyethylamino)methyl]morpholin-4-yl]methyl]phenyl]methoxy]-1-oxo-isoindolin-2-yl]-5-oxo-pentanoate (130 mg, 212.86 μmol) in CH₃CN (3 mL) was added TsOH (91.64 mg, 532.14 μmol). The mixture was stirred at 55° C. for 12 h. LCMS showed the desired mass. The reaction mixture was diluted with saturated NaHCO₃aqueous (20 mL) and extracted with DCM (30 mL×3). The combined organic layers were dried over anhydrous Na₂SO₄, filtered and concentrated under reduced pressure. The residue was purified by prep-HPLC (formic acid condition; column: Phenomenex luna C18 150*25 mm*10 μm; mobile phase: [water(0.225% formic acid)-MeCN]; B %:6%-36%, 10 min) to afford (3S)-3-[4-[[4-[[3-[(2-methoxy-ethylamino)methyl]morpholin-4-yl]methyl]phenyl]methoxy]-1-oxo-isoindolin-2-yl]piperidine-2,6-dione (23.20 mg, 40.99 μmol, 19.26% yield, 94.8% purity) as an off-white gum. HNMR: CDCl₃, 400 MHz. δ 8.40 (br s, 1H), 7.53-7.48 (m, 1H), 7.47-7.41 (m, 1H), 7.40-7.34 (m, 4H), 7.13-7.09 (m, 1H), 5.24-5.17 (m, 1H), 5.14 (s, 2H), 4.45-4.27 (m, 3H), 4.09-3.87 (m, 2H), 3.81-3.53 (m, 6H), 3.39-3.35 (m, 3H), 3.26-3.17 (m, 1H), 3.12-3.02 (m, 2H), 2.93-2.76 (m, 4H), 2.43-2.28 (m, 2H), 2.24-2.15 (m, 1H). LCMS: RT=2.269 min, m/z 537.3 [M+H]⁺.

### Example 13: Synthesis of INT-3

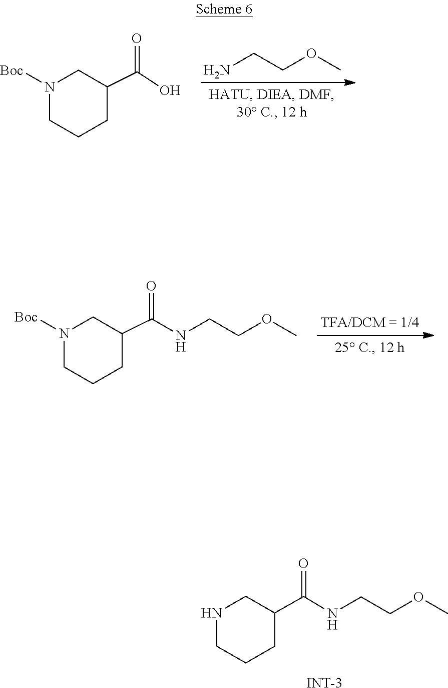

### Step 1: tert-Butyl 3-(2-methoxyethylcarbamoyl)piperidine-1-carboxylate

To a solution of 1-tert-butoxycarbonylpiperidine-3-carboxylic acid (500 mg, 2.18 mmol) and 2-methoxyethanamine (196.56 mg, 2.62 mmol) in DMF (5 mL) was added HATU (829.21 mg, 2.18 mmol) and DIEA (845.57 mg, 6.54 mmol) in one portion under nitrogen. The mixture was stirred at 30° C. for 12 hours. TLC (Petroleum ether:Ethyl acetate=1:1, Rf=0.2) showed the reaction was completed. The reaction mixture was diluted with water (80 mL) and extracted with Ethyl acetate (30 mL×4). The combined organic layers were washed with brine (50 mL), dried over anhydrous sodium sulfate, filtered and concentrated under reduced pressure. The residue was purified by flash silica gel chromatography (ISCO; 12 g SepaFlash Silica Flash Column, Eluent of 0 to 50% Ethyl acetate/Petroleum ether gradient @ 40 mL/min) to afford tert-butyl 3-(2-methoxyethylcarbamoyl)piperidine-1-carboxylate (600 mg, 2.10 mmol, 96.08% yield) as a colorless oil. HNMR: DMSO-d₆, 400 MHz. δ 7.96 (br t, J=4.8 Hz, 1H), 3.85 (br d, J=12.8 Hz, 2H), 3.32-3.29 (m, 2H), 3.23 (s, 3H), 3.19 (br d, J=4.8 Hz, 2H), 2.69 (s, 2H), 2.22 (tt, J=4.0, 11.2 Hz, 1H), 1.77 (br d, J=12.0 Hz, 1H), 1.62 (br d, J=13.2 Hz, 1H), 1.55-1.46 (m, 1H), 1.39 (s, 9H), 1.28 (tt, J=4.0, 12.4 Hz, 1H).

### Step 2: N-(2-Ethoxyethyl)piperidine-3-carboxamide

To a solution of tert-butyl 3-(2-methoxyethylcarbamoyl)piperidine-1-carboxylate (600 mg, 2.10 mmol) in DCM (8 mL) was added TFA (3.08 g, 27.01 mmol, 2 mL) in one portion. The mixture was stirred at 25° C. for 12 hours. TLC (Dichloromethane:Methanol=10:1, Rf=0.05) showed the reaction was completed. The reaction mixture was diluted with saturated sodium bicarbonate solution (100 mL) and then lyophilized to give a solid. The solid was diluted with Ethyl Acetate (30 mL) and then filtered. The filtrate was concentrated under reduced pressure to give (INT-3) N-(2-ethoxyethyl)piperidine-3-carboxamide (4.32 g, crude) as a light yellow oil, which was used in the next step directly without further purification.

### Example 14: Synthesis of 1-(4-(((2-((S)-2,6-dioxopiperidin-3-yl)-1-oxoisoindolin-4-yl)oxy)methyl)benzyl)-N-(2-methoxyethyl)piperidine-3-carboxamide

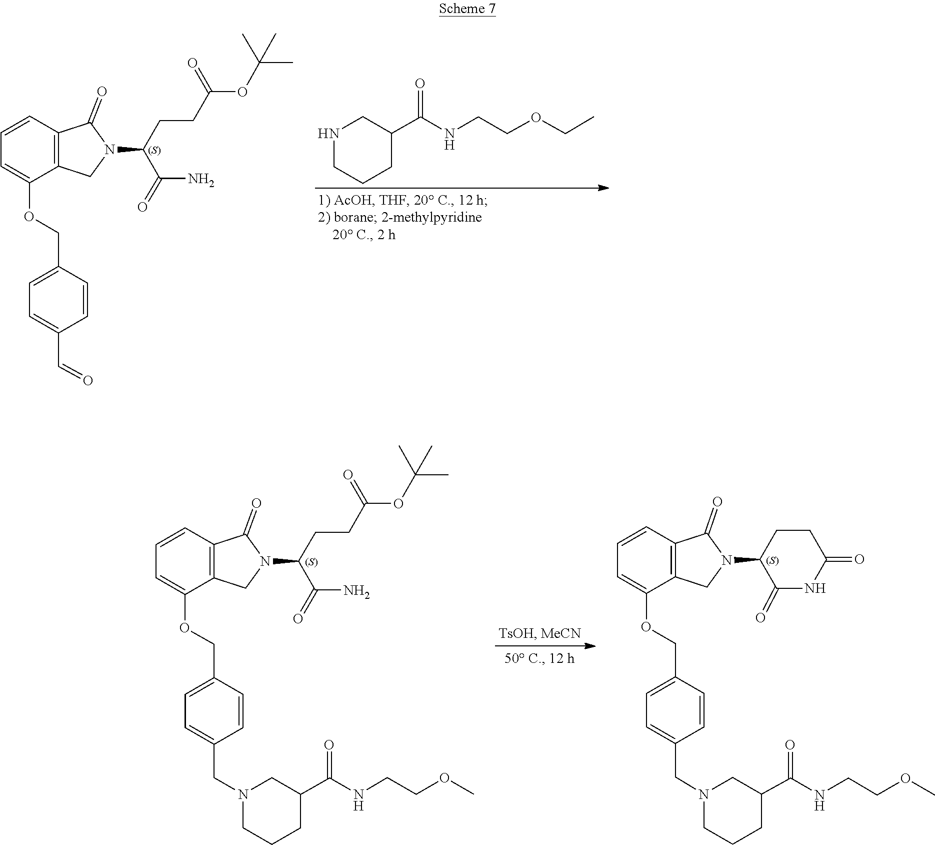

### Step 1: (4S)-tert-Butyl 5-amino-4-(4-((4-((3-((2-methoxyethyl)carbamoyl) piperidin-1-yl)methyl)benzyl)oxy)-1-oxoisoindolin-2-yl)-5-oxopentanoate

To a solution of (S)-tert-butyl 5-amino-4-(4-((4-formylbenzyl)oxy)-1-oxoisoindolin-2-yl)-5-oxopentanoate (INT-1) (300 mg, 662.99 μmol) and N-(2-ethoxyethyl)piperidine-3-carboxamide (INT-3) (1.36 g, 7.29 mmol) in THF (10 mL) was added AcOH (1 mL) in one portion under nitrogen. The mixture was stirred at 20° C. for 12 hours. To the reaction mixture was added borane; 2-methylpyridine (212.74 mg, 1.99 mmol) and the final mixture was stirred for another 2 hours. LCMS showed the desired mass. The reaction mixture was diluted with water (150 mL) and extracted with Ethyl Acetate (40 mL×4). The combined organic layers were washed with brine (80 mL), dried over anhydrous sodium sulfate, filtered and concentrated under reduced pressure. The residue was purified by semi-preparative reverse phase HPLC (15-45% acetonitrile+0.225% formic acid in water, over 22 min). The collected fraction was concentrated to remove most of the acetonitrile. The aqueous phase was extracted with Ethyl acetate (50 mL×4). The combined organic layers were washed with brine (80 mL), dried over anhydrous sodium sulfate, filtered and concentrated under reduced pressure to afford (4S)-tert-butyl 5-amino-4-(4-((4-((3-((2-methoxyethyl)-carbamoyl)piperidin-1-yl)methyl)benzyl)oxy)-1-oxoisoindolin-2-yl)-5-oxopen-tanoate (240 mg, 385.39 μmol, 58.13% yield) as a white solid. LCMS: RT=0.807 min, m/z 623.2 [M+H]⁺.

### Step 2: 1-(4-(((2-((S)-2,6-Dioxopiperidin-3-yl)-1-oxoisoindolin-4-yl)oxy)methyl) benzyl)-N-(2-methoxyethyl)piperidine-3-carboxamide

To a solution of (4S)-tert-butyl 5-amino-4-(4-((4-((3-((2-methoxyethyl)carbamoyl)piperidin-1-yl)methyl)benzyl)oxy)-1-oxoisoindolin-2-yl)-5-oxopentanoate (60 mg, 96.35 μmol) in MeCN (2 mL) was added TsOH (19.91 mg, 115.62 μmol) in one portion under nitrogen. The mixture was stirred at 50° C. for 12 hours. LCMS showed the desired mass. The reaction mixture was diluted with water (80 mL) and extracted with Ethyl acetate (30 mL×4). The combined organic layers were washed with brine (50 mL), dried over anhydrous sodium sulfate, filtered, and concentrated under reduced pressure. The residue was purified by semi-preparative reverse phase HPLC (23-53% acetonitrile+10 mM NH₄HCO₃ in water, over 9 min) to give the desired product 1-(4-(((2-((S)-2,6-dioxopiperidin-3-yl)-1-oxoisoindolin-4-yl)oxy)methyl)benzyl)-N-(2-methoxyethyl)-piperidine-3-carboxamide (15.55 mg, 27.96 μmol, 29.02% yield, 98.66% purity) as a white solid. HNMR: DMSO-d₆, 400 MHz. δ 10.96 (s, 1H), 7.92 (br s, 1H), 7.52-7.41 (m, 3H), 7.33 (br d, J=8.0 Hz, 3H), 5.28-5.19 (m, 2H), 5.11 (dd, J=5.2, 13.2 Hz, 1H), 4.46-4.38 (m, 1H), 4.29-4.22 (m, 1H), 3.45 (br d, J=7.2 Hz, 1H), 3.29 (br t, J=5.6 Hz, 3H), 3.21 (s, 3H), 3.19-3.12 (m, 2H), 2.95-2.86 (m, 1H), 2.67 (br d, J=2.0 Hz, 2H), 2.59 (br s, 1H), 2.54 (br s, 1H), 2.44 (br dd, J=4.4, 13.2 Hz, 1H), 2.09-1.90 (m, 3H), 1.73-1.56 (m, 2H), 1.50-1.30 (m, 2H). LCMS: RT=0.843 min, m/z 549.3 [M+H]⁺. QC_LCMS: RT=2.223 min, m/z 549.2 [M+H]⁺. SFC: 100 ee %.

### Example 15: Synthesis of INT-4

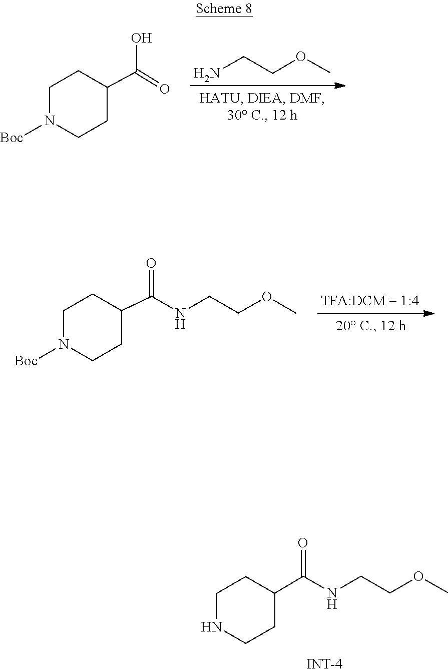

### Step 1: tert-Butyl 4-((2-methoxyethyl)carbamoyl)piperidine-1-carboxylate

To a solution of 1-(tert-butoxycarbonyl)piperidine-4-carboxylic acid (500 mg, 2.18 mmol) and 2-methoxyethanamine (180.18 mg, 2.40 mmol) in DMF (5 mL) was added HATU (912.13 mg, 2.40 mmol) and DIEA (845.57 mg, 6.54 mmol) in one portion under nitrogen. The mixture was stirred at 30° C. for 12 hours. TLC (Petroleum ether:Ethyl acetate=3:1, Rf=0.2) showed the reaction was completed. The reaction mixture was diluted with water (80 mL) and extracted with Ethyl acetate (30 mL×4). The combined organic layers were washed with brine (50 mL), dried over anhydrous sodium sulfate, filtered, and concentrated under reduced pressure. The residue was purified by flash silica gel chromatography (ISCO; 12 g SepaFlash Silica Flash Column, Eluent of 0˜50% Ethyl acetate/Petroleum ether gradient @ 40 mL/min) to give tert-butyl 4-((2-methoxyethyl)carbamoyl)piperidine-1-carboxylate (610 mg, 2.13 mmol, 97.68% yield) as a light yellow oil. HNMR: DMSO-d₆, 400 MHz. δ 7.86 (br t, J=5.2 Hz, 1H), 3.92 (br d, J=12.0 Hz, 2H), 3.32-3.28 (m, 2H), 3.23 (s, 3H), 3.18 (q, J=5.6 Hz, 2H), 2.74-2.67 (m, 2H), 2.28 (tt, J=4.0, 11.6 Hz, 1H), 1.61 (br dd, J=2.4, 12.8 Hz, 2H), 1.42 (br d, J=4.4 Hz, 1H), 1.39 (s, 9H), 1.36-1.31 (m, 1H).

### Step 2: N-(2-Ethoxyethyl)piperidine-4-carboxamide

To a solution of tert-butyl 4-((2-methoxyethyl)carbamoyl)piperidine-1-carboxylate (610 mg, 2.13 mmol) in DCM (8 mL) was added TFA (3.08 g, 27.01 mmol, 2.00 mL) in one portion. The mixture was stirred at 25° C. for 12 hours. TLC (Dichloromethane:Methanol=10:1, Rf=0.05) showed the reaction was completed. The reaction mixture was diluted with saturated sodium bicarbonate solution (100 mL) and then lyophilized to give a solid. The solid was diluted with Ethyl acetate (10 mL) and then filtered. The filtrate was concentrated under reduced pressure to give N-(2-ethoxyethyl)piperidine-4-carboxamide (INT-4) (3.0 g, crude) as a light yellow oil, which was used in the next step directly without further purification.

### Example 16: Synthesis of (S)-1-(4-(((2-(2,6-dioxopiperidin-3-yl)-1-oxoisoindolin-4-yl)oxy)methyl)benzyl)-N-(2-methoxyethyl)piperidine-4-carboxamide

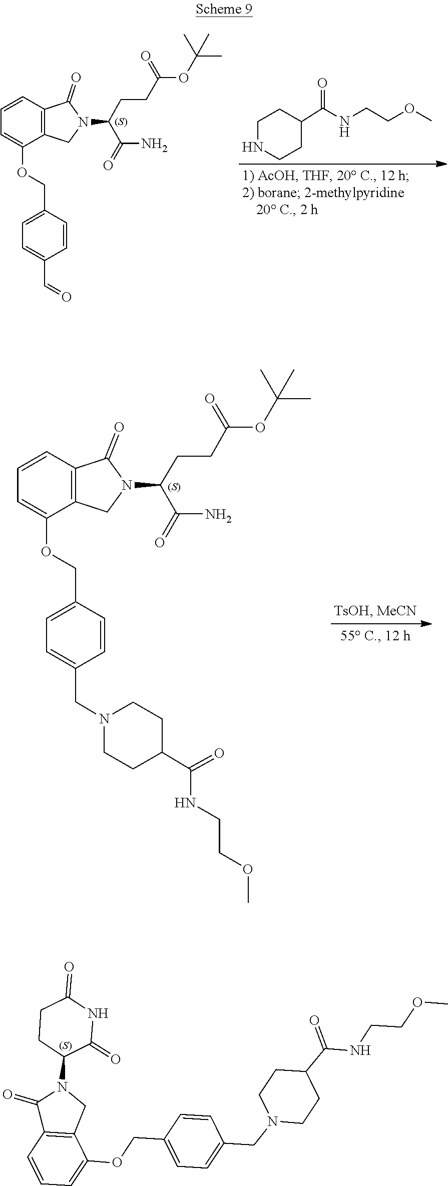

### Step 1: (S)-tert-Butyl 5-amino-4-(4-((4-((4-((2-methoxyethyl)carbamoyl)piperidin-1-yl)methyl)benzyl)oxy)-1-oxoisoindolin-2-yl)-5-oxopentanoate

To a solution of (S)-tert-butyl 5-amino-4-(4-((4-formylbenzyl)oxy)-1-oxoisoindolin-2-yl)-5-oxopentanoate (300 mg, 662.99 μmol) and N-(2-ethoxyethyl)piperidine-4-carboxamide (INT-4) (1.11 g, 5.97 mmol) in THF (10 mL) was added HOAc (1 mL) in one portion under nitrogen. The mixture was stirred at 20° C. for 12 hours. To the reaction mixture was added borane; 2-methylpyridine (212.74 mg, 1.99 mmol) and the final mixture was stirred for another 2 hours. LCMS showed the desired mass. The reaction mixture was diluted with water (150 mL) and extracted with Ethyl acetate (40 mL×4). The combined organic layers were washed with brine (80 mL), dried over anhydrous sodium sulfate, filtered, and concentrated under reduced pressure. The residue was purified by semi-preparative reverse phase HPLC (15-45% acetonitrile+0.225% formic acid in water, over 22 min). The collected fraction was concentrated to remove most of acetonitrile. The aqueous phase was extracted with Ethyl acetate (50 mL×4). The combined organic layers were washed with brine (80 mL), dried over anhydrous sodium sulfate, filtered and concentrated under reduced pressure to give the desired product (S)-tert-butyl 5-amino-4-(4-((4-((4-((2-methoxyethyl)carbamoyl)piperidin-1-yl)methyl)benzyl)oxy)-1-oxoisoindolin-2-yl)-5-oxopentanoate (230 mg, 369.33 μmol, 55.71% yield) as a white solid. LCMS: RT=0.782 min, m/z 623.3 [M+H]⁺.

### Step 2: (S)-1-(4-(((2-(2,6-Dioxopiperidin-3-yl)-1-oxoisoindolin-4-yl)oxy)methyl) benzyl)-N-(2-methoxyethyl)piperidine-4-carboxamide

To a solution of (S)-tert-butyl 5-amino-4-(4-((4-((4-((2-methoxyethyl)carbamoyl)piperidin-1-yl)methyl)benzyl)oxy)-1-oxoisoindolin-2-yl)-5-oxopentanoate (80 mg, 128.46 μmol) in MeCN (2 mL) was added TsOH (29.32 mg, 154.16 μmol) in one portion under nitrogen. The mixture was stirred at 50° C. for 12 hours. LCMS showed the desired mass. The reaction mixture was filtered and concentrated to dryness. The crude was purified by semi-preparative reverse phase HPLC (15-45% acetonitrile+10 mM NH₄HCO₃ in water, over 2 min) to afford (S)-1-(4-(((2-(2,6-dioxopiperidin-3-yl)-1-oxoisoindolin-4-yl)oxy)methyl)benzyl)-N-(2-methoxyethyl)-piperidine-4-carboxamide (29.63 mg, 53.87 μmol, 41.93% yield, 99.7% purity) as a white solid. HNMR: DMSO-d₆, 400 MHz. δ 10.97 (s, 1H), 7.78 (br s, 1H), 7.51-7.46 (m, 1H), 7.43 (br d, J=6.8 Hz, 2H), 7.33 (br d, J=8.4 Hz, 4H), 5.23 (s, 2H), 5.11 (dd, J=5.2, 13.6 Hz, 1H), 4.44-4.38 (m, 1H), 4.29-4.23 (m, 1H), 3.43 (br s, 2H), 3.30 (br d, J=3.2 Hz, 3H), 3.22 (s, 3H), 3.17 (q, J=5.6 Hz, 2H), 2.96-2.86 (m, 1H), 2.79 (br d, J=10.0 Hz, 2H), 2.59 (br s, 1H), 2.57-2.54 (m, 1H), 2.44 (br s, 1H), 2.08 (br s, 1H), 2.02-1.94 (m, 1H), 1.93-1.83 (m, 2H), 1.64-1.50 (m, 4H). LCMS: RT=0.858 min, m z 549.2 [M+H]⁺. QC_LCMS: RT=2.178 min, m/z 549.2 [M+H]⁺. SFC: 100 ee %.

### Example 17: Synthesis of INT-5: N-(2-ethoxyethyl)piperidine-3-carboxamide

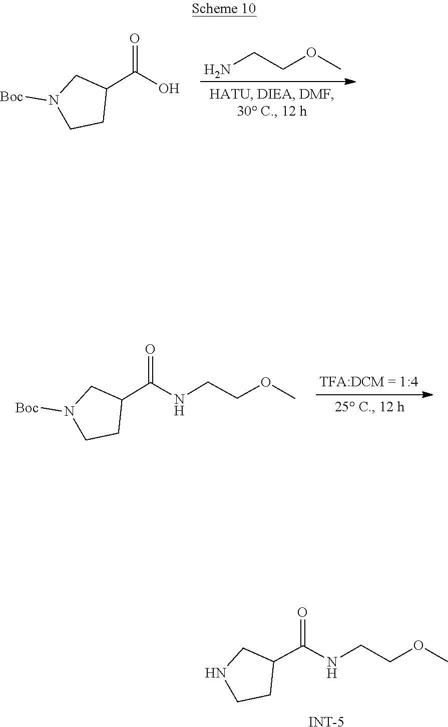

### Step 1: tert-Butyl 3-((2-methoxyethyl)carbamoyl)pyrrolidine-1-carboxylate

To a solution of 1-(tert-butoxycarbonyl)pyrrolidine-3-carboxylic acid (500 mg, 2.32 mmol) and 2-methoxyethanamine (191.92 mg, 2.56 mmol) in DMF (5 mL) was added HATU (883.25 mg, 2.32 mmol) and DIEA (900.67 mg, 6.97 mmol) in one portion under nitrogen. The mixture was stirred at 30° C. for 12 h. TLC (Petroleum ether:Ethyl acetate=1:1, Rf=0.2) showed the reaction was completed. The reaction mixture was diluted with water (80 mL) and extracted with Ethyl acetate (30 mL×4). The combined organic layers were washed with brine (50 mL), dried over anhydrous sodium sulfate, filtered, and concentrated under reduced pressure. The residue was purified by flash silica gel chromatography (ISCO; 12 g SepaFlash Silica Flash Column, Eluent of 0 to 50% Ethyl acetate/Petroleum ether gradient @ 40 mL/min) to give tert-butyl 3-((2-methoxy-ethyl)carbamoyl)pyrrolidine-1-carboxylate (580 mg, 2.13 mmol) as a light yellow oil. HNMR: DMSO-d₆, 400 MHz. δ 8.04 (br t, J=5.2 Hz, 1H), 3.44-3.37 (m, 1H), 3.37-3.34 (m, 1H), 3.32-3.29 (m, 1H), 3.24 (s, 3H), 3.23-3.13 (m, 4H), 2.94-2.86 (m, 1H), 2.73-2.68 (m, 1H), 1.98-1.81 (m, 2H), 1.39 (s, 9H).

### Step 2: N-(2-Ethoxyethyl)piperidine-3-carboxamide

To a solution of tert-butyl 3-((2-methoxyethyl)carbamoyl)pyrrolidine-1-carboxylate (580 mg, 2.13 mmol) in DCM (8 mL) was added TFA (3.08 g, 27.01 mmol, 2 mL) in one portion. The mixture was stirred at 25° C. for 12 h. TLC (Dichloromethane:Methanol=10:1, Rf=0.05) showed the reaction was completed. The reaction mixture was diluted with saturated sodium bicarbonate solution (100 mL) and then lyophilized to give a solid. The solid was diluted with Ethyl acetate (50 mL) and then filtered. The filtrate was concentrated under reduced pressure to give the desired product N-(2-ethoxyethyl)piperidine-3-carboxamide (INT-5) (1.9 g, crude) as a light yellow oil, which was used in the next step directly without further purification.

### Example 18: Synthesis of 1-(4-(((2-((S)-2,6-dioxopiperidin-3-yl)-1-oxoisoindolin-4-yl)oxy)methyl)benzyl)-N-(2-methoxyethyl)pyrrolidine-3-carboxamide

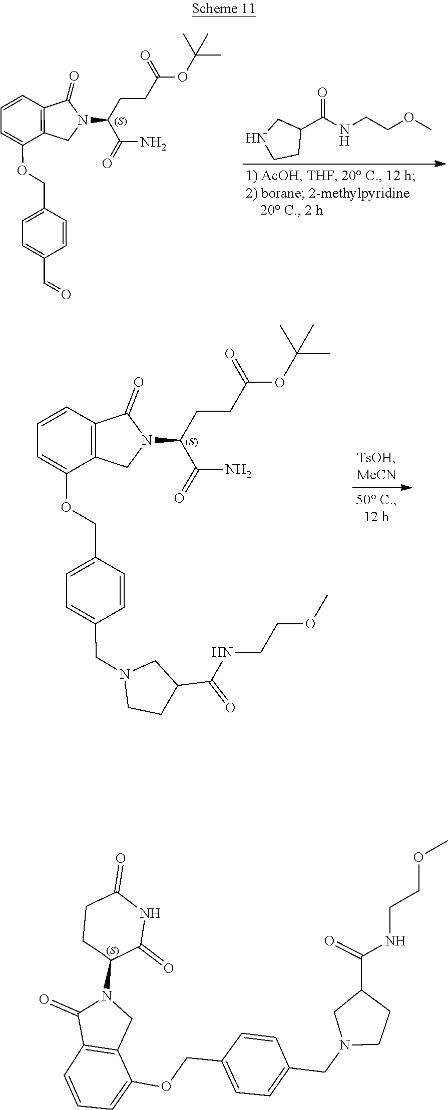

### Step 1: (4S)-tert-Butyl 5-amino-4-(4-((4-((3-((2-methoxyethyl)carbamoyl)pyrrolidin-1-yl)methyl)benzyl)oxy)-1-oxoisoindolin-2-yl)-5-oxopentanoate

To a solution of (S)-tert-butyl 5-amino-4-(4-((4-formylbenzyl)oxy)-1-oxoisoindolin-2-yl)-5-oxopentanoate (300 mg, 662.99 μmol) and N-(2-ethoxyethyl)pyrrolidine-3-carboxamide (INT-5) (1.36 g, 7.29 mmol) in THF (10 mL) was added HOAc (1 mL) in one portion under nitrogen. The mixture was stirred at 20° C. for 12 hours. Then, to the reaction mixture was added borane; 2-methylpyridine (212.74 mg, 1.99 mmol) and the final mixture was stirred for another 2 hours. LCMS showed the desired mass. The reaction mixture was diluted with water (150 mL) and extracted with Ethyl acetate (40 mL×4). The combined organic layers were washed with brine (80 mL), dried over anhydrous sodium sulfate, filtered, and concentrated under reduced pressure. The crude was purified by semi-preparative reverse phase HPLC (20-45% acetonitrile+0.225% formic acid in water, over 15 min). The collected fraction was concentrated to remove most of acetonitrile. Then, the aqueous phase was extracted with Ethyl acetate (50 mL×4). The combined organic layers were washed with brine (80 mL), dried over anhydrous sodium sulfate, filtered and concentrated under reduced pressure to give (4S)-tert-butyl 5-amino-4-(4-((4-((3-((2-methoxyethyl)carbamoyl)pyrrolidin-1-yl)methyl)benzyl)oxy)-1-oxoisoindolin-2-yl)-5-oxopentanoate (170 mg, 279.27 μmol, 42.12% yield) as a white solid. LCMS: RT=0.773 min, m/z 609.3 [M+H]⁺.

### Step 2: 1-(4-(((2-((S)-2,6-Dioxopiperidin-3-yl)-1-oxoisoindolin-4-yl)oxy)methyl) benzyl)-N-(2-methoxyethyl)pyrrolidine-3-carboxamide

To a solution of (4S)-tert-butyl 5-amino-4-(4-((4-((3-((2-methoxyethyl)carbamoyl)pyrrolidin-1-yl)methyl)benzyl)oxy)-1-oxoisoindolin-2-yl)-5-oxo-pentanoate (60.00 mg, 98.57 μmol) in MeCN (2 mL) was added TsOH (20.37 mg, 118.28 μmol) in one portion under nitrogen. The mixture was stirred at 50° C. for 12 hours. LCMS showed the desired mass. The reaction mixture was diluted with water (80 mL) and extracted with Ethyl acetate (30 mL×4). The combined organic layers were washed with brine (50 mL), dried over anhydrous sodium sulfate, filtered, and concentrated under reduced pressure. The crude was purified by semi-preparative reverse phase HPLC (19-49% acetonitrile+10 mM NH₄HCO₃ in water, over 9 min) to give the desired product 1-(4-(((2-((S)-2,6-dioxopiperidin-3-yl)-1-oxoisoindolin-4-yl)oxy)methyl)benzyl)-N-(2-methoxyethyl)pyrrolidine-3-carboxamide (13.59 mg, 25.37 μmol, 25.74% yield, 99.79% purity) as a white solid. HNMR: DMSO-d₆, 400 MHz. δ 10.96 (br s, 1H), 7.84 (br s, 1H), 7.51-7.40 (m, 3H), 7.32 (br d, J=7.8 Hz, 4H), 5.23 (s, 2H), 5.11 (dd, J=5.1, 13.3 Hz, 1H), 4.45-4.38 (m, 1H), 4.28-4.22 (m, 1H), 3.57 (br s, 2H), 3.22 (s, 3H), 3.20-3.15 (m, 2H), 2.96-2.79 (m, 2H), 2.74 (br d, J=9.2 Hz, 1H), 2.68-2.57 (m, 2H), 2.54 (br s, 1H), 2.46-2.35 (m, 4H), 2.02-1.94 (m, 1H), 1.91-1.83 (m, 2H). LCMS: RT=0.817 min, m/z 535.3 [M+H]⁺. QC_LCMS: RT=2.199 min, m/z 535.2 [M+H]⁺. SFC: 99.9 ee %.

### Example 19: Synthesis of INT-6

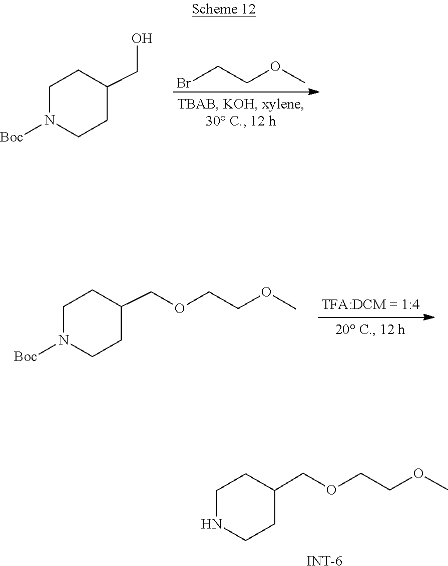

### Step 1: tert-Butyl 4-((2-methoxyethoxy)methyl)piperidine-1-carboxylate

To a solution of tert-butyl 4-(hydroxymethyl)piperidine-1-carboxylate (500 mg, 2.18 mmol) and 1-bromo-2-methoxyethane (497.11 mg, 3.58 mmol) in xylene (10 mL) was added TBAB (104.82 mg, 325.15 μmol) in one portion under nitrogen. KOH (547.27 mg, 9.75 mmol) was added to the mixture and the mixture was stirred at 30° C. for 12 hours. TLC (Petroleum ether:Ethyl acetate=3:1, Rf=0.6) showed the reaction was completed. The reaction mixture was diluted with water (80 mL) and extracted with Ethyl acetate (30 mL×4). The combined organic layers were washed with brine (50 mL), dried over anhydrous sodium sulfate, filtered, and concentrated under reduced pressure. The residue was purified by flash silica gel chromatography (ISCO; 12 g SepaFlash Silica Flash Column, Eluent of 0 to 30% Ethyl acetate/Petroleum ether gradient @ 40 mL/min) to give tert-butyl 4-((2-methoxyethoxy)methyl)piperidine-1-carboxylate (500 mg, 1.83 mmol, 56.25% yield) as a light yellow oil. HNMR: DMSO-d₆, 400 MHz. δ 3.99-3.85 (m, 2H), 3.50-3.44 (m, 2H), 3.44-3.39 (m, 2H), 3.27-3.18 (m, 5H), 2.67 (br d, J=2.0 Hz, 2H), 1.72-1.58 (m, 3H), 1.38 (s, 9H), 1.06-0.93 (m, 2H).

### Step 2: tert-Butyl 4-((2-methoxyethoxy)methyl)piperidine-1-carboxylate

To a solution of tert-butyl 4-((2-methoxyethoxy)methyl)piperidine-1-carboxylate (500 mg, 1.83 mmol) in DCM (8 mL) was added TFA (814.45 mg, 7.14 mmol, 2 mL) in one portion. The mixture was stirred at 25° C. for 12 hours. TLC (Dichloromethane:Methanol=10:1, Rf=0.05) showed the reaction was completed. The reaction mixture was diluted with saturated sodium bicarbonate solution (100 mL) and then lyophilized to give a solid. The solid was diluted with Ethyl acetate (50 mL) and then filtered. The filtrate was concentrated under reduced pressure to give 4-((2-ethoxyethoxy)methyl)piperidine (INT-6) (840 mg, crude) as a light yellow oil, which was used in the next step directly without further purification.

### Example 20: Synthesis of (S)-3-(4-((4-((4-((2-methoxyethoxy)methyl)piperidin-1-yl)methyl)-benzyl)oxy)-1-oxoisoindolin-2-yl)piperidine-2,6-dione

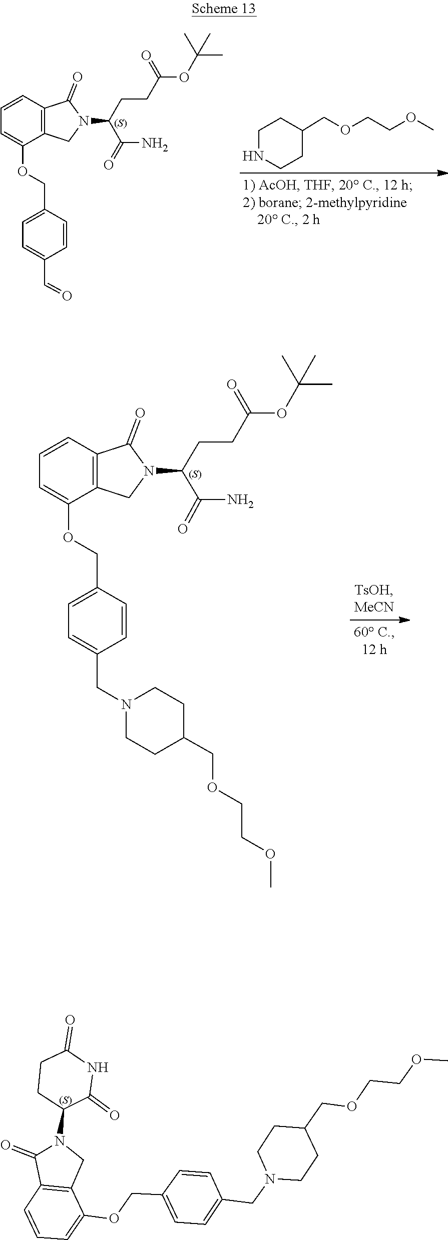

### Step 1: tert-Butyl 4-((2-methoxyethoxy)methyl)piperidine-1-carboxylate

To a solution of (S)-tert-butyl 5-amino-4-(4-((4-formylbenzyl)oxy)-1-oxoisoindolin-2-yl)-5-oxopentanoate (300 mg, 662.99 μmol) and 4-((2-ethoxy-ethoxy)methyl)piperidine (INT-6) (459.45 mg, 2.65 mmol) in THF (10 mL) was added HOAc (1 mL) in one portion under nitrogen. The mixture was stirred at 20° C. for 12 hours. To the reaction mixture was added borane; 2-methylpyridine (212.74 mg, 1.99 mmol) and the final mixture was stirred for another 2 hours. LCMS showed the desired mass. The reaction mixture was diluted with water (150 mL) and extracted with Ethyl acetate (40 mL×4). The combined organic layers were washed with brine (80 mL), dried over anhydrous sodium sulfate, filtered, and concentrated under reduced pressure. The crude was purified by semi-preparative reverse phase HPLC (20-45% acetonitrile+0.225% formic acid in water, over 15 min). The collected fraction was concentrated to remove most of the acetonitrile. The aqueous phase was extracted with ethyl acetate (50 mL×4). The combined organic layers were washed with brine (80 mL), dried over anhydrous sodium sulfate, filtered and concentrated under reduced pressure to give (S)-tert-butyl 5-amino-4-(4-((4-((4-((2-methoxyethoxy)methyl)piperidin-1-yl)methyl)benzyl)oxy)-1-oxoisoindolin-2-yl)-5-oxo-pentanoate (250 mg, 410.00 μmol, 61.84% yield) as a colorless oil. LCMS: RT=0.815 min, m z 610.3 [M+H]⁺.

### Step 2: (S)-3-(4-((4-((4-((2-Methoxyethoxy)methyl)piperidin-1-yl)methyl)benzyl) oxy)-1-oxoisoindolin-2-yl)piperidine-2,6-dione

To a solution of (S)-tert-butyl 5-amino-4-(4-((4-((4-((2-methoxyethoxy)methyl)piperidin-1-yl)methyl)benzyl)oxy)-1-oxoisoindolin-2-yl)-5-oxopentanoate (150 mg, 246.00 μmol) in MeCN (3 mL) was added TsOH (50.83 mg, 295.20 μmol) in one portion under nitrogen. The mixture was stirred at 55° C. for 12 hours. LCMS showed the desired mass. The reaction mixture was filtered and concentrated to dryness. The residue was purified by semi-preparative reverse phase HPLC (26-56% acetonitrile+10 mM NH₄HCO₃ in water, over 2 min) to afford (S)-3-(4-((4-((4-((2-methoxyethoxy)methyl)piperidin-1-yl)methyl)benzyl)oxy)-1-oxoisoindolin-2-yl)piperidine-2,6-dione (20.61 mg, 37.52 μmol, 15.25% yield, 97.51% purity) as a white solid. HNMR: DMSO-d₆, 400 MHz. δ 10.96 (s, 1H), 7.51-7.46 (m, 1H), 7.43 (d, J=8.0 Hz, 2H), 7.35-7.28 (m, 4H), 5.22 (s, 2H), 5.11 (dd, J=5.2, 13.2 Hz, 1H), 4.45-4.38 (m, 1H), 4.29-4.21 (m, 1H), 3.48-3.44 (m, 2H), 3.44-3.40 (m, 4H), 3.25-3.19 (m, 5H), 2.96-2.85 (m, 1H), 2.77 (br d, J=11.2 Hz, 2H), 2.59 (br s, 1H), 2.48-2.39 (m, 1H), 2.03-1.94 (m, 1H), 1.89 (br t, J=10.8 Hz, 2H), 1.60 (br d, J=12.4 Hz, 2H), 1.49 (br s, 1H), 1.20-1.09 (m, 2H). LCMS: RT=0.933 min, m/z 536.2 [M+H]⁺. QC_LCMS: RT=2.292 min, m/z 536.2 [M+H]⁺. SFC: 100 de %.

### Example 21: Synthesis of (3S)-3-[6-(2-methoxyethoxy)-4-[[4-(morpholinomethyl)-phenyl]methoxy]-1-oxo-isoindolin-2-yl]piperidine-2,6-dione

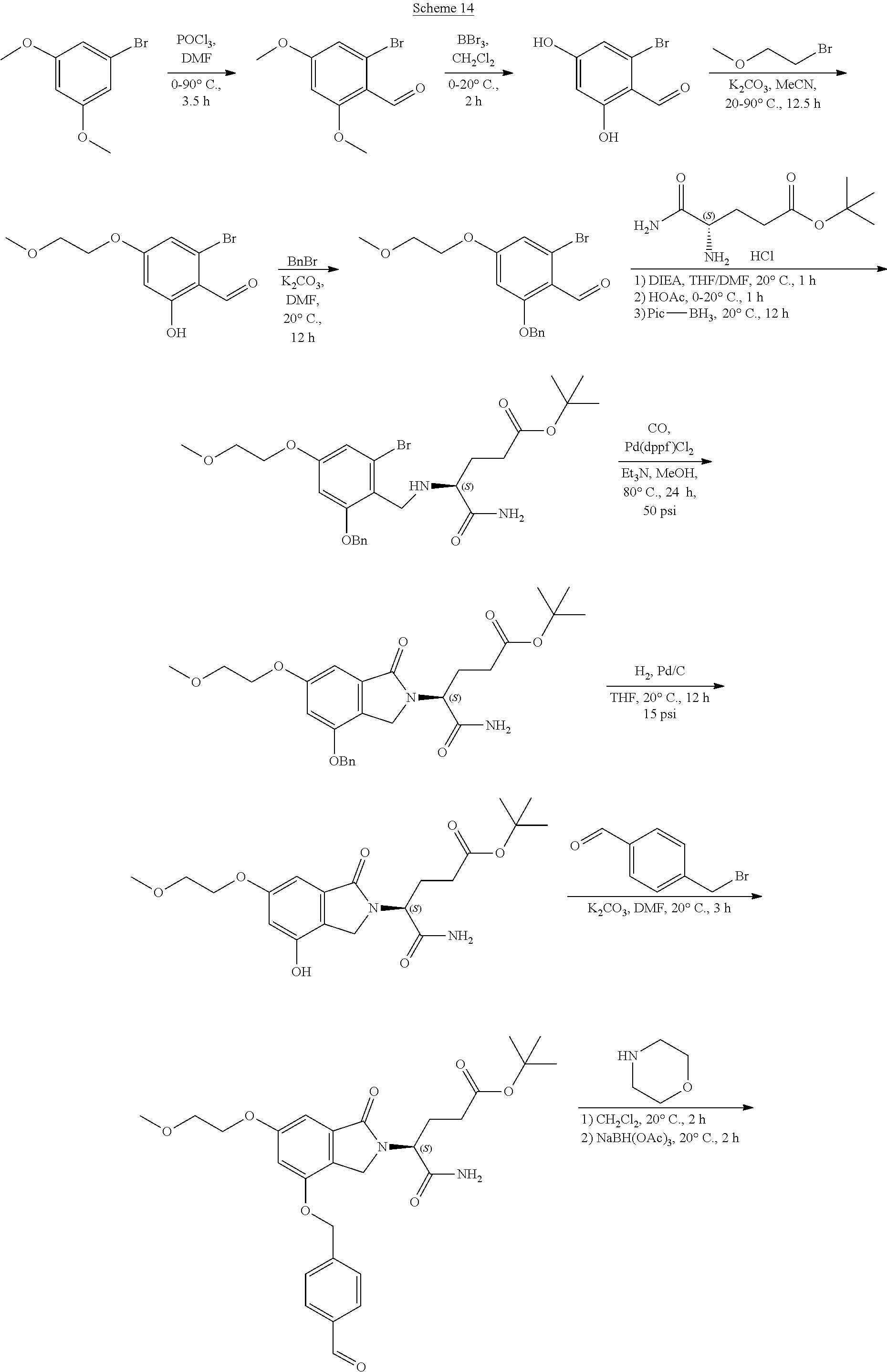

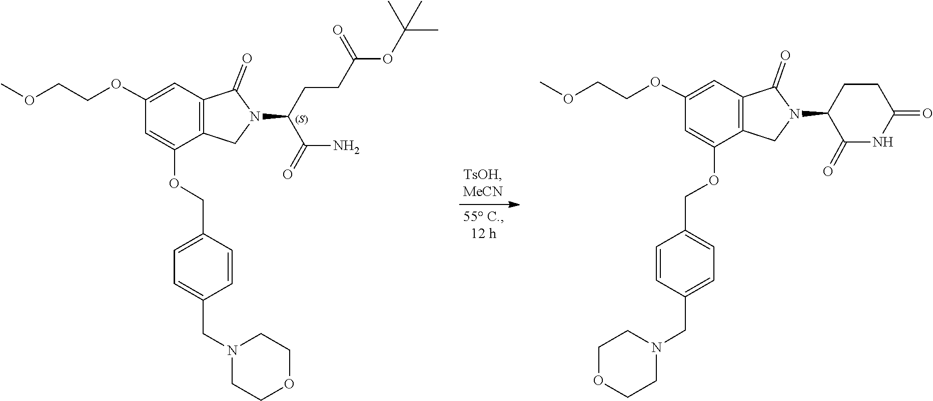

### Step 1: tert-Butyl 4-((2-methoxyethoxy)methyl)piperidine-1-carboxylate

To a solution of 1-bromo-3,5-dimethoxy-benzene (20 g, 92.14 mmol) in DMF (40 mL) was drop-wise added POCl₃ (42.38 g, 276.42 mmol, 25.69 mL) at 0° C. and the mixture was allowed to react at 0° C. for 90 minutes. Then the resulting mixture was stirred at 90° C. for 2 hours. LCMS showed the reaction was completed. The reaction contents were poured into ice water (200 mL), treated with solid KOH until pH-14, and allowed to stir for 1 hour. The crude material was extracted with Ethyl acetate (100 mL×3). The combined organic phases were dried over anhydrous sodium sulfate, filtered, and concentrated to give a residue. The residue was purified by flash silica gel chromatography (5-20% Ethyl acetate in Petroleum ether) to give 2-bromo-4,6-dimethoxy-benzaldehyde (21.3 g, 86.91 mmol, 94.33% yield) as a white solid. HNMR: CDCl₃, 400 MHz. δ 10.32 (s, 1H), 6.79 (d, J=2.4 Hz, 1H), 6.44 (d, J=2.4 Hz, 1H), 3.90 (s, 3H), 3.88 (s, 3H). LCMS: RT=0.806 min, m/z 245.0 [M+H]⁺.

### Step 2: 2-Bromo-4,6-dihydroxy-benzaldehyde

To a solution of 2-bromo-4,6-dimethoxy-benzaldehyde (21.3 g, 86.91 mmol) in DCM (200 mL) was added dropwise BBr₃ (65.32 g, 260.74 mmol, 25.12 mL) at 0° C. After addition, the mixture was stirred at 20° C. for 2 hours. TLC (Petroleum ether:Ethyl acetate=2:1) showed the reaction was completed. The mixture was quenched with water (200 mL) and extracted with Ethyl acetate (300 mL×2). The combined organic phases were washed with brine (50 mL×2), dried over anhydrous sodium sulfate, filtered, and concentrated under vacuum. The residue was purified by flash silica gel chromatography (2-15% Ethyl acetate in Petroleum ether) to give the desired product 2-bromo-4,6-dihydroxy-benzaldehyde (15.5 g, 71.42 mmol, 82.18% yield) as a white solid. HNMR: MeOD, 400 MHz. δ 10.09 (s, 1H), 6.70 (d, J=2.0 Hz, 1H), 6.28 (d, J=2.0 Hz, 1H). LCMS: RT=0.771 min, m/z 219.1 [M+H]⁺.

### Step 3: 2-Bromo-6-hydroxy-4-(2-methoxyethoxy) benzaldehyde

To a solution of 2-bromo-4,6-dihydroxy-benzaldehyde (15.5 g, 71.42 mmol) in CH₃CN (200 mL) was added K₂CO₃ (4.94 g, 35.71 mmol) and the mixture was stirred at 20° C. for 0.5 hour. Then 1-bromo-2-methoxy-ethane (10.92 g, 78.57 mmol, 7.38 mL) was added and the resulting mixture was stirred at 90° C. for 12 hours. LCMS showed the reaction was completed. The mixture was filtered, and the filtrate was concentrated under vacuum to give a residue. The residue was purified by preparative HPLC (46-76% acetonitrile+0.225% formic acid in water, 35 min) to give 2-bromo-6-hydroxy-4-(2-methoxyethoxy) benzaldehyde (4.8 g, 17.45 mmol, 24.43% yield) as a brown solid. 2-bromo-4,6-dihydroxy-benzaldehyde (8.3 g, 38.25 mmol, 53.55% yield) was recovered as a yellow solid. HNMR: CDCl₃, 400 MHz. δ 12.46 (s, 1H), 10.12 (s, 1H), 6.80 (d, J=2.4 Hz, 1H), 6.39 (d, J=2.0 Hz, 1H), 4.13-4.19 (m, 2H), 3.72-3.80 (m, 2H), 3.46 (s, 3H). LCMS: RT=0.858 min, m/z 275.1 [M+H]⁺.

### Step 4: 2-Benzyloxy-6-bromo-4-(2-methoxyethoxy)benzaldehyde

To a mixture of 2-bromo-6-hydroxy-4-(2-methoxyethoxy) benzaldehyde (2.1 g, 7.63 mmol) and K₂CO₃ (2.11 g, 15.27 mmol) in DMF (30 mL) was added bromomethylbenzene (1.58 g, 9.26 mmol, 1.10 mL). The mixture was then stirred at room temperature (20° C.) for 12 hours. TLC (Petroleum ether:Ethyl acetate=5:1) indicated the starting material was consumed completely and one new spot formed. The reaction mixture was quenched with water (100 mL), extracted with Ethyl acetate (20 mL×2). The combined organic phases were washed with brine (100 mL×3), dried over Na₂SO₄, filtered, and concentrated to give a residue. The residue was purified by column chromatography (SiO₂, Petroleum ether:Ethyl acetate=1/0 to 1/1) to give 2-benzyloxy-6-bromo-4-(2-methoxyethoxy)benzaldehyde (2.7 g, 7.39 mmol, 96.84% yield) as a white solid. HNMR: CDCl₃, 400 MHz. δ 10.41 (s, 1H), 7.32-7.50 (m, 5H), 6.84 (d, J=2.4 Hz, 1H), 6.59 (d, J=2.4 Hz, 1H), 5.17 (s, 2H), 4.13-4.19 (m, 2H), 3.72-3.80 (m, 2H), 3.47 (s, 3H).

### Step 5: tert-Butyl (4S)-5-amino-4-[[2-benzyloxy-6-bromo-4-(2-methoxyethoxy)phenyl]-methylamino]-5-oxo-pentanoate

A mixture of tert-butyl (4S)-4,5-diamino-5-oxo-pentanoate (720 mg, 3.02 mmol, HCl) and DIEA (425 mg, 3.29 mmol, 572.78 μL) in THE (10 mL) and DMF (2 mL) was stirred at room temperature (20° C.) for 1 hour. Then HOAc (247 mg, 4.11 mmol, 235.24 μL) was added at 0° C., followed by 2-benzyloxy-6-bromo-4-(2-methoxyethoxy)benzaldehyde (1 g, 2.74 mmol). The resulting mixture was then stirred at room temperature (20° C.) for 2 hours. Pic-BH₃ (880.00 mg, 8.23 mmol) was added, and the mixture was stirred at room temperature (20° C.) for 12 hours. TLC (Petroleum ether:Ethyl acetate=2:1) indicated the starting material was consumed completely and two new spots formed. LCMS showed the desired mass. The reaction mixture was quenched with water (20 mL) and extracted with Ethyl acetate (5 mL×3). The combined organic phases were dried over anhydrous sodium sulfate, filtered, and concentrated to dryness. The residue was purified by column chromatography (SiO₂, Petroleum ether:Ethyl acetate=1/0 to 1/1) to give the desired product tert-butyl (4S)-5-amino-4-[[2-benzyloxy-6-bromo-4-(2-methoxyethoxy)phenyl]methylamino]-5-oxo-pentanoate (1 g, 1.81 mmol, 66.23% yield) as a white solid. HNMR: CDCl₃, 400 MHz. δ 7.34-7.48 (m, 5H), 7.13 (s, 1H), 6.75 (d, J=1.2 Hz, 1H), 6.60 (d, J=1.2 Hz, 1H), 5.02 (s, 2H), 4.81-4.91 (m, 1H), 4.07-4.12 (m, 2H), 3.81-3.92 (m, 2H), 3.70-3.78 (m, 2H), 3.47 (s, 3H), 3.11 (t, J=6.4 Hz, 1H), 2.23-2.32 (m, 2H), 1.94-2.04 (m, 1H), 1.79-1.89 (m, 1H), 1.41 (s, 9H). LCMS: RT=0.813 min, m/z 553.1 [M+H]⁺.

### Step 6: tert-Butyl (4S)-5-amino-4-[4-benzyloxy-6-(2-methoxyethoxy)-1-oxo-isoindolin-2-yl]-5-oxo-pentanoate

To a mixture of tert-butyl (4S)-5-amino-4-[[2-benzyloxy-6-bromo-4-(2-methoxyethoxy)-phenyl]methylamino]-5-oxo-pentanoate (1 g, 1.81 mmol) and Et₃N (727.00 mg, 7.18 mmol, 1 mL) in MeOH (20 mL) was added Pd(dppf)Cl₂ (265 mg, 362.17 μmol). The mixture was degassed and purged with CO three times, and then stirred at 80° C. for 24 hours under CO at 50 psi. LCMS showed the desired mass and the starting material was consumed completely. The mixture was concentrated to dryness. The residue was purified by column chromatography (SiO₂, Petroleum ether:Ethyl acetate=1/0 to 1/1) to afford tert-butyl (4S)-5-amino-4-[4-benzyloxy-6-(2-methoxyethoxy)-1-oxo-isoindolin-2-yl]-5-oxo-pentanoate (840 mg, 1.68 mmol, 92.91% yield) as a yellow gum. HNMR: CDCl₃, 400 MHz. δ 7.34-7.49 (m, 5H), 6.94 (d, J=2.0 Hz, 1H), 6.78 (d, J=2.0 Hz, 1H), 6.31 (br s, 1H), 5.37 (br s, 1H), 5.11 (s, 2H), 4.85-4.94 (m, 1H), 4.33-4.47 (m, 2H), 4.16-4.20 (m, 2H), 3.75-3.82 (m, 2H), 3.48 (s, 3H), 2.13-2.45 (m, 4H), 1.44 (s, 9H). LCMS: RT=0.927 min, m/z 499.1 [M+H]⁺.

### Step 7: tert-Butyl (4S)-5-amino-4-[4-hydroxy-6-(2-methoxyethoxy)-1-oxo-isoindolin-2-yl]-5-oxo-pentanoate

To a mixture of Pd/C (100 mg, 10% purity) in THE (30 mL) was added tert-butyl (4S)-5-amino-4-[4-benzyloxy-6-(2-methoxyethoxy)-1-oxo-isoindolin-2-yl]-5-oxo-pentanoate (840 mg, 1.68 mmol). The mixture was degassed and purged with H₂ three times, and then stirred at room temperature (20° C.) for 12 hours under H₂ at 15 psi. LCMS showed the desired mass and the starting material was consumed completely. The mixture was filtered and the filtrate was concentrated to give tert-butyl (4S)-5-amino-4-[4-hydroxy-6-(2-methoxyethoxy)-1-oxo-iso-indolin-2-yl]-5-oxo-pentanoate (600 mg, 1.47 mmol, 87.19% yield) as a white solid. LCMS: RT=0.796 min, m/z 409.2 [M+H]⁺.

### Step 8: tert-Butyl (4S)-5-amino-4-[4-[(4-formylphenyl)methoxy]-6-(2-methoxyethoxy)-1-oxo-isoindolin-2-yl]-5-oxo-pentanoate

To a mixture of tert-butyl (4S)-5-amino-4-[4-hydroxy-6-(2-methoxyethoxy)-1-oxo-isoindolin-2-yl]-5-oxo-pentanoate (500 mg, 1.22 mmol) and K₂CO₃ (338.38 mg, 2.45 mmol) in DMF (10 mL) was added 4-(bromomethyl)benzaldehyde (316.76 mg, 1.59 mmol). The mixture was then stirred at room temperature (20° C.) for 3 hours. LCMS showed the desired mass and the starting material was consumed completely. The reaction mixture was quenched with water (50 mL), extracted with Ethyl acetate (20 mL×3). The combined organic phases were dried over anhydrous sodium sulfate, filtered and concentrated to dryness. The residue was purified by column chromatography (SiO₂, Petroleum ether:Ethyl acetate=10:1 to 0:1) to give tert-butyl (4S)-5-amino-4-[4-[(4-formylphenyl)methoxy]-6-(2-methoxyethoxy)-1-oxo-isoindolin-2-yl]-5-oxo-pentanoate (380 mg, 721.64 μmol, 58.95% yield) as a white solid. HNMR: CDCl₃, 400 MHz. δ 9.97 (s, 1H), 7.86 (d, J=8.0 Hz, 1H), 7.50 (d, J=8.0 Hz, 1H), 6.86 (d, J=1.6 Hz, 1H), 6.64 (d, J=2.0 Hz, 1H), 6.28 (br s, 1H), 5.41 (br s, 1H), 5.11 (s, 2H), 4.78-4.87 (m, 1H), 4.27-4.45 (m, 2H), 4.05-4.11 (m, 2H), 3.65-3.72 (m, 2H), 3.38 (s, 3H), 2.01-2.37 (m, 4H), 1.35 (s, 9H). LCMS: RT=0.887 min, m/z 527.3 [M+H]⁺.

### Step 9: tert-Butyl (4S)-5-amino-4-[6-(2-methoxyethoxy)-4-[[4-(morpholinomethyl)-phenyl]methoxy]-1-oxo-isoindolin-2-yl]-5-oxo-pentanoate

A mixture of tert-butyl (4S)-5-amino-4-[4-[(4-formylphenyl)methoxy]-6-(2-methoxyethoxy)-1-oxo-isoindolin-2-yl]-5-oxo-pentanoate (350 mg, 664.67 μmol) and morpholine (116 mg, 1.33 mmol, 117.17 μL) in DCM (5 mL) was stirred at room temperature (20° C.) for 2 hours. NaBH(OAc)₃ (423 mg, 2.00 mmol) was added and the mixture was then stirred at room temperature (20° C.) for 2 hours. TLC (Ethyl acetate:Petroleum ether=2:1) showed the starting material was consumed completely and one new spot formed. The reaction mixture was quenched with water (50 mL), extracted with Ethyl acetate (20 mL×3). The combined organic phases were dried over anhydrous sodium sulfate, filtered and concentrated to give a residue. The residue was purified by column chromatography (SiO₂, Petroleum ether:Ethyl acetate=10/1 to 0/1) to afford tert-butyl (4S)-5-amino-4-[6-(2-methoxyethoxy)-4-[[4-(morpholinomethyl)phenyl]methoxy]-1-oxo-isoindolin-2-yl]-5-oxo-pentanoate (350 mg, 585.58 μmol, 88.10% yield) as a white solid. Used without further purification.

### Step 10: (3S)-3-[6-(2-Methoxyethoxy)-4-[[4-(morpholinomethyl)phenyl]methoxy]-1-oxo-isoindolin-2-yl]piperidine-2,6-dione

A mixture of tert-butyl (4S)-5-amino-4-[6-(2-methoxyethoxy)-4-[[4-(morpholinomethyl)-phenyl]methoxy]-1-oxo-isoindolin-2-yl]-5-oxo-pentanoate (150 mg, 250.96 μmol) and TsOH (108 mg, 627.17 μmol) in MeCN (3 mL) was stirred at 55° C. for 12 hours. LCMS showed the desired mass and the starting material was consumed completely. The mixture was cooled to room temperature and poured into saturated NaHCO₃aqueous (10 mL), extracted with Ethyl acetate (5 mL×3). The combined organic phases were dried over Na₂SO₄, filtered, and concentrated to dryness. The residue was purified by prep-HPLC (column: Phenomenex luna C18 150*25 mm*10 μm; mobile phase: [water (0.225% formic acid)-MeCN]; B %: 4%-34%, 10 min) to give (3S)-3-[6-(2-methoxyethoxy)-4-[[4-(morpholinomethyl)phenyl]methoxy]-1-oxo-isoindolin-2-yl]piperidine-2,6-dione (43.8 mg, 83.57 μmol, 33.30% yield, 99.9% purity) as a white solid. HNMR: DMSO-d₆, 400 MHz. δ 10.96 (s, 1H), 7.38-7.46 (m, 2H), 7.29-7.36 (m, 2H), 6.87-6.92 (m, 1H) 6.81-6.86 (m, 1H), 5.21 (s, 2H), 5.03-5.13 (m, 1H), 4.27-4.38 (m, 1H), 4.10-4.22 (m, 3H), 3.63-3.71 (m, 2H), 3.52-3.60 (m, 4H), 3.32 (s, 3H), 2.83-2.97 (m, 1H), 2.53-2.61 (m, 1H), 2.40-2.47 (m, 1H), 2.30-2.37 (m, 4H), 1.91-2.03 (m, 1H). LCMS: RT=2.206 min, m/z 524.3 [M+H]⁺. SFC: ee %=91.918%.

### Example 22: Synthesis of (3S)-3-[4-[[3-(2-methoxyethylamino)-4-(morpholinomethyl)-phenyl]methoxy]-1-oxo-isoindolin-2-yl]piperidine-2,6-dione

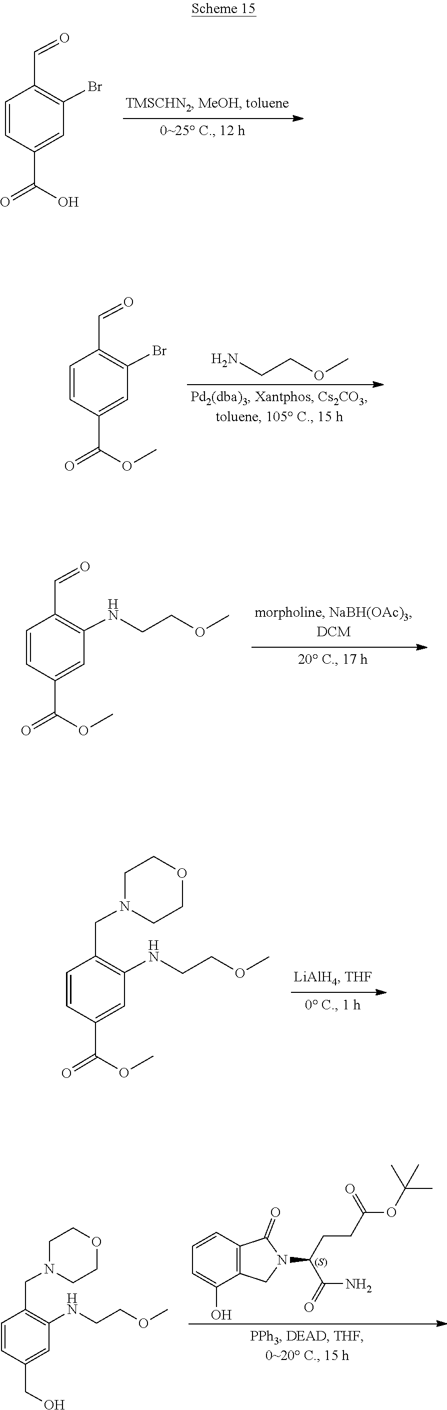

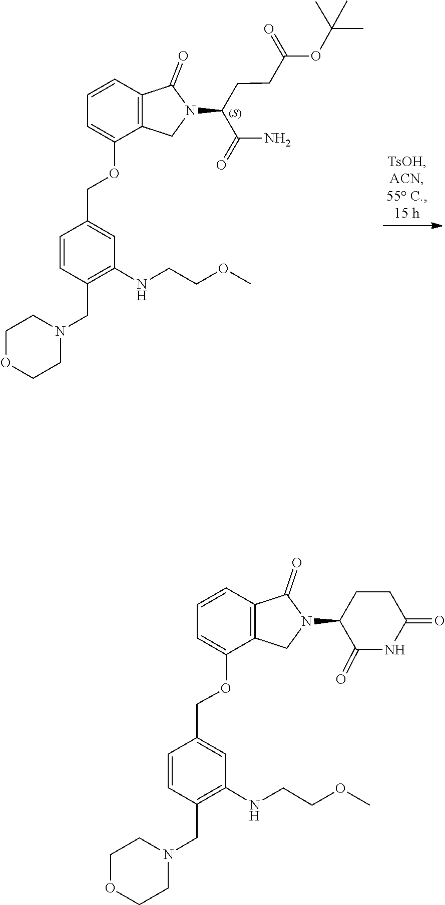

### Step 1: Methyl 3-Bromo-4-formyl-benzoate

To a solution of 3-bromo-4-formyl-benzoic acid (1 g, 4.37 mmol) in MeOH (25 mL) and toluene (25 mL) was added TMSCHN₂ (2 M, 4.37 mL) at 0° C. Then the reaction mixture was stirred at 25° C. for 12 hours. TLC (Petroleum ether:EtOAc=3:1) showed one new spot and the starting material was consumed. The reaction mixture was concentrated under reduced pressure. The residue was purified by flash silica gel column chromatography (EtOAc/Petroleum ether: 0-5%) to give methyl 3-bromo-4-formyl-benzoate (1.4 g, crude) as a white solid. HNMR: CDCl₃, 400 MHz. δ 10.34 (s, 1H), 8.25 (s, 1H), 7.99 (d, J=8.0 Hz, 1H), 7.89 (d, J=8.0 Hz, 1H), 3.90 (s, 3H).

### Step 2: Methyl 4-formyl-3-(2-methoxyethylamino)benzoate

The mixture of methyl 3-bromo-4-formyl-benzoate (1.26 g, 5.18 mmol), 2-methoxyethanamine (1.17 g, 15.55 mmol, 1.35 mL), Pd₂(dba)₃ (474.71 mg, 518.40 μmol), Xantphos (599.92 mg, 1.04 mmol) and Cs₂CO₃ (5.07 g, 15.55 mmol) in toluene (20 mL) was degassed and purged with N2 three times. Then the mixture was stirred at 105° C. for 15 hours. TLC (Petroleum ether:EtOAc=2:1) showed the starting material was consumed completely and new spots formed. The mixture was added into water (100 mL). To the mixture was added HCl aqueous (1 M) to adjust pH=3-4. Then the mixture was extracted with EtOAc (100 mL). The organic phase was washed with brine (100 mL×2), dried over anhydrous sodium sulfate, filtered and concentrated in vacuo to give the crude product. The residue was purified by flash silica gel chromatography (ISCO®; 20 g SepaFlash® Silica Flash Column, Eluent of 0-15% EtOAc/Petroleum ether gradient @ 50 mL/min) to give methyl 4-formyl-3-(2-methoxyethyl-amino)benzoate (265 mg, 1.12 mmol, 21.52% yield, 99.9% purity) as a yellow solid. HNMR: CDCl₃, 400 MHz. δ 9.92 (s, 1H), 8.56-8.42 (m, 1H), 7.56 (d, J=8.4 Hz, 1H), 7.40 (s, 1H), 7.34-7.29 (m, 1H), 3.94 (s, 3H), 3.69-3.65 (m, 2H), 3.55-3.48 (m, 2H), 3.44 (s, 3H). LCMS: RT=0.839 min, m/z 238.2 [M+H]⁺.

### Step 3: Methyl 3-(2-methoxyethylamino)-4-(morpholinomethyl)benzoate

The mixture of methyl 4-formyl-3-(2-methoxyethylamino)-benzoate (265 mg, 1.12 mmol) and morpholine (194.62 mg, 2.23 mmol, 196.58 μL) in DCM (5 mL) was stirred at 20° C. for 2 hours. To the mixture was added NaBH(OAc)₃ (710.19 mg, 3.35 mmol). The mixture was stirred at 20° C. for 15 hours. TLC (Petroleum ether:EtOAc=3:1) showed the starting material aldehyde was consumed and one major new spot formed. The mixture was added into saturated NaHCO₃aqueous (20 mL) and extracted with DCM (15 mL). The organic phase was washed with brine (20 mL×2), dried over anhydrous sodium sulfate, filtered and concentrated in vacuo to give the crude product. The residue was purified by flash silica gel chromatography (Biotage®; pH=8.0, 12 g SepaFlash® Silica Flash Column, Eluent of 0˜9% EtOAc/Petroleum ether gradient @ 30 mL/min) to give methyl 3-(2-methoxyethylamino)-4-(morpholinomethyl)benzoate (274 mg, 887.65 μmol, 79.47% yield, 99.9% purity) as a yellow oil. HNMR: CDCl₃, 400 MHz. δ 7.31 (dd, J=1.2, 7.6 Hz, 1H), 7.26 (s, 1H), 7.05 (d, J=7.6 Hz, 1H), 6.78-6.70 (m, 1H), 3.90 (s, 3H), 3.74-3.65 (m, 6H), 3.55 (s, 2H), 3.43 (s, 3H), 3.38-3.31 (m, 2H), 2.47-2.36 (m, 4H). LCMS: RT=0.833 min, m/z 309.1 [M+H]⁺.

### Step 4: [3-(2-Methoxyethylamino)-4-(morpholinomethyl)phenyl]methanol

To a solution of methyl 3-(2-methoxyethylamino)-4-(morpholinomethyl)benzoate (254 mg, 823.68 μmol) in THE (3 mL) was added LiAlH₄ (62.52 mg, 1.65 mmol) at 0° C. The mixture was stirred at 0° C. under N2 for 1 hour. TLC (DCM:MeOH=20:1) showed the starting material consumed and a new spot was observed. To the mixture was added water (0.06 mL), 15% NaOH aqueous (0.06 mL), water (0.06 mL). The mixture was dried over anhydrous sodium sulfate, filtered and washed with EtOAc (10 mL). The filtrate was concentrated in vacuo. The residue was purified by flash silica gel chromatography (ISCO®; pH=8, 12 g SepaFlash® Silica Flash Column, Eluent of 0-4% MeOH/DCM gradient @ 40 mL/min) to give 3-(2-methoxyethylamino)-4-(morpholinomethyl)phenyl]methanol (total 223 mg) as a yellow oil. HNMR: CDCl₃, 400 MHz. δ 6.98 (d, J=7.2 Hz, 1H), 6.71-6.59 (m, 3H), 4.64 (s, 2H), 3.82-3.63 (m, 7H), 3.51 (s, 2H), 3.42 (s, 3H), 3.35-3.27 (m, 2H), 2.48-2.36 (m, 4H).

### Step 5 tert-Butyl (4S)-5-amino-4-[4-[[3-(2-methoxyethylamino)-4-(morpholino-methyl)phenyl]methoxy]-1-oxo-isoindolin-2-yl]-5-oxo-pentanoate

To a mixture of [3-(2-methoxyethylamino)-4-(morpholinomethyl)phenyl]methanol (200 mg, 713.36 μmol) and tert-butyl (4S)-5-amino-4-(4-hydroxy-1-oxo-isoindolin-2-yl)-5-oxo-pentanoate (238.52 mg, 713.36 μmol) in THE (5 mL) were added PPh₃ (280.66 mg, 1.07 mmol) and DEAD (186.35 mg, 1.07 mmol, 194.52 μL) at 0° C. The mixture was stirred at 20° C. under N₂ for 15 hours. LCMS showed the desired mass. The mixture was concentrated in vacuo. The residue was purified by prep-HPLC (column: Unisil 3-100 C18 Ultra 150*50 mm*3 μm; mobile phase: [water(0.225% formic acid)-MeCN]; B %: 15%-45%, 10 min) to give tert-butyl (4S)-5-amino-4-[4-[[3-(2-methoxyethylamino)-4-(morpholinomethyl)phenyl]methoxy]-1-oxo-isoindolin-2-yl]-5-oxo-pentanoate (total 362 mg, 96.0% purity) as an off-white solid. HNMR: CDCl₃, 400 MHz. δ 7.46-7.39 (m, 2H), 7.10 (d, J=6.8 Hz, 1H), 7.05-6.98 (m, 1H), 6.80-6.57 (m, 3H), 6.42-6.30 (m, 1H), 5.38-5.32 (m, 1H), 5.08 (s, 2H), 4.93-4.87 (m, 1H), 4.53-4.38 (m, 2H), 3.74-3.62 (m, 6H), 3.53 (s, 2H), 3.42 (s, 3H), 3.30 (t, J=4.8 Hz, 2H), 2.47-2.17 (m, 8H), 1.43 (s, 9H). LCMS: RT=0.819 min, m/z 597.5 [M+H]⁺.

### Step 6: (3S)-3-[4-[[3-(2-Methoxyethylamino)-4-(morpholinomethyl)phenyl]methoxy]-1-oxo-isoindolin-2-yl]piperidine-2,6-dione

To a solution of tert-butyl (4S)-5-amino-4-[4-[[3-(2-methoxyethylamino)-4-(morpholino-methyl)phenyl]methoxy]-1-oxo-isoindolin-2-yl]-5-oxo-pentanoate (150 mg, 251.38 μmol) in MeCN (2 mL) was added TsOH (108.22 mg, 628.44 μmol) under N₂. The mixture was stirred at 55° C. under N2 for 15 hours. LCMS showed the desired mass. The mixture was added into 5% NaHCO₃aqueous (20 mL) and extracted with DCM (20 mL). The organic phase was washed with brine (20 mL×2), dried over anhydrous sodium sulfate, filtered and concentrated in vacuo. The residue was purified by prep-HPLC (column: Phenomenex luna C18 150*25 mm*10 μm; mobile phase: [water(0.225% formic acid)-MeCN]; B %: 4%-34%, 10 min) to give the desired product (3S)-3-[4-[[3-(2-methoxyethylamino)-4-(morpholinomethyl)phenyl]methoxy]-1-oxo-isoindolin-2-yl]piperidine-2,6-dione (36.94 mg, 70.62 μmol, 28.09% yield, 99.9% purity) as a white solid. HNMR: DMSO-d₆, 400 MHz. δ 10.98 (s, 1H), 7.51-7.45 (m, 1H), 7.32 (d, J=7.6 Hz, 2H), 6.98 (d, J=7.6 Hz, 1H), 6.70-6.64 (m, 2H), 6.59-6.52 (m, 1H), 5.17-5.06 (m, 3H), 4.45-4.21 (m, 2H), 3.60-3.54 (m, 6H), 3.43 (s, 2H), 3.31 (s, 3H), 3.23-3.18 (m, 2H), 2.97-2.87 (m, 1H), 2.62-2.55 (m, 1H), 2.48-2.42 (m, 1H), 2.35-2.25 (m, 4H), 2.03-1.94 (m, 1H). LCMS: RT=2.270 min, m/z 523.3 [M+H]⁺. SFC: ee %=96.564%.

### Example 23: Synthesis of (S)-3-(4-((2-((2-methoxyethyl)amino)-4-(morpholinomethyl)-benzyl)oxy)-1-oxoisoindolin-2-yl)piperidine-2,6-dione

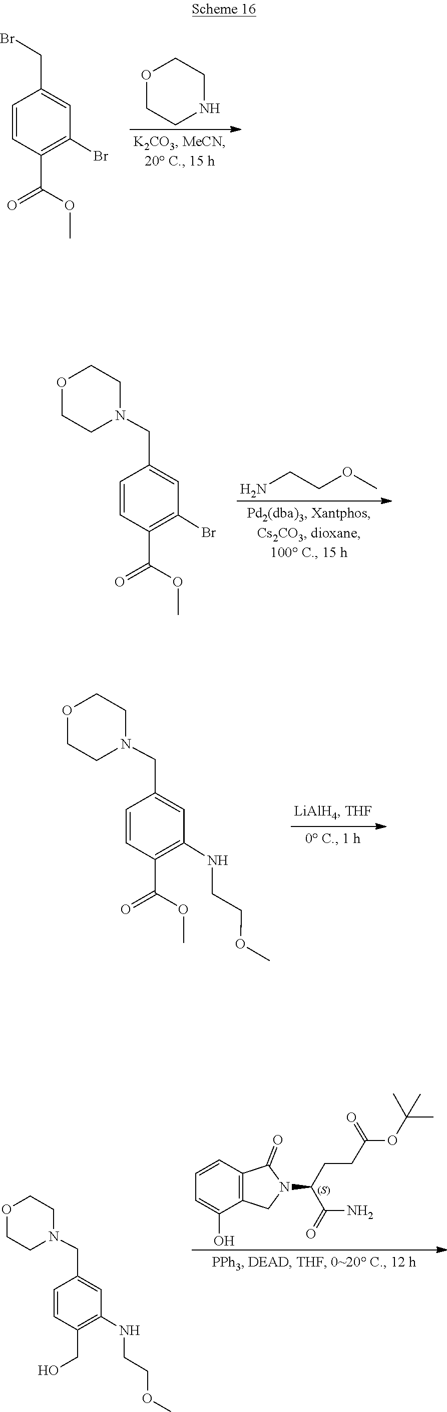

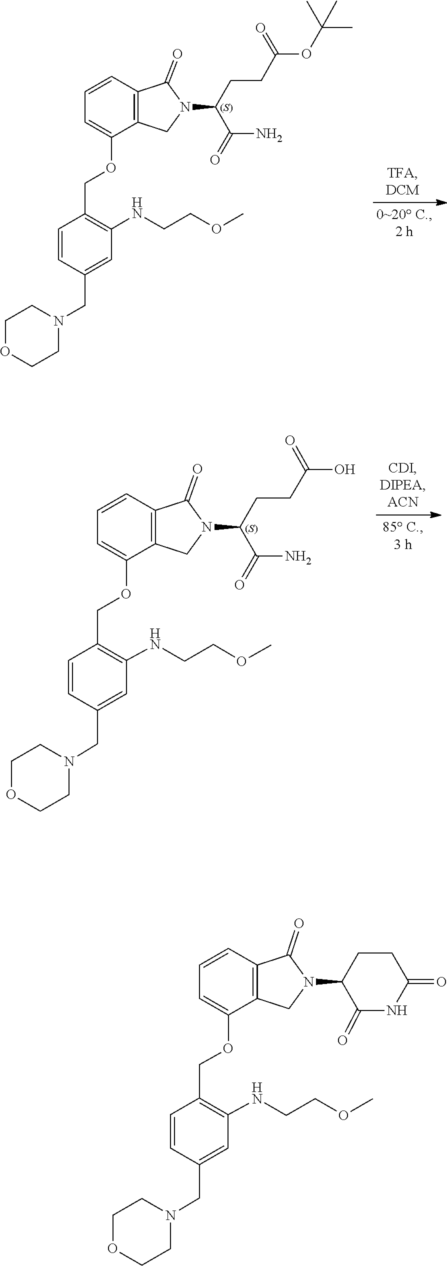

### Step 1: Methyl 2-bromo-4-(morpholinomethyl)benzoate

A mixture of methyl 2-bromo-4-(bromomethyl)benzoate (4 g, 12.99 mmol), morpholine (1.36 g, 15.59 mmol, 1.37 mL) and K₂CO₃ (3.59 g, 25.98 mmol) in MeCN (40 mL) was stirred at 20° C. for 15 hours. TLC (Petroleum ether:Ethyl acetate=2:1) showed new spots. The mixture was added into water (250 mL) and extracted with Ethyl acetate (250 mL). The organic phase was washed with brine (250 mL×2), dried over anhydrous sodium sulfate, filtered and concentrated in vacuo. The crude was purified by flash silica gel chromatography (Biotage®; 25 g SepaFlash® Silica Flash Column, Eluent of 0-27% Ethyl acetate/Petroleum ether gradient @ 40 mL/min) to give methyl 2-bromo-4-(morpholinomethyl)benzoate (1.8 g, 5.71 mmol, 43.93% yield, 99.6% purity) as a yellow oil and the impure methyl 2-bromo-4-(morpholinomethyl)benzoate (1.3 g, 3.00 mmol, 23.13% yield, 72.6% purity) as a yellow oil. HNMR: CDCl₃, 400 MHz. δ 7.76 (d, J=8.0 Hz, 1H), 7.67 (s, 1H), 7.34 (d, J=7.6 Hz, 1H), 3.93 (s, 3H), 3.77-3.69 (m, 4H), 3.50 (s, 2H), 2.50-2.42 (m, 4H). LCMS: RT=0.639 min, m z 314.0 [M+H]⁺.

### Step 2: Methyl 2-(2-methoxyethylamino)-4-(morpholinomethyl)benzoate

To a mixture of methyl 2-bromo-4-(morpholinomethyl)benzoate (1.95 g, 6.21 mmol), 2-methoxyethanamine (699.28 mg, 9.31 mmol, 809.35 μL), Xantphos (718.27 mg, 1.24 mmol) and Cs₂CO₃ (6.07 g, 18.62 mmol) in dioxane (20 mL) was added Pd₂(dba)₃ (568.36 mg, 620.67 μmol) under N₂. The mixture was stirred at 100° C. under N2 for 15 hours. LCMS showed the desired mass. The mixture was added into water (250 mL) and extracted with Ethyl acetate (250 mL). The organic phase was washed with brine (250 mL×2), dried over anhydrous sodium sulfate, filtered and concentrated in vacuo. The crude was purified by flash silica gel chromatography (Biotage®; pH=8.0, 25 g SepaFlash® Silica Flash Column, Eluent of 0-30% Ethyl acetate/Petroleum ether gradient @ 50 mL/min) to afford methyl 2-(2-methoxyethylamino)-4-(morpholinomethyl)-benzoate (1.74 g, 5.41 mmol, 87.09% yield, 95.8% purity) as a yellow oil. HNMR: CDCl₃, 400 MHz. δ 7.90-7.80 (m, 2H), 6.69 (s, 1H), 6.62-6.56 (m, 1H), 3.85 (s, 3H), 3.72 (t, J=4.4 Hz, 4H), 3.66 (t, J=5.6 Hz, 2H), 3.49-3.35 (m, 7H), 2.48-2.42 (m, 4H). LCMS: RT=0.478 min, m/z 309.3 [M+H]⁺.

### Step 3: [2-(2-Methoxyethylamino)-4-(morpholinomethyl)phenyl]methanol

To a solution of methyl 2-(2-methoxyethylamino)-4-(morpholinomethyl)benzoate (500 mg, 1.62 mmol) in THE (5 mL) was added LiAlH₄ (123.08 mg, 3.24 mmol) at 0° C. under N₂. The mixture was stirred at 0° C. under N2 for 1 hour. TLC (Petroleum ether:Ethyl acetate=1:1) showed the reaction was completed. To the mixture was added water (0.12 mL), 15% NaOH aqueous solution (0.12 mL), water (0.36 mL). The mixture was dried over anhydrous Na₂SO₄, filtered and washed with EtOAc (20 mL). The filtrate was concentrated in vacuo. The crude material was purified by flash silica gel chromatography (Biotage®; pH=8.0, 24 g SepaFlash® Silica Flash Column, eluent of 0˜5% MeOH/DCM gradient @ 40 mL/min) to give [2-(2-methoxyethylamino)-4-(morpholinomethyl)phenyl]methanol (92.2% purity) (total 482 mg) as a yellow oil. HNMR: CDCl₃, 400 MHz. δ 7.02 (d, J=7.6 Hz, 1H), 6.70 (s, 1H), 6.66-6.61 (m, 1H), 5.06-4.86 (m, 1H), 4.66 (s, 2H), 3.74-3.69 (m, 4H), 3.66 (t, J=5.2 Hz, 2H), 3.47 (s, 2H), 3.41 (s, 3H), 3.36 (t, J=5.6 Hz, 2H), 2.48-2.43 (m, 4H). LCMS: RT=0.399 min, m/z 281.3 [M+H]⁺.

### Step 4: [2-(2-Methoxyethylamino)-4-(morpholinomethyl)phenyl]methanol

To a mixture of [2-(2-methoxyethylamino)-4-(morpholinomethyl)phenyl]methanol compound (460 mg, 1.64 mmol), tert-butyl (4S)-5-amino-4-(4-hydroxy-1-oxo-isoindolin-2-yl)-5-oxo-pentanoate compound (548.61 mg, 1.64 mmol) and PPh₃ (645.51 mg, 2.46 mmol) in THE (10 mL) was added DEAD (428.61 mg, 2.46 mmol, 447.41 μL) at 0° C. under N₂. Then the mixture was stirred at 20° C. under N2 for 12 hours. LCMS showed the desired mass of the product. The mixture was concentrated in vacuo. The crude was purified by prep-HPLC (column: Phenomenex luna C18 150*40 mm*15 μm; mobile phase: [water(0.225% FORMIC ACID)-MeCN]; B %: 10%-40%, 10 min) to give tert-butyl (4S)-5-amino-4-[4-[[2-(2-methoxyethylamino)-4-(morpholino-methyl)phenyl]methoxy]-1-oxo-isoindolin-2-yl]-5-oxo-pentanoate (250 mg, 387.96 μmol, 23.65% yield, 92.6% purity) as a yellow solid. HNMR: CDCl₃, 400 MHz. δ 7.48-7.42 (m, 2H), 7.21-7.13 (m, 2H), 6.76-6.68 (m, 2H), 6.31 (s, 1H), 5.33-5.27 (m, 1H), 5.13 (s, 2H), 4.88 (dd, J=6.4, 8.8 Hz, 1H), 4.79 (s, 1H), 4.48-4.37 (m, 2H), 3.80-3.70 (m, 4H), 3.62 (t, J=5.2 Hz, 2H), 3.48 (d, J=2.4 Hz, 2H), 3.36-3.27 (m, 5H), 2.52-2.10 (m, 8H), 1.42 (s, 9H). LCMS: RT=0.811 min, m/z 597.5 [M+H]⁺.

### Step 5: (4S)-5-Amino-4-[4-[[2-(2-methoxyethylamino)-4-(morpholinomethyl)-phenyl]methoxy]-1-oxo-isoindolin-2-yl]-5-oxo-pentanoic acid

To a solution of tert-butyl (4S)-5-amino-4-[4-[[2-(2-methoxyethylamino)-4-(morpholino-methyl)phenyl]methoxy]-1-oxo-isoindolin-2-yl]-5-oxo-pentanoate (190 mg, 318.41 μmol) in DCM (2 mL) was added TFA (1 mL) at 0° C. The mixture was stirred at 20° C. for 2 hours. LCMS showed the desired mass of the product. The mixture was concentrated in vacuo to give the desired product (4S)-5-amino-4-[4-[[2-(2-methoxyethylamino)-4-(morpholinomethyl)-phenyl]methoxy]-1-oxo-isoindolin-2-yl]-5-oxo-pentanoic acid (172 mg, 318.16 μmol, 99.92% yield) as a yellow oil, which was used in next step without further purification. LCMS: RT=0.728 min, m/z 541.4 [M+H]⁺.

### Step 6: (S)-3-(4-((2-((2-Methoxyethyl)amino)-4-(morpholinomethyl)benzyl)oxy)-1-oxoisoindolin-2-yl)piperidine-2,6-dione

To a solution of (4S)-5-amino-4-[4-[[2-(2-methoxyethylamino)-4-(morpholinomethyl)phenyl]-methoxy]-1-oxo-isoindolin-2-yl]-5-oxo-pentanoic acid (170 mg, 314.46 μmol) in MeCN (5 mL) were added CDI (101.98 mg, 628.92 μmol) and DIPEA (81.28 mg, 628.92 μmol, 109.55 μL). The mixture was stirred at 85° C. for 3 hours. LCMS showed the desired mass. The mixture was concentrated in vacuo. The crude was purified by prep-HPLC (column: Phenomenex Luna C18 150*25 mm*10 μm; mobile phase: [water(0.225% FORMIC ACID)-MeCN]; B %: 6%-36%, 10 min) to give (S)-3-[4-[[2-(2-methoxyethylamino)-4-(morpholinomethyl)phenyl]methoxy]-1-oxo-isoindolin-2-yl]-piperidine-2,6-dione 6 (87.60 mg, 156.23 μmol, 49.68% yield, 93.2% purity) as an off-white solid. HNMR: DMSO-d₆, 400 MHz. δ 10.97 (s, 1H), 7.53-7.47 (m, 1H), 7.39 (d, J=8.0 Hz, 1H), 7.33 (d, J=7.2 Hz, 1H), 7.24 (d, J=7.6 Hz, 1H), 6.65 (s, 1H), 6.59 (d, J=7.6 Hz, 1H), 5.19-5.05 (m, 4H), 4.40-4.20 (m, 2H), 3.62-3.57 (m, 4H), 3.54-3.48 (m, 4H), 3.26-3.23 (m, 2H), 3.21 (s, 3H), 2.96-2.87 (m, 1H), 2.62-2.54 (m, 1H), 2.47-2.31 (m, 5H), 2.03-1.93 (m, 1H). LCMS: RT=2.225 min, m/z 523.3 [M+H]⁺.

### Example 24: Synthesis of (S)-5-(((2-(2,6-dioxopiperidin-3-yl)-1-oxoisoindolin-4-yl)oxy)methyl)-N-(2-methoxyethyl)-2-(morpholinomethyl)benzamide

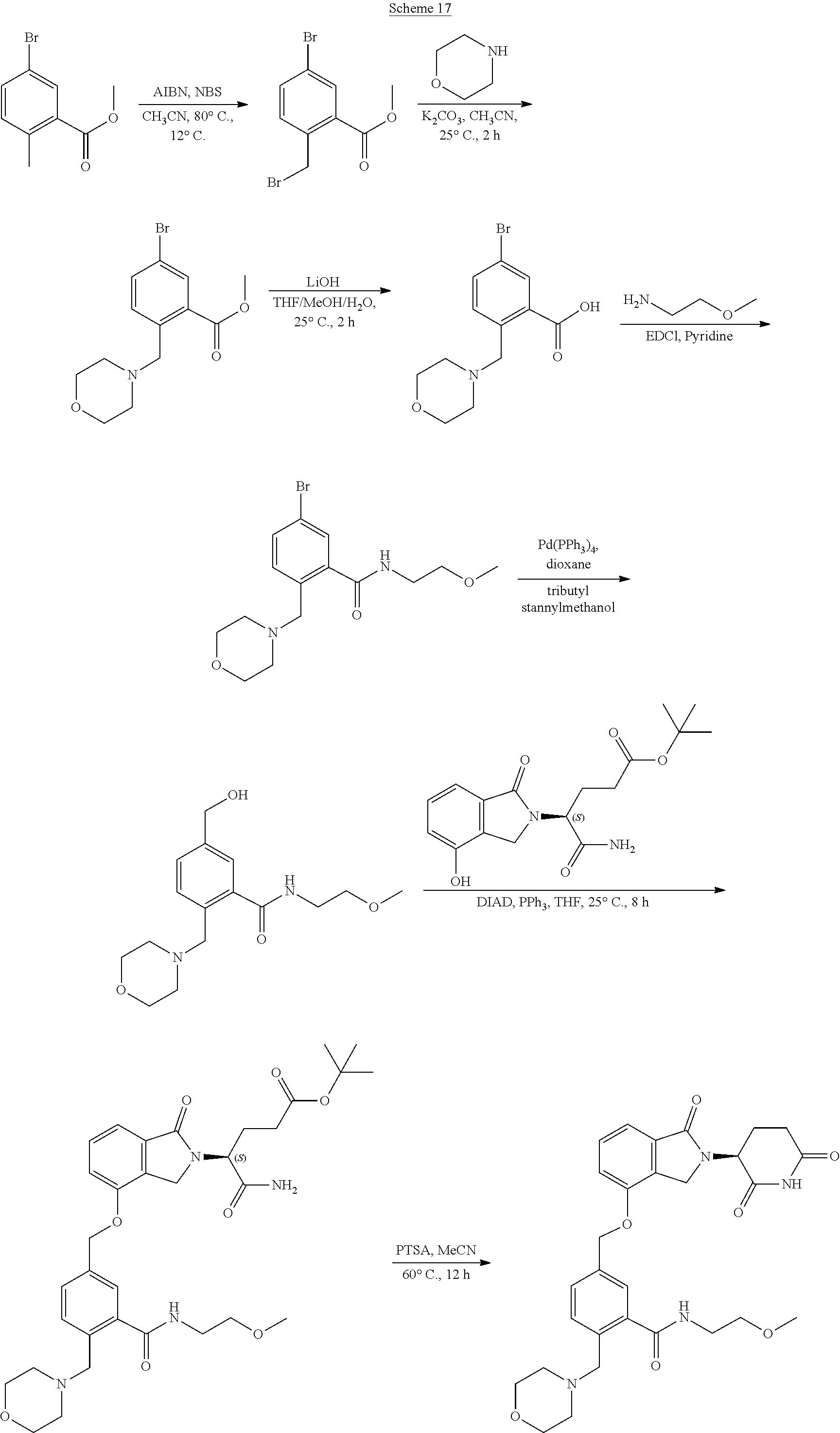

### Step 1: Methyl 5-bromo-2-(bromomethyl)benzoate

To a solution of methyl 5-bromo-2-methylbenzoate (5.3 g, 23.14 mmol) in CH₃CN (50 mL) were added NBS (6.18 g, 34.71 mmol) and AIBN (759.86 mg, 4.63 mmol). The mixture was stirred at 80° C. for 12 hours. TLC (Petroleum ether:Ethyl Acetate=10:1) indicated part of starting material was remained, and one major new spot (Rf=0.65) with larger polarity was detected. The reaction mixture was concentrated under reduced pressure to remove CH₃CN. The residue was diluted with water (80 mL) and extracted with Ethyl Acetate (10 mL×3). The combined organic layers were washed with saturated brine (60 mL), dried over anhydrous sodium sulfate, filtered, and concentrated under reduced pressure. The residue was purified by prep-HPLC (column: Kromasil Eternity XT 250*80 mm*10 μm; mobile phase: [water(0.225% FORMIC ACID)-MeCN]; B %: 58%-78%, 20 min). Then the collected fraction was concentrated to remove most of the acetonitrile and then extracted with Ethyl Acetate (30 mL×3). The combined organic phase was washed with brine (100 mL), dried with anhydrous sodium sulfate, filtered, and concentrated under vacuum to give the desired product methyl 5-bromo-2-(bromomethyl)-benzoate (2.5 g, 8.12 mmol, 35.09% yield) as a colorless oil. HNMR: CDCl₃, 400 MHz. δ 8.12 (d, J=2.0 Hz, 1H), 7.63 (dd, J=2.0, 8.0 Hz, 1H), 7.35 (d, J=8.0 Hz, 1H), 4.91 (s, 2H), 3.96 (s, 3H).

### Step 2: Methyl 5-bromo-2-(morpholinomethyl)benzoate

To a solution of methyl 5-bromo-2-(bromomethyl)benzoate (2.5 g, 8.12 mmol) in CH₃CN (25 mL) was added morpholine (848.66 mg, 9.74 mmol, 857.24 μL) and K₂CO₃ (2.24 g, 16.24 mmol). The mixture was stirred at 25° C. for 2 hours. LCMS showed the desired mass. The reaction mixture was concentrated under reduced pressure to remove CH₃CN. Then the mixture was diluted with water (100 mL) and extracted with Ethyl Acetate (30 mL×3). The combined organic layers were washed with saturated brine (60 mL), dried over anhydrous sodium sulfate, filtered, and concentrated under reduced pressure. The residue was purified by flash silica gel chromatography (20 g SepaFlash® Silica Flash Column, Eluent of 0-11% Ethyl acetate/Petroleum ether gradient @ 80 mL/min) to give methyl 5-bromo-2-(morpholinomethyl)benzoate (2.22 g, 6.97 mmol, 85.90% yield, 98.68% purity) as a colorless oil. HNMR: CDCl₃, 400 MHz. δ 7.85 (d, J=1.6 Hz, 1H), 7.55 (d, J=8.0 Hz, 1H), 7.34 (d, J=8.4 Hz, 1H), 3.90 (s, 3H), 3.71 (s, 2H), 3.68-3.64 (m, 4H), 2.44-2.40 (m, 4H). LCMS: RT=0.432 min, m/z 315.8 [M+H]⁺.

### Step 3: 5-Bromo-2-(morpholinomethyl)benzoic acid

To a solution of methyl 5-bromo-2-(morpholinomethyl)benzoate (2 g, 6.37 mmol) in H₂O (5 mL), MeOH (5 mL) and THE (5 mL) was added LiOH (457.36 mg, 19.10 mmol), the mixture was stirred at 25° C. for 2 hours. LCMS showed the desired mass. The reaction mixture was concentrated under reduced pressure to give the desired product 5-bromo-2-(morpholinomethyl)benzoic acid (1.91 g, 6.36 mmol, quantitative) as a white solid. LCMS: RT=0.671 min, m/z 301.7 [M+H]⁺.

### Step 4: 5-Bromo-N-(2-methoxyethyl)-2-(morpholinomethyl)benzamide

To a solution of 5-bromo-2-(morpholinomethyl)benzoic acid (3.3 g, 7.96 mmol, 72.41% purity) and 2-methoxyethanamine (896.97 mg, 11.94 mmol, 1.04 mL) in Pyridine (20 mL) was added EDCI (2.29 g, 11.94 mmol). The mixture was stirred at 25° C. for 8 hours. LCMS showed the desired mass. The reaction mixture was diluted with water (150 mL) and extracted with Ethyl Acetate (30 mL×3). The combined organic layers were washed with saturated brine (80 mL), dried over anhydrous sodium sulfate, filtered, and concentrated under reduced pressure. The residue was purified by flash silica gel chromatography (40 g SepaFlash® Silica Flash Column, Eluent of 0-40% Ethyl acetate/Petroleum ether gradient @ 80 mL/min) to give the desired product 5-bromo-N-(2-methoxyethyl)-2-(morpholinomethyl)benzamide (1.86 g, 5.05 mmol, 63.46% yield, 97.01% purity) as a yellow oil. LCMS: RT=0.556 min, m/z 356.9 [M+H]⁺.

### Step 5: 5-(Hydroxymethyl)-N-(2-methoxyethyl)-2-(morpholinomethyl)benzamide

To a solution of 5-bromo-N-(2-methoxyethyl)-2-(morpholinomethyl)benzamide (0.5 g, 1.40 mmol) in dioxane (2 mL) was added Pd(PPh₃)₄ (161.73 mg, 139.96 μmol) and tributylstannylmethanol (1.35 g, 4.20 mmol) and the mixture was stirred at 110° C. for 12 hours. LCMS showed the desired mass. The reaction mixture was quenched by addition of saturated KF aqueous (3 mL) at 25° C., and then diluted with water (50 mL) and extracted with Ethyl Acetate (3 mL×3). The combined organic layers were washed with saturated brine (50 mL), dried over anhydrous sodium sulfate, filtered, and concentrated under reduced pressure. The residue was purified by prep-HPLC (column: Phenomenex Synergi Max-RP 250*50 mm*10 μm; mobile phase: [water(0.225% formic acid)-MeCN]; B %: 10%-40%, 35 min). Then the collected fraction was concentrated to remove most of acetonitrile and then the mixture was adjusted till pH=7 with saturated NaHCO₃aqueous, and extracted with Ethyl Acetate (30 mL×3). The combined organic phase was washed with saturated brine (100 mL), dried with anhydrous sodium sulfate, filtered and concentrated under vacuum to give the desired product 5-(hydroxymethyl)-N-(2-methoxyethyl)-2-(morpholinomethyl)benzamide (0.261 g, 846.38 μmol, 60.47% yield) as a yellow oil. HNMR: DMSO, 400 MHz. δ 9.60 (s, 1H), 7.59 (d, J=1.6 Hz, 1H), 7.36-7.32 (m, 1H), 7.30-7.25 (m, 1H), 5.25 (t, J=6.0 Hz, 1H), 4.51 (d, J=5.6 Hz, 2H), 3.59-3.55 (m, 4H), 3.53 (s, 2H), 3.48-3.45 (m, 4H), 3.28 (s, 3H), 2.39 (s, 4H). LCMS: RT=0.490 min, m/z 309.0 [M+H]⁺.

### Step 6: (S)-tert-Butyl 5-amino-4-(4-((3-((2-methoxyethyl)carbamoyl)-4-(morpholino-methyl)benzyl)oxy)-1-oxoisoindolin-2-yl)-5-oxopentanoate

To a mixture of 5-(hydroxymethyl)-N-(2-methoxyethyl)-2-(morpholinomethyl)benzamide (0.1 g, 324.28 μmol) and (S)-tert-butyl 5-amino-4-(4-hydroxy-1-oxoisoindolin-2-yl)-5-oxopentanoate (130.12 mg, 389.14 μmol) in THE (0.5 mL) was added DIAD (196.72 mg, 972.85 μmol, 189.15 μL) and PPh₃ (85.06 mg, 324.28 μmol). The mixture was stirred at 25° C. for 8 hours. LCMS showed the desired mass. The mixture was diluted with water (100 mL) and extracted with Ethyl Acetate (20 mL×3). The combined organic layers were washed with saturated brine (80 mL), dried over anhydrous sodium sulfate, filtered, and concentrated under reduced pressure to give a residue. The residue was purified by prep-TLC (SiO₂, Ethyl Acetate 100%) to afford (S)-tert-butyl 5-amino-4-(4-((3-((2-methoxyethyl)carbamoyl)-4-(morpholinomethyl)benzyl)oxy)-1-oxo-isoindolin-2-yl)-5-oxopentanoate (0.133 g, 212.89 μmol, 65.65% yield) as a white solid. LCMS: RT=0.611 min, m/z 625.3 [M+H]⁺.

### Step 7: (S)-5-(((2-(2,6-Dioxopiperidin-3-yl)-1-oxoisoindolin-4-yl)oxy)methyl)-N-(2-methoxyethyl)-2-(morpholinomethyl)benzamide

To a mixture of (S)-tert-butyl 5-amino-4-(4-((3-((2-methoxyethyl)carbamoyl)-4-(morpholino-methyl)benzyl)oxy)-1-oxoisoindolin-2-yl)-5-oxopentanoate (0.133 g, 212.89 μmol) in CH₃CN (2 mL) was added 4-methylbenzenesulfonic acid (109.98 mg, 638.68 μmol). The mixture was stirred at 60° C. for 12 hours. LCMS showed the desired mass. The reaction mixture was diluted with water (60 mL) and adjusted till pH=7 with saturated NaHCO₃aqueous, then extracted with Ethyl Acetate (30 mL×3). The combined organic layers were washed with saturated brine (100 mL), dried over anhydrous sodium sulfate, filtered, and concentrated under reduced pressure. The residue was purified by prep-HPLC (column: Phenomenex Gemini-NX C18 75*30 mm*3 μm; mobile phase: [water(10 mM NH₄HCO₃)-MeCN]; B %: 15%-45%, 8 min). The collected fraction was concentrated to remove most of acetonitrile and the solution was lyophilized to afford (S)-5-(((2-(2,6-dioxopiperidin-3-yl)-1-oxoisoindolin-4-yl)oxy)methyl)-N-(2-methoxyethyl)-2-(morpholinomethyl)benzamide (0.02884 g, 52.38 μmol, 24.60% yield) as a white solid. HNMR: DMSO, 400 MHz. δ 10.98 (m, 1H), 9.66-9.55 (m, 1H), 7.72 (s, 1H), 7.58-7.46 (m, 2H), 7.40-7.31 (m, 3H), 5.28 (s, 2H), 5.11 (dd, J=4.8, 12.8 Hz, 1H), 4.47-4.24 (m, 2H), 3.56 (s, 6H), 3.47 (s, 4H), 3.28 (s, 3H), 2.97-2.85 (m, 1H), 2.59 (d, J=2.0 Hz, 1H), 2.46-2.43 (m, 1H), 2.39 (s, 4H), 2.04-1.96 (m, 1H). LCMS: RT=2.159 min, m/z 511.2 [M+H]⁺.

### Example 25: Evaluation of Ligand Binding Affinity

The binding affinity of the described test compounds was determined using RED-tris-NTA His-tag labeled Cereblon ligand binding domain. The labeling of the Cereblon ligand binding domain was performed according to manufacturer's protocol in assay buffer (50 nM protein: 10 nM dye, 5:1 protein to dye ratio). 200 μM compound in assay buffer (10 mM HEPES pH 7.4, 300 mM NaCl, 0.1% Pluronic-147, 10% Glycerol, 5 mM DTT) was created from 10 mM compound stock in DMSO. A 16-point serial dilution was achieved by taking 10 μL of the 200 μM first well and diluting into 10 μL of assay buffer in the second well, which was diluted in 10 μL assay buffer in the third well, and so on. DMSO content of each well matched to 2%. 10 μL of 50 nM CRBN was added to each well and mixed thoroughly by pipetting. Final assay concentration of 25 nM labeled protein and highest compound concentration of 100 μM, 1% DMSO.

Samples were incubated at room temperature in the dark for five minutes, then collected in standard Monolith capillaries. Assay was performed on the Monolith NT.155Pico using automated excitation level and medium power on M.O. Control software. Analysis of MST traces and determination of dissociation constants and was conducted in M.O. Affinity Analysis software, and the results are shown in Table 2 below.

TABLE 2
List of compounds from Examples 11-25

| Example | | | Plant |
|---|---|---|---|
| # | Structure | Compound Name | CRBN K_{d}^{a} |
| 11 | 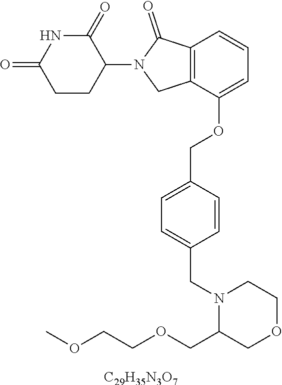 | (3S)-3-(4-((4-((3-((2- methoxyethoxy)methyl) morpholino)methyl)benzyl)oxy)-1- oxoisoindolin-2-yl)piperidine- 2,6-dione | +++ |
| 12 | 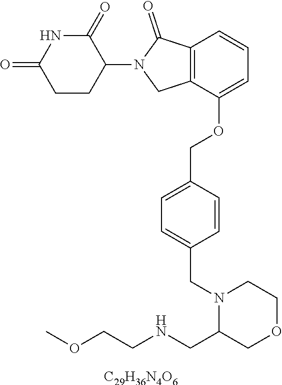 | (3S)-3-(4-((4-((3-(((2- methoxyethyl)amino)methyl) morpholino)methyl)benzyl)oxy)- 1-oxoisoindolin-2- yl)piperidine-2,6-dione | ++ |
| 14 | 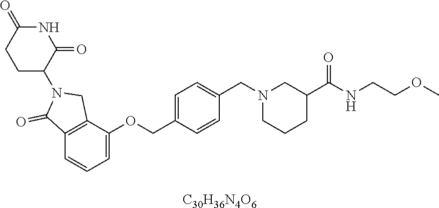 [SMILES: COCCNC(=O)C1CCCN(Cc2ccc(COc3cccc4c3CN(C3CCC(=O)NC3=O)C4=O)cc2)C1 ; "C30H36N4O6"] | 1-(4-(((2-((S)-2,6- dioxopiperidin-3-yl)-1- oxoisoindolin-4- yl)oxy)methyl)benzyl)-N-(2- methoxyethyl)piperidine-3- carboxamide | ++ |
| 16 | 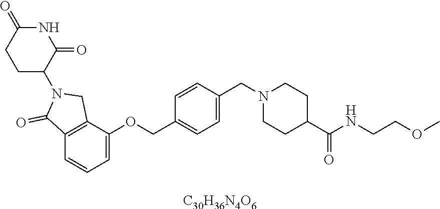 [SMILES: COCCNC(=O)C1CCN(Cc2ccc(COc3cccc4c3CN(C3CCC(=O)NC3=O)C4=O)cc2)CC1 ; "C30H36N4O6"] | (S)-1-(4-(((2-(2,6- dioxopiperidin-3-yl)-1- oxoisoindolin-4- yl)oxy)methyl)benzyl)-N-(2- methoxyethyl)piperidine-4- carboxamide | ++ |
| 18 | 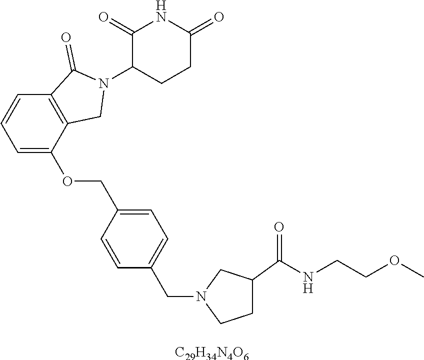 [SMILES: COCCNC(=O)C1CCN(Cc2ccc(COc3cccc4c3CN(C3CCC(=O)NC3=O)C4=O)cc2)C1 ; "C29H34N4O6"] | 1-(4-(((2-((S)-2,6- dioxopiperidin-3-yl)-1- oxoisoindolin-4- yl)oxy)methyl)benzyl)-N-(2- methoxyethyl)pyrrolidine-3- carboxamide | ++ |
| 20 | 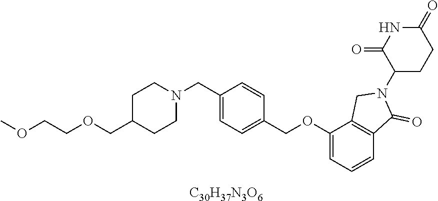 [SMILES: COCCOCC1CCN(Cc2ccc(COc3cccc4c3CN(C3CCC(=O)NC3=O)C4=O)cc2)CC1 ; "C30H37N3O6"] | (S)-3-(4-((4-((4-((2- methoxyethoxy)methyl)piperidin- 1-yl)methyl)benzyl)oxy)-1- oxoisoindolin-2-yl)piperidine- 2,6-dione | ++ |
| 21 | 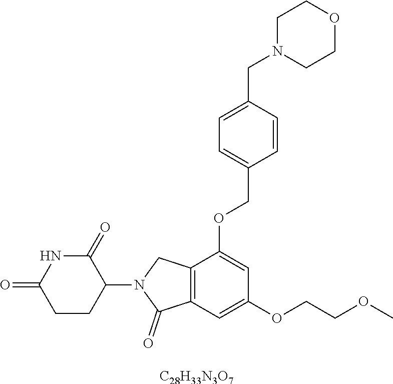 | (S)-3-(6-(2-methoxyethoxy)-4- ((4- (morpholinomethyl)benzyl) oxy)-1-oxoisoindolin-2- yl)piperidine-2,6-dione | +++ |
| 22 | 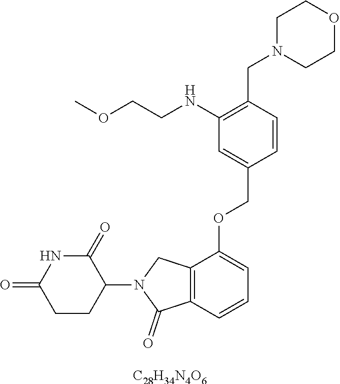 | (S)-3-(4-((3-((2- methoxyethyl)amino)-4- (morpholinomethyl)benzyl) oxy)-1-oxoisoindolin-2- yl)piperidine-2,6-dione | +++ |
| 23 | 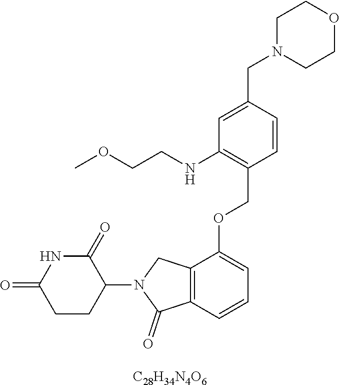 | (S)-3-(4-((2-((2- methoxyethyl)amino)-4- (morpholinomethyl)benzyl) oxy)-1-oxoisoindolin-2- yl)piperidine-2,6-dione | ++ |
| 24 | 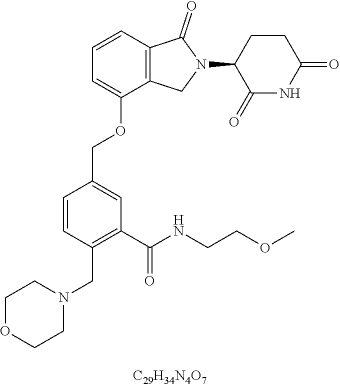 [SMILES: COCCNC(=O)c1cc(COc2cccc3c2CN([C@H]2CCC(=O)NC2=O)C3=O)ccc1CN1CCOCC1 ; "C29H34N4O7"] | (S)-5-(((2-(2,6-dioxopiperidin- 3-yl)-1-oxoisoindolin-4- yl)oxy)methyl)-N-(2- methoxyethyl)-2- (morpholinomethyl)benzamide | ++ |
| Ref 1 | 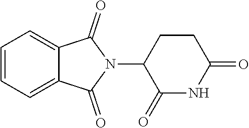 [SMILES: O=C1CCC(N2C(=O)c3ccccc3C2=O)C(=O)N1] | Thalidomide | + |
| Ref 2 | 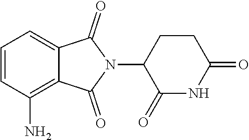 [SMILES: Nc1cccc2c1C(=O)N(C1CCC(=O)NC1=O)C2=O] | Pomalidomide | + |

^{a}+++: K_{d} < 100 nM
++: 100 nM ≤ K_{d} ≤ 1 μM
+: 1 μM < K_{d}
K_{d} is based on binding to plant cereblon

### Example 26: Uptake in Plant Cells

### Protocol:

Abutilon plants were sown in 3 inch pots and allowed to germinate and reach the two leaf stage. At the two leaf stage, plants were separated into groups of 8 plants and had one of their first true leaves labeled with a black marker. Formulation treatment samples were prepared using the following protocol. All treatment samples were prepared to 400 μM. Just before treatment, samples were emulsified and 10 μL of formulated compound was pipetted onto leaves marked for treatment in small droplets evenly across the leaf surface. Once all leaves were treated, they were allowed to sit undisturbed in a growth chamber for 24 hours. After 24 hours plants were removed from the chamber for sample collection.

### Sample Collection:

Using clean scissors treated leaves were removed from the plants, and washed in 20 mL of LCMS grade acetonitrile. A sample of the wash solution was collected and set aside for analysis. The washed leaves were then stored in 7 mL centrifuge tubes and set aside. 8 treated leaves from a grouping were stored in the same sample tube. The untreated leaves were collected and stored in separate 7 mL tubes. The same was done with new growth.

### Sample Preparation:

The samples were stored in a −80° C. freezer for 24 hours before milling. Fresh stock of 80:20 LCMS grade acetonitrile and water was prepared as a milling solution. 3 to 4 ceramic milling beads were added to each tube along with 300 μL of milling solution. Tubes were sealed and put into a benchtop homogenizer. Samples were milled for two 12 second intervals at maximum speed with a 30 second rest period in between. Samples were then moved to a 4° C. centrifuge where they were centrifuged at 15000 rpm for 6 minutes. Supernatant was collected and put into 2 mL microcentrifuge tubes. These samples were then centrifuged for 5 minutes at 12000 rpm and supernatant was collected for use in clean vials.

### Unknown Samples:

50 μL of supernatant was added to 1.5 mL HPLC vials fitted with 250 μL inserts along with 50 μL of LCMS grade water.

### Calibration Samples:

Untreated plant leaf samples were collected and prepared as outlined in the above sections. Calibration samples (0.05 μM-5 μM) were prepared in 1.5 mL HPLC vials fitted with 250 μL inserts.

### LCMS Conditions:

A Shimadzu MS 2020 and Nexeria-i LC-2040C was employed using a Mobile Phase A (LCMS grade water+0.10% LCMS grade formic acid), a Mobile Phase B (LCMS acetonitrile+0.10% LCMS grade formic acid), and a Waters XSelect® HSS T3 XP Column, 100 Å, 2.5 μm, 2.1 mm×50 mm in conjunction with a Thermo Fisher Javelin™ Guard Column. Mobile Phase B was ramped from 10% to 65% over 1 minute, from 65% to 95% over 3 subsequent minutes, and held at 95% for 1 subsequent minute. The mass spectrometer was set to scan from m/z 200 to 2000 in positive ion mode and also single ion mode for [M+H], also in positive ion mode, for the specific compound of interest.

Samples were run with injections of 10 μL. Data was analyzed and calibration curves were built for known samples, using area under the curve for [M+H]. Unknown concentrations were calculated with the calibration curves to determine the amount of PROTAC® compound present at 24 hours in treated leaves and in the leaf washings. Results are shown below and demonstrate that Compound 4-B was effectively taken up by leaves.

| | Average | Standard |
|---|---|---|
| Treatment | Concentration μM | Deviation |
| Compound 4-B Treated | 0.491 | 0.271 |

| Treatment | Average Concentration μM | Standard Deviation |
|---|---|---|
| Compound 4-B Wash | 0.341620004 | 0.091196601 |

### Example 27: Protoplast Assay for Screening PROTAC® Compounds

PROTAC® compound mediated degradation of target proteins in plant protoplasts is evaluated by expressing a tagged version of the target protein and treating with PROTAC®. Exogenous target proteins are easily adapted to this strategy by expressing the protein as a fusion to the HiBit tag, which allows for the luminescence signal to be easily measured over time. Arabidopsis protoplasts are isolated from leaves of 3-4 weeks old seedlings according to Yoo S-D., Cho Y—H. & Sheen J., Nature Protocol, 2007 (2) 7: 1565-1573. Plasmids containing the HiBit-target protein and a co-expressed firefly luciferase are transfected into protoplasts using standard PEG-mediated transformation techniques. Transfected protoplasts are plated at approximately ˜5000 cells/well in a 384-well plate and incubated at 25° C. overnight. Then PROTAC® compounds are added at varying concentrations (0-20 μM). The protoplasts are incubated for an additional 24-hours at 25° C. The target protein is measured using the Nano-Glo© Hibit DLR System.

### Example 28: Endogenous Target Degradation Assays

### PROTAC® Compound Application on Arabidopsis Protoplasts.

PROTAC® compound mediated degradation of target proteins in plant protoplasts is assessed by quantitative western blot. Arabidopsis protoplasts are isolated from leaves of 3-4 weeks old seedlings according to Yoo S-D., Cho Y—H. & Sheen J., Nature Protocol, 2007 (2) 7: 1565-1573. Protoplasts are plated at approximately 250,000 cells/well in a 24-well plate. Then PROTAC® compounds are added at varying concentrations (0-20 μM). The protoplasts are incubated for an 18-24-hours at 25° C. in dark. Proteins (˜15 μg) were separated by SDS-PAGE and transferred to PVDF membrane using Invitrogen iBlot 2 Western Blot Transfer system. These membranes are probed with primary antibodies normally used at a dilution of 1:200-1:5000 (4° C. 12-16 h) whereas secondary antibodies are routinely used at a dilution of 1:10000 (22° C. 2-3 h). Proteins are normalized to total protein or housekeeping protein.

### Global Proteomic Protoplast Degradation Assays

PROTAC® compound mediated protein degradation and off-target effects in plant protoplasts is evaluated by global proteomics. Arabidopsis protoplasts are treated and prepared as above. Protoplasts are lysed and digested with trypsin at 37° C. overnight, extracted and dried prior to solubilization and subjected to liquid chromatography with tandem mass spectrometry (LC-MS/MS) set for data independent acquisition (DIA). The resulting MS/MS spectra from each LC-MS/MS run were searched against the TAIR10 database using Spectronaut to investigate PROTAC® compound dependent changes to the proteome.

### Example 29: Plant Phenotyping Assay

Seeds of Matricaria recutita, Diplotaxis tenuifolia, Agrostis spp., Poa annua, or Arabidopsis thaliana were sown into 96-well plates containing 200 μl of growth media [0.5× Murashige and Skoog (MS) basal media (Sigma-Aldrich), 2.5 mM 2-(N-morpholino) ethanesulfonic acid (MES—Sigma-Aldrich), 0.7 g/L Phytagar (Invitrogen); pH 5.7]. Three to five seeds were distributed per well. Plates were placed under fifteen hours of light at 23° C., 65% humidity in a controlled environment growth chamber. After approximately three days, test compounds and controls were mixed with acetone and emulsified with an equal volume of 3% methyl ester of rapeseed oil (Mero), and applied to plants at concentrations of 238, 475, 950, and 1900 g ai/ha (50-400 uM). After seven days plates were imaged, and plants were visually scored for herbicidal phenotypes, where “+++” is highly active, “++” is moderately active, “+” is weakly active, and “−” is inactive.

TABLE 3
Plant activity data.

| Compound | Plant Activity | Compound | Plant Activity |
|---|---|---|---|
| 3 | ++ | 4-B | +++ |
| 8-C | + | 4-A | +++ |
| 4-F | +++ | 4-D | +++ |
| 4-H | +++ | 4-O | − |
| 4-G | ++ | 4-J | − |
| 4-E | ++ | 4-K | + |
| 1-A | + | 4-C | +++ |
| 1 | ++ | 4-I | + |
| 7-C | − | 4-M | + |
| 7 | + | 4-N | − |
| 7-B | + | 5-D | + |
| 7-A | − | 5 | + |
| 8 | + | 5-C | + |
| 8-B | + | 2-A | + |
| 8-A | + | 5-B | + |
| 4-S | ++ | 6 | + |
| 5-A | − | 6-C | − |
| 4 | + | 2-B | + |
| 4-R | − | 2 | + |
| 4-Q | ++ | 6-B | + |
| 4-P | + | 6-A | + |
| 4-L | − | | |

### Example 30: Nanobody Assays

Transient Expression Assay by Agroinfiltration of Nicotiana benthamiana Leaves.

GFP is expressed as a fusion protein with a Halotag® protein tag (Promega) under an estradiol inducible promoter. The Halotag® protein tag is used to make the protein bigger. There are several reports showing that GFP alone is very stable and hardly degraded via the proteasome. This may be prevented by using GFP fusion proteins or less stable GFP versions.

An overnight (ON) culture of Agrobacterium tumefaciens (strain: GV3101 pMIP90) was grown in LB medium at 28° C. with appropriate selection (Rif 100, Gent 30, Spec 100), carrying E3-Nanobody fusion, Nanobody only, GFP-target and the silencing suppressor P19 (Danielson & Pezacki (2013) FEBS Letters 587(8): 1198-1205). Typically, a 20 ml culture reached approx. OD₆₀₀ of 4.

The cultures were adjusted to OD₆₀₀ of 1.5 for nanobody constructs and GFP and OD₆₀₀ of 1 for the P19 suppressor. Equal volumes were mixed as follows: Test: E3-Nanobody, GFP-target, P19. Control: Nanobody, GFP target, P19. 10 ml of cell mixture was used to infiltrate at least 5 leaves. The Agrobacteria mixtures were spun at 4000 rpm, 10 min and resuspended in original volume using H2O. They were spun down again and the cell pellet was resuspended in 10 mM MgCl₂ and 200 μM Acetosyringone.

Nicotiana benthamiana leaves were infiltrated from the lower site of the leaves through the stomata using 1 mL Tuberculin Syringes: One half of the leaf with the negative control (Nanobody, GFP, P19), the other with E3 ligase construct (E3-Nanobody, GFT, P19). The same leaf was used for the test sample as well as the control. 2-3 days after infiltration, expression of the target protein was induced with 25 μM β-estradiol in water with 0,01% Silwet. The plants were dried before moving to the next step.

### GFP Measurements in Plate Reader.

Measurements were taken on day 3 (one night after the induction or right after the induction). 6 leaf disks were collected (using a punch hole with a 7 mm hole) for each sample. Samples avoided the infiltration site, since these cells were dead, and also avoided big leaf veins. The leaf discs were distributed with the bottom of the leaf facing upwards in a black 96 well plate, that was filled with H₂O (200 μl per well. GFP signal was measured using a fluorescence reader that has a filter that blocks chlorophyll autofluorescence. The GFP fluorescence was measured over several hours using the same plate/leaf disks.

Protein Extraction from Tobacco Leaves Followed by Western Blot.

Expression of the E3/Nanobody was measured by HA-Antibody. GFP degradation was measured via detection with a GFP antibody. For each sample, a 2 ml Safe Lock tube with 4-6 glass beads (about 3 mm diameter) was prepared. A punch hole (7 mm hole) was used to collect 3 leaf disks for each sample. The plant material was transferred to the prepared tubes and frozen in liquid nitrogen or stored at −80° C. A Qiagen TissueLyser with the 2×24 adapter was used to disrupt the tissue. The tubes were placed in a precooled (at −80° C.) adapters and shaken for 1 min at max frequency. 1× Loading dye was immediately added to each tube. The samples were then boiled for 5-10 min @ 95° C., then spun down at maximum speed for 5 min.

10 μl supernatant was loaded on an SDS-PAGE gel (NuPAGE™ 4-12% Bis-Tris Gel; ThermoFisher NPO323BOX with NuPAGE MOPS SDS Running Buffer, ThermoFisher NP0001) together with a size ladder (PageRuler, ThermoFisher #26616). The gel was run using the iBlot® 2 Dry Blotting system with nitrocellulose transfer stacks (ThermoFisher IB23002 or IB23001). Ponceau staining was performed to assess even transfer by incubating for about 1 min in Ponceau S solution (Sigma, P7170-1L), washing off excess staining with distilled water, and scanning the blot. The membrane was blocked with 5% nonfat dry milk powder in 1×TBST (Blotting-Grade Blocker, Bio-Rad #1706404) for 1 hr at room temperature or overnight at 4° C. The sample was incubated with primary antibody in 5% nonfat dry milk powder in 1×TBST for 2-4 hr at room temperature or overnight at 4° C. Following incubation, the membrane was washed 3× for min 5 minutes with TBST and incubated with a secondary antibody in 1×TBST for 1 h @ room temperature. The membrane was then washed 3 times for 5 minutes with TBST. This was followed by the addition of 4 ml ECL solution, and chemiluminescence was detected with a suitable detection device.

Data from the nanobody experiments indicated that GFP degradation was induced by E3 ligases fused to GFP-nanobody. Several ligase binding moieties, including ones for cereblon, were used to direct the nanobody to ligases for degradation demonstrating that proteins in plants may be directed to ligases for degradation.

It is recognized that those of ordinary skill in the art may make modifications and variations in the embodiments described herein without departing from the spirit or scope of the present disclosure. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Where applicable or not specifically disclaimed, any one of the embodiments described herein are contemplated to be able to combine with any other one or more embodiments, even though the embodiments are described, under different aspects of the invention. As such, the preceding general areas of utility are given by way of example only and are not intended to be limiting on the scope of the present disclosure and appended claims. Additional objects and advantages associated, with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention.

1. A method for controlling the level of a target protein in a plant cell, the method comprising contacting the plant cell with an effective amount of a compound according to Formula I:
PTM-L-LTM (I),
or a salt or hydrate thereof, wherein:
PTM is a targeting moiety that binds the target protein;
L is a covalent bond or linker moiety; and
LTM is a ubiquitin ligase binding moiety that binds a plant ubiquitin ligase.

2. The method of claim 1, wherein controlling the level of the target protein comprises targeted degradation of the target protein.

3. The method of claim 1, wherein the target protein is hydroxyphenylpyruvate dioxygenase, acetolactate synthase, or acetyl CoA carboxylase.

4. The method of claim 1, wherein the plant ubiquitin ligase is a plant cereblon.

5. The method of claim 1, wherein PTM is:
a halo-(2,6-dioxocyclohexane-carbonyl)phenyl moiety,
an N-(5,8-dimethoxy-[1,2,4]triazolo[1,5-c]pyrimidin-2-yl)-6-(trifluoromethyl)benz-2-yl-sulfonamide moiety,
a 1-oxo-2-(1-(ethoxyimino)propyl)-3-hydroxy-cyclohex-2-en-5-yl moiety,
a 6-((4-oxy)phenoxy)pyridin-3-yl moiety, or
a 4-(oxy)phenoxy)acetyl moiety,
each of which is optionally substituted with one or more substituents that are each independently C₁₋₆ alkyl, halo, hydroxy, amino, C₁₋₆ alkylamino, C₁₋₆ amido, C₁₋₆ acyl, nitro, cyano, or C₁₋₆ alkoxy.

6. (canceled)

7. The method of claim 1, wherein LTM is:
(a) an N-substituted 1,3-dioxoisoindolinyl moiety, which is optionally substituted with one or more substituents that are each independently C₁₋₆ alkyl, halo, hydroxy, amino, C₁₋₆ alkylamino, C₁₋₆ amido, C₁₋₆ acyl, nitro, cyano, or C₁₋₆ alkoxy; or
(b) a moiety of Formula II: 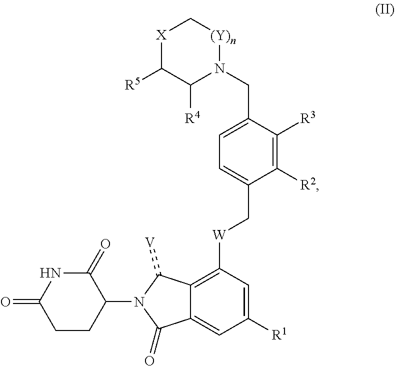
wherein:
subscript n is an integer ranging from 0 to 3;
V is absent or (O);
R¹ is H or —Z—R^{1a};
W, X, Y, and Z are each independently O, NR⁶, or CHR⁷, provided that Y is CHR⁷ when subscript n is 0 or 1;
R^{1a} is -L-PTM, H, or —(CH₂—CH₂—O)ₘ—CH₃;
R² and R³ are each independently -L-PTM, H, —NH—(CH₂—CH₂—O)ₘ—CH₃, or —CONH—(CH₂—CH₂—O)ₘ—CH₃;
R⁴ is -L-PTM, H, —CH₂—O—(CH₂—CH₂—O)ₘ—CH₃, —CH₂NH—(CH₂—CH₂—O)ₘ—CH₃, or —CONH—(CH₂—CH₂—O)ₘ—CH₃;
R⁵ and R⁷ are each independently -L-PTM, H, or —CONH—(CH₂—CH₂—O)ₘ—CH₃;
R⁶ is -L-PTM, H, C₁₋₆ acyl, or —CH₂—O—(CH₂—CH₂—O)ₘ—CH₃, provided that R⁶ is other than H when X is N;
each subscript m is independently an integer ranging from 1 to 10;
each L is an independently-selected linker moiety;
each PTM is an independently-selected protein targeting moiety; and
at least one of R¹-R⁷ is other than H.

8. (canceled)

9. A compound according to Formula I:
PTM-L-LTM (I),
or a salt or hydrate thereof, wherein:
PTM is a targeting moiety that binds with a protein in a plant cell;
L is a covalent bond or linker moiety; and
LTM is a ubiquitin ligase binding moiety that binds a plant ubiquitin ligase.

10-11. (canceled)

12. The compound of claim 9, wherein LTM is:
(a) an N-substituted 1,3-dioxoisoindolinyl moiety, which is optionally substituted with one or more substituents that are each independently C₁₋₆ alkyl, halo, hydroxy, amino, C₁₋₆ alkylamino, C₁₋₆ amido, C₁₋₆ acyl, nitro, cyano, or C₁₋₆ alkoxy; or
(b) a moiety of Formula II: 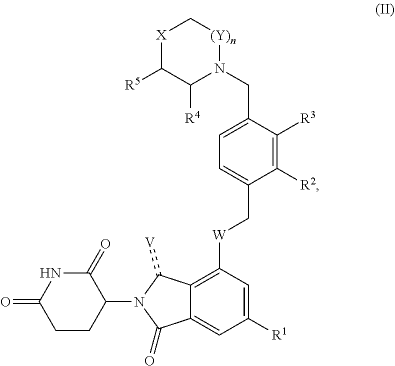
wherein:
subscript n is an integer ranging from 0 to 3;
V is absent or (O);
R¹ is H or —Z—R^{1a};
W, X, Y, and Z are each independently O, NR⁶, or CHR⁷, provided that Y is CHR⁷ when subscript n is 0 or 1;
R^{1a} is -L-PTM, H, or —(CH₂—CH₂—O)ₘ—CH₃;
R² and R³ are each independently -L-PTM, H, —NH—(CH₂—CH₂—O)ₘ—CH₃, or —CONH—(CH₂—CH₂—O)ₘ—CH₃;
R⁴ is -L-PTM, H, —CH₂—O—(CH₂—CH₂—O)ₘ—CH₃, —CH₂NH—(CH₂—CH₂—O)ₘ—CH₃, or —CONH—(CH₂—CH₂—O)ₘ—CH₃;
R⁵ and R⁷ are each independently -L-PTM, H, or —CONH—(CH₂—CH₂—O)ₘ—CH₃;
R⁶ is -L-PTM, H, C₁₋₆ acyl, or —CH₂—O—(CH₂—CH₂—O)ₘ—CH₃, provided that R⁶ is other than H when X is N;
each subscript m is independently an integer ranging from 1 to 10;
each L is an independently-selected linker moiety;
each PTM is an independently-selected protein targeting moiety; and
at least one of R¹-R⁷ is other than H.

13-16. (canceled)

17. The compound of claim 12, or a salt or hydrate thereof, wherein one and only one of R^{1a} and R²-R⁷ is -L-PTM.

18. The compound of claim 12, or a salt or hydrate thereof, wherein:
V is absent;
W is O;
R² and R³ are H;
R³ is -L-PTM, —NH—(CH₂—CH₂—O)ₘ—CH₃, or —CONH—(CH₂—CH₂—O)ₘ—CH₃, and R² is H;
R² is -L-PTM, —NH—(CH₂—CH₂—O)ₘ—CH₃, or —CONH—(CH₂—CH₂—O)ₘ—CH₃, and R³ is H; or each subscript m is independently 1 or 2.

19-24. (canceled)

25. The compound of claim 12, or a salt or hydrate thereof, wherein X is O, subscript n is 1, Y is —CHR⁷, and R⁷ is H.

26. The compound of claim 25, or a salt or hydrate thereof, wherein:
R⁵ is H; or
R⁴ is -L-PTM, —CH₂—O—(CH₂—CH₂—O)ₘ—CH₃, —CH₂NH—(CH₂—CH₂—O)ₘ—CH₃, or —CONH—(CH₂—CH₂—O)ₘ—CH.

27. (canceled)

28. The compound of claim 12, or a salt or hydrate thereof, wherein X is —CHR⁷, subscript n is 0 or 1, Y is —CHR⁷, and R⁷ is H.

29. The compound of claim 28, or a salt or hydrate thereof, wherein:
R⁴ is H; or
R⁵ is -L-PTM, or —CONH—(CH₂—CH₂—O)ₘ—CH₃.

30. (canceled)

31. (canceled)

32. The compound of claim 9, or a salt or hydrate thereof, wherein the protein targeting moiety targets a plant protein.

33. The compound of claim 32, or a salt or hydrate thereof, wherein the plant protein is a hydroxyphenylpyruvate dioxygenase, an acetolactate synthase, or an acetyl CoA carboxylase.

34. The compound of claim 12, or a salt or hydrate thereof, wherein the PTM is:
a halo-(2,6-dioxocyclohexane-carbonyl)phenyl moiety,
an N-(5,8-dimethoxy-[1,2,4]triazolo[1,5-c]pyrimidin-2-yl)-6-(trifluoromethyl)benz-2-yl-sulfonamide moiety,
a 1-oxo-2-(1-(ethoxyimino)propyl)-3-hydroxy-cyclohex-2-en-5-yl moiety,
a 6-((4-oxy)phenoxy)pyridin-3-yl moiety, or
a 4-(oxy)phenoxy)acetyl moiety,
each of which is optionally substituted with one or more substituents that are each independently C₁₋₆ alkyl, halo, hydroxy, amino, C₁₋₆ alkylamino, C₁₋₆ amido, C₁₋₆ acyl, nitro, cyano, or C₁₋₆ alkoxy.

35. (canceled)

36. A composition comprising the compound of claim 9, or a salt or a salt or hydrate thereof, and an agriculturally acceptable carrier.

37-40. (canceled)

41. A plant cell comprising the compound of claim 9, or a salt or hydrate thereof.

42. The plant cell of claim 41, wherein the compound is present in the plant cell in an amount sufficient to cause degradation of a protein of interest.
